(19) United States
(12) Patent Application Publication
(10) Pub. No.: US 2019/0273210 A1
(43) Pub. Date: Sep. 5, 2019

(54) HETEROAROMATIC COMPOUND AND ORGANIC ELECTROLUMINESCENCE DEVICE USING THE SAME

(71) Applicants: FENG-WEN YEN, TAIPEI (TW); LI-CHIEH CHUANG, NANTOU (TW)

(72) Inventors: FENG-WEN YEN, TAIPEI (TW); LI-CHIEH CHUANG, NANTOU (TW)

(73) Assignee: LUMINESCENCE TECHNOLOGY CORPORATION, HSINCHU (TW)

(21) Appl. No.: 15/908,840

(22) Filed: Mar. 1, 2018

(51) Int. Cl. H01L 51/00; C07D 219/02; C09K 11/06; C07D 495/04

(52) U.S. Cl. CPC H01L 51/0072; C07D 219/02; H01L 51/0055; H01L 51/0058; H01L 51/5028; H01L 51/0054; C07D 495/04; H01L 51/0071; H01L 51/0052; C09K 11/06

(57) ABSTRACT

A heteroaromatic compound which can be used as the fluorescent guest material in the light emitting layer of the organic electroluminescence device is disclosed. The organic electroluminescence device employing the heteroaromatic compound of the present invention can operate under reduced driving voltage, increase current efficiency, and prolong half-life time.

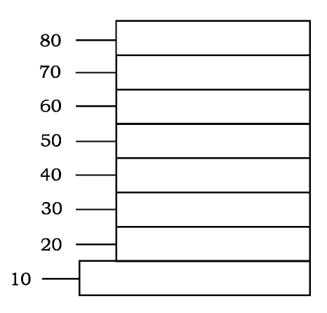

### FIELD OF INVENTION

The present invention relates to a heteroaromatic compound and, more particularly, to an organic electroluminescence device using the heteroaromatic compound.

### BACKGROUND OF THE INVENTION

An organic electroluminescence (organic EL) device is an organic light-emitting diode (OLED) in which the light emitting layer is a film made from organic compounds, which emits light in response to the electric current. The light emitting layer containing the organic compound is sandwiched between two electrodes. The organic EL device is applied to flat panel displays due to its high illumination, low weight, ultra-thin profile, self-illumination without back light, low power consumption, wide viewing angle, high contrast, simple fabrication methods and rapid response time.

Typically, the organic EL device is composed of organic material layers sandwiched between two electrodes. The organic material layers include, e.g., hole injection layer (HIL), hole transporting layer (HTL), emitting layer (EML), electron transporting layer (ETL), and electron injection layer (EIL). The basic mechanism of organic EL involves the injection, transport, and recombination of carriers as well as exciton formation for emitting light. When an external voltage is applied across the organic EL device, electrons and holes are injected from the cathode and the anode, respectively. Electrons will be injected from the cathode into a LUMO (lowest unoccupied molecular orbital) and holes will be injected from the anode into a HOMO (highest occupied molecular orbital). Subsequently, the electrons recombine with holes in the light emitting layer to form excitons, which then deactivate to emit light. When luminescent molecules absorb energy to achieve an excited state, the exciton may either be in a singlet state or a triplet state, depending on how the spins of the electrons and holes have been combined. It is well known that the excitons formed under electrical excitation typically include 25% singlet excitons and 75% triplet excitons. In the fluorescence materials, however, the electrically generated energy in the 75% triplet excitons will be dissipated as heat for decay from the triplet state is spin forbidden. Therefore, a fluorescent electroluminescence device has only 25% internal quantum efficiency, which leads to the theoretically highest external quantum efficiency (EQE) of only 5% due to only ˜20% of the light out-coupling efficiency of the device. In contrast to fluorescent electroluminescence devices, phosphorescent organic EL devices make use of spin-orbit interactions to facilitate intersystem crossing between singlet and triplet states, thus obtaining emission from both singlet and triplet states and the internal quantum efficiency of electroluminescence devices from 25% to 100%.

For full-colored displays using organic EL devices, the organic materials used in the organic EL devices are still unsatisfactory in half-life time, driving voltage, and current efficiency. Therefore, there is still a need for an organic compound that can lower the driving voltage, increase the current efficiency, and prolong the half-life time for the organic EL device.

### SUMMARY OF THE INVENTION

Accordingly, an object of the present invention is to provide a novel heteroaromatic compound, which can be used as a fluorescenct guest material in the emitting layer of the organic EL device to improve the power consumption, current efficiency, and life time of the device.

Another object of the invention is to provide a heteroaromatic compound and an organic EL device using the same, which can operate under reduced voltage and exhibit higher current efficiency and longer half-life time.

According to the present invention, a heteroaromatic compound which can be used in organic EL devices is disclosed. The heteroaromatic compound is represented by the following formula (1):

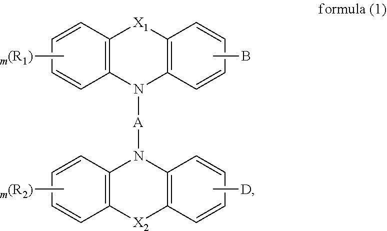

wherein X₁ and X₂ independently represent a divalent bridge selected from the group consisting of O, S, Se, CR₃R₄, NR₅, and SiR₆R₇; A represents a substituted or unsubstituted fused ring hydrocarbon unit with two to nine rings; m is an integer of 0 to 4; B and D independently represent formula (2) below:

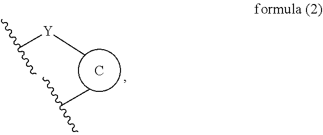

wherein ring C represents a phenyl ring or a fused ring hydrocarbon unit with two to five rings; Y represents a divalent bridge selected from the group consisting of O, S, Se, CR₈R₉, NR₁₀, and SiR₁₁R₁₂; and R₁ to R₁₂ are independently a hydrogen atom, a halide, a substituted or unsubstituted alkyl group having 1 to 60 carbon atoms, a substituted or unsubstituted aryl group having 6 to 60 carbon atoms, a substituted or unsubstituted aralkyl group having 6 to 60 carbon atoms, a substituted or unsubstituted heteroaryl group having 3 to 60 carbon atoms, or a substituted or unsubstituted arylamine group having 6 to 60 carbon atoms.

The present invention further discloses an organic electroluminescence device. The organic electroluminescence device comprises a pair of electrodes composed of a cathode and an anode, and a light emitting layer between the pair of electrodes. The light emitting layer comprises the heteroaromatic compound of formula (1).

### BRIEF DESCRIPTION OF THE DRAWINGS

The FIGURE is a schematic view showing an organic EL device according to an embodiment of the present invention.

### DESCRIPTION OF THE PREFERRED EMBODIMENTS

What probed into the invention is the heteroaromatic compound and organic EL device using the heteroaromatic compound. Detailed descriptions of the production, structure and elements will be provided as follows such that the invention can be fully understood. Obviously, the application of the invention is not confined to specific details familiar to those skilled in the art. On the other hand, the common elements and procedures that are known to everyone are not described in details to avoid unnecessary limits of the invention. Some preferred embodiments of the present invention will now be described in greater detail as follows. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In one embodiment of the present invention, a heteroaromatic compound which can be used as the fluorescent guest material of the light emitting layer in the organic EL device is disclosed. The heteroaromatic compound is represented by the following formula (1):

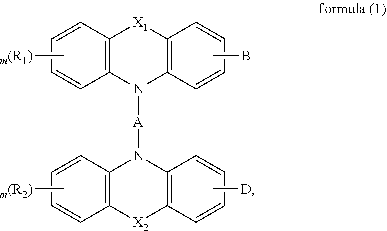

wherein X₁ and X₂ independently represent a divalent bridge selected from the group consisting of O, S, Se, CR₃R₄, NR₅, and SiR₆R₇; A represents a substituted or unsubstituted fused ring hydrocarbon unit with two to nine rings; m is an integer of 0 to 4; B and D independently represent formula (2) below:

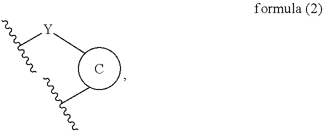

wherein ring C represents a phenyl ring or a fused ring hydrocarbon unit with two to five rings; Y represents a divalent bridge selected from the group consisting of O, S, Se, CR₈R₉, NR₁₀, and SiR₁₁R₁₂; and R₁ to R₁₂ are independently a hydrogen atom, a halide, a substituted or unsubstituted alkyl group having 1 to 60 carbon atoms, a substituted or unsubstituted aryl group having 6 to 60 carbon atoms, a substituted or unsubstituted aralkyl group having 6 to 60 carbon atoms, a substituted or unsubstituted heteroaryl group having 3 to 60 carbon atoms, or a substituted or unsubstituted arylamine group having 6 to 60 carbon atoms.

In some embodiments, A represents a substituted or unsubstituted naphthyl group, a substituted or unsubstituted phenanthrenyl group, a substituted or unsubstituted anthracenyl group, a substituted or unsubstituted pyrenyl group, a substituted or unsubstituted chrysenyl group, a substituted or unsubstituted triphenylenyl group, a substituted or unsubstituted perylenyl group, a substituted or unsubstituted tetraphenylenyl group, or a substituted or unsubstituted 7,14-diphenylacenaphtho[1,2-k]fluoranthene group.

In some embodiments, A represents one of the following formulas:

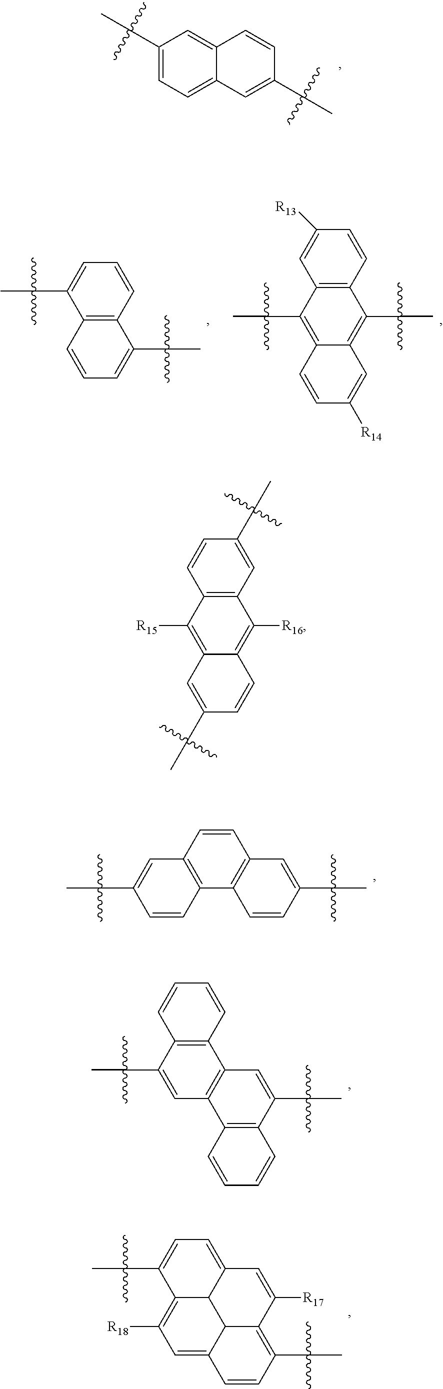

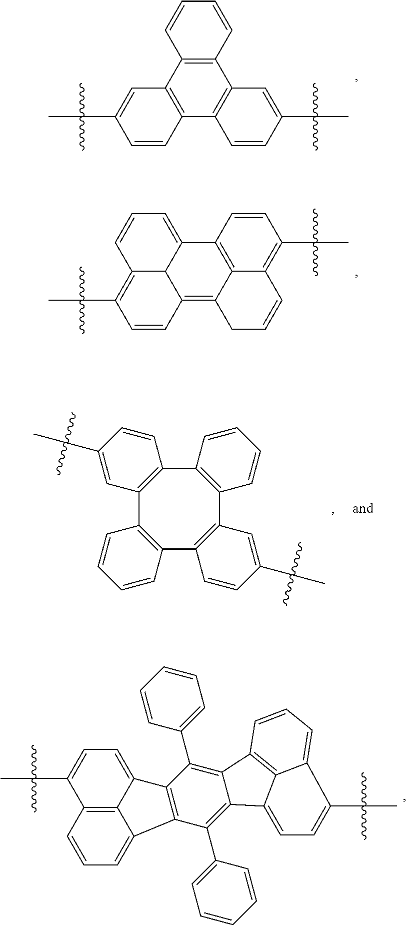 [SMILES: *c1ccc2c3ccc(*)cc3c3ccccc3c2c1 ; *C1=C2C=CC=C3c4ccc(*)c5c4C(=C(C=C1)C32)CC=C5 ; *c1ccc2c(c1)-c1ccccc1-c1ccc(*)cc1-c1ccccc1-2 ; *c1ccc2c3c(-c4ccccc4)c4c5cccc6c(*)ccc(c4c(-c4ccccc4)c3c3cccc1c32)c65]

wherein R₁₃ to R₁₈ are independently a hydrogen atom, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, a substituted or unsubstituted heteroaryl group having 3 to 30 carbon atoms, or a substituted or unsubstituted arylamine group having 6 to 30 carbon atoms.

In some embodiments, the heteroaromatic compound is represented by one of the following formula (3) to formula (13):

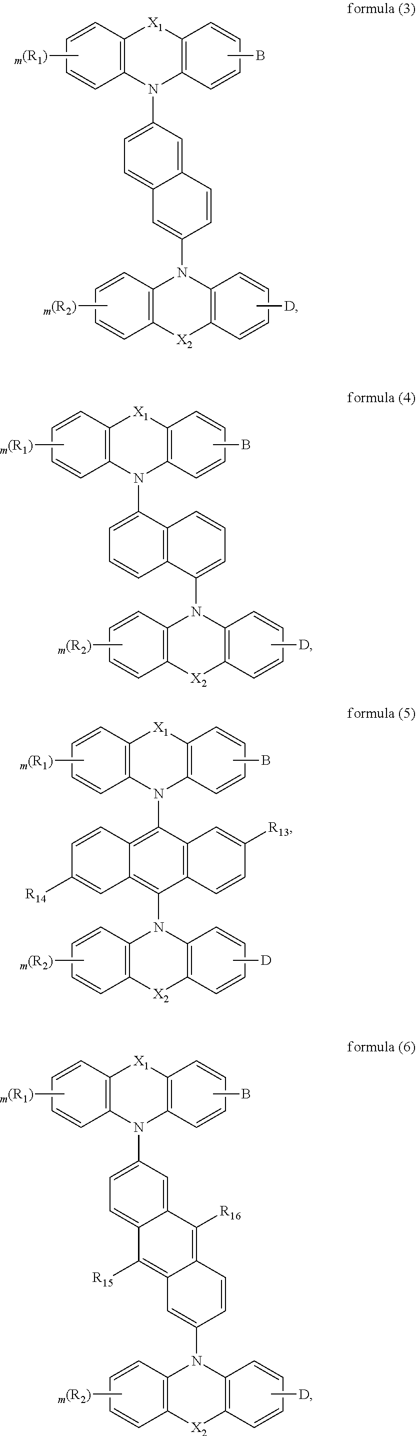

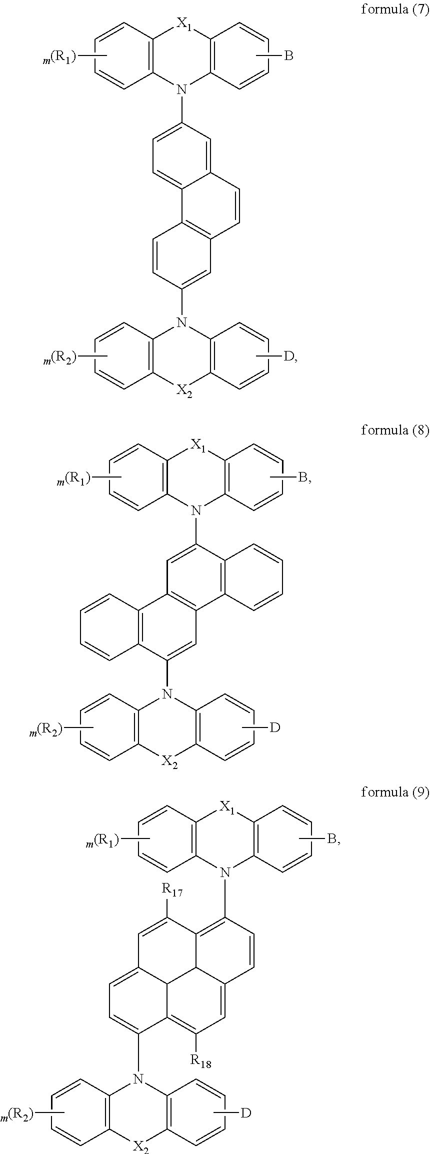

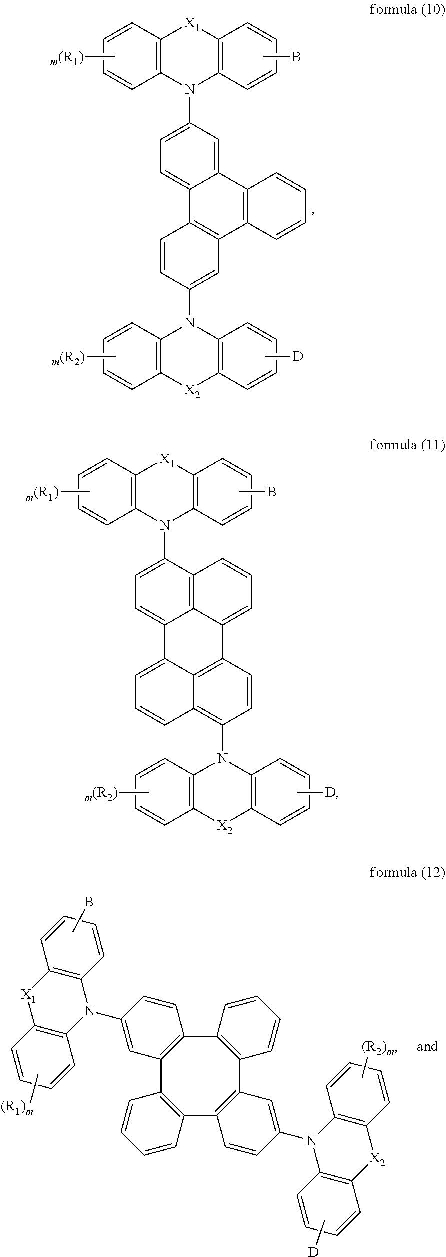

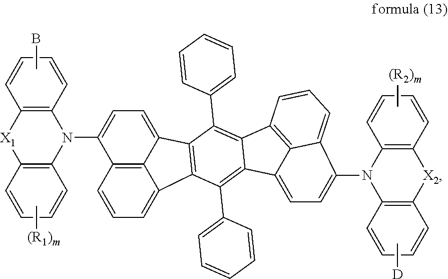

wherein X₁ and X₂ independently represent a divalent bridge selected from the group consisting of O, S, Se, CR₃R₄, NR₅, and SiR₆R₇; m is an integer of 0 to 4; B and D independently represent formula (2) below:

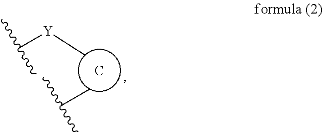

wherein ring C represents a phenyl ring or a fused ring hydrocarbon unit with two to five rings; Y represents a divalent bridge selected from the group consisting of O, S, Se, CR₈R₉, NR₁₀, and SiR₁₁R₁₂; R₁ to R₁₂ are independently a hydrogen atom, a halide, a substituted or unsubstituted alkyl group having 1 to 60 carbon atoms, a substituted or unsubstituted aryl group having 6 to 60 carbon atoms, a substituted or unsubstituted aralkyl group having 6 to 60 carbon atoms, a substituted or unsubstituted heteroaryl group having 3 to 60 carbon atoms, or a substituted or unsubstituted arylamine group having 6 to 60 carbon atoms; and R₁₃ to R₁₈ are independently a hydrogen atom, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, a substituted or unsubstituted heteroaryl group having 3 to 30 carbon atoms, or a substituted or unsubstituted arylamine group having 6 to 30 carbon atoms.

Preferably, the heteroaromatic compound is one of the following compounds:

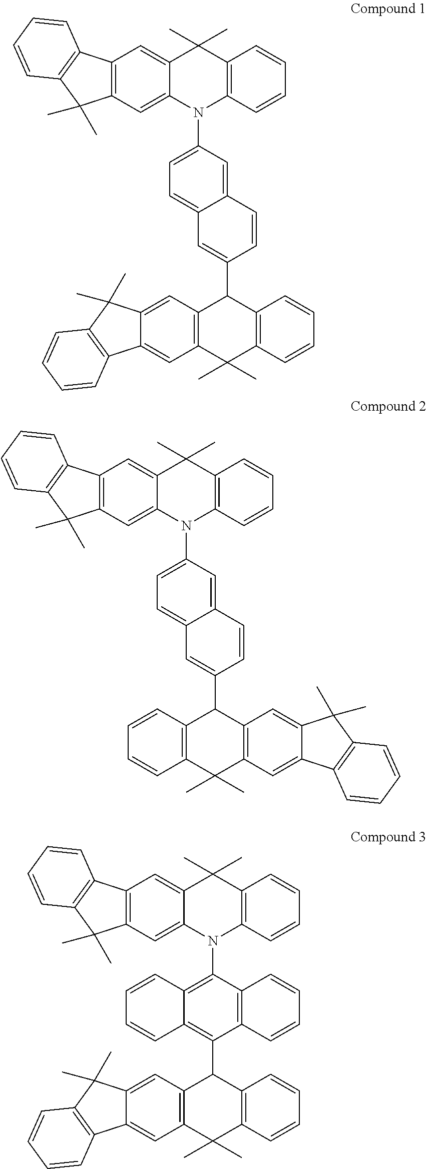

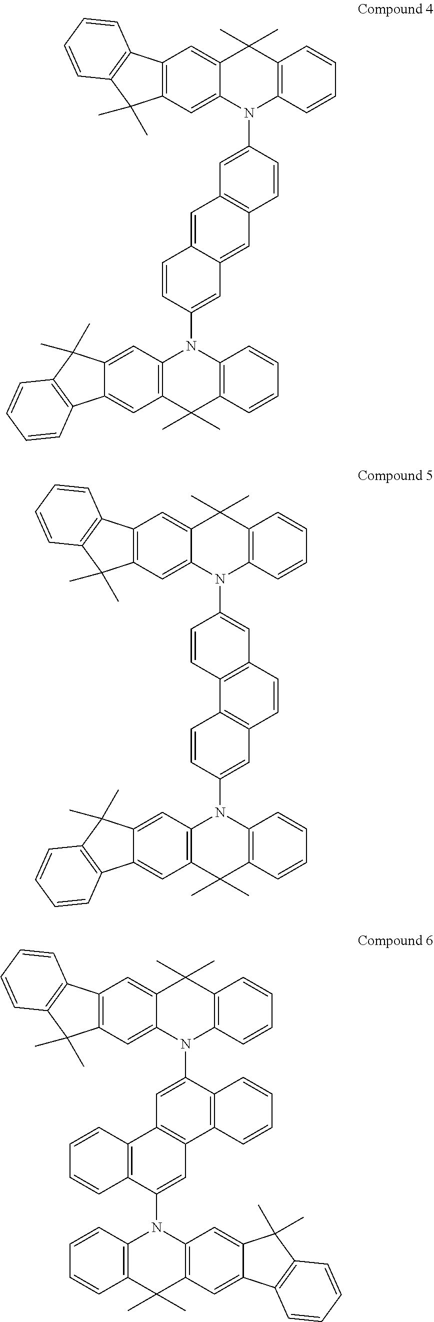 [SMILES: CC1(C)c2ccccc2-c2cc3c(cc21)N(c1ccc2cc4cc(N5c6ccccc6C(C)(C)c6cc7c(cc65)C(C)(C)c5ccccc5-7)ccc4cc2c1)c1ccccc1C3(C)C ; CC1(C)c2ccccc2-c2cc3c(cc21)N(c1ccc2c(ccc4cc(N5c6ccccc6C(C)(C)c6cc7c(cc65)C(C)(C)c5ccccc5-7)ccc42)c1)c1ccccc1C3(C)C ; CC1(C)c2ccccc2-c2cc3c(cc21)N(c1cc2c4ccccc4c(N4c5ccccc5C(C)(C)c5cc6c(cc54)C(C)(C)c4ccccc4-6)cc2c2ccccc12)c1ccccc1C3(C)C]

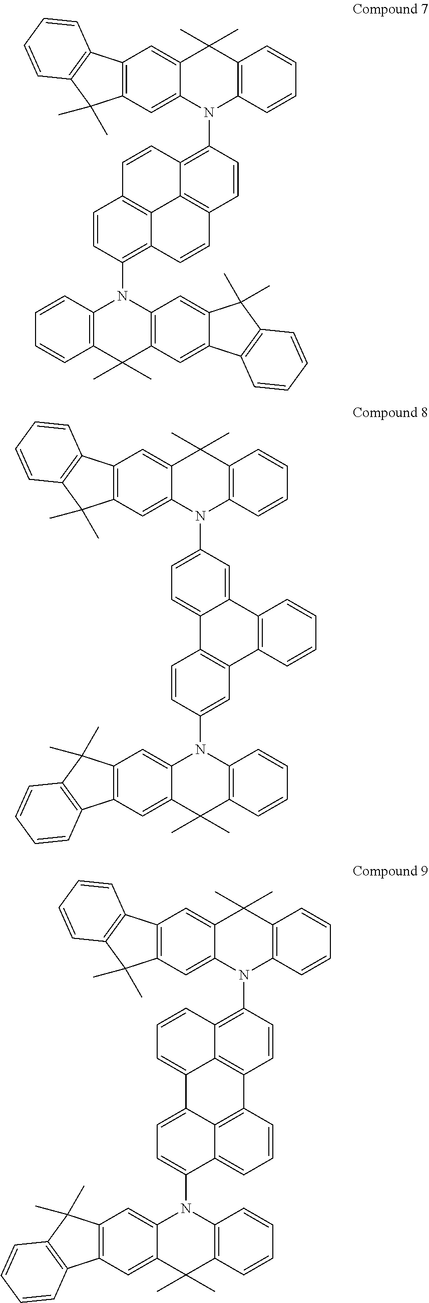

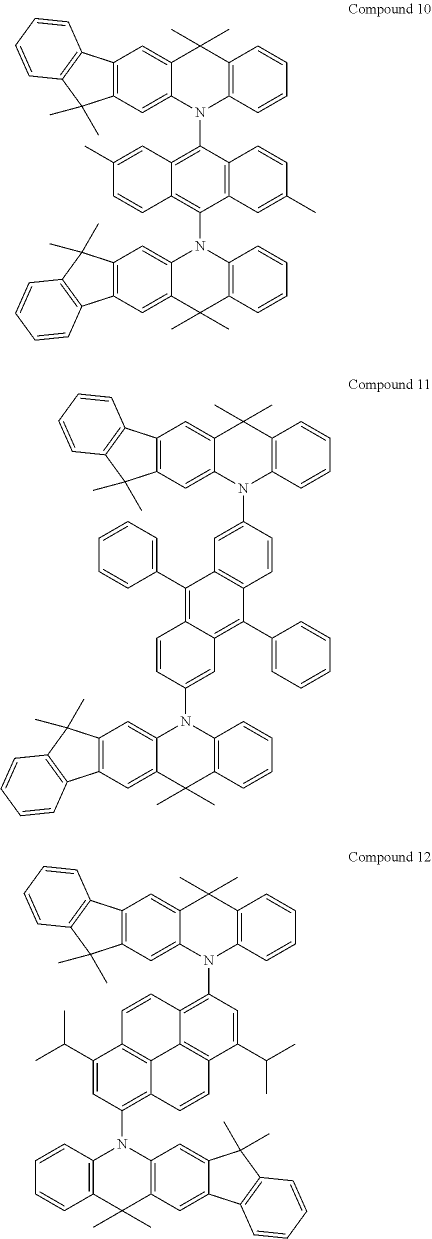 [SMILES: Cc1ccc2c(N3c4ccccc4C(C)(C)c4cc5c(cc43)C(C)(C)c3ccccc3-5)c3cc(C)ccc3c(N3c4ccccc4C(C)(C)c4cc5c(cc43)C(C)(C)c3ccccc3-5)c2c1 ; CC1(C)c2ccccc2-c2cc3c(cc21)N(c1ccc2c(-c4ccccc4)c4cc(N5c6ccccc6C(C)(C)c6cc7c(cc65)C(C)(C)c5ccccc5-7)ccc4c(-c4ccccc4)c2c1)c1ccccc1C3(C)C ; CC(C)c1cc(N2c3ccccc3C(C)(C)c3cc4c(cc32)C(C)(C)c2ccccc2-4)c2ccc3c(C(C)C)cc(N4c5ccccc5C(C)(C)c5cc6c(cc54)C(C)(C)c4ccccc4-6)c4ccc1c2c34]

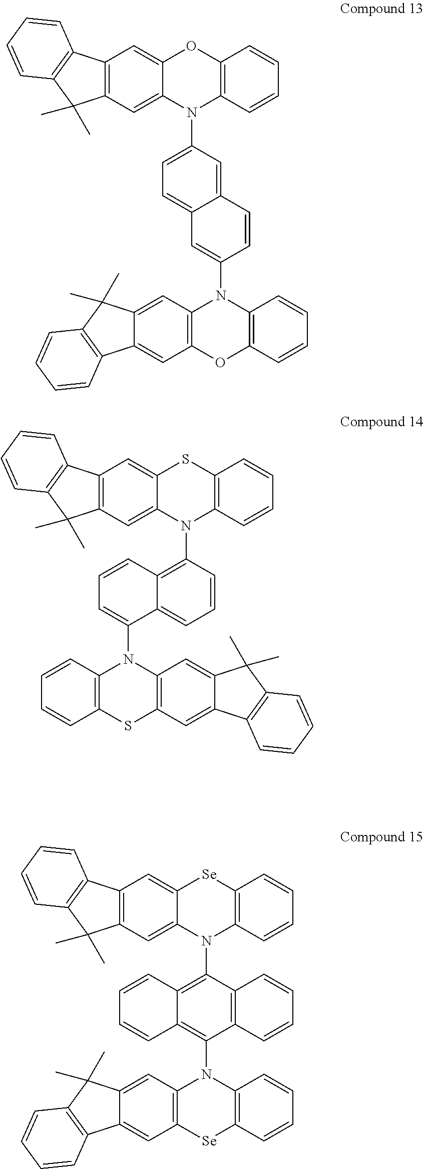

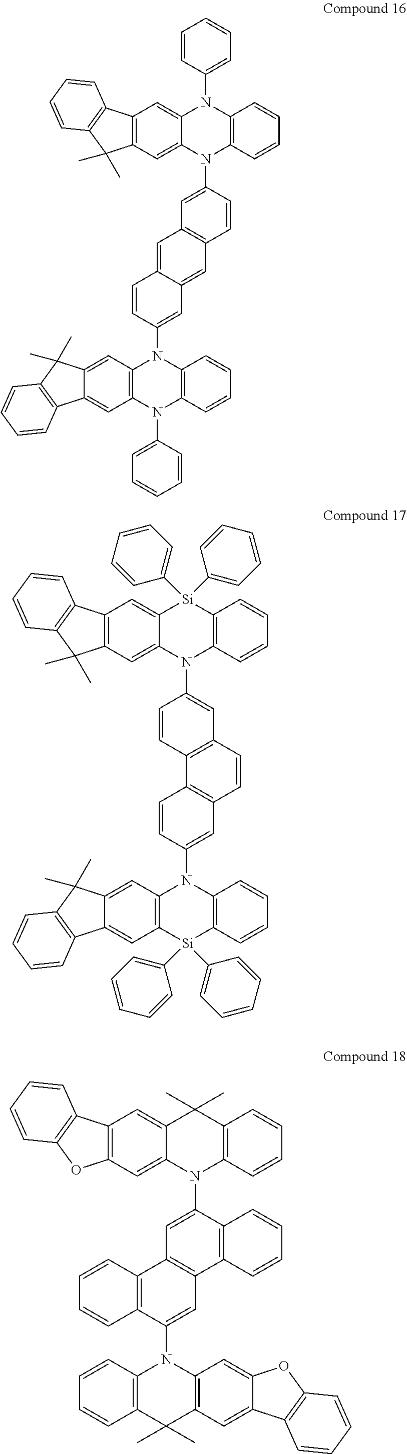

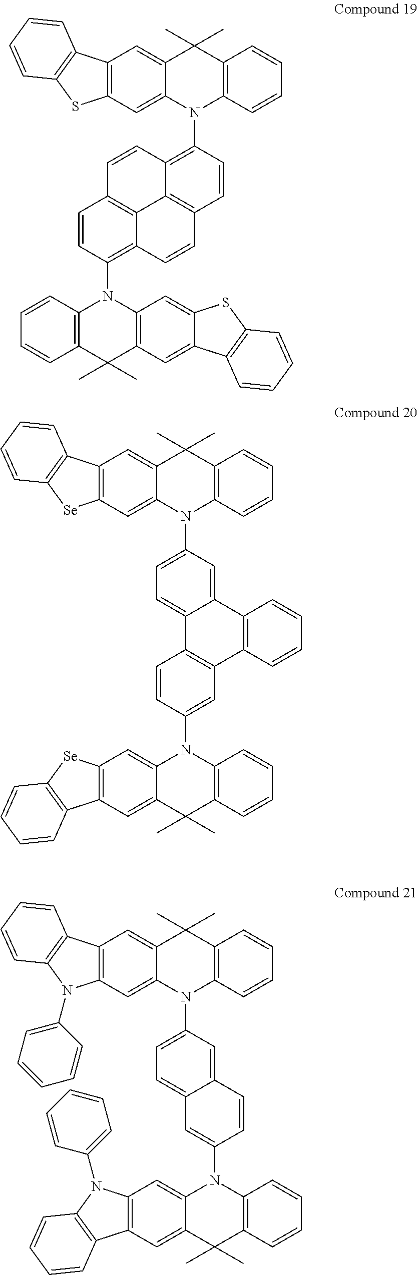 [SMILES: CC1(C)c2ccccc2N(c2ccc3ccc4c(N5c6ccccc6C(C)(C)c6cc7c(cc65)sc5ccccc57)ccc5ccc2c3c54)c2cc3sc4ccccc4c3cc21 ; CC1(C)c2ccccc2N(c2ccc3c4ccc(N5c6ccccc6C(C)(C)c6cc7c(cc65)[se]c5ccccc57)cc4c4ccccc4c3c2)c2cc3[se]c4ccccc4c3cc21 ; CC1(C)c2ccccc2N(c2ccc3cc(N4c5ccccc5C(C)(C)c5cc6c7ccccc7n(-c7ccccc7)c6cc54)ccc3c2)c2cc3c(cc21)c1ccccc1n3-c1ccccc1]

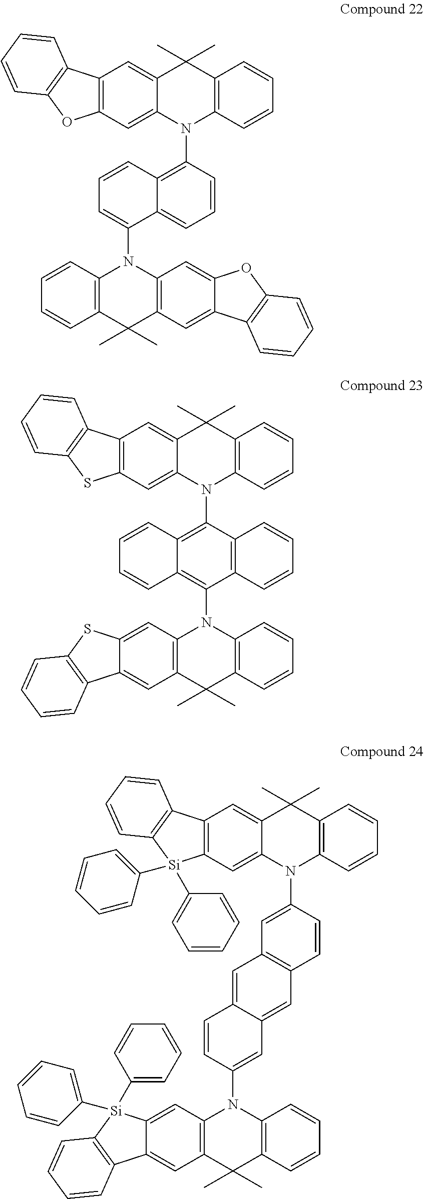 [SMILES: CC1(C)c2ccccc2N(c2cccc3c(N4c5ccccc5C(C)(C)c5cc6c(cc54)oc4ccccc46)cccc23)c2cc3oc4ccccc4c3cc21 ; CC1(C)c2ccccc2N(c2c3ccccc3c(N3c4ccccc4C(C)(C)c4cc5c(cc43)sc3ccccc35)c3ccccc23)c2cc3sc4ccccc4c3cc21 ; CC1(C)c2ccccc2N(c2ccc3cc4cc(N5c6ccccc6C(C)(C)c6cc7c(cc65)[Si](c5ccccc5)(c5ccccc5)c5ccccc5-7)ccc4cc3c2)c2cc3c(cc21)-c1ccccc1[Si]3(c1ccccc1)c1ccccc1]

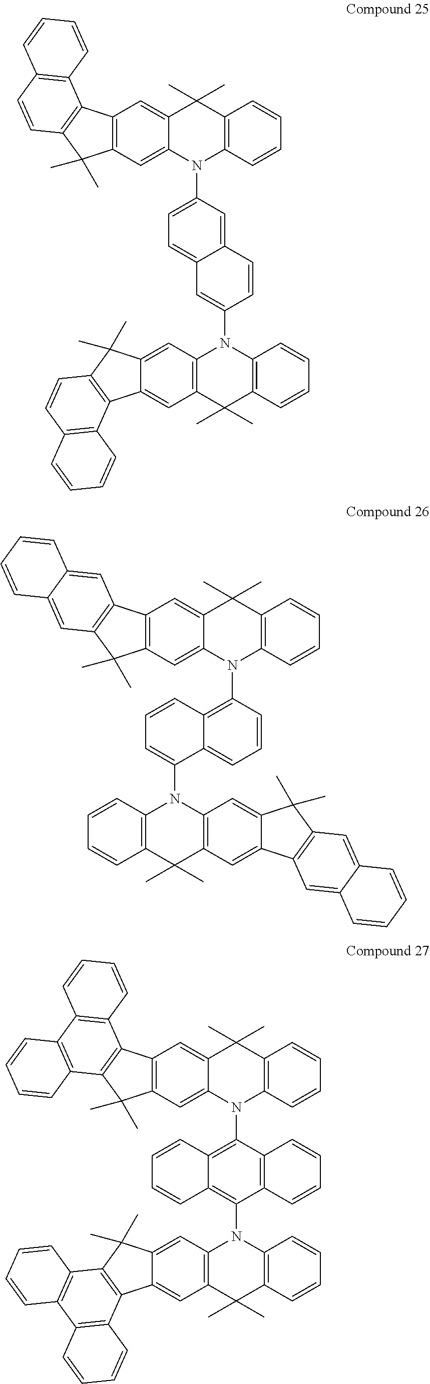 [SMILES: CC1(C)c2ccccc2N(c2ccc3cc(N4c5ccccc5C(C)(C)c5cc6c(cc54)C(C)(C)c4ccc5ccccc5c4-6)ccc3c2)c2cc3c(cc21)-c1c(ccc2ccccc12)C3(C)C ; CC1(C)c2cc3c(cc2-c2cc4ccccc4cc21)C(C)(C)c1ccccc1N3c1cccc2c(N3c4ccccc4C(C)(C)c4cc5c(cc43)C(C)(C)c3cc4ccccc4cc3-5)cccc12 ; CC1(C)c2ccccc2N(c2c3ccccc3c(N3c4ccccc4C(C)(C)c4cc5c(cc43)C(C)(C)c3c-5c4ccccc4c4ccccc34)c3ccccc23)c2cc3c(cc21)-c1c(c2ccccc2c2ccccc12)C3(C)C]

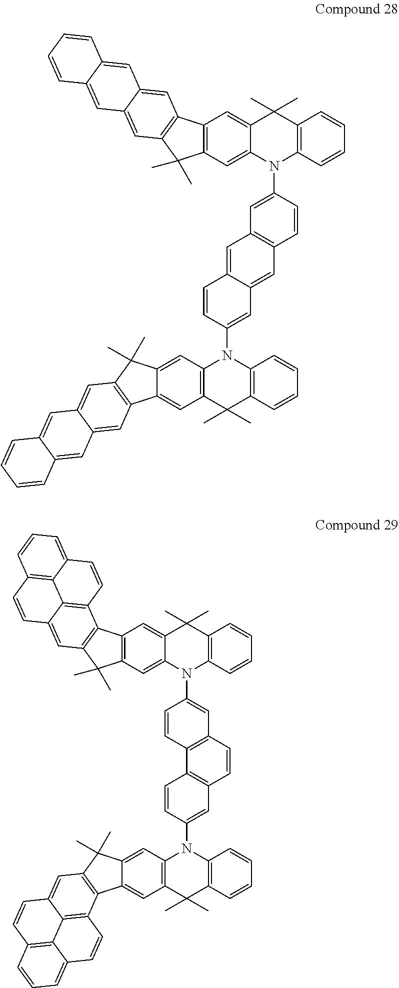 [SMILES: CC1(C)c2cc3c(cc2-c2cc4cc5ccccc5cc4cc21)C(C)(C)c1ccccc1N3c1ccc2cc3cc(N4c5ccccc5C(C)(C)c5cc6c(cc54)C(C)(C)c4cc5cc7ccccc7cc5cc4-6)ccc3cc2c1 ; CC1(C)c2ccccc2N(c2ccc3c(ccc4cc(N5c6ccccc6C(C)(C)c6cc7c(cc65)C(C)(C)c5cc6ccc8cccc9ccc(c5-7)c6c89)ccc43)c2)c2cc3c(cc21)-c1c(cc2ccc4cccc5ccc1c2c45)C3(C)C]

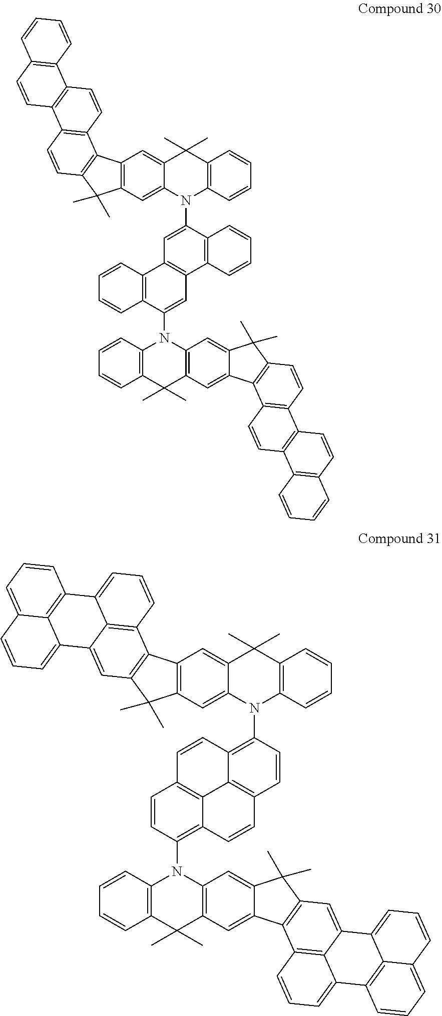 [SMILES: CC1(C)c2ccccc2N(c2cc3c4ccccc4c(N4c5ccccc5C(C)(C)c5cc6c(cc54)C(C)(C)c4ccc5c(ccc7c8ccccc8ccc57)c4-6)cc3c3ccccc23)c2cc3c(cc21)-c1c(ccc2c1ccc1c4ccccc4ccc21)C3(C)C ; CC1(C)c2ccccc2N(c2ccc3ccc4c(N5c6ccccc6C(C)(C)c6cc7c(cc65)C(C)(C)c5cc6c8cccc9cccc(c%10cccc(c5-7)c%106)c98)ccc5ccc2c3c54)c2cc3c(cc21)-c1c(cc2c4cccc5cccc(c6cccc1c62)c54)C3(C)C]

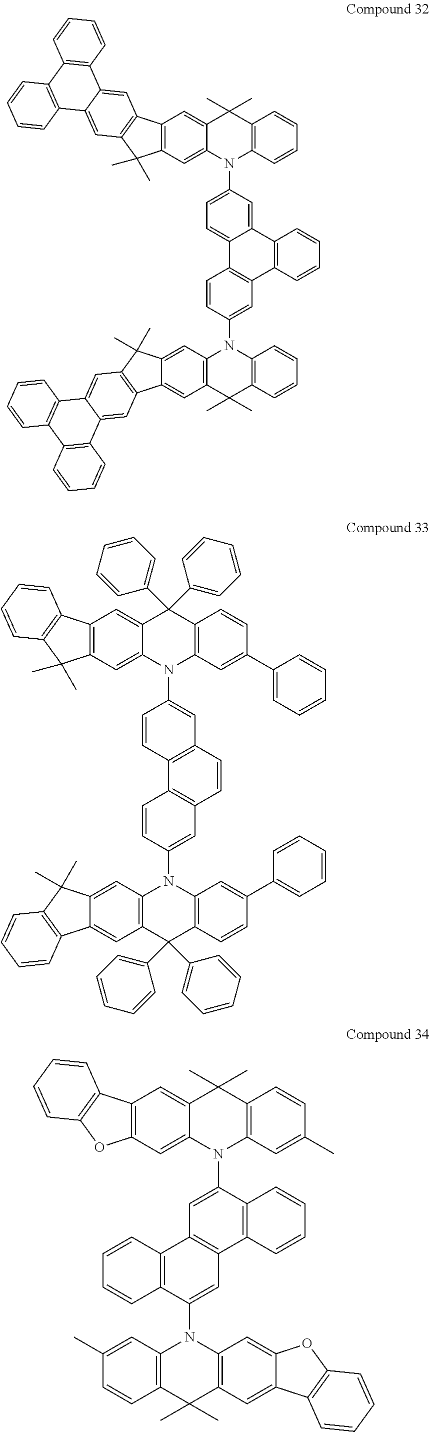 [SMILES: CC1(C)c2cc3c(cc2-c2cc4c5ccccc5c5ccccc5c4cc21)C(C)(C)c1ccccc1N3c1ccc2c3ccc(N4c5ccccc5C(C)(C)c5cc6c(cc54)C(C)(C)c4cc5c7ccccc7c7ccccc7c5cc4-6)cc3c3ccccc3c2c1 ; CC1(C)c2ccccc2-c2cc3c(cc21)N(c1ccc2c(ccc4cc(N5c6cc(-c7ccccc7)ccc6C(c6ccccc6)(c6ccccc6)c6cc7c(cc65)C(C)(C)c5ccccc5-7)ccc42)c1)c1cc(-c2ccccc2)ccc1C3(c1ccccc1)c1ccccc1 ; Cc1ccc2c(c1)N(c1cc3c4ccccc4c(N4c5cc(C)ccc5C(C)(C)c5cc6c(cc54)oc4ccccc46)cc3c3ccccc13)c1cc3oc4ccccc4c3cc1C2(C)C]

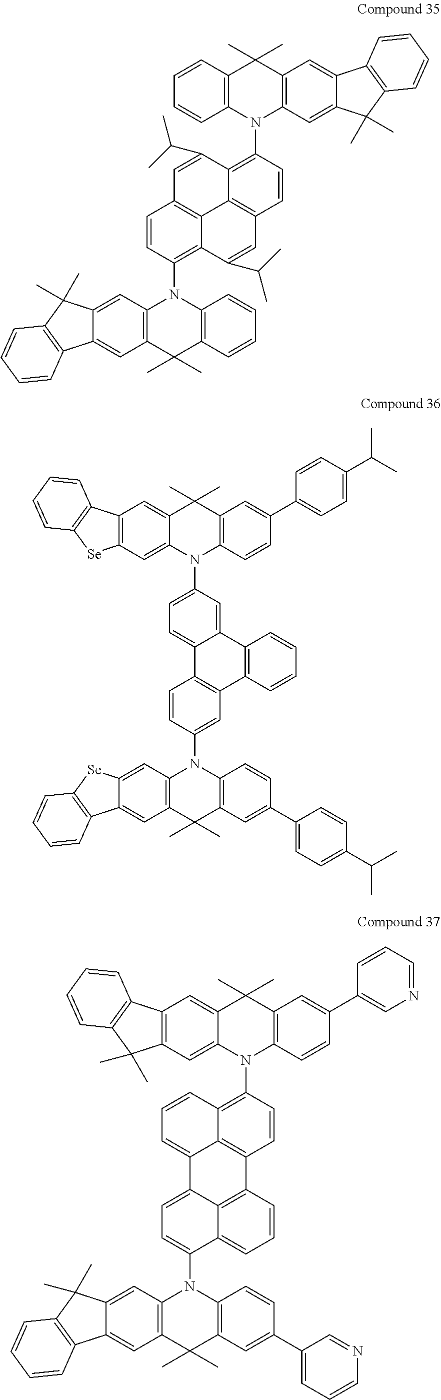 [SMILES: CC(C)c1cc2ccc(N3c4ccccc4C(C)(C)c4cc5c(cc43)C(C)(C)c3ccccc3-5)c3c(C(C)C)cc4ccc(N5c6ccccc6C(C)(C)c6cc7c(cc65)C(C)(C)c5ccccc5-7)c1c4c23 ; CC(C)c1ccc(-c2ccc3c(c2)C(C)(C)c2cc4c(cc2N3c2ccc3c5ccc(N6c7ccc(-c8ccc(C(C)C)cc8)cc7C(C)(C)c7cc8c(cc76)[se]c6ccccc68)cc5c5ccccc5c3c2)[se]c2ccccc24)cc1 ; CC1(C)c2ccccc2-c2cc3c(cc21)N(c1ccc2c4cccc5c(N6c7ccc(-c8cccnc8)cc7C(C)(C)c7cc8c(cc76)C(C)(C)c6ccccc6-8)ccc(c6cccc1c62)c54)c1ccc(-c2cccnc2)cc1C3(C)C]

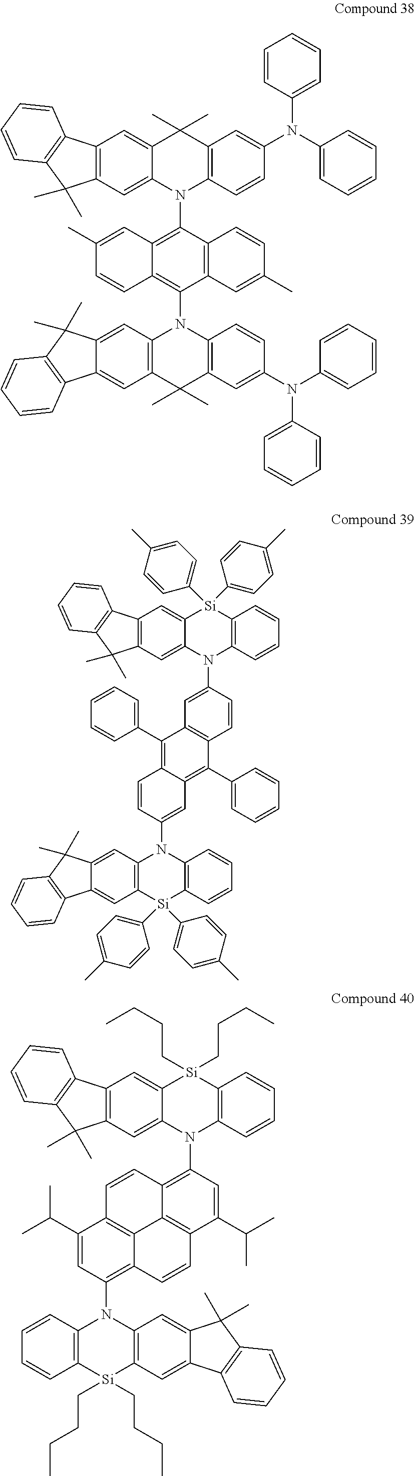

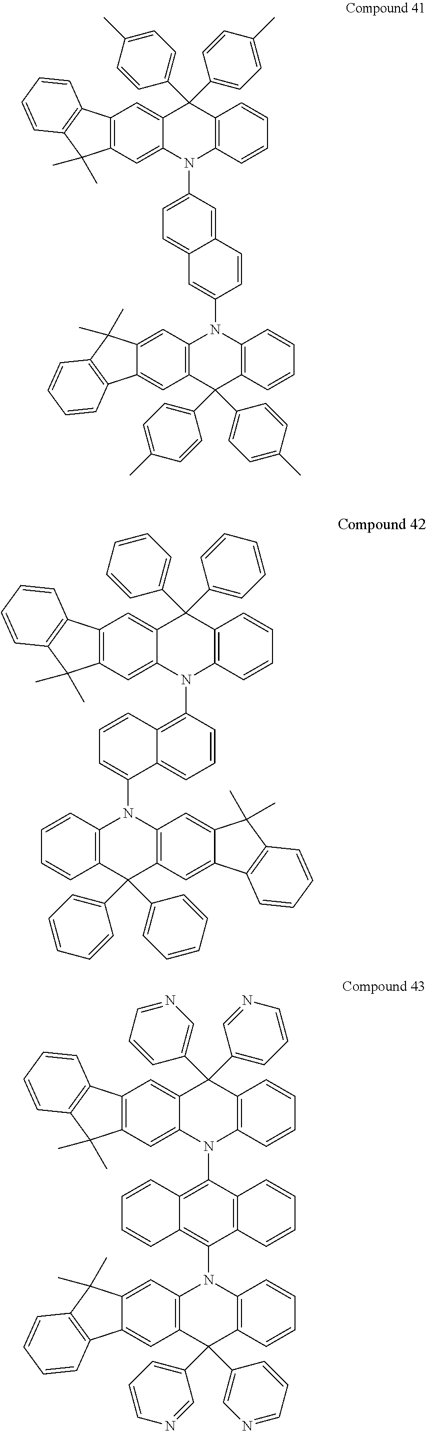

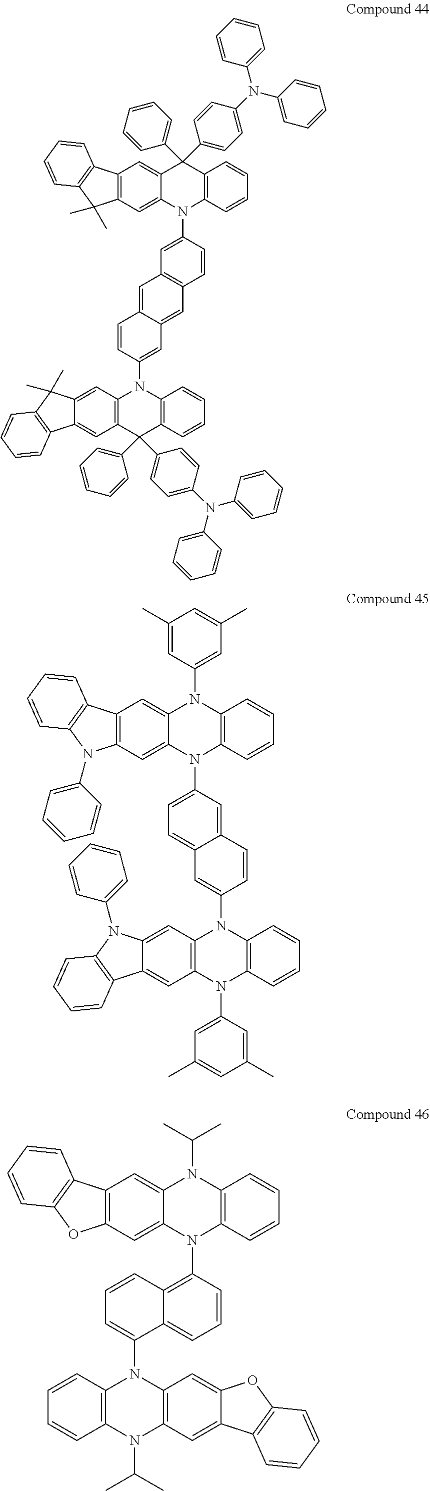

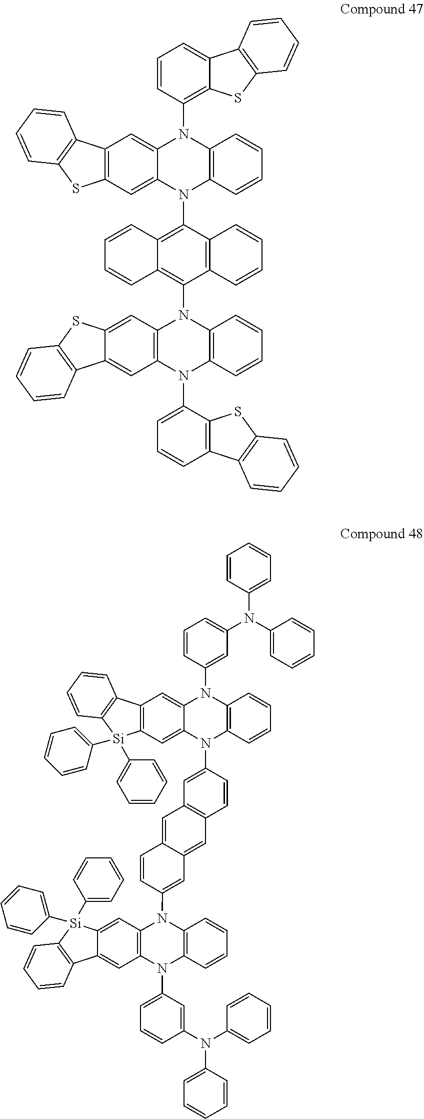 [SMILES: c1ccc2c(c1)N(c1c3ccccc3c(N3c4ccccc4N(c4cccc5c4sc4ccccc45)c4cc5c(cc43)sc3ccccc35)c3ccccc13)c1cc3sc4ccccc4c3cc1N2c1cccc2c1sc1ccccc12 ; c1ccc(N(c2ccccc2)c2cccc(N3c4ccccc4N(c4ccc5cc6cc(N7c8ccccc8N(c8cccc(N(c9ccccc9)c9ccccc9)c8)c8cc9c(cc87)[Si](c7ccccc7)(c7ccccc7)c7ccccc7-9)ccc6cc5c4)c4cc5c(cc43)-c3ccccc3[Si]5(c3ccccc3)c3ccccc3)c2)cc1]

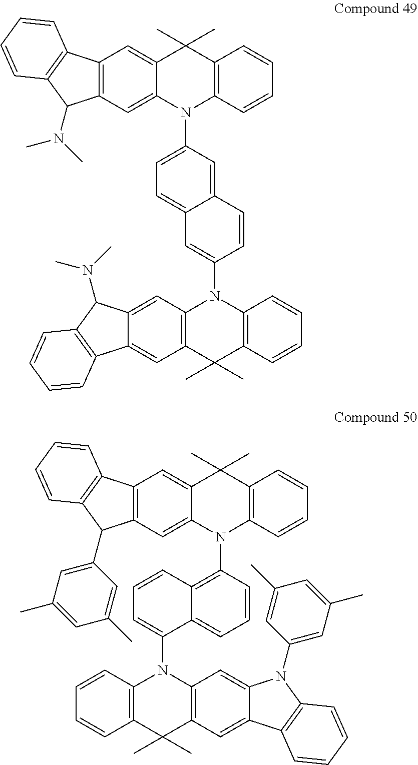

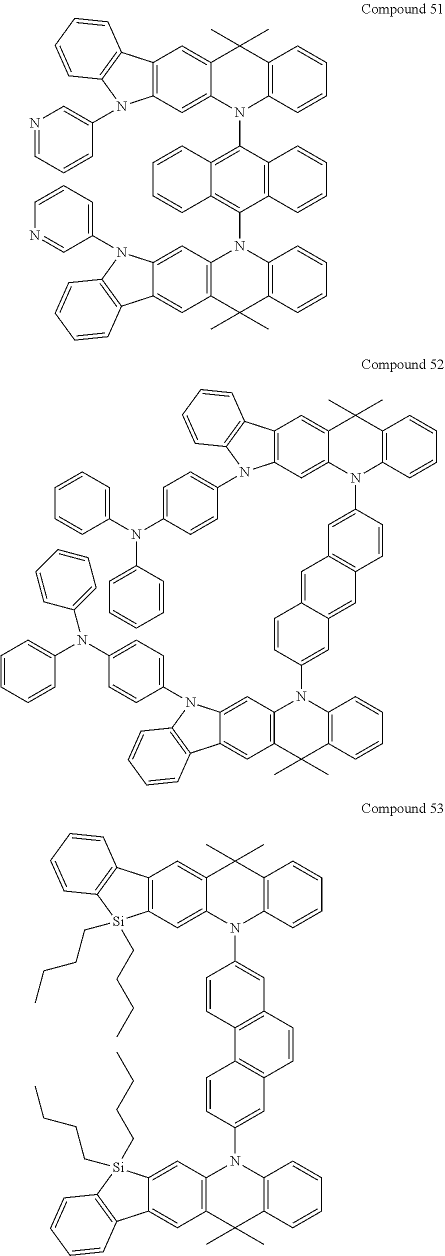 [SMILES: CC1(C)c2ccccc2N(c2c3ccccc3c(N3c4ccccc4C(C)(C)c4cc5c6ccccc6n(-c6cccnc6)c5cc43)c3ccccc23)c2cc3c(cc21)c1ccccc1n3-c1cccnc1 ; CC1(C)c2ccccc2N(c2ccc3cc4cc(N5c6ccccc6C(C)(C)c6cc7c8ccccc8n(-c8ccc(N(c9ccccc9)c9ccccc9)cc8)c7cc65)ccc4cc3c2)c2cc3c(cc21)c1ccccc1n3-c1ccc(N(c2ccccc2)c2ccccc2)cc1 ; CCCC[Si]1(CCCC)c2ccccc2-c2cc3c(cc21)N(c1ccc2c(ccc4cc(N5c6ccccc6C(C)(C)c6cc7c(cc65)[Si](CCCC)(CCCC)c5ccccc5-7)ccc42)c1)c1ccccc1C3(C)C]

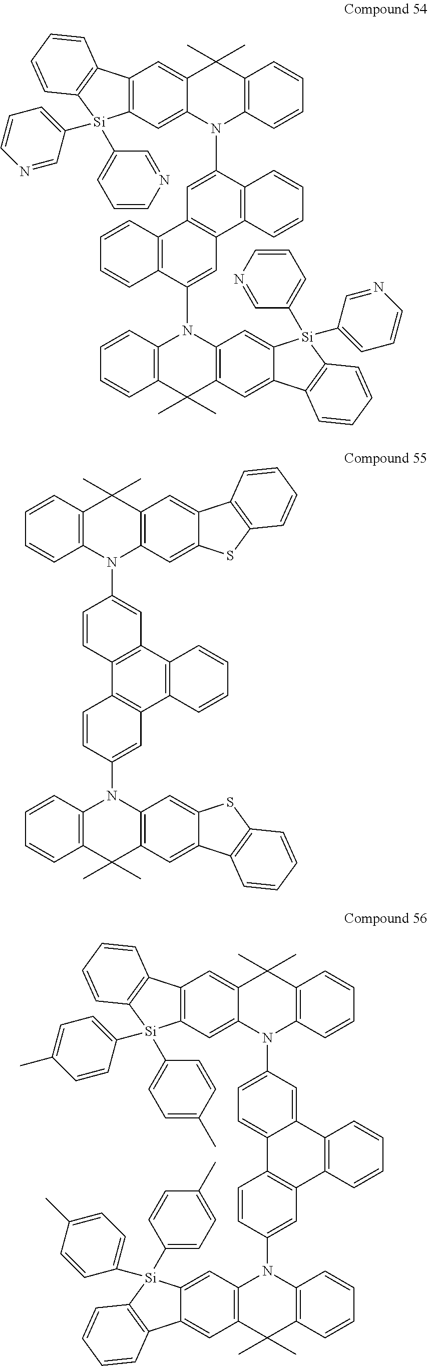 [SMILES: CC1(C)c2ccccc2N(c2cc3c4ccccc4c(N4c5ccccc5C(C)(C)c5cc6c(cc54)[Si](c4cccnc4)(c4cccnc4)c4ccccc4-6)cc3c3ccccc23)c2cc3c(cc21)-c1ccccc1[Si]3(c1cccnc1)c1cccnc1 ; CC1(C)c2ccccc2N(c2ccc3c4ccc(N5c6ccccc6C(C)(C)c6cc7c(cc65)sc5ccccc57)cc4c4ccccc4c3c2)c2cc3sc4ccccc4c3cc21 ; Cc1ccc([Si]2(c3ccc(C)cc3)c3ccccc3-c3cc4c(cc32)N(c2ccc3c5ccc(N6c7ccccc7C(C)(C)c7cc8c(cc76)[Si](c6ccc(C)cc6)(c6ccc(C)cc6)c6ccccc6-8)cc5c5ccccc5c3c2)c2ccccc2C4(C)C)cc1]

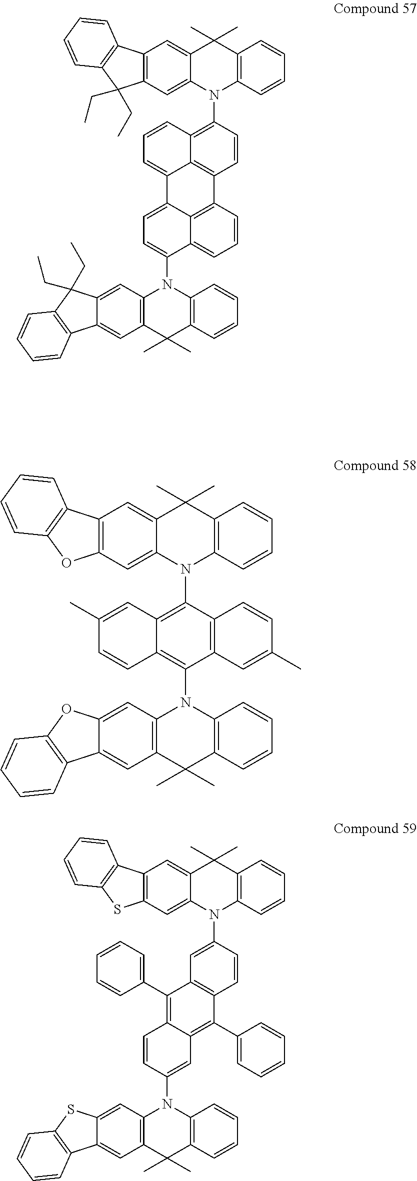 [SMILES: CCC1(CC)c2ccccc2-c2cc3c(cc21)N(c1ccc2c4cccc5c(N6c7ccccc7C(C)(C)c7cc8c(cc76)C(CC)(CC)c6ccccc6-8)ccc(c6cccc1c62)c54)c1ccccc1C3(C)C ; Cc1ccc2c(N3c4ccccc4C(C)(C)c4cc5c(cc43)oc3ccccc35)c3cc(C)ccc3c(N3c4ccccc4C(C)(C)c4cc5c(cc43)oc3ccccc35)c2c1 ; CC1(C)c2ccccc2N(c2ccc3c(-c4ccccc4)c4cc(N5c6ccccc6C(C)(C)c6cc7c(cc65)sc5ccccc57)ccc4c(-c4ccccc4)c3c2)c2cc3sc4ccccc4c3cc21]

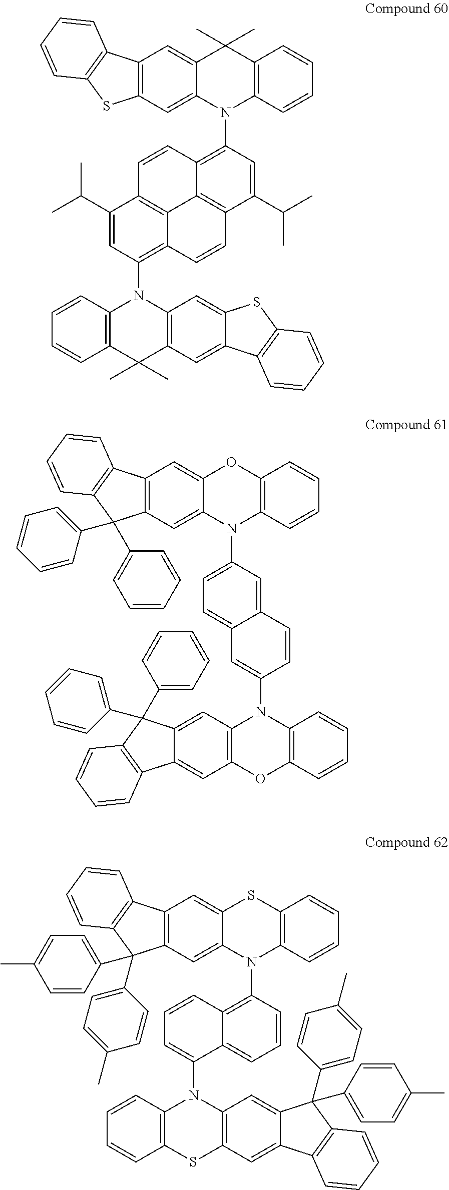

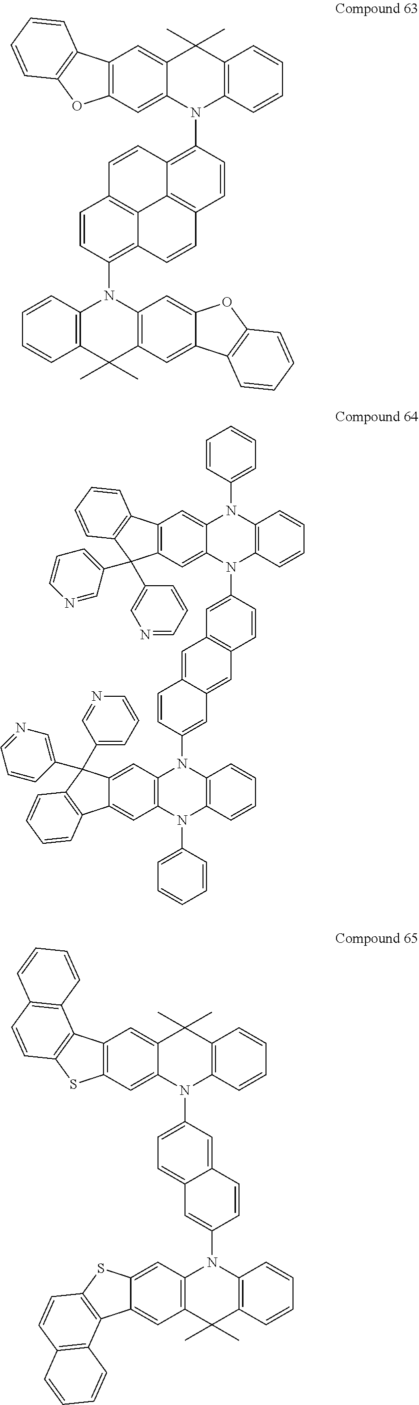

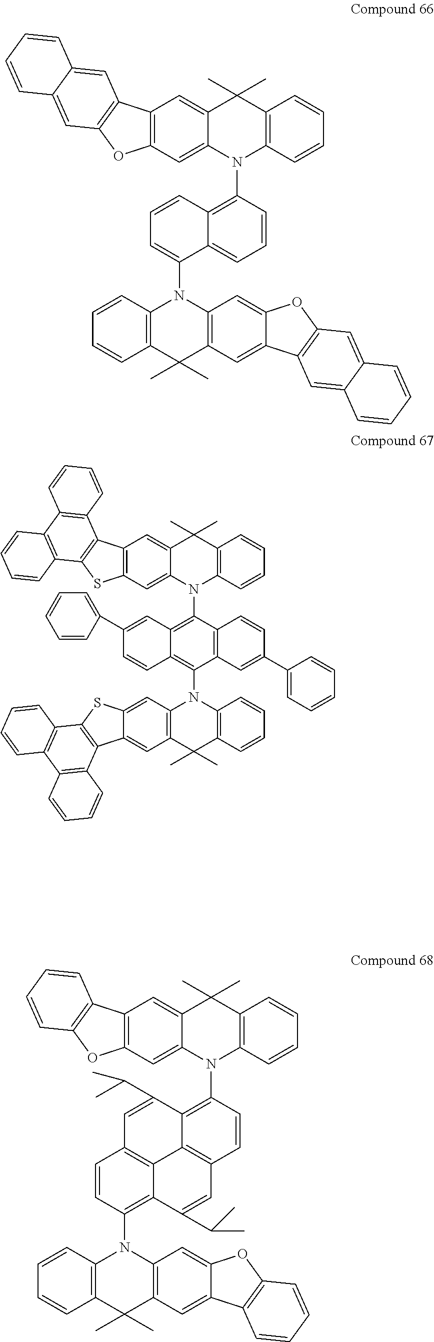 [SMILES: CC1(C)c2ccccc2N(c2cccc3c(N4c5ccccc5C(C)(C)c5cc6c(cc54)oc4cc5ccccc5cc46)cccc23)c2cc3oc4cc5ccccc5cc4c3cc21 ; CC1(C)c2ccccc2N(c2c3ccc(-c4ccccc4)cc3c(N3c4ccccc4C(C)(C)c4cc5c(cc43)sc3c4ccccc4c4ccccc4c53)c3ccc(-c4ccccc4)cc23)c2cc3sc4c5ccccc5c5ccccc5c4c3cc21 ; CC(C)c1cc2ccc(N3c4ccccc4C(C)(C)c4cc5c(cc43)oc3ccccc35)c3c(C(C)C)cc4ccc(N5c6ccccc6C(C)(C)c6cc7c(cc65)oc5ccccc57)c1c4c23]

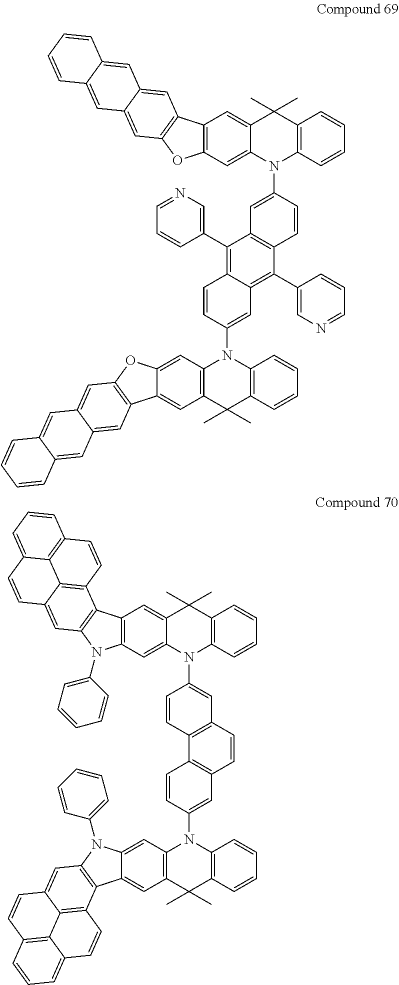

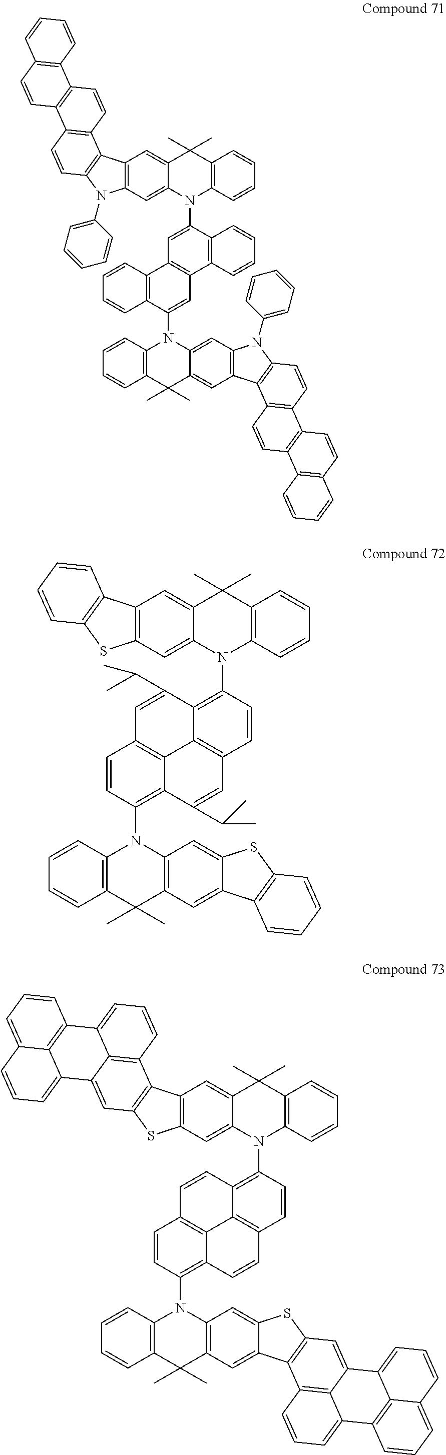 [SMILES: CC1(C)c2ccccc2N(c2cc3c4ccccc4c(N4c5ccccc5C(C)(C)c5cc6c7c8ccc9c%10ccccc%10ccc9c8ccc7n(-c7ccccc7)c6cc54)cc3c3ccccc23)c2cc3c(cc21)c1c2ccc4c5ccccc5ccc4c2ccc1n3-c1ccccc1 ; CC(C)c1cc2ccc(N3c4ccccc4C(C)(C)c4cc5c(cc43)sc3ccccc35)c3c(C(C)C)cc4ccc(N5c6ccccc6C(C)(C)c6cc7c(cc65)sc5ccccc57)c1c4c23 ; CC1(C)c2ccccc2N(c2ccc3ccc4c(N5c6ccccc6C(C)(C)c6cc7c(cc65)sc5cc6c8cccc9cccc(c%10cccc(c57)c%106)c98)ccc5ccc2c3c54)c2cc3sc4cc5c6cccc7cccc(c8cccc(c4c3cc21)c85)c76]

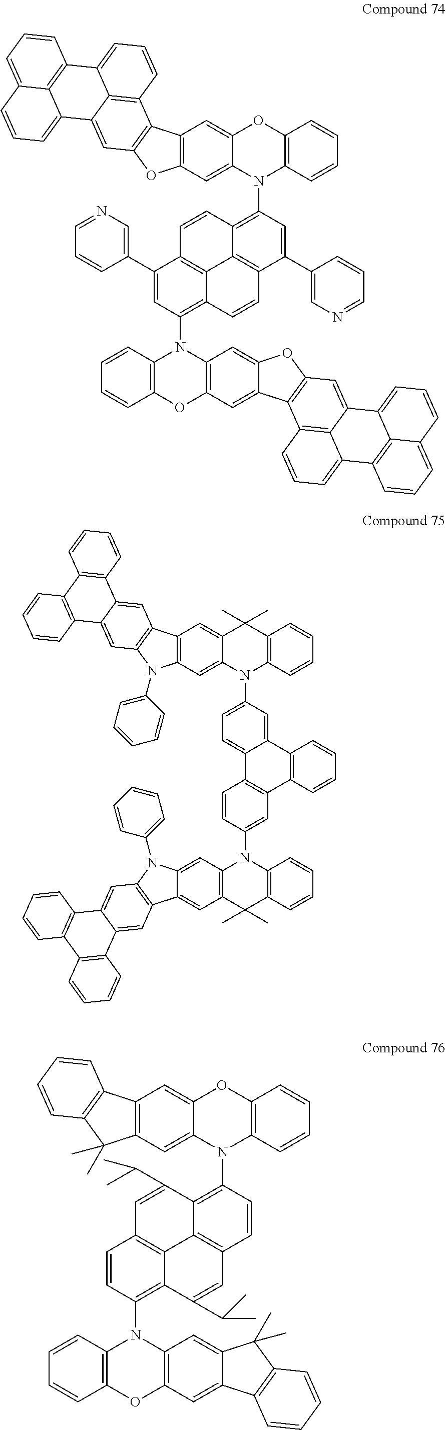 [SMILES: c1cncc(-c2cc(-n3c4ccccc4oc4cc5c(cc43)oc3cc4c6cccc7cccc(c8cccc(c35)c84)c76)c3ccc4c(-c5cccnc5)cc(-n5c6ccccc6oc6cc7c(cc65)oc5cc6c8cccc9cccc(c%10cccc(c57)c%106)c98)c5ccc2c3c45)c1 ; CC1(C)c2ccccc2N(c2ccc3c4ccc(N5c6ccccc6C(C)(C)c6cc7c8cc9c%10ccccc%10c%10ccccc%10c9cc8n(-c8ccccc8)c7cc65)cc4c4ccccc4c3c2)c2cc3c(cc21)c1cc2c4ccccc4c4ccccc4c2cc1n3-c1ccccc1 ; CC(C)c1cc2ccc(N3c4ccccc4Oc4cc5c(cc43)C(C)(C)c3ccccc3-5)c3c(C(C)C)cc4ccc(N5c6ccccc6Oc6cc7c(cc65)C(C)(C)c5ccccc5-7)c1c4c23]

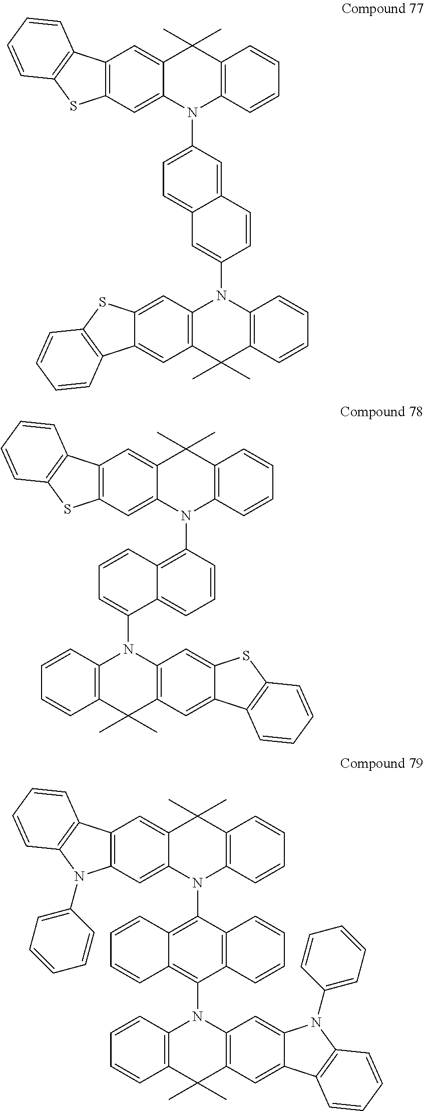

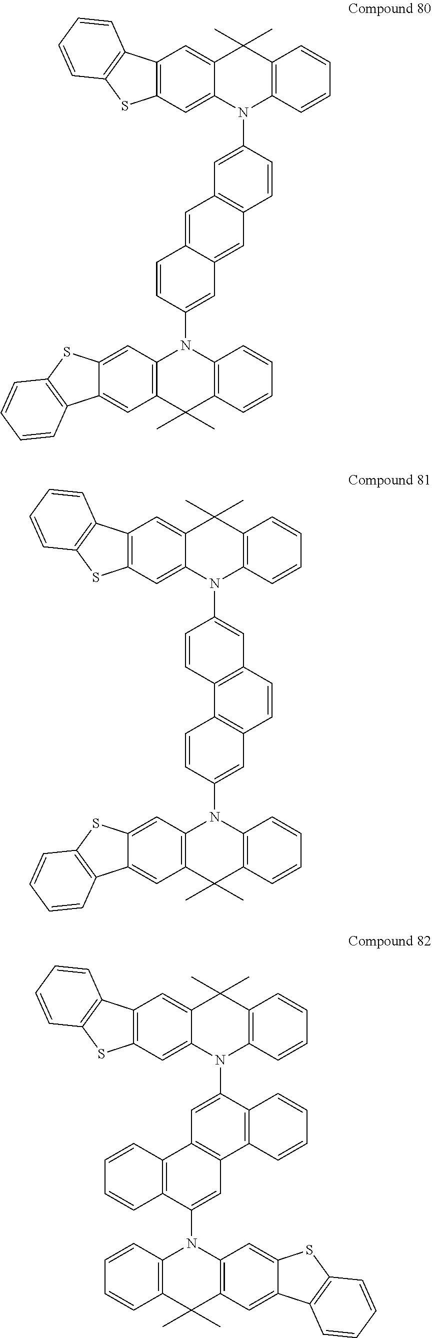 [SMILES: CC1(C)c2ccccc2N(c2ccc3cc4cc(N5c6ccccc6C(C)(C)c6cc7c(cc65)sc5ccccc57)ccc4cc3c2)c2cc3sc4ccccc4c3cc21 ; CC1(C)c2ccccc2N(c2ccc3c(ccc4cc(N5c6ccccc6C(C)(C)c6cc7c(cc65)sc5ccccc57)ccc43)c2)c2cc3sc4ccccc4c3cc21 ; CC1(C)c2ccccc2N(c2cc3c4ccccc4c(N4c5ccccc5C(C)(C)c5cc6c(cc54)sc4ccccc46)cc3c3ccccc23)c2cc3sc4ccccc4c3cc21]

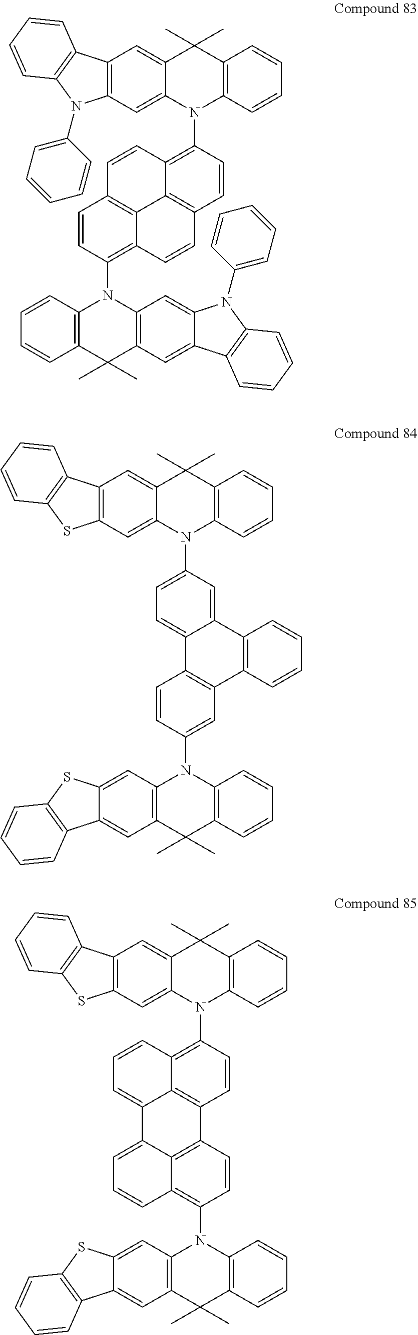 [SMILES: CC1(C)c2ccccc2N(c2ccc3ccc4c(N5c6ccccc6C(C)(C)c6cc7c8ccccc8n(-c8ccccc8)c7cc65)ccc5ccc2c3c54)c2cc3c(cc21)c1ccccc1n3-c1ccccc1 ; CC1(C)c2ccccc2N(c2ccc3c4ccc(N5c6ccccc6C(C)(C)c6cc7c(cc65)sc5ccccc57)cc4c4ccccc4c3c2)c2cc3sc4ccccc4c3cc21 ; CC1(C)c2ccccc2N(c2ccc3c4ccc(N5c6ccccc6C(C)(C)c6cc7c(cc65)sc5ccccc57)c5cccc(c6cccc2c63)c54)c2cc3sc4ccccc4c3cc21]

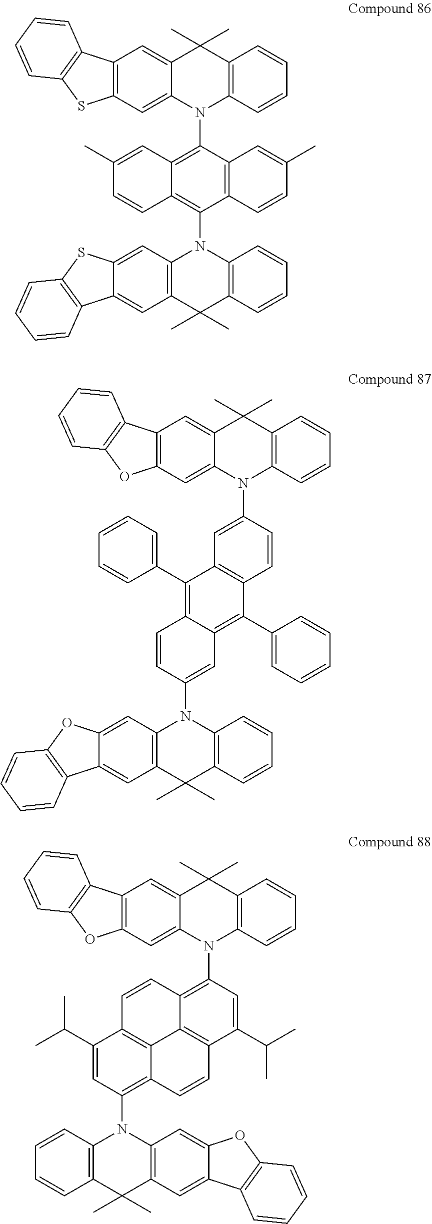

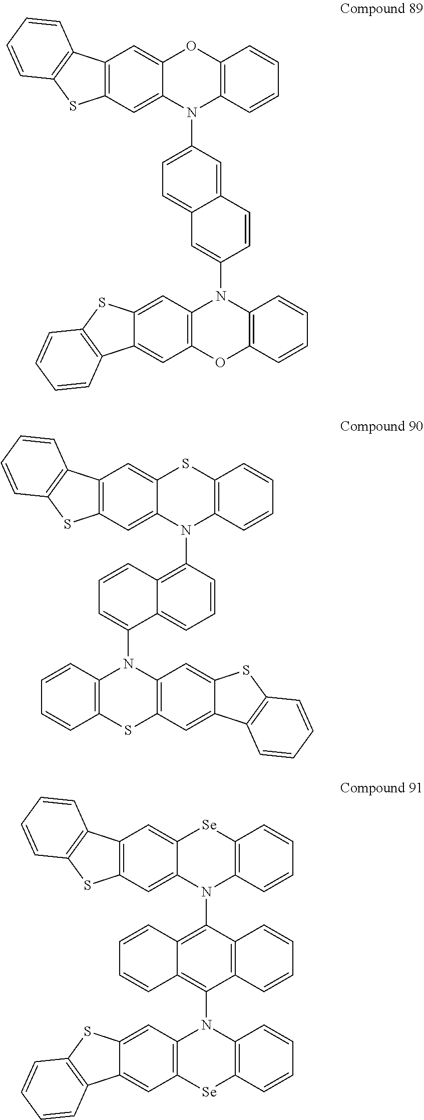 [SMILES: c1ccc2c(c1)Oc1cc3c(cc1N2c1ccc2cc(N4c5ccccc5Oc5cc6c(cc54)sc4ccccc46)ccc2c1)sc1ccccc13 ; c1ccc2c(c1)Sc1cc3c(cc1N2c1cccc2c(N4c5ccccc5Sc5cc6c(cc54)sc4ccccc46)cccc12)sc1ccccc13 ; c1ccc2c(c1)[Se]c1cc3c(cc1N2c1c2ccccc2c(N2c4ccccc4[Se]c4cc5c(cc42)sc2ccccc25)c2ccccc12)sc1ccccc13]

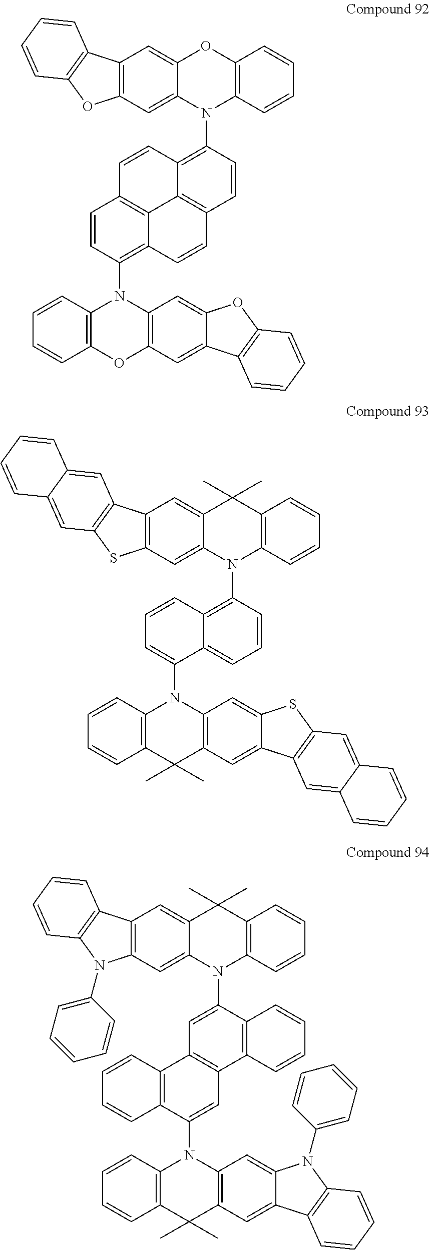 [SMILES: c1ccc2c(c1)Oc1cc3c(cc1N2c1ccc2ccc4c(N5c6ccccc6Oc6cc7c(cc65)oc5ccccc57)ccc5ccc1c2c54)oc1ccccc13 ; CC1(C)c2ccccc2N(c2cccc3c(N4c5ccccc5C(C)(C)c5cc6c(cc54)sc4cc5ccccc5cc46)cccc23)c2cc3sc4cc5ccccc5cc4c3cc21 ; CC1(C)c2ccccc2N(c2cc3c4ccccc4c(N4c5ccccc5C(C)(C)c5cc6c7ccccc7n(-c7ccccc7)c6cc54)cc3c3ccccc23)c2cc3c(cc21)c1ccccc1n3-c1ccccc1]

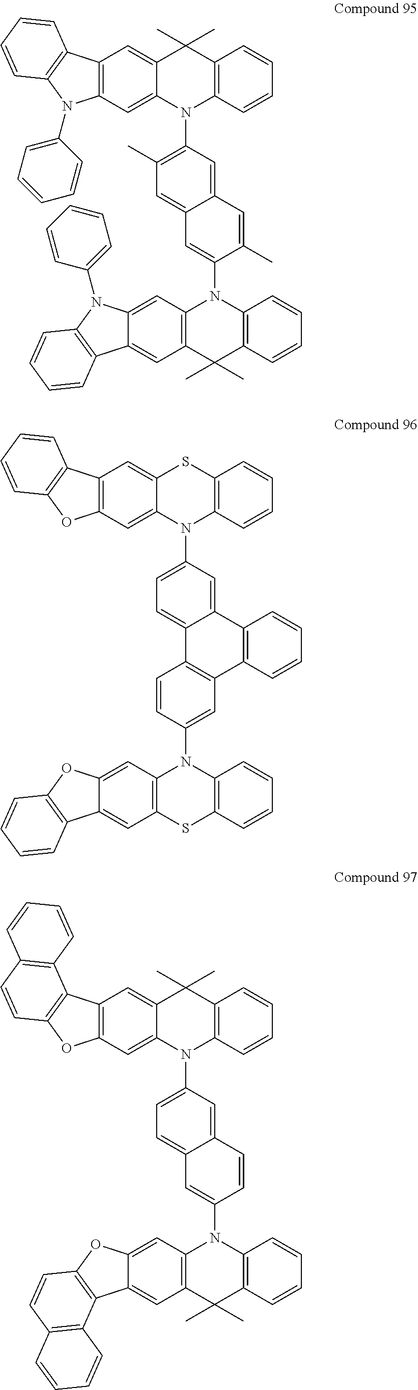 [SMILES: Cc1cc2cc(N3c4ccccc4C(C)(C)c4cc5c6ccccc6n(-c6ccccc6)c5cc43)c(C)cc2cc1N1c2ccccc2C(C)(C)c2cc3c4ccccc4n(-c4ccccc4)c3cc21 ; c1ccc2c(c1)Sc1cc3c(cc1N2c1ccc2c4ccc(N5c6ccccc6Sc6cc7c(cc65)oc5ccccc57)cc4c4ccccc4c2c1)oc1ccccc13 ; CC1(C)c2ccccc2N(c2ccc3cc(N4c5ccccc5C(C)(C)c5cc6c(cc54)oc4ccc5ccccc5c46)ccc3c2)c2cc3oc4ccc5ccccc5c4c3cc21]

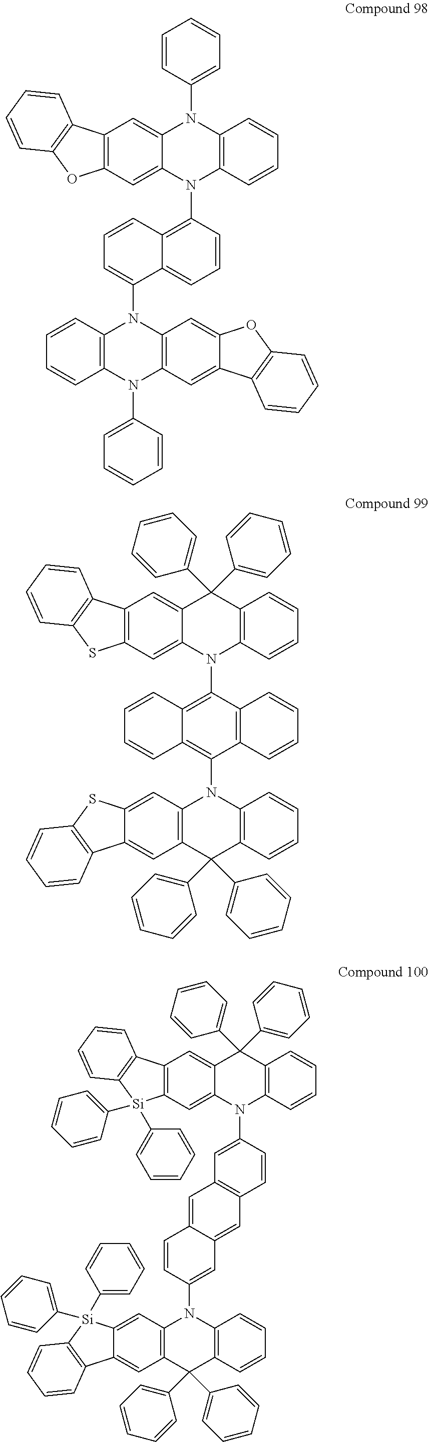

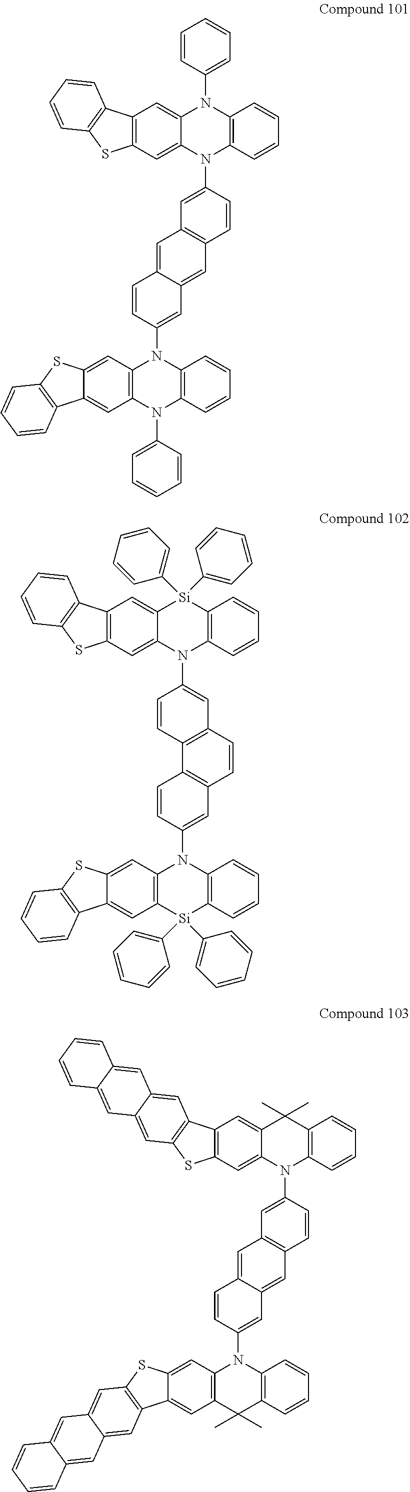

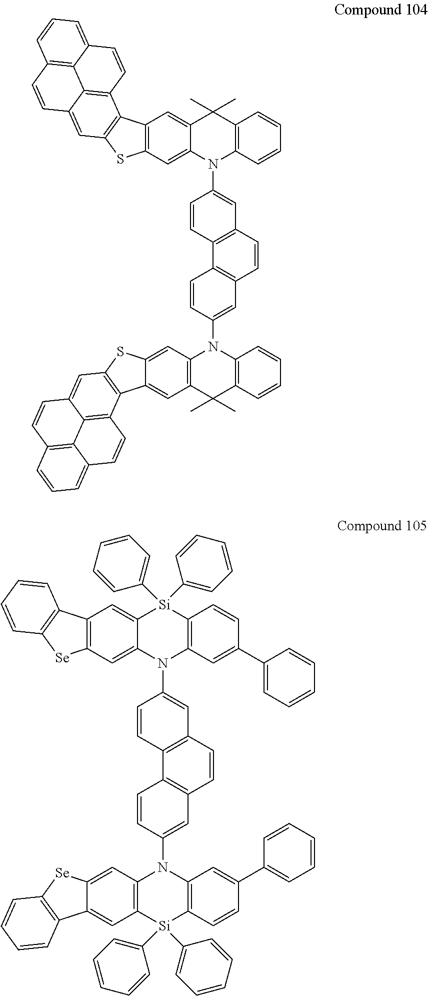 [SMILES: CC1(C)c2ccccc2N(c2ccc3c(ccc4cc(N5c6ccccc6C(C)(C)c6cc7c(cc65)sc5cc6ccc8cccc9ccc(c57)c6c89)ccc43)c2)c2cc3sc4cc5ccc6cccc7ccc(c4c3cc21)c5c67 ; c1ccc(-c2ccc3c(c2)N(c2ccc4c(ccc5cc(N6c7cc(-c8ccccc8)ccc7[Si](c7ccccc7)(c7ccccc7)c7cc8c(cc76)[se]c6ccccc68)ccc54)c2)c2cc4[se]c5ccccc5c4cc2[Si]3(c2ccccc2)c2ccccc2)cc1]

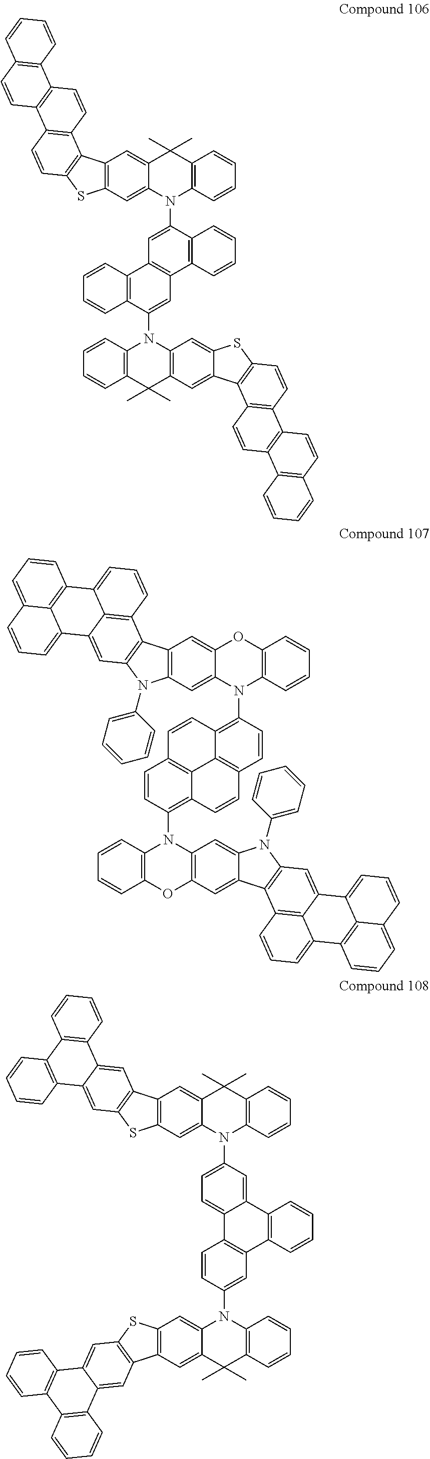 [SMILES: CC1(C)c2ccccc2N(c2cc3c4ccccc4c(N4c5ccccc5C(C)(C)c5cc6c(cc54)sc4ccc5c7ccc8ccccc8c7ccc5c46)cc3c3ccccc23)c2cc3sc4ccc5c6ccc7ccccc7c6ccc5c4c3cc21 ; c1ccc(-n2c3cc4c(cc3c3c5cccc6c7cccc8cccc(c(cc32)c65)c87)oc2ccccc2n4-c2ccc3ccc4c(-n5c6ccccc6oc6cc7c8c9cccc%10c%11cccc%12cccc(c(cc8n(-c8ccccc8)c7cc65)c%109)c%12%11)ccc5ccc2c3c54)cc1 ; CC1(C)c2ccccc2N(c2ccc3c4ccc(N5c6ccccc6C(C)(C)c6cc7c(cc65)sc5cc6c8ccccc8c8ccccc8c6cc57)cc4c4ccccc4c3c2)c2cc3sc4cc5c6ccccc6c6ccccc6c5cc4c3cc21]

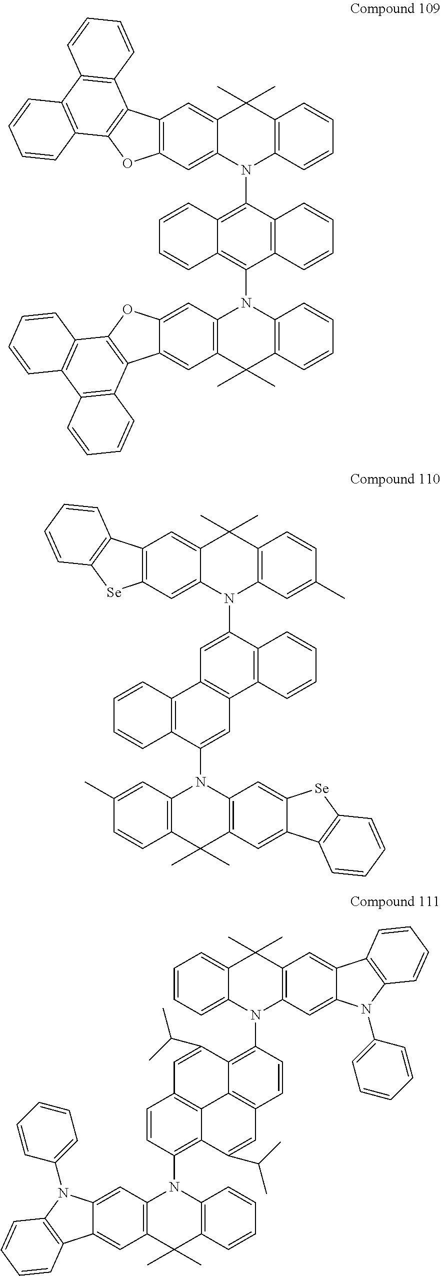 [SMILES: CC1(C)c2ccccc2N(c2c3ccccc3c(N3c4ccccc4C(C)(C)c4cc5c(cc43)oc3c4ccccc4c4ccccc4c53)c3ccccc23)c2cc3oc4c5ccccc5c5ccccc5c4c3cc21 ; Cc1ccc2c(c1)N(c1cc3c4ccccc4c(N4c5cc(C)ccc5C(C)(C)c5cc6c(cc54)[se]c4ccccc46)cc3c3ccccc13)c1cc3[se]c4ccccc4c3cc1C2(C)C ; CC(C)c1cc2ccc(N3c4ccccc4C(C)(C)c4cc5c6ccccc6n(-c6ccccc6)c5cc43)c3c(C(C)C)cc4ccc(N5c6ccccc6C(C)(C)c6cc7c8ccccc8n(-c8ccccc8)c7cc65)c1c4c23]

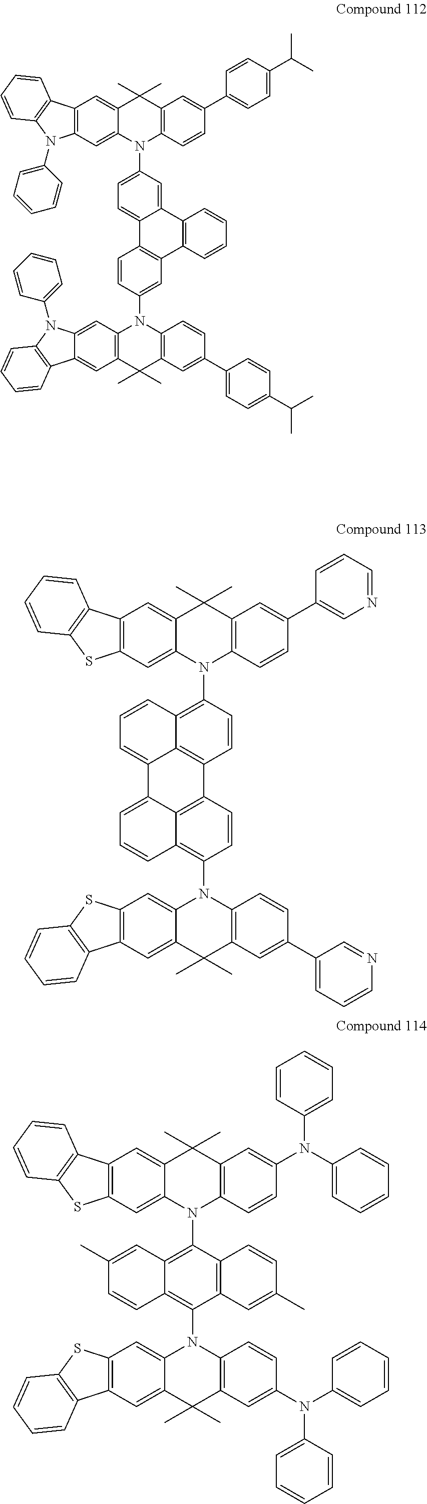 [SMILES: CC(C)c1ccc(-c2ccc3c(c2)C(C)(C)c2cc4c5ccccc5n(-c5ccccc5)c4cc2N3c2ccc3c4ccc(N5c6ccc(-c7ccc(C(C)C)cc7)cc6C(C)(C)c6cc7c8ccccc8n(-c8ccccc8)c7cc65)cc4c4ccccc4c3c2)cc1 ; CC1(C)c2cc(-c3cccnc3)ccc2N(c2ccc3c4ccc(N5c6ccc(-c7cccnc7)cc6C(C)(C)c6cc7c(cc65)sc5ccccc57)c5cccc(c6cccc2c63)c54)c2cc3sc4ccccc4c3cc21 ; Cc1ccc2c(N3c4ccc(N(c5ccccc5)c5ccccc5)cc4C(C)(C)c4cc5c(cc43)sc3ccccc35)c3cc(C)ccc3c(N3c4ccc(N(c5ccccc5)c5ccccc5)cc4C(C)(C)c4cc5c(cc43)sc3ccccc35)c2c1]

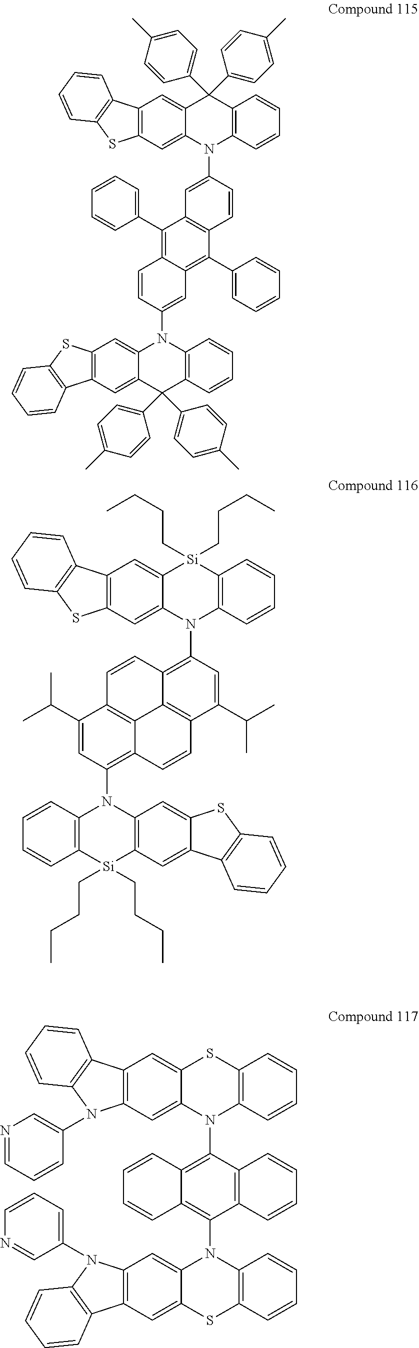

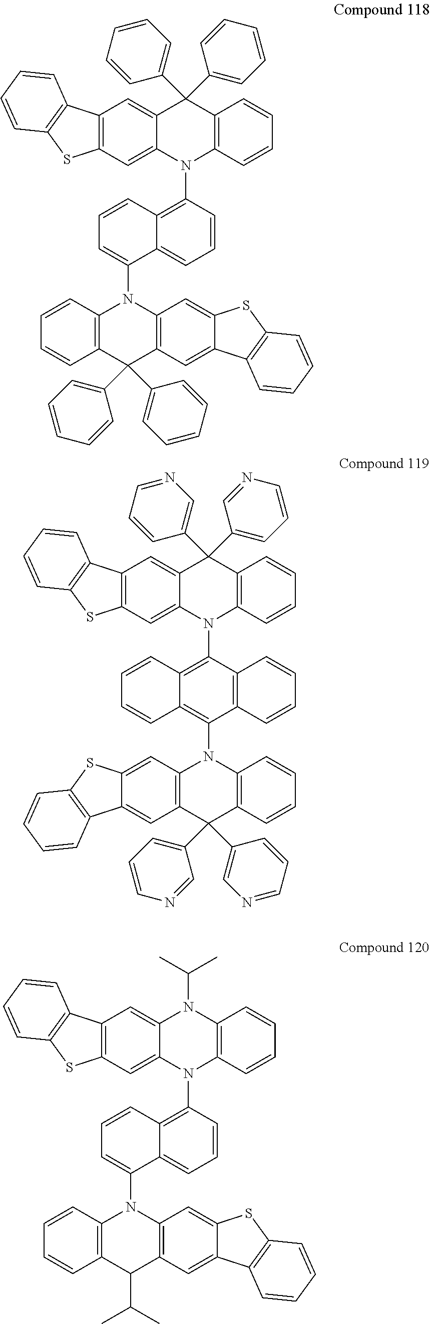

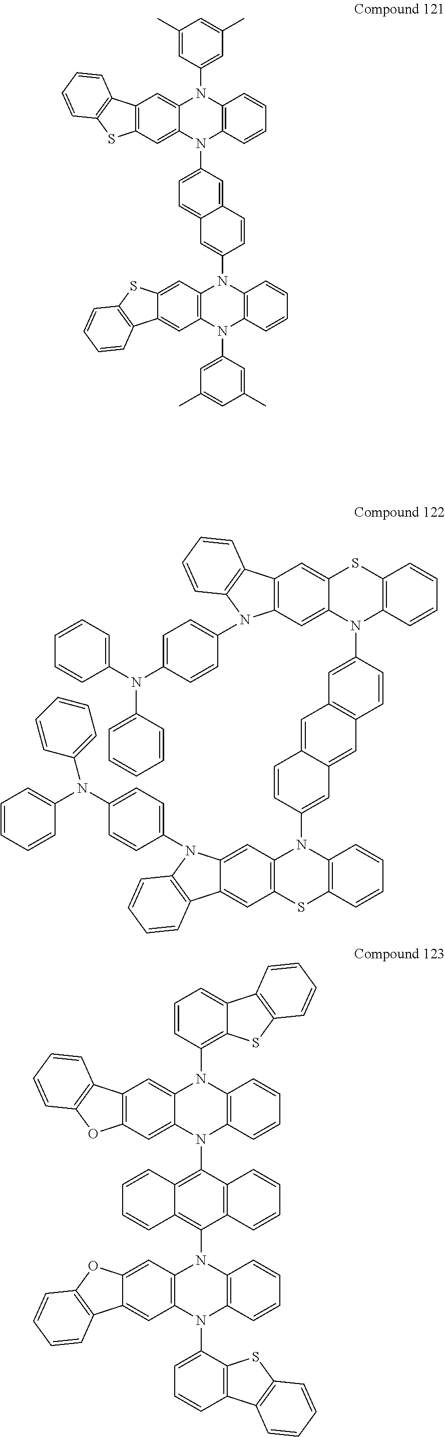 [SMILES: Cc1cc(C)cc(N2c3ccccc3N(c3ccc4cc(N5c6ccccc6N(c6cc(C)cc(C)c6)c6cc7c(cc65)sc5ccccc57)ccc4c3)c3cc4sc5ccccc5c4cc32)c1 ; c1ccc(N(c2ccccc2)c2ccc(-n3c4ccccc4c4cc5c(cc43)N(c3ccc4cc6cc(N7c8ccccc8Sc8cc9c%10ccccc%10n(-c%10ccc(N(c%11ccccc%11)c%11ccccc%11)cc%10)c9cc87)ccc6cc4c3)c3ccccc3S5)cc2)cc1 ; c1ccc2c(c1)N(c1c3ccccc3c(N3c4ccccc4N(c4cccc5c4sc4ccccc45)c4cc5c(cc43)oc3ccccc35)c3ccccc13)c1cc3oc4ccccc4c3cc1N2c1cccc2c1sc1ccccc12]

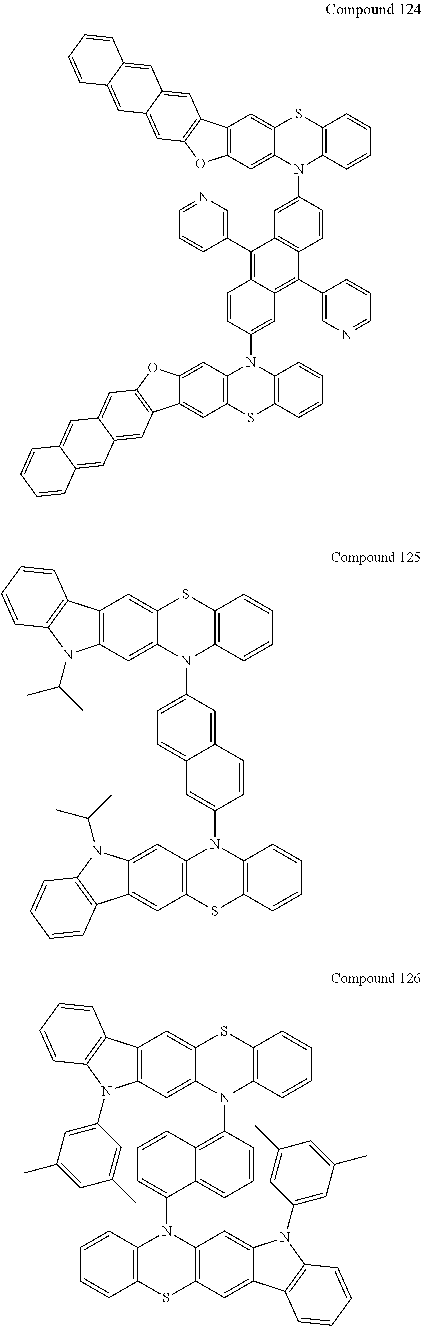

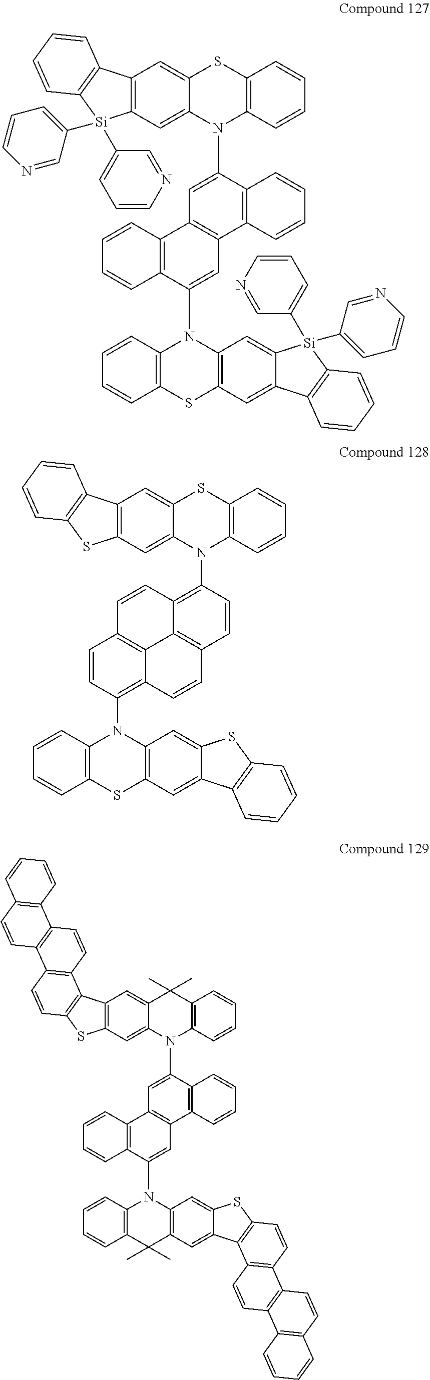 [SMILES: c1cncc([Si]2(c3cccnc3)c3ccccc3-c3cc4c(cc32)N(c2cc3c5ccccc5c(N5c6ccccc6Sc6cc7c(cc65)[Si](c5cccnc5)(c5cccnc5)c5ccccc5-7)cc3c3ccccc23)c2ccccc2S4)c1 ; c1ccc2c(c1)Sc1cc3c(cc1N2c1ccc2ccc4c(N5c6ccccc6Sc6cc7c(cc65)sc5ccccc57)ccc5ccc1c2c54)sc1ccccc13 ; CC1(C)c2ccccc2N(c2cc3c4ccccc4c(N4c5ccccc5C(C)(C)c5cc6c(cc54)sc4ccc5c7ccc8ccccc8c7ccc5c46)cc3c3ccccc23)c2cc3sc4ccc5c6ccc7ccccc7c6ccc5c4c3cc21]

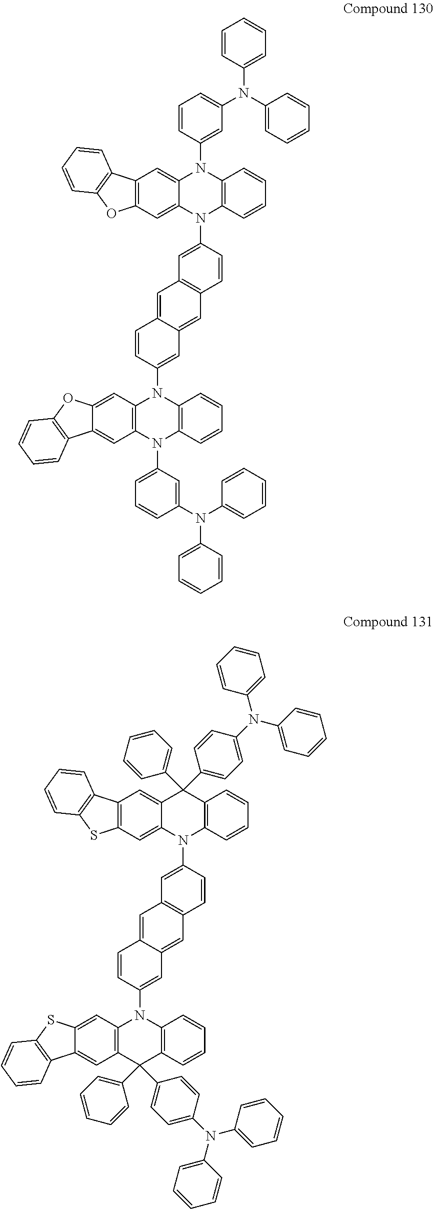 [SMILES: c1ccc(N(c2ccccc2)c2cccc(N3c4ccccc4N(c4ccc5cc6cc(N7c8ccccc8N(c8cccc(N(c9ccccc9)c9ccccc9)c8)c8cc9c(cc87)oc7ccccc79)ccc6cc5c4)c4cc5oc6ccccc6c5cc43)c2)cc1 ; c1ccc(N(c2ccccc2)c2ccc(C3(c4ccccc4)c4ccccc4N(c4ccc5cc6cc(N7c8ccccc8C(c8ccccc8)(c8ccc(N(c9ccccc9)c9ccccc9)cc8)c8cc9c(cc87)sc7ccccc79)ccc6cc5c4)c4cc5sc6ccccc6c5cc43)cc2)cc1]

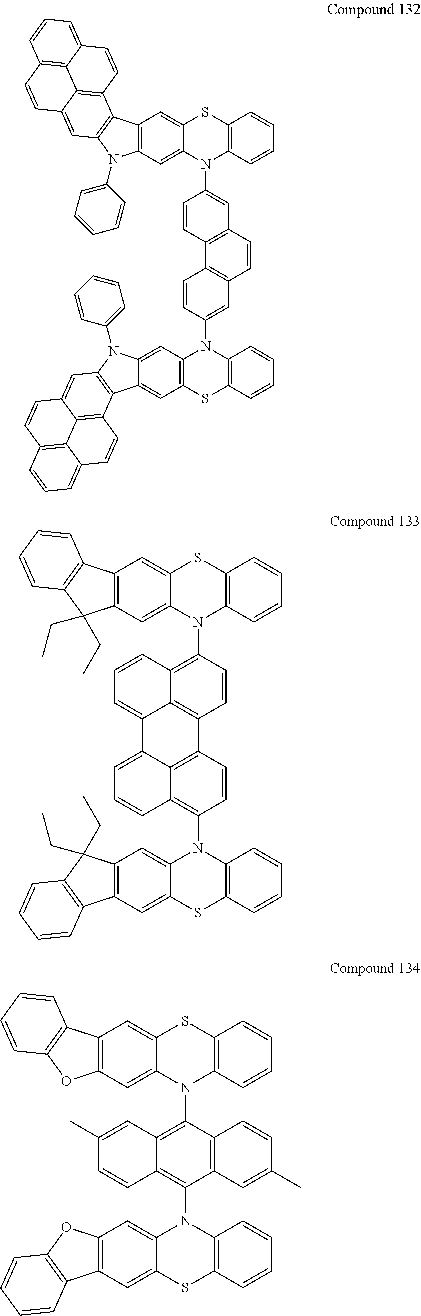

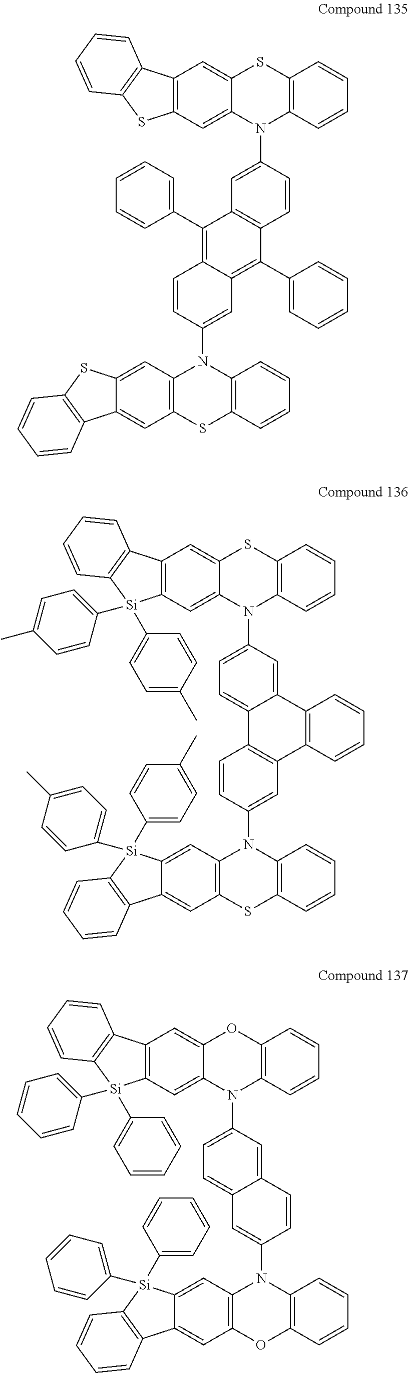 [SMILES: c1ccc(-c2c3ccc(N4c5ccccc5Sc5cc6c(cc54)sc4ccccc46)cc3c(-c3ccccc3)c3ccc(N4c5ccccc5Sc5cc6c(cc54)sc4ccccc46)cc23)cc1 ; Cc1ccc([Si]2(c3ccc(C)cc3)c3ccccc3-c3cc4c(cc32)N(c2ccc3c5ccc(N6c7ccccc7Sc7cc8c(cc76)[Si](c6ccc(C)cc6)(c6ccc(C)cc6)c6ccccc6-8)cc5c5ccccc5c3c2)c2ccccc2S4)cc1 ; c1ccc([Si]2(c3ccccc3)c3ccccc3-c3cc4c(cc32)N(c2ccc3cc(N5c6ccccc6Oc6cc7c(cc65)[Si](c5ccccc5)(c5ccccc5)c5ccccc5-7)ccc3c2)c2ccccc2O4)cc1]

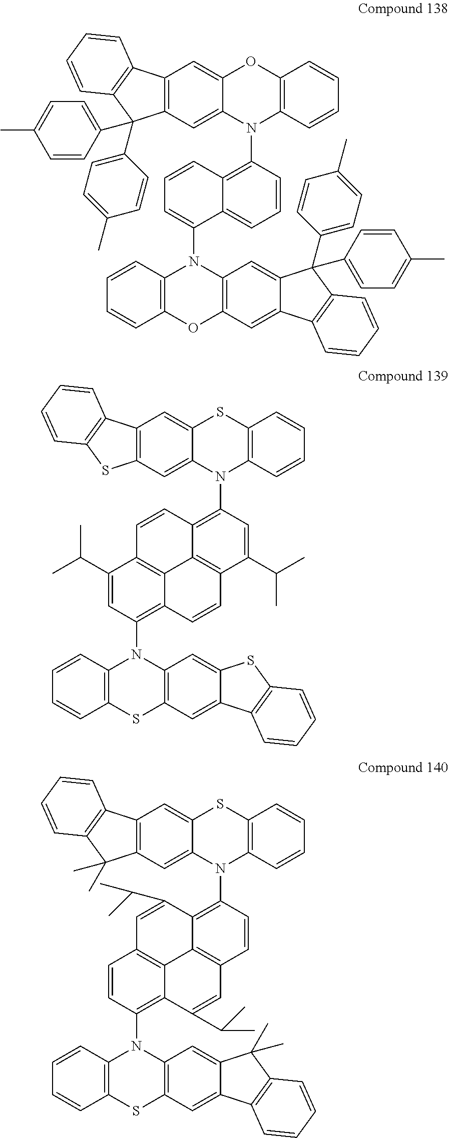

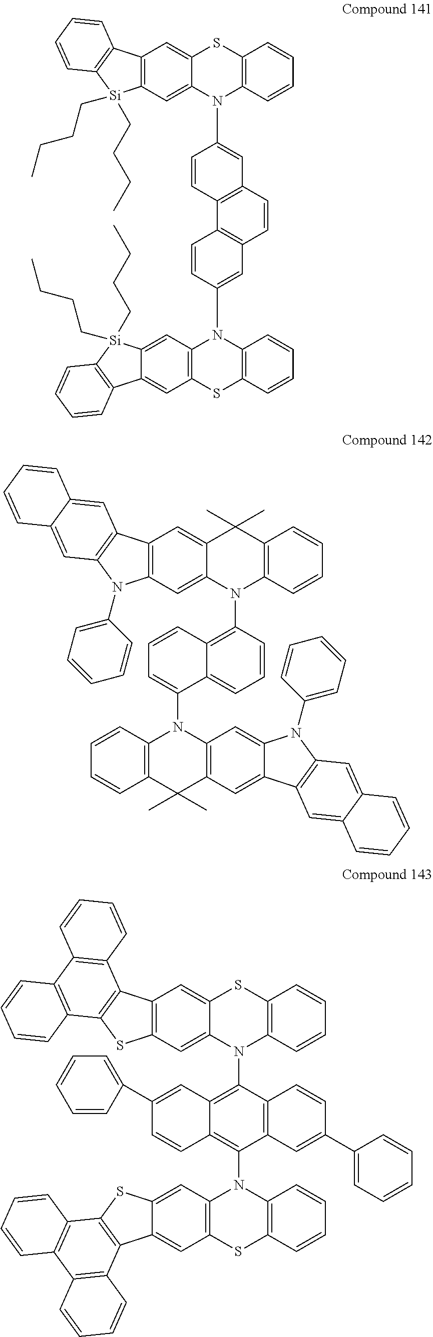 [SMILES: CCCC[Si]1(CCCC)c2ccccc2-c2cc3c(cc21)N(c1ccc2c(ccc4cc(N5c6ccccc6Sc6cc7c(cc65)[Si](CCCC)(CCCC)c5ccccc5-7)ccc42)c1)c1ccccc1S3 ; CC1(C)c2ccccc2N(c2cccc3c(N4c5ccccc5C(C)(C)c5cc6c7cc8ccccc8cc7n(-c7ccccc7)c6cc54)cccc23)c2cc3c(cc21)c1cc2ccccc2cc1n3-c1ccccc1 ; c1ccc(-c2ccc3c(N4c5ccccc5Sc5cc6c(cc54)sc4c5ccccc5c5ccccc5c64)c4cc(-c5ccccc5)ccc4c(N4c5ccccc5Sc5cc6c(cc54)sc4c5ccccc5c5ccccc5c64)c3c2)cc1]

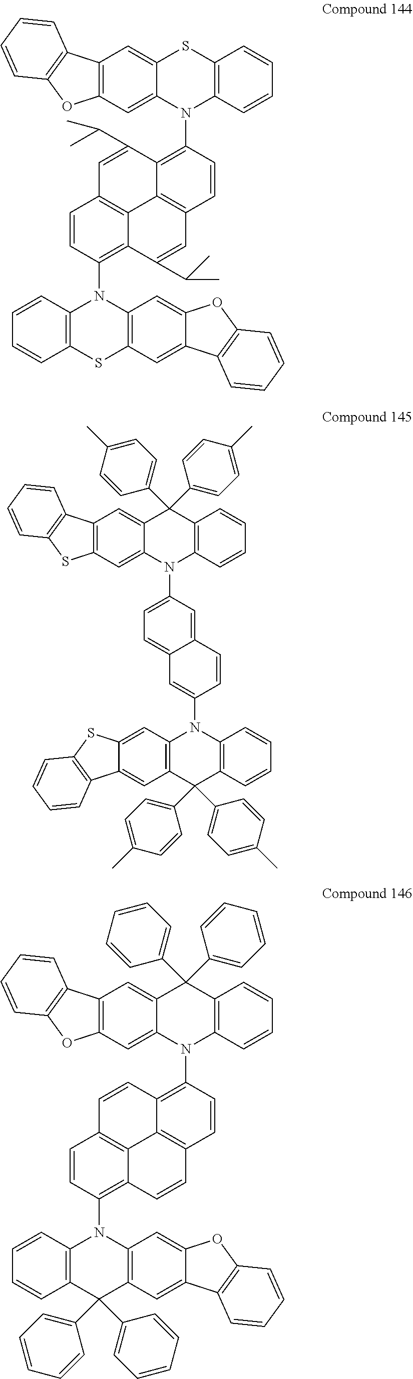

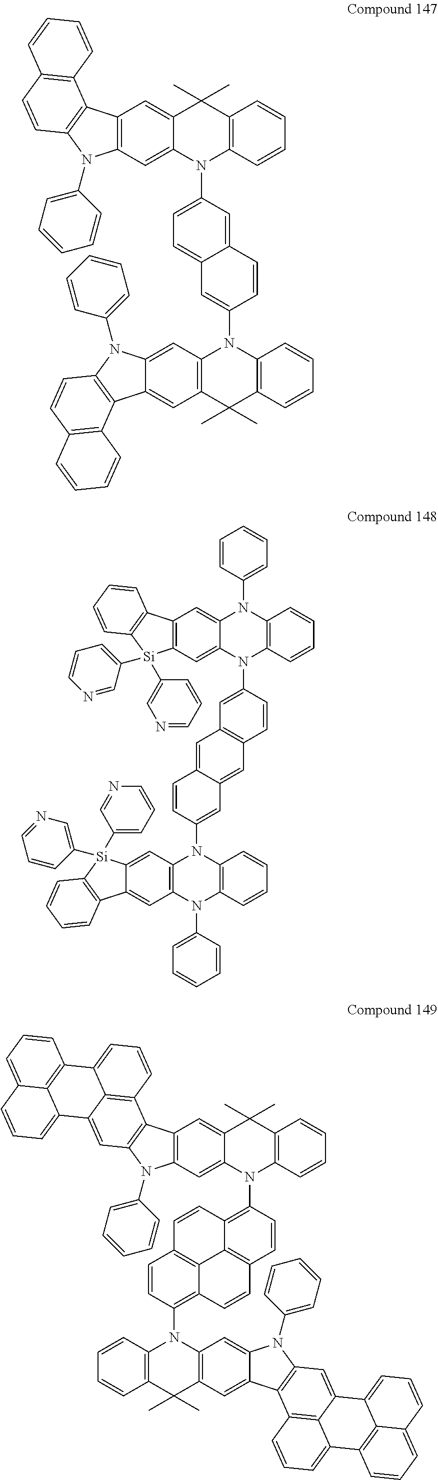 [SMILES: CC1(C)c2ccccc2N(c2ccc3cc(N4c5ccccc5C(C)(C)c5cc6c7c8ccccc8ccc7n(-c7ccccc7)c6cc54)ccc3c2)c2cc3c(cc21)c1c2ccccc2ccc1n3-c1ccccc1 ; c1ccc(N2c3ccccc3N(c3ccc4cc5cc(N6c7ccccc7N(c7ccccc7)c7cc8c(cc76)[Si](c6cccnc6)(c6cccnc6)c6ccccc6-8)ccc5cc4c3)c3cc4c(cc32)-c2ccccc2[Si]4(c2cccnc2)c2cccnc2)cc1 ; CC1(C)c2ccccc2N(c2ccc3ccc4c(N5c6ccccc6C(C)(C)c6cc7c8c9cccc%10c%11cccc%12cccc(c(cc8n(-c8ccccc8)c7cc65)c%109)c%12%11)ccc5ccc2c3c54)c2cc3c(cc21)c1c2cccc4c5cccc6cccc(c(cc1n3-c1ccccc1)c42)c65]

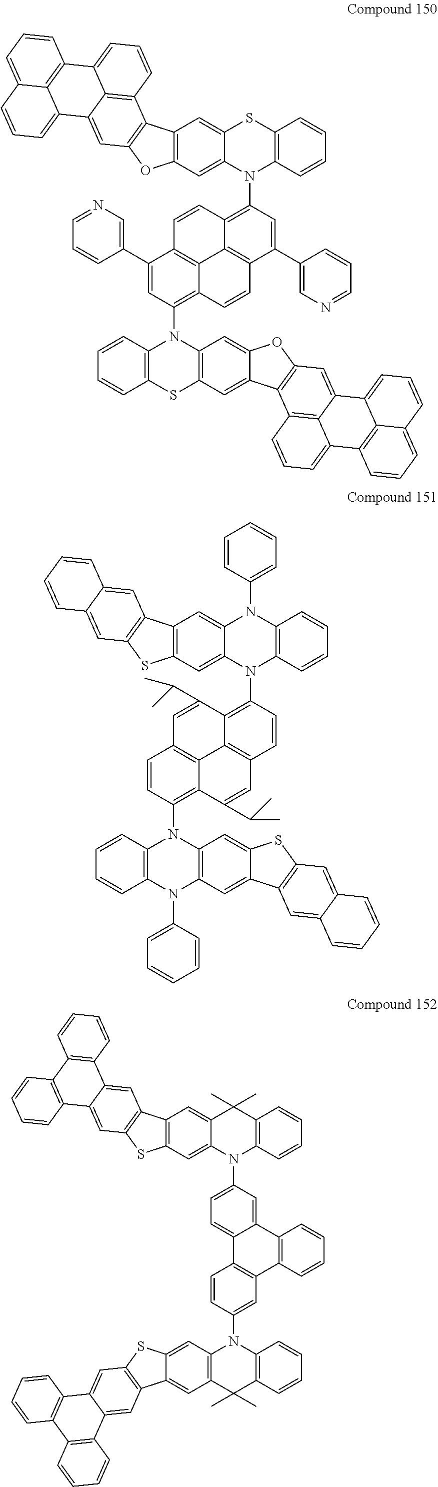 [SMILES: c1cncc(-c2cc(-n3c4ccccc4sc4cc5c(cc43)oc3cc4c6cccc7cccc(c8cccc(c35)c84)c76)c3ccc4c(-c5cccnc5)cc(-n5c6ccccc6sc6cc7c(cc65)oc5cc6c8cccc9cccc(c%10cccc(c57)c%106)c98)c5ccc2c3c45)c1 ; CC(C)c1cc2ccc(N3c4ccccc4N(c4ccccc4)c4cc5c(cc43)sc3cc4ccccc4cc35)c3c(C(C)C)cc4ccc(N5c6ccccc6N(c6ccccc6)c6cc7c(cc65)sc5cc6ccccc6cc57)c1c4c23 ; CC1(C)c2ccccc2N(c2ccc3c4ccc(N5c6ccccc6C(C)(C)c6cc7c(cc65)sc5cc6c8ccccc8c8ccccc8c6cc57)cc4c4ccccc4c3c2)c2cc3sc4cc5c6ccccc6c6ccccc6c5cc4c3cc21]

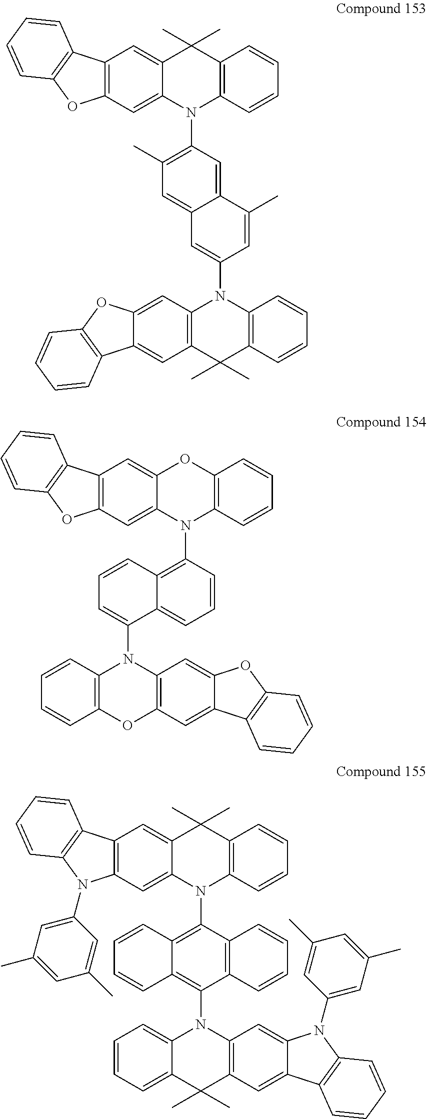

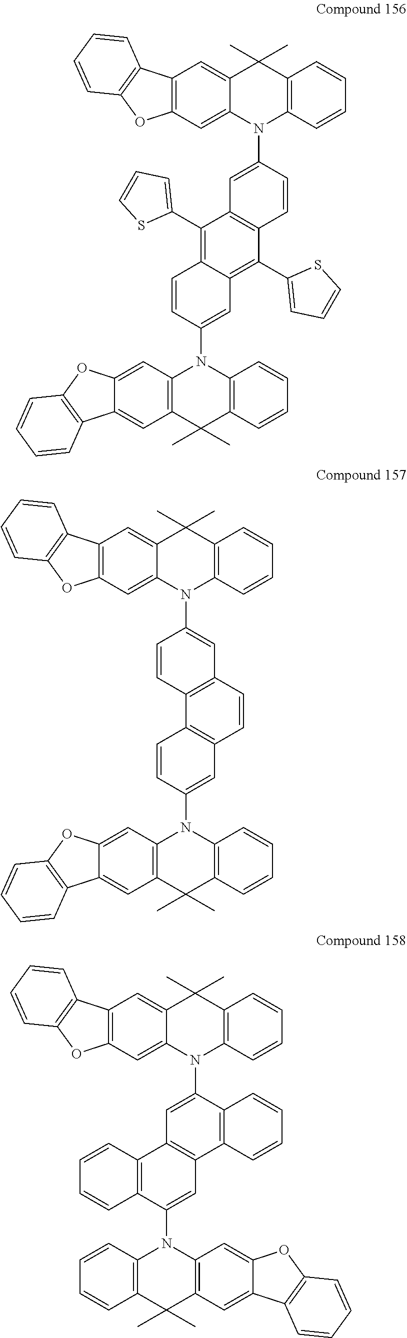

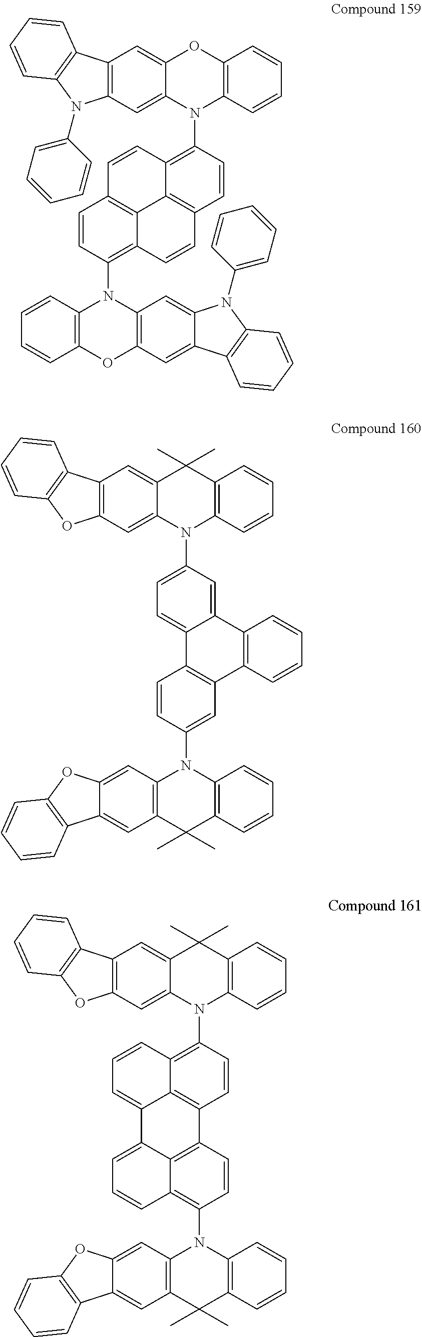 [SMILES: c1ccc(-n2c3ccccc3c3cc4c(cc32)N(c2ccc3ccc5c(N6c7ccccc7Oc7cc8c9ccccc9n(-c9ccccc9)c8cc76)ccc6ccc2c3c65)c2ccccc2O4)cc1 ; CC1(C)c2ccccc2N(c2ccc3c4ccc(N5c6ccccc6C(C)(C)c6cc7c(cc65)oc5ccccc57)cc4c4ccccc4c3c2)c2cc3oc4ccccc4c3cc21 ; CC1(C)c2ccccc2N(c2ccc3c4ccc(N5c6ccccc6C(C)(C)c6cc7c(cc65)oc5ccccc57)c5cccc(c6cccc2c63)c54)c2cc3oc4ccccc4c3cc21]

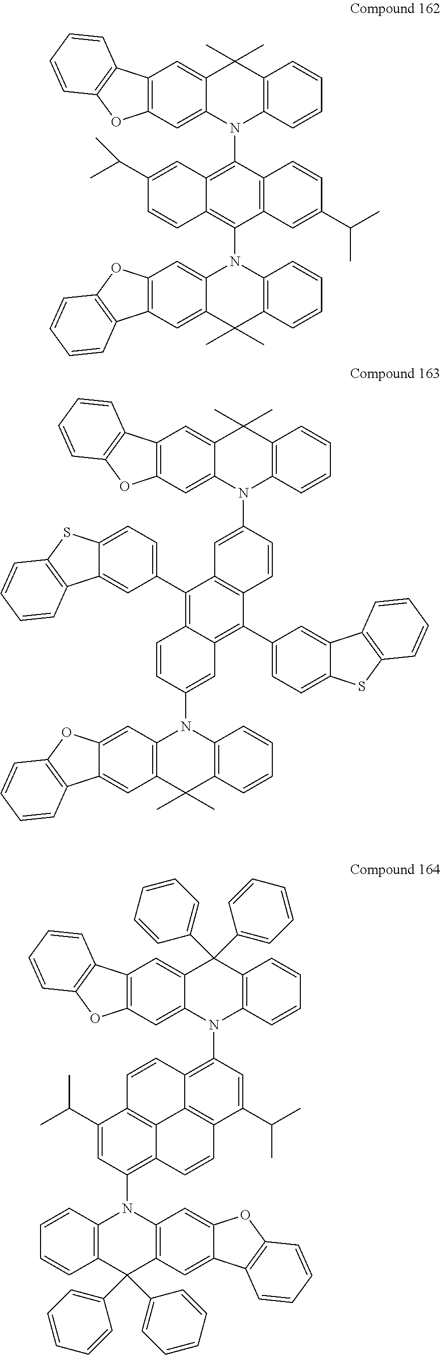

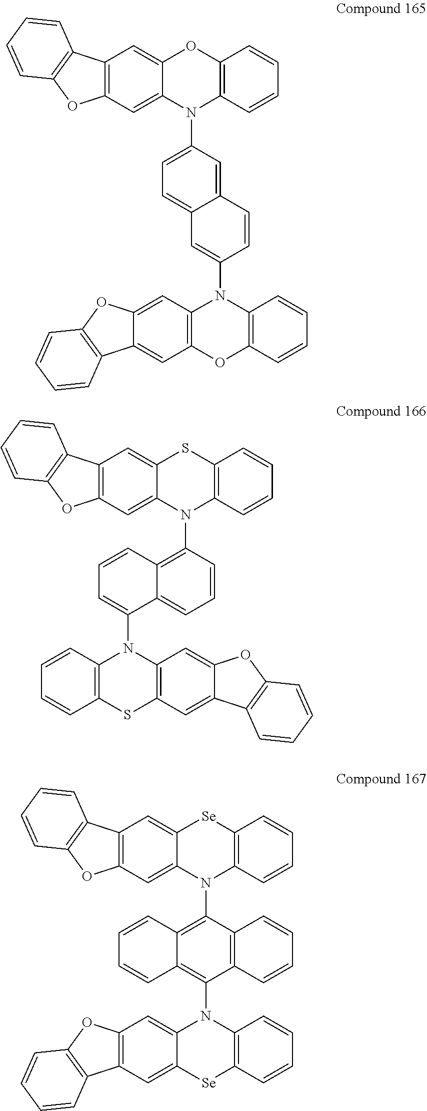

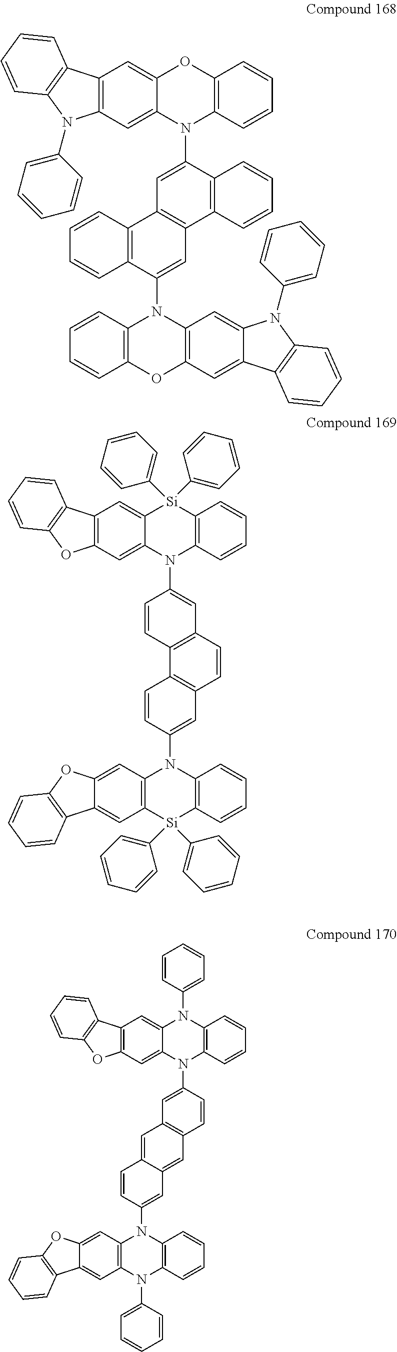 [SMILES: c1ccc(-n2c3ccccc3c3cc4c(cc32)N(c2cc3c5ccccc5c(N5c6ccccc6Oc6cc7c8ccccc8n(-c8ccccc8)c7cc65)cc3c3ccccc23)c2ccccc2O4)cc1 ; c1ccc([Si]2(c3ccccc3)c3ccccc3N(c3ccc4c(ccc5cc(N6c7ccccc7[Si](c7ccccc7)(c7ccccc7)c7cc8c(cc76)oc6ccccc68)ccc54)c3)c3cc4oc5ccccc5c4cc32)cc1 ; c1ccc(N2c3ccccc3N(c3ccc4cc5cc(N6c7ccccc7N(c7ccccc7)c7cc8c(cc76)oc6ccccc68)ccc5cc4c3)c3cc4oc5ccccc5c4cc32)cc1]

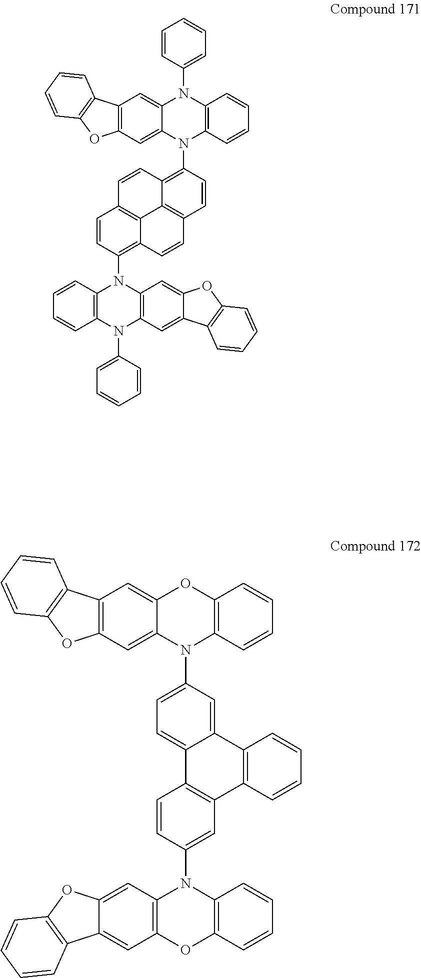 [SMILES: c1ccc(N2c3ccccc3N(c3ccc4ccc5c(N6c7ccccc7N(c7ccccc7)c7cc8c(cc76)oc6ccccc68)ccc6ccc3c4c65)c3cc4oc5ccccc5c4cc32)cc1 ; c1ccc2c(c1)Oc1cc3c(cc1N2c1ccc2c4ccc(N5c6ccccc6Oc6cc7c(cc65)oc5ccccc57)cc4c4ccccc4c2c1)oc1ccccc13]

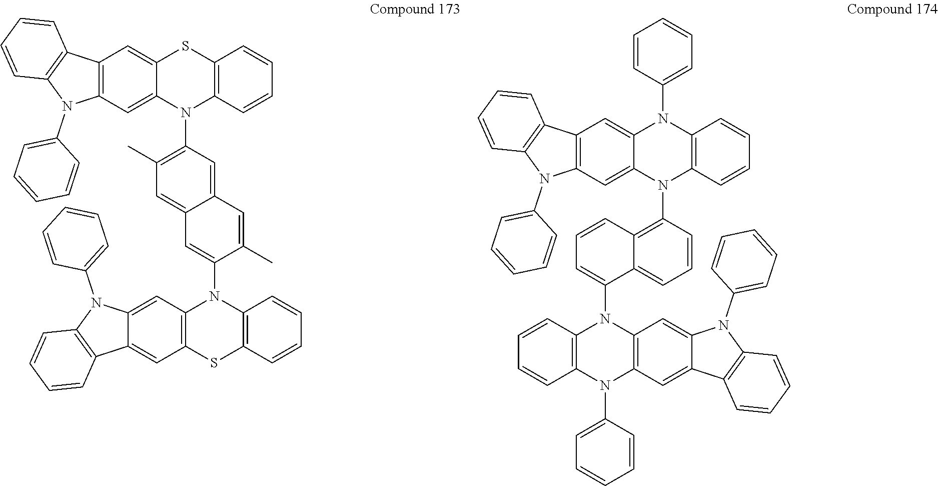 [SMILES: Cc1cc2cc(N3c4ccccc4Sc4cc5c6ccccc6n(-c6ccccc6)c5cc43)c(C)cc2cc1N1c2ccccc2Sc2cc3c4ccccc4n(-c4ccccc4)c3cc21 ; c1ccc(N2c3ccccc3N(c3cccc4c(N5c6ccccc6N(c6ccccc6)c6cc7c8ccccc8n(-c8ccccc8)c7cc65)cccc34)c3cc4c(cc32)c2ccccc2n4-c2ccccc2)cc1]

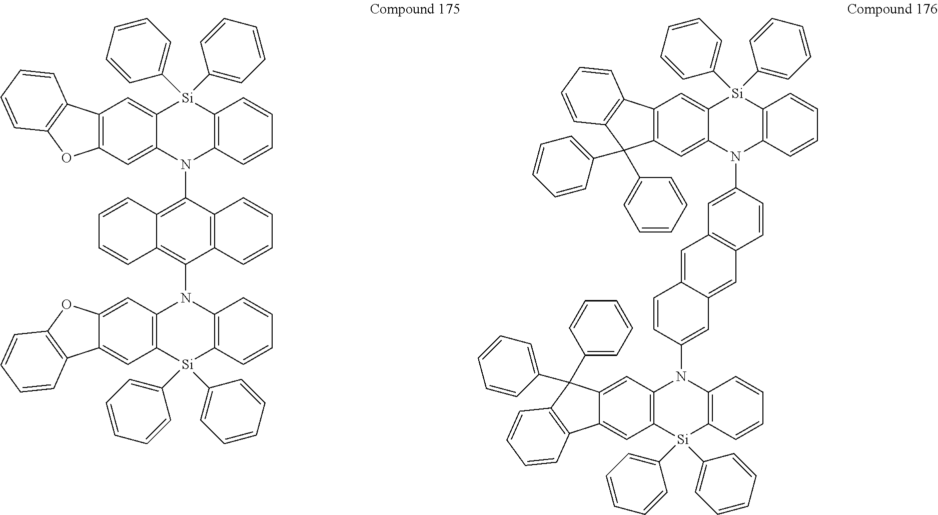 [SMILES: c1ccc([Si]2(c3ccccc3)c3ccccc3N(c3c4ccccc4c(N4c5ccccc5[Si](c5ccccc5)(c5ccccc5)c5cc6c(cc54)oc4ccccc46)c4ccccc34)c3cc4oc5ccccc5c4cc32)cc1 ; c1ccc(C2(c3ccccc3)c3ccccc3-c3cc4c(cc32)N(c2ccc3cc5cc(N6c7ccccc7[Si](c7ccccc7)(c7ccccc7)c7cc8c(cc76)C(c6ccccc6)(c6ccccc6)c6ccccc6-8)ccc5cc3c2)c2ccccc2[Si]4(c2ccccc2)c2ccccc2)cc1]

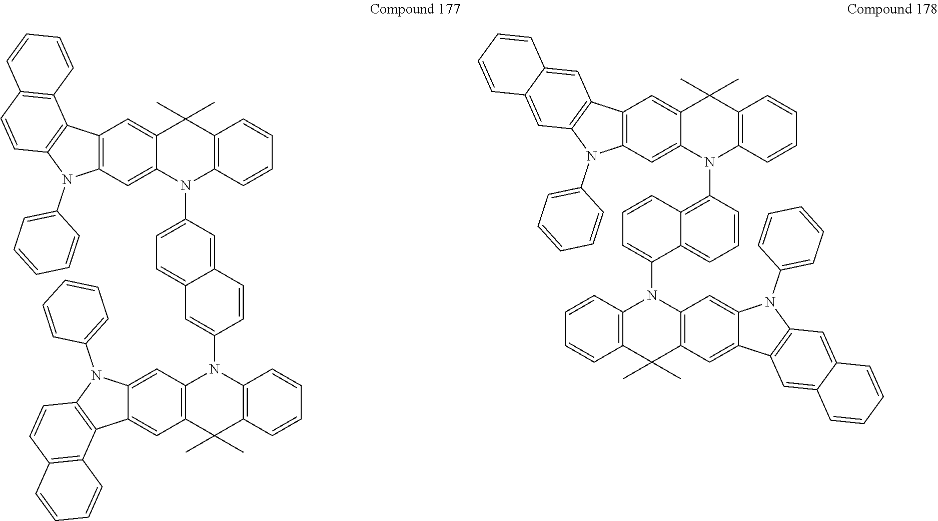 [SMILES: CC1(C)c2ccccc2N(c2ccc3cc(N4c5ccccc5C(C)(C)c5cc6c7c8ccccc8ccc7n(-c7ccccc7)c6cc54)ccc3c2)c2cc3c(cc21)c1c2ccccc2ccc1n3-c1ccccc1 ; CC1(C)c2ccccc2N(c2cccc3c(N4c5ccccc5C(C)(C)c5cc6c7cc8ccccc8cc7n(-c7ccccc7)c6cc54)cccc23)c2cc3c(cc21)c1cc2ccccc2cc1n3-c1ccccc1]

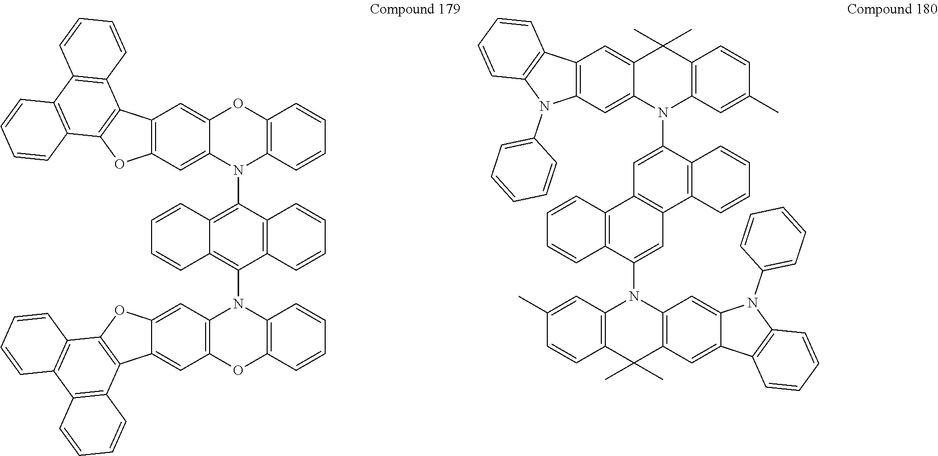 [SMILES: c1ccc2c(c1)Oc1cc3c(cc1N2c1c2ccccc2c(N2c4ccccc4Oc4cc5c(cc42)oc2c4ccccc4c4ccccc4c52)c2ccccc12)oc1c2ccccc2c2ccccc2c31 ; Cc1ccc2c(c1)N(c1cc3c4ccccc4c(N4c5cc(C)ccc5C(C)(C)c5cc6c7ccccc7n(-c7ccccc7)c6cc54)cc3c3ccccc13)c1cc3c(cc1C2(C)C)c1ccccc1n3-c1ccccc1]

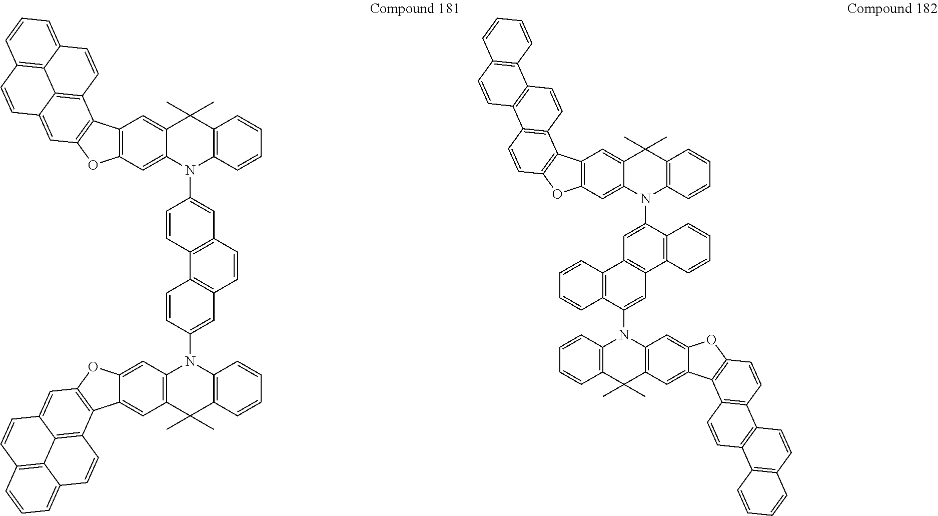 [SMILES: CC1(C)c2ccccc2N(c2ccc3c(ccc4cc(N5c6ccccc6C(C)(C)c6cc7c(cc65)oc5cc6ccc8cccc9ccc(c57)c6c89)ccc43)c2)c2cc3oc4cc5ccc6cccc7ccc(c4c3cc21)c5c67 ; CC1(C)c2ccccc2N(c2cc3c4ccccc4c(N4c5ccccc5C(C)(C)c5cc6c(cc54)oc4ccc5c7ccc8ccccc8c7ccc5c46)cc3c3ccccc23)c2cc3oc4ccc5c6ccc7ccccc7c6ccc5c4c3cc21]

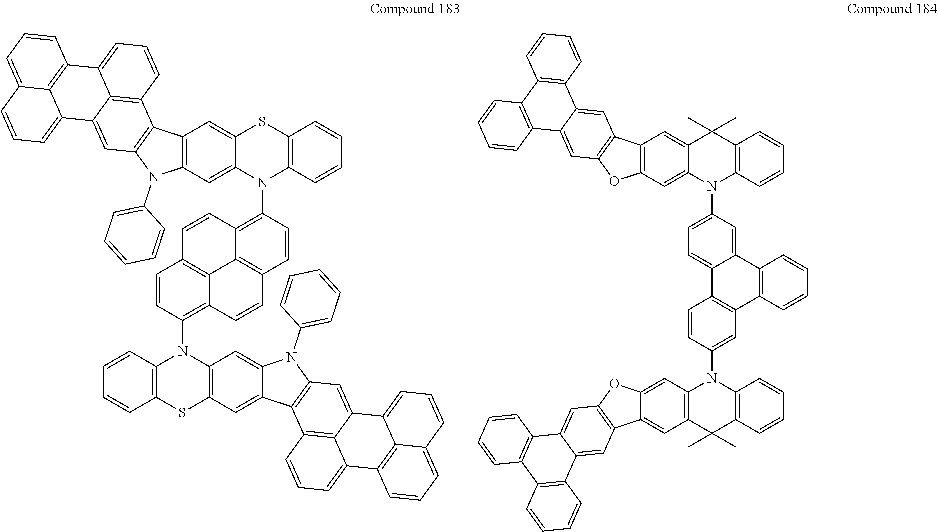

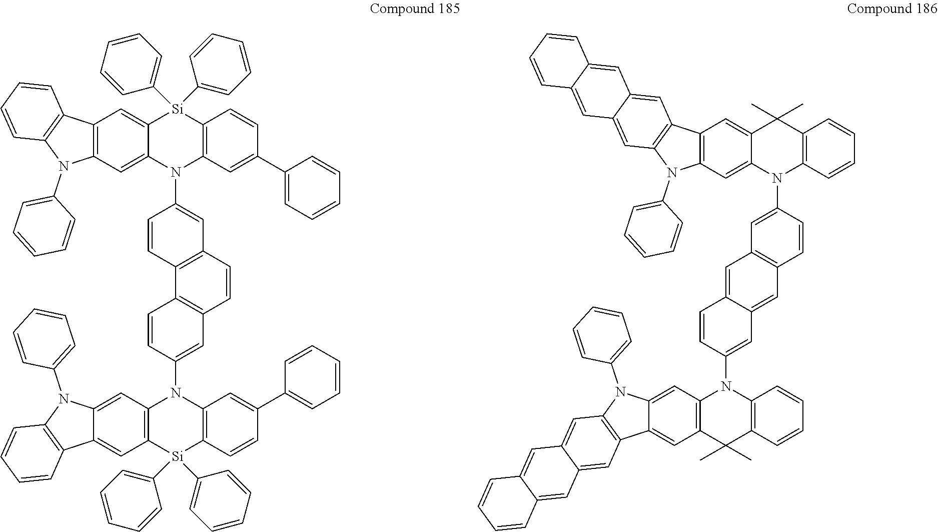 [SMILES: c1ccc(-c2ccc3c(c2)N(c2ccc4c(ccc5cc(N6c7cc(-c8ccccc8)ccc7[Si](c7ccccc7)(c7ccccc7)c7cc8c9ccccc9n(-c9ccccc9)c8cc76)ccc54)c2)c2cc4c(cc2[Si]3(c2ccccc2)c2ccccc2)c2ccccc2n4-c2ccccc2)cc1 ; CC1(C)c2ccccc2N(c2ccc3cc4cc(N5c6ccccc6C(C)(C)c6cc7c8cc9cc%10ccccc%10cc9cc8n(-c8ccccc8)c7cc65)ccc4cc3c2)c2cc3c(cc21)c1cc2cc4ccccc4cc2cc1n3-c1ccccc1]

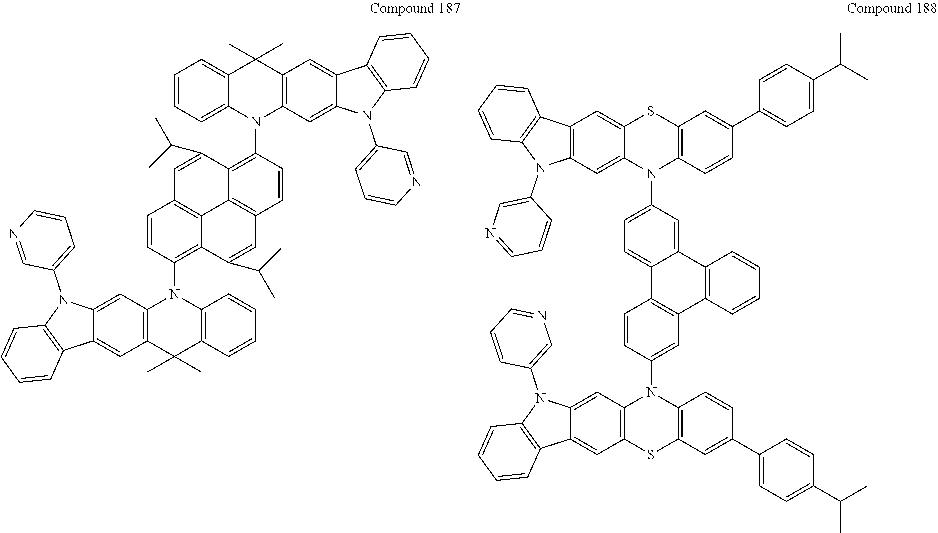 [SMILES: CC(C)c1cc2ccc(N3c4ccccc4C(C)(C)c4cc5c6ccccc6n(-c6cccnc6)c5cc43)c3c(C(C)C)cc4ccc(N5c6ccccc6C(C)(C)c6cc7c8ccccc8n(-c8cccnc8)c7cc65)c1c4c23 ; CC(C)c1ccc(-c2ccc3c(c2)Sc2cc4c5ccccc5n(-c5cccnc5)c4cc2N3c2ccc3c4ccc(N5c6ccc(-c7ccc(C(C)C)cc7)cc6Sc6cc7c8ccccc8n(-c8cccnc8)c7cc65)cc4c4ccccc4c3c2)cc1]

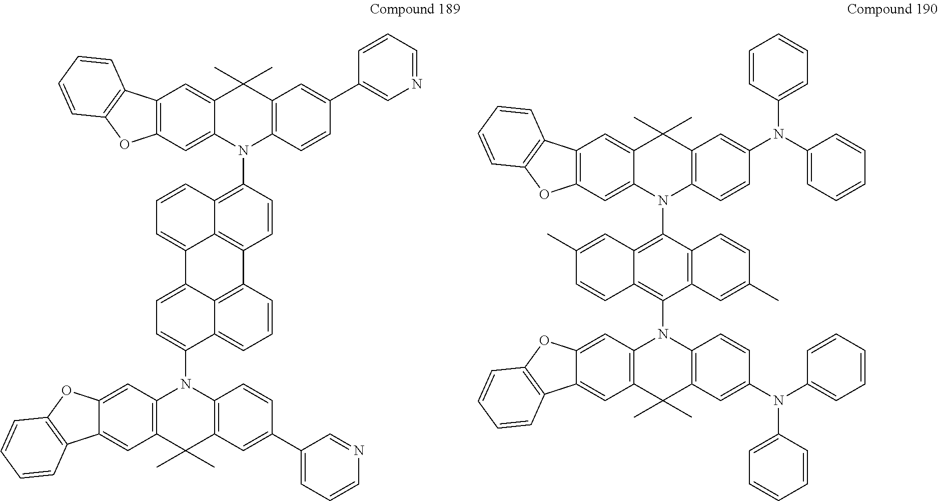 [SMILES: CC1(C)c2cc(-c3cccnc3)ccc2N(c2ccc3c4cccc5c(N6c7ccc(-c8cccnc8)cc7C(C)(C)c7cc8c(cc76)oc6ccccc68)ccc(c6cccc2c63)c54)c2cc3oc4ccccc4c3cc21 ; Cc1ccc2c(N3c4ccc(N(c5ccccc5)c5ccccc5)cc4C(C)(C)c4cc5c(cc43)oc3ccccc35)c3cc(C)ccc3c(N3c4ccc(N(c5ccccc5)c5ccccc5)cc4C(C)(C)c4cc5c(cc43)oc3ccccc35)c2c1]

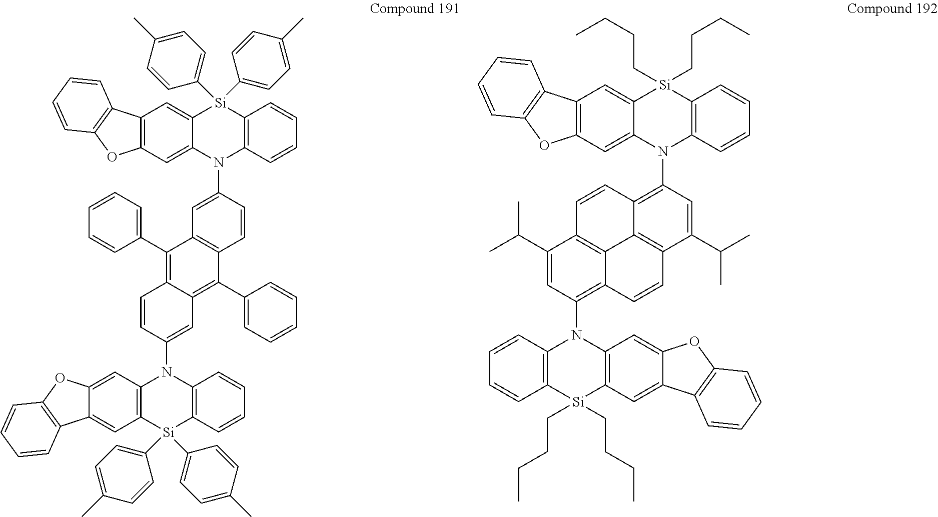 [SMILES: Cc1ccc([Si]2(c3ccc(C)cc3)c3ccccc3N(c3ccc4c(-c5ccccc5)c5cc(N6c7ccccc7[Si](c7ccc(C)cc7)(c7ccc(C)cc7)c7cc8c(cc76)oc6ccccc68)ccc5c(-c5ccccc5)c4c3)c3cc4oc5ccccc5c4cc32)cc1 ; CCCC[Si]1(CCCC)c2ccccc2N(c2cc(C(C)C)c3ccc4c(N5c6ccccc6[Si](CCCC)(CCCC)c6cc7c(cc65)oc5ccccc57)cc(C(C)C)c5ccc2c3c54)c2cc3oc4ccccc4c3cc21]

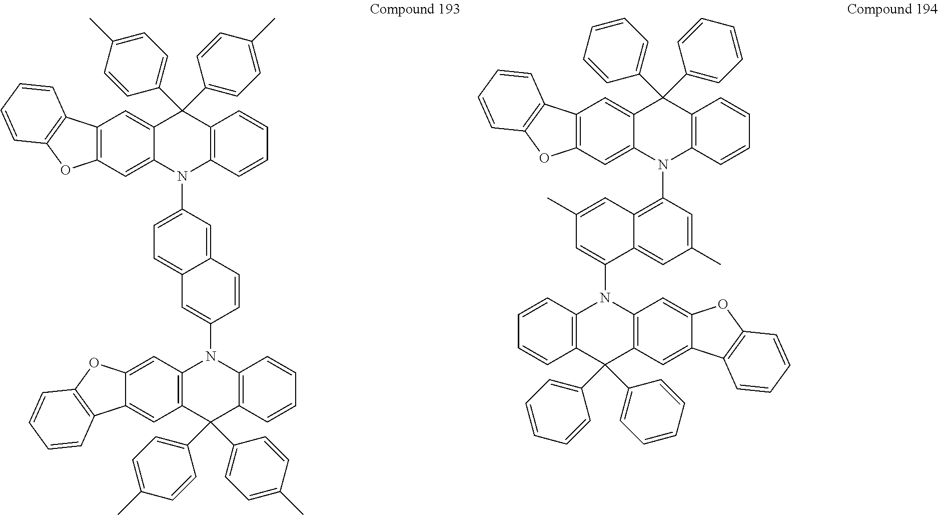 [SMILES: Cc1ccc(C2(c3ccc(C)cc3)c3ccccc3N(c3ccc4cc(N5c6ccccc6C(c6ccc(C)cc6)(c6ccc(C)cc6)c6cc7c(cc65)oc5ccccc57)ccc4c3)c3cc4oc5ccccc5c4cc32)cc1 ; Cc1cc(N2c3ccccc3C(c3ccccc3)(c3ccccc3)c3cc4c(cc32)oc2ccccc24)c2cc(C)cc(N3c4ccccc4C(c4ccccc4)(c4ccccc4)c4cc5c(cc43)oc3ccccc35)c2c1]

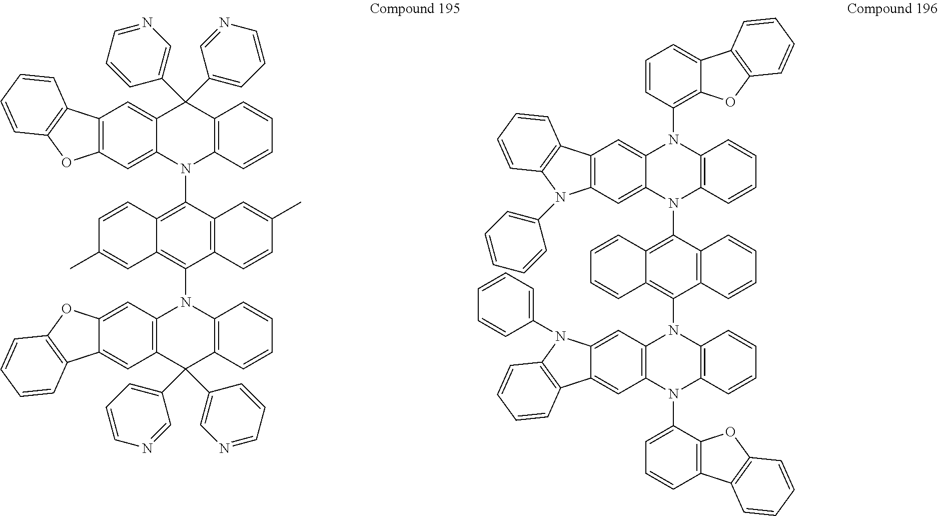 [SMILES: Cc1ccc2c(N3c4ccccc4C(c4cccnc4)(c4cccnc4)c4cc5c(cc43)oc3ccccc35)c3cc(C)ccc3c(N3c4ccccc4C(c4cccnc4)(c4cccnc4)c4cc5c(cc43)oc3ccccc35)c2c1 ; c1ccc(-n2c3ccccc3c3cc4c(cc32)N(c2c3ccccc3c(N3c5ccccc5N(c5cccc6c5oc5ccccc56)c5cc6c7ccccc7n(-c7ccccc7)c6cc53)c3ccccc23)c2ccccc2N4c2cccc3c2oc2ccccc23)cc1]

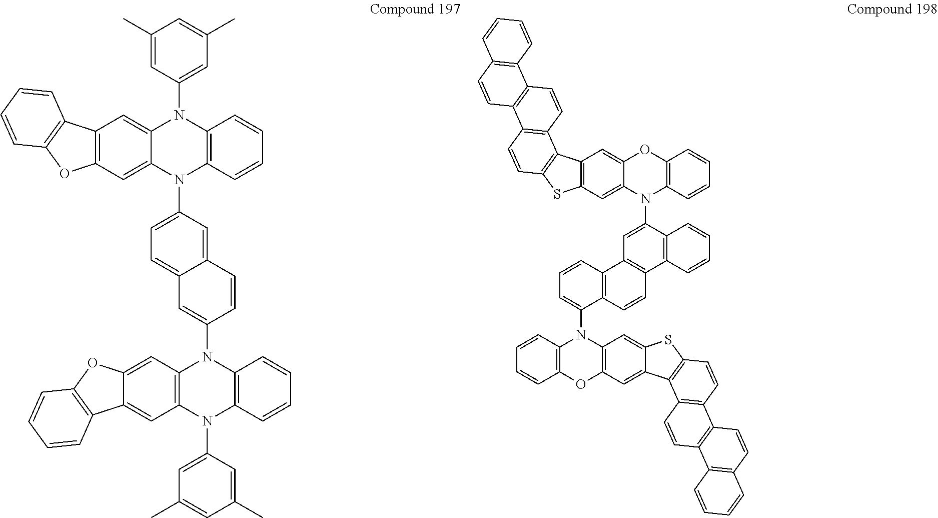 [SMILES: Cc1cc(C)cc(N2c3ccccc3N(c3ccc4cc(N5c6ccccc6N(c6cc(C)cc(C)c6)c6cc7c(cc65)oc5ccccc57)ccc4c3)c3cc4oc5ccccc5c4cc32)c1 ; c1ccc2c(c1)Oc1cc3c(cc1N2c1cc2c4cccc(N5c6ccccc6Oc6cc7c(cc65)sc5ccc6c8ccc9ccccc9c8ccc6c57)c4ccc2c2ccccc12)sc1ccc2c4ccc5ccccc5c4ccc2c13]

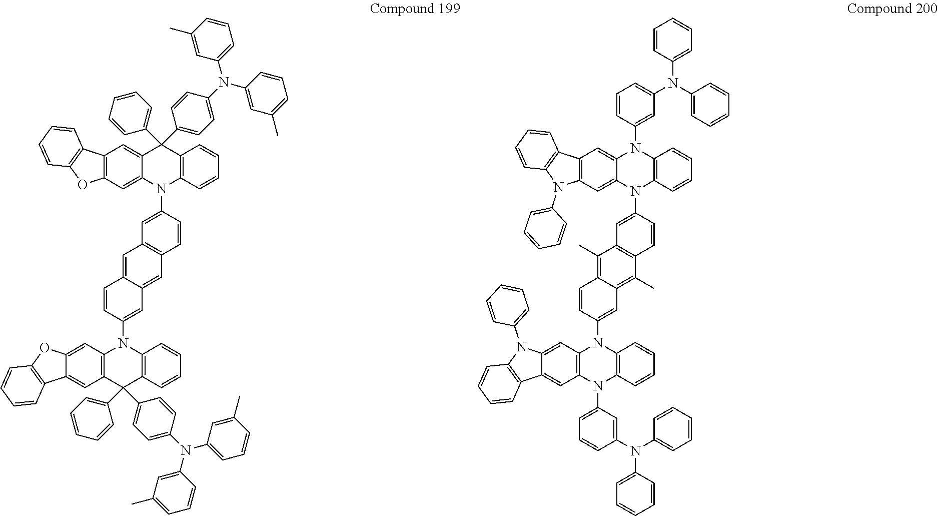 [SMILES: Cc1cccc(N(c2ccc(C3(c4ccccc4)c4ccccc4N(c4ccc5cc6cc(N7c8ccccc8C(c8ccccc8)(c8ccc(N(c9cccc(C)c9)c9cccc(C)c9)cc8)c8cc9c(cc87)oc7ccccc79)ccc6cc5c4)c4cc5oc6ccccc6c5cc43)cc2)c2cccc(C)c2)c1 ; Cc1c2ccc(N3c4ccccc4N(c4cccc(N(c5ccccc5)c5ccccc5)c4)c4cc5c6ccccc6n(-c6ccccc6)c5cc43)cc2c(C)c2ccc(N3c4ccccc4N(c4cccc(N(c5ccccc5)c5ccccc5)c4)c4cc5c6ccccc6n(-c6ccccc6)c5cc43)cc12]

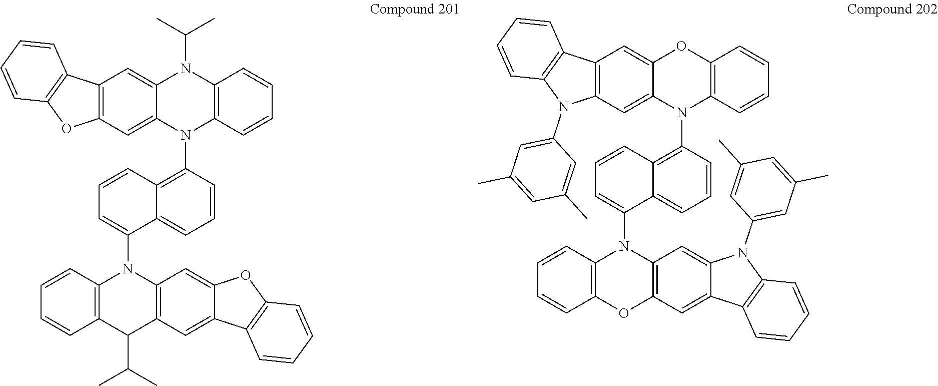

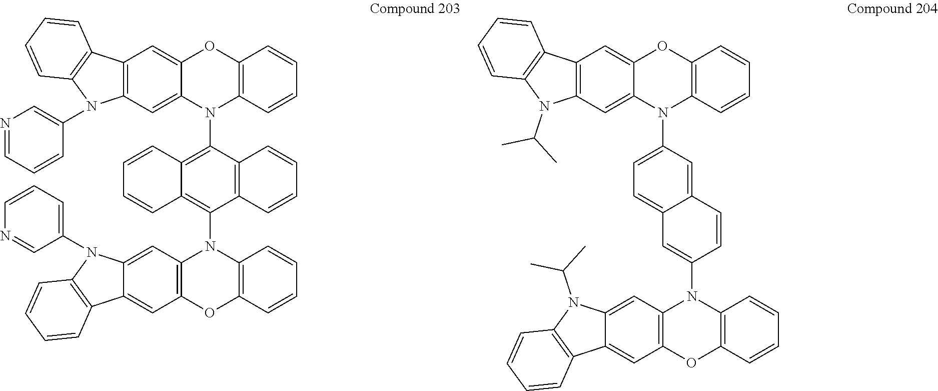 [SMILES: c1cncc(-n2c3ccccc3c3cc4c(cc32)N(c2c3ccccc3c(N3c5ccccc5Oc5cc6c7ccccc7n(-c7cccnc7)c6cc53)c3ccccc23)c2ccccc2O4)c1 ; CC(C)n1c2ccccc2c2cc3c(cc21)N(c1ccc2cc(N4c5ccccc5Oc5cc6c7ccccc7n(C(C)C)c6cc54)ccc2c1)c1ccccc1O3]

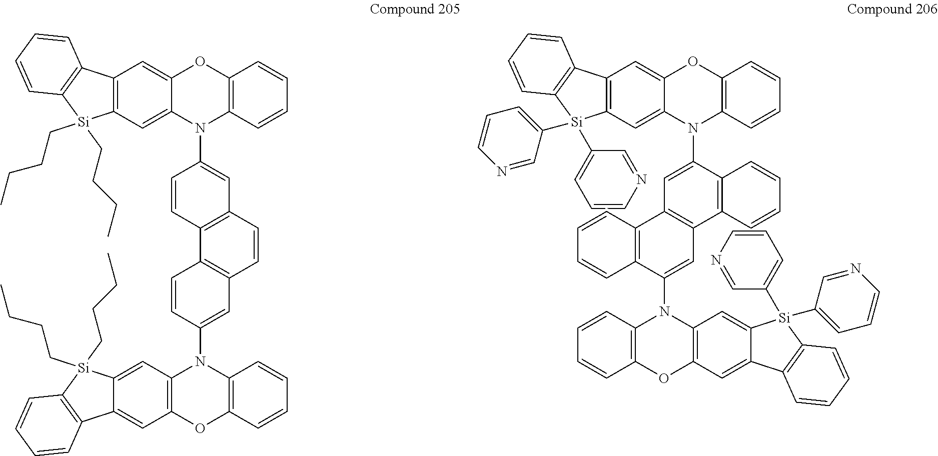 [SMILES: CCCC[Si]1(CCCC)c2ccccc2-c2cc3c(cc21)N(c1ccc2c(ccc4cc(N5c6ccccc6Oc6cc7c(cc65)[Si](CCCC)(CCCC)c5ccccc5-7)ccc42)c1)c1ccccc1O3 ; c1cncc([Si]2(c3cccnc3)c3ccccc3-c3cc4c(cc32)N(c2cc3c5ccccc5c(N5c6ccccc6Oc6cc7c(cc65)[Si](c5cccnc5)(c5cccnc5)c5ccccc5-7)cc3c3ccccc23)c2ccccc2O4)c1]

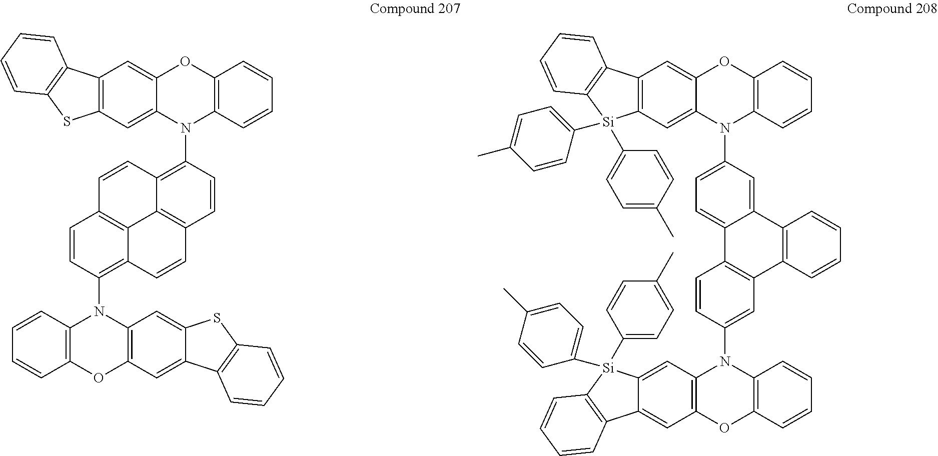 [SMILES: c1ccc2c(c1)Oc1cc3c(cc1N2c1ccc2ccc4c(N5c6ccccc6Oc6cc7c(cc65)sc5ccccc57)ccc5ccc1c2c54)sc1ccccc13 ; Cc1ccc([Si]2(c3ccc(C)cc3)c3ccccc3-c3cc4c(cc32)N(c2ccc3c5ccc(N6c7ccccc7Oc7cc8c(cc76)[Si](c6ccc(C)cc6)(c6ccc(C)cc6)c6ccccc6-8)cc5c5ccccc5c3c2)c2ccccc2O4)cc1]

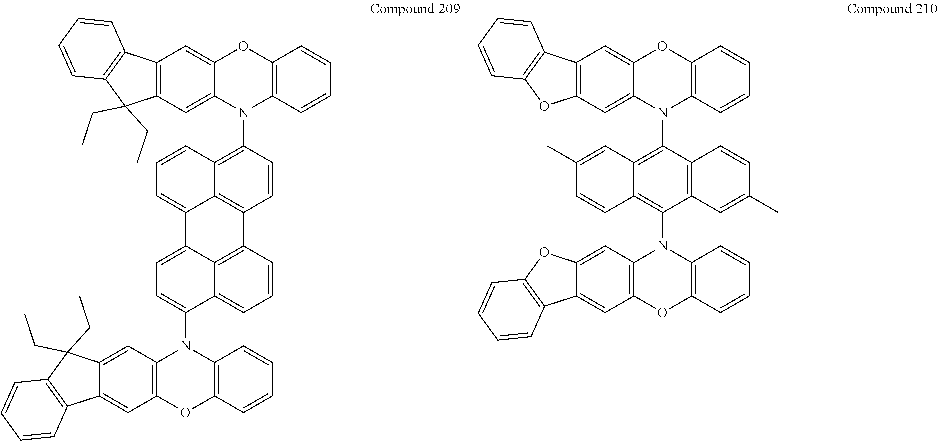 [SMILES: CCC1(CC)c2ccccc2-c2cc3c(cc21)N(c1ccc2c4cccc5c(N6c7ccccc7Oc7cc8c(cc76)C(CC)(CC)c6ccccc6-8)ccc(c6cccc1c62)c54)c1ccccc1O3 ; Cc1ccc2c(N3c4ccccc4Oc4cc5c(cc43)oc3ccccc35)c3cc(C)ccc3c(N3c4ccccc4Oc4cc5c(cc43)oc3ccccc35)c2c1]

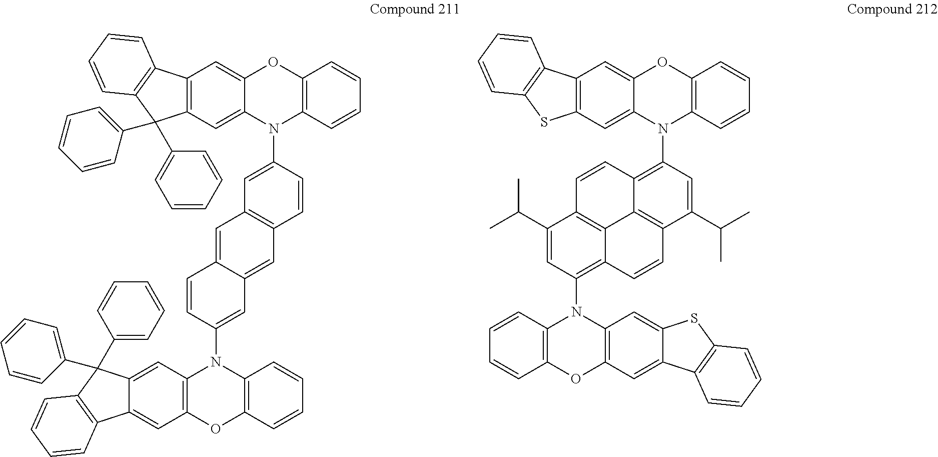 [SMILES: c1ccc(C2(c3ccccc3)c3ccccc3-c3cc4c(cc32)N(c2ccc3cc5cc(N6c7ccccc7Oc7cc8c(cc76)C(c6ccccc6)(c6ccccc6)c6ccccc6-8)ccc5cc3c2)c2ccccc2O4)cc1 ; CC(C)c1cc(N2c3ccccc3Oc3cc4c(cc32)sc2ccccc24)c2ccc3c(C(C)C)cc(N4c5ccccc5Oc5cc6c(cc54)sc4ccccc46)c4ccc1c2c34]

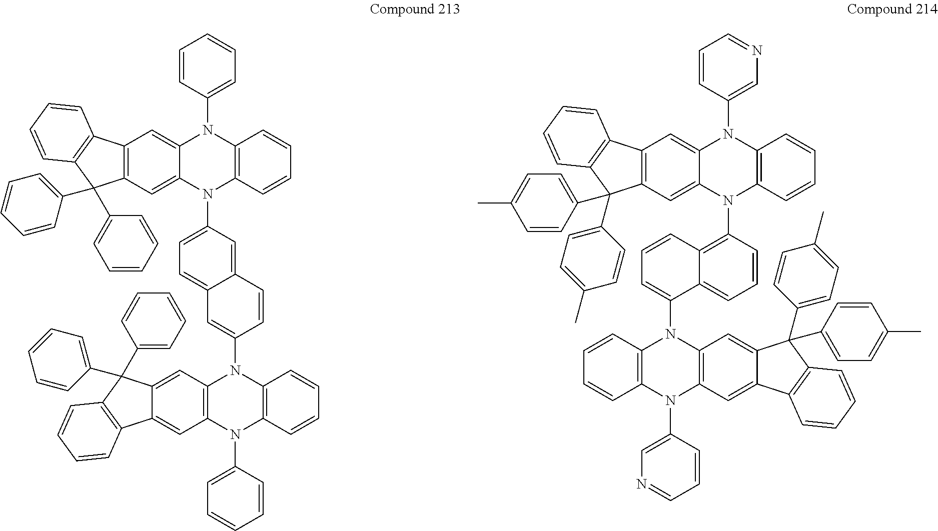

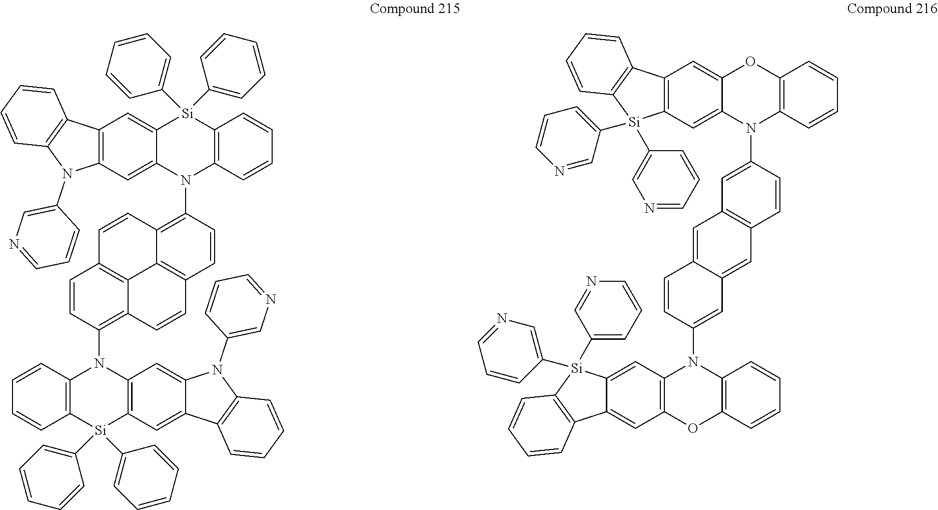 [SMILES: c1ccc([Si]2(c3ccccc3)c3ccccc3N(c3ccc4ccc5c(N6c7ccccc7[Si](c7ccccc7)(c7ccccc7)c7cc8c9ccccc9n(-c9cccnc9)c8cc76)ccc6ccc3c4c65)c3cc4c(cc32)c2ccccc2n4-c2cccnc2)cc1 ; c1cncc([Si]2(c3cccnc3)c3ccccc3-c3cc4c(cc32)N(c2ccc3cc5cc(N6c7ccccc7Oc7cc8c(cc76)[Si](c6cccnc6)(c6cccnc6)c6ccccc6-8)ccc5cc3c2)c2ccccc2O4)c1]

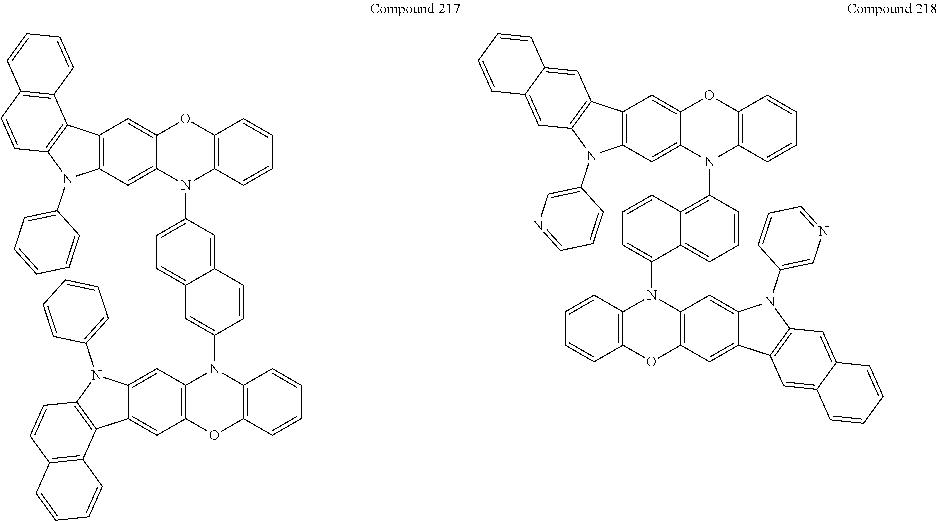 [SMILES: c1ccc(-n2c3cc4c(cc3c3c5ccccc5ccc32)Oc2ccccc2N4c2ccc3cc(N4c5ccccc5Oc5cc6c7c8ccccc8ccc7n(-c7ccccc7)c6cc54)ccc3c2)cc1 ; c1cncc(-n2c3cc4c(cc3c3cc5ccccc5cc32)Oc2ccccc2N4c2cccc3c(N4c5ccccc5Oc5cc6c7cc8ccccc8cc7n(-c7cccnc7)c6cc54)cccc23)c1]

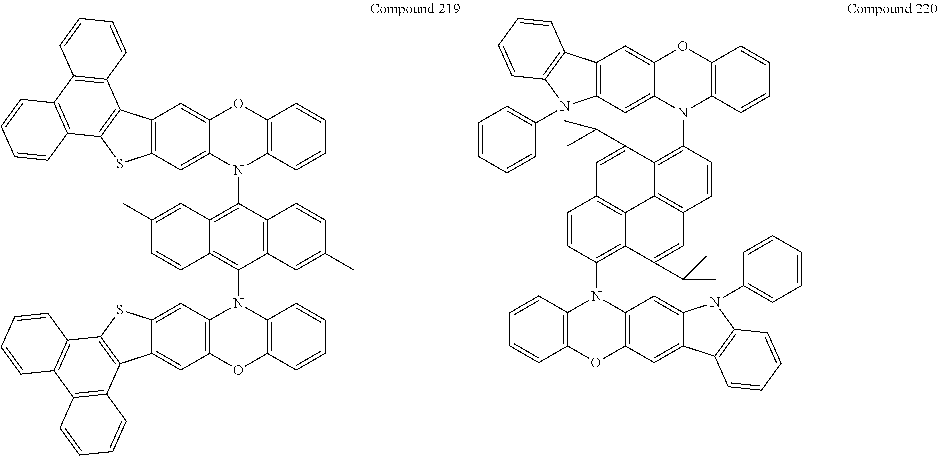 [SMILES: Cc1ccc2c(N3c4ccccc4Oc4cc5c(cc43)sc3c4ccccc4c4ccccc4c53)c3cc(C)ccc3c(N3c4ccccc4Oc4cc5c(cc43)sc3c4ccccc4c4ccccc4c53)c2c1 ; CC(C)c1cc2ccc(N3c4ccccc4Oc4cc5c6ccccc6n(-c6ccccc6)c5cc43)c3c(C(C)C)cc4ccc(N5c6ccccc6Oc6cc7c8ccccc8n(-c8ccccc8)c7cc65)c1c4c23]

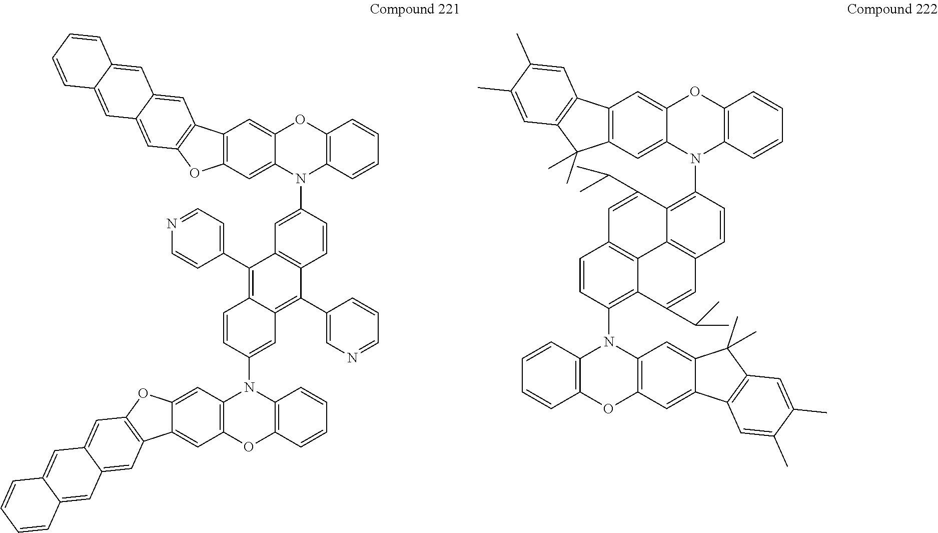

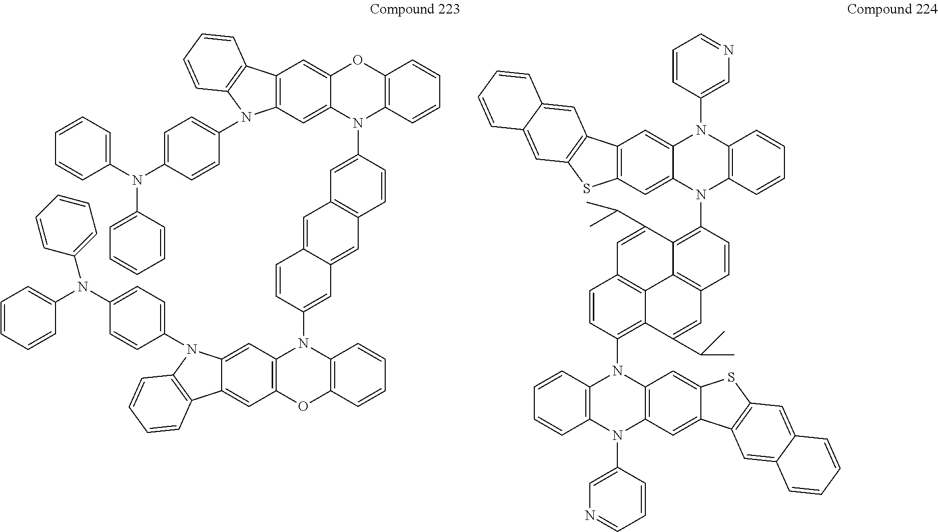

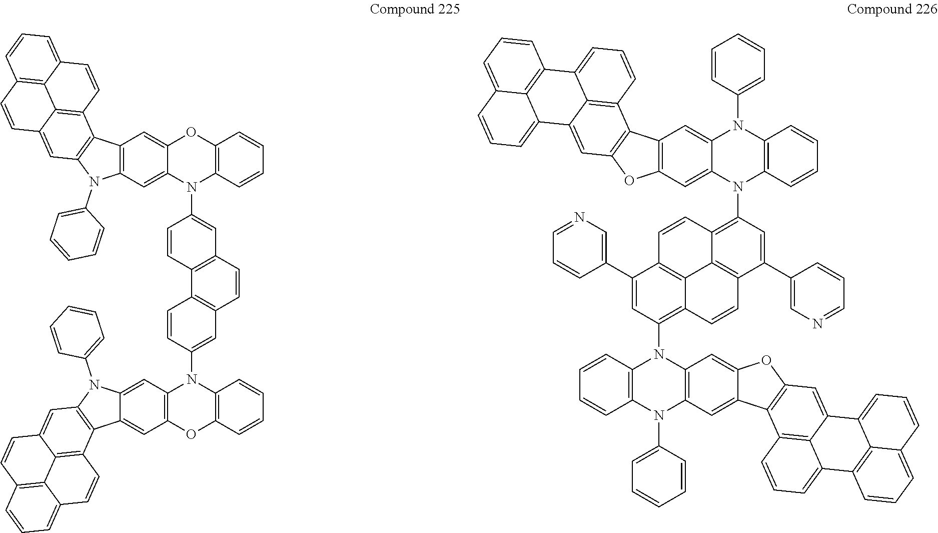 [SMILES: c1ccc(-n2c3cc4c(cc3c3c5ccc6cccc7ccc(cc32)c5c76)oc2ccccc2n4-c2ccc3c(ccc4cc(-n5c6ccccc6oc6cc7c8c9ccc%10cccc%11ccc(cc8n(-c8ccccc8)c7cc65)c9c%11%10)ccc43)c2)cc1 ; c1ccc(-n2c3ccccc3n(-c3cc(-c4cccnc4)c4ccc5c(-n6c7ccccc7n(-c7ccccc7)c7cc8c(cc76)oc6cc7c9cccc%10cccc(c%11cccc(c68)c%117)c%109)cc(-c6cccnc6)c6ccc3c4c65)c3cc4oc5cc6c7cccc8cccc(c9cccc(c5c4cc32)c96)c87)cc1]

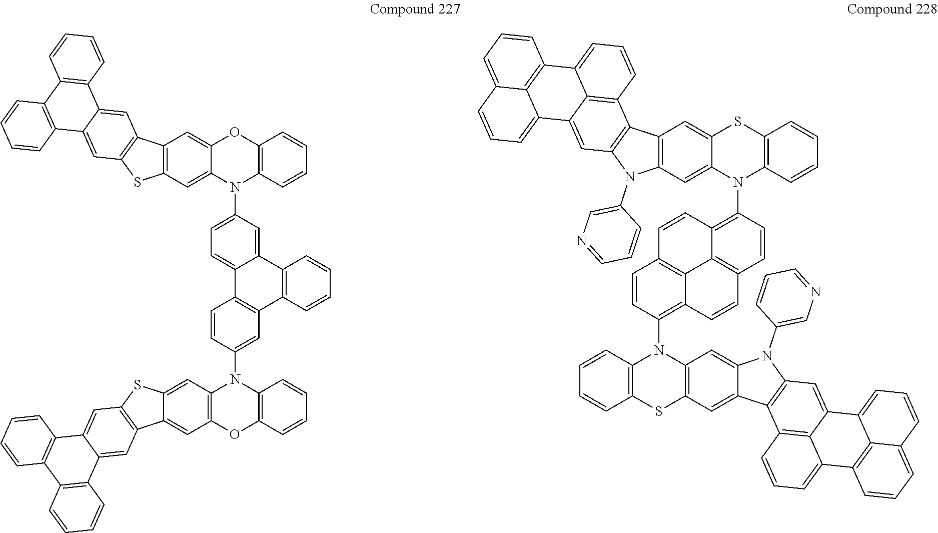 [SMILES: c1ccc2c(c1)Oc1cc3c(cc1N2c1ccc2c4ccc(N5c6ccccc6Oc6cc7c(cc65)sc5cc6c8ccccc8c8ccccc8c6cc57)cc4c4ccccc4c2c1)sc1cc2c4ccccc4c4ccccc4c2cc13 ; c1cncc(-n2c3cc4c(cc3c3c5cccc6c7cccc8cccc(c(cc32)c65)c87)sc2ccccc2n4-c2ccc3ccc4c(-n5c6ccccc6sc6cc7c8c9cccc%10c%11cccc%12cccc(c(cc8n(-c8cccnc8)c7cc65)c%109)c%12%11)ccc5ccc2c3c54)c1]

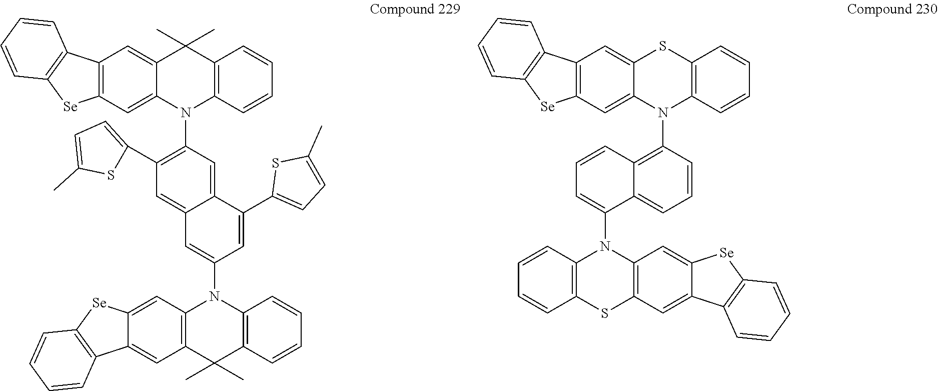 [SMILES: Cc1ccc(-c2cc3cc(N4c5ccccc5C(C)(C)c5cc6c(cc54)[se]c4ccccc46)cc(-c4ccc(C)s4)c3cc2N2c3ccccc3C(C)(C)c3cc4c(cc32)[se]c2ccccc24)s1 ; c1ccc2c(c1)Sc1cc3c(cc1N2c1cccc2c(N4c5ccccc5Sc5cc6c(cc54)[se]c4ccccc46)cccc12)[se]c1ccccc13]

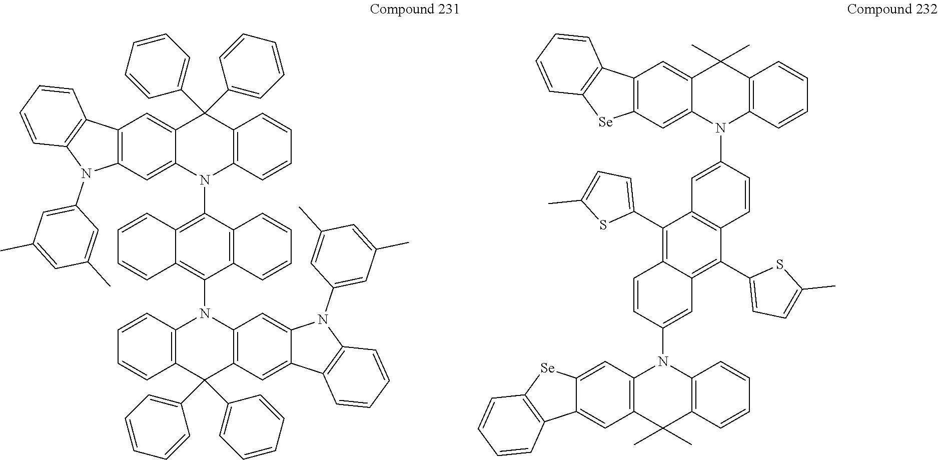

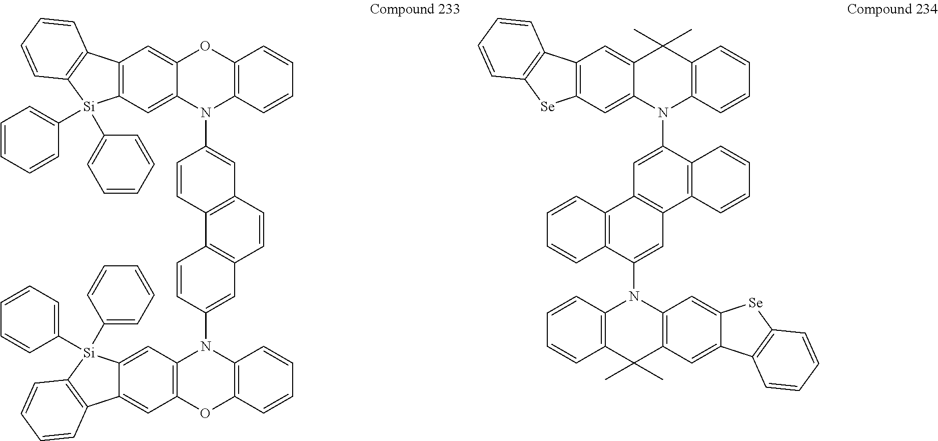 [SMILES: c1ccc([Si]2(c3ccccc3)c3ccccc3-c3cc4c(cc32)N(c2ccc3c(ccc5cc(N6c7ccccc7Oc7cc8c(cc76)[Si](c6ccccc6)(c6ccccc6)c6ccccc6-8)ccc53)c2)c2ccccc2O4)cc1 ; CC1(C)c2ccccc2N(c2cc3c4ccccc4c(N4c5ccccc5C(C)(C)c5cc6c(cc54)[se]c4ccccc46)cc3c3ccccc23)c2cc3[se]c4ccccc4c3cc21]

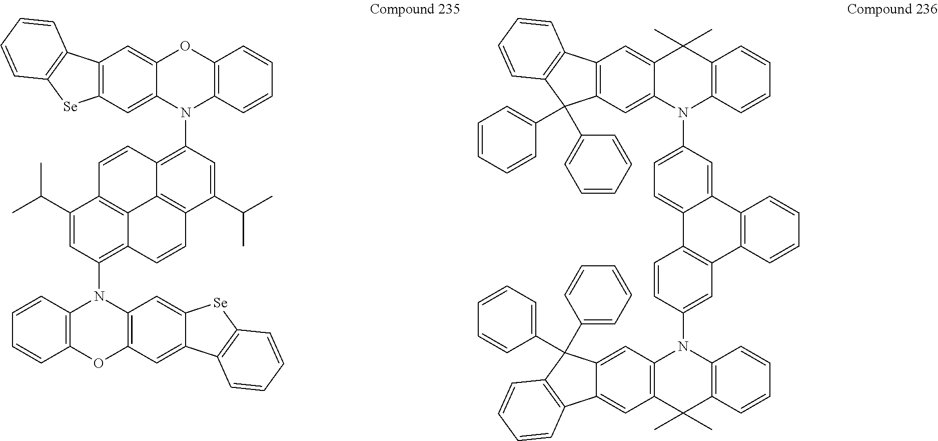 [SMILES: CC(C)c1cc(N2c3ccccc3Oc3cc4c(cc32)[se]c2ccccc24)c2ccc3c(C(C)C)cc(N4c5ccccc5Oc5cc6c(cc54)[se]c4ccccc46)c4ccc1c2c34 ; CC1(C)c2ccccc2N(c2ccc3c4ccc(N5c6ccccc6C(C)(C)c6cc7c(cc65)C(c5ccccc5)(c5ccccc5)c5ccccc5-7)cc4c4ccccc4c3c2)c2cc3c(cc21)-c1ccccc1C3(c1ccccc1)c1ccccc1]

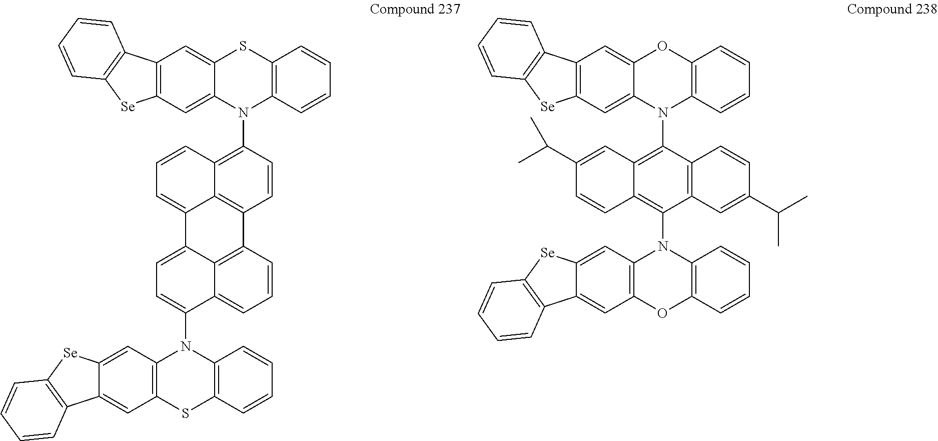 [SMILES: c1ccc2c(c1)Sc1cc3c(cc1N2c1ccc2c4cccc5c(N6c7ccccc7Sc7cc8c(cc76)[se]c6ccccc68)ccc(c6cccc1c62)c54)[se]c1ccccc13 ; CC(C)c1ccc2c(N3c4ccccc4Oc4cc5c(cc43)[se]c3ccccc35)c3cc(C(C)C)ccc3c(N3c4ccccc4Oc4cc5c(cc43)[se]c3ccccc35)c2c1]

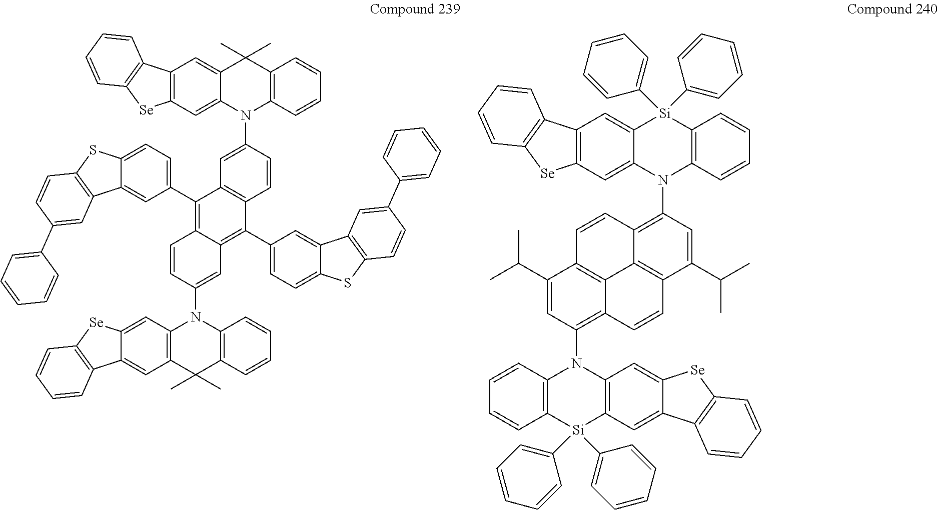 [SMILES: CC1(C)c2ccccc2N(c2ccc3c(-c4ccc5sc6ccc(-c7ccccc7)cc6c5c4)c4cc(N5c6ccccc6C(C)(C)c6cc7c(cc65)[se]c5ccccc57)ccc4c(-c4ccc5sc6ccc(-c7ccccc7)cc6c5c4)c3c2)c2cc3[se]c4ccccc4c3cc21 ; CC(C)c1cc(N2c3ccccc3[Si](c3ccccc3)(c3ccccc3)c3cc4c(cc32)[se]c2ccccc24)c2ccc3c(C(C)C)cc(N4c5ccccc5[Si](c5ccccc5)(c5ccccc5)c5cc6c(cc54)[se]c4ccccc46)c4ccc1c2c34]

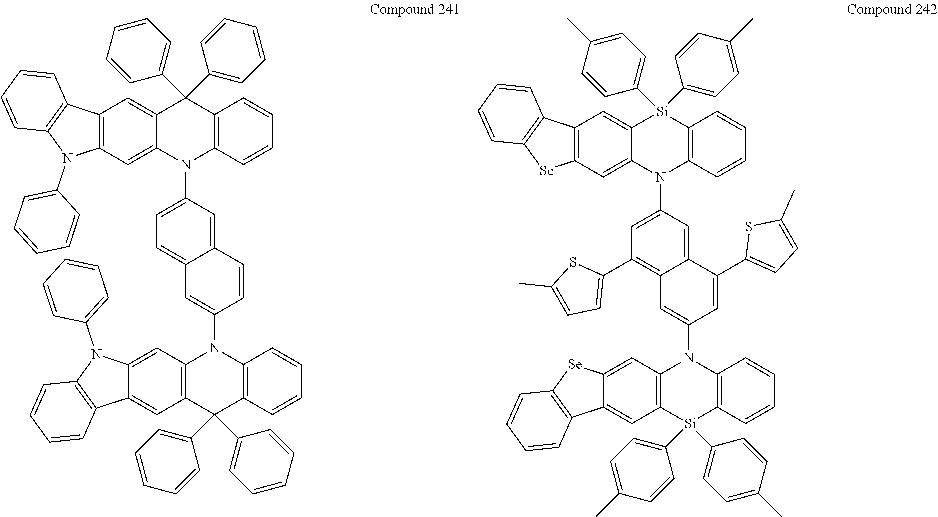 [SMILES: c1ccc(-n2c3ccccc3c3cc4c(cc32)N(c2ccc3cc(N5c6ccccc6C(c6ccccc6)(c6ccccc6)c6cc7c8ccccc8n(-c8ccccc8)c7cc65)ccc3c2)c2ccccc2C4(c2ccccc2)c2ccccc2)cc1 ; Cc1ccc([Si]2(c3ccc(C)cc3)c3ccccc3N(c3cc(-c4ccc(C)s4)c4cc(N5c6ccccc6[Si](c6ccc(C)cc6)(c6ccc(C)cc6)c6cc7c(cc65)[se]c5ccccc57)cc(-c5ccc(C)s5)c4c3)c3cc4[se]c5ccccc5c4cc32)cc1]

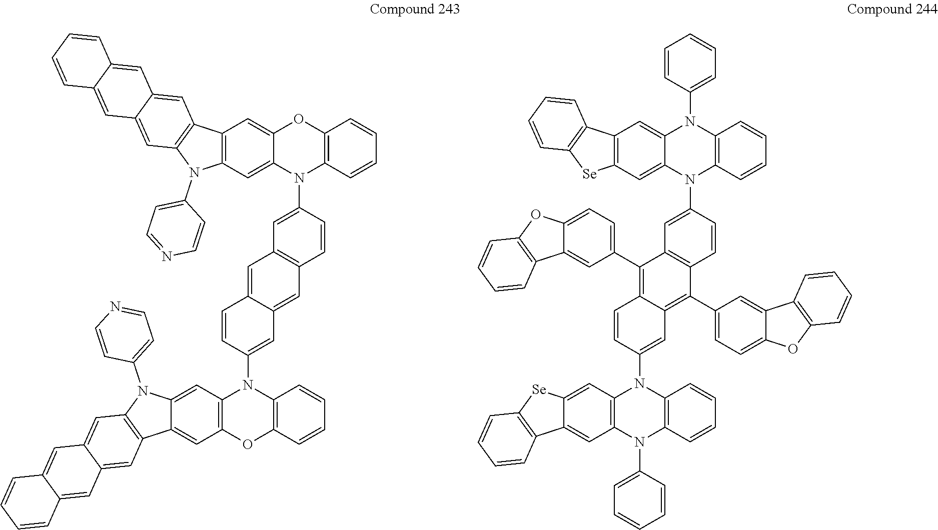 [SMILES: c1ccc2c(c1)Oc1cc3c4cc5cc6ccccc6cc5cc4n(-c4ccncc4)c3cc1N2c1ccc2cc3cc(N4c5ccccc5Oc5cc6c7cc8cc9ccccc9cc8cc7n(-c7ccncc7)c6cc54)ccc3cc2c1 ; c1ccc(N2c3ccccc3N(c3ccc4c(-c5ccc6oc7ccccc7c6c5)c5cc(N6c7ccccc7N(c7ccccc7)c7cc8c(cc76)[se]c6ccccc68)ccc5c(-c5ccc6oc7ccccc7c6c5)c4c3)c3cc4[se]c5ccccc5c4cc32)cc1]

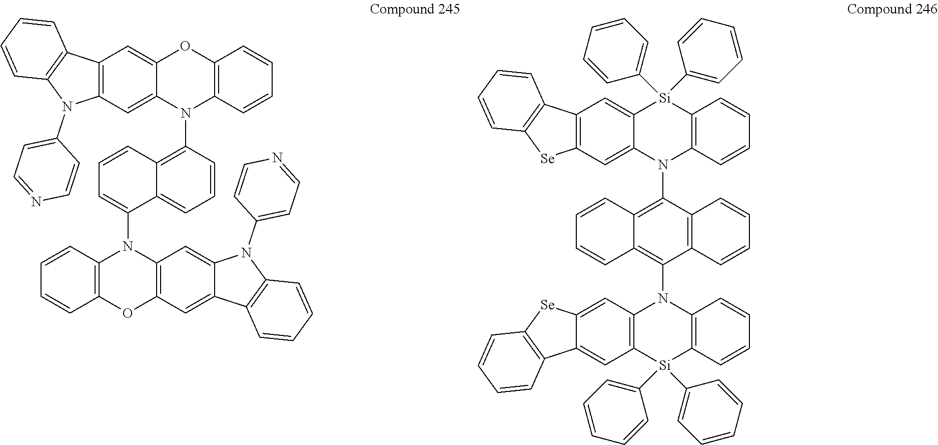 [SMILES: c1ccc2c(c1)Oc1cc3c4ccccc4n(-c4ccncc4)c3cc1N2c1cccc2c(N3c4ccccc4Oc4cc5c6ccccc6n(-c6ccncc6)c5cc43)cccc12 ; c1ccc([Si]2(c3ccccc3)c3ccccc3N(c3c4ccccc4c(N4c5ccccc5[Si](c5ccccc5)(c5ccccc5)c5cc6c(cc54)[se]c4ccccc46)c4ccccc34)c3cc4[se]c5ccccc5c4cc32)cc1]

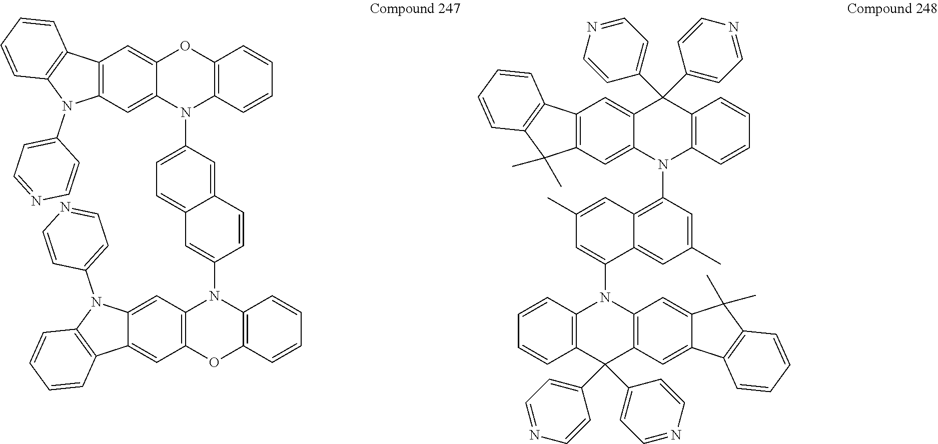 [SMILES: c1ccc2c(c1)Oc1cc3c4ccccc4n(-c4ccncc4)c3cc1N2c1ccc2cc(N3c4ccccc4Oc4cc5c6ccccc6n(-c6ccncc6)c5cc43)ccc2c1 ; Cc1cc(N2c3ccccc3C(c3ccncc3)(c3ccncc3)c3cc4c(cc32)C(C)(C)c2ccccc2-4)c2cc(C)cc(N3c4ccccc4C(c4ccncc4)(c4ccncc4)c4cc5c(cc43)C(C)(C)c3ccccc3-5)c2c1]

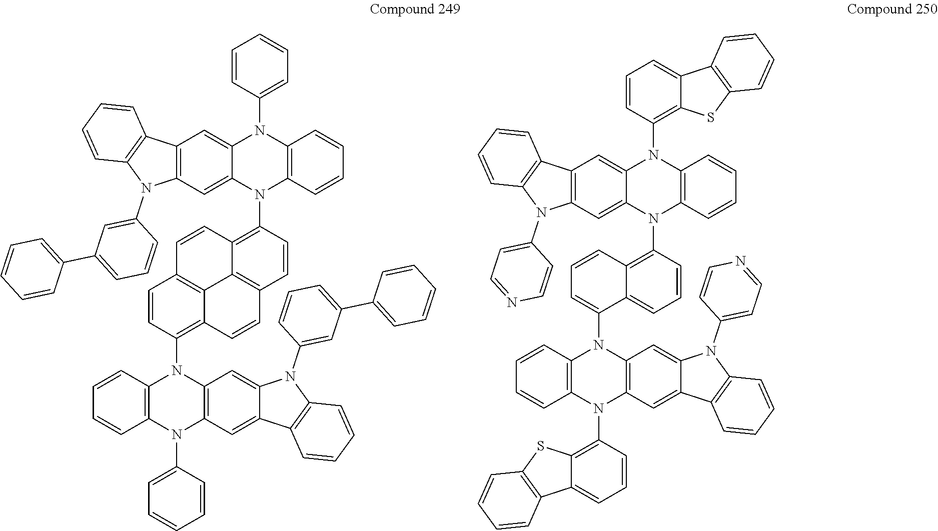 [SMILES: c1ccc(-c2cccc(-n3c4ccccc4c4cc5c(cc43)N(c3ccc4ccc6c(N7c8ccccc8N(c8ccccc8)c8cc9c%10ccccc%10n(-c%10cccc(-c%11ccccc%11)c%10)c9cc87)ccc7ccc3c4c76)c3ccccc3N5c3ccccc3)c2)cc1 ; c1ccc2c(c1)N(c1cccc3c(N4c5ccccc5N(c5cccc6c5sc5ccccc56)c5cc6c7ccccc7n(-c7ccncc7)c6cc54)cccc13)c1cc3c(cc1N2c1cccc2c1sc1ccccc12)c1ccccc1n3-c1ccncc1]

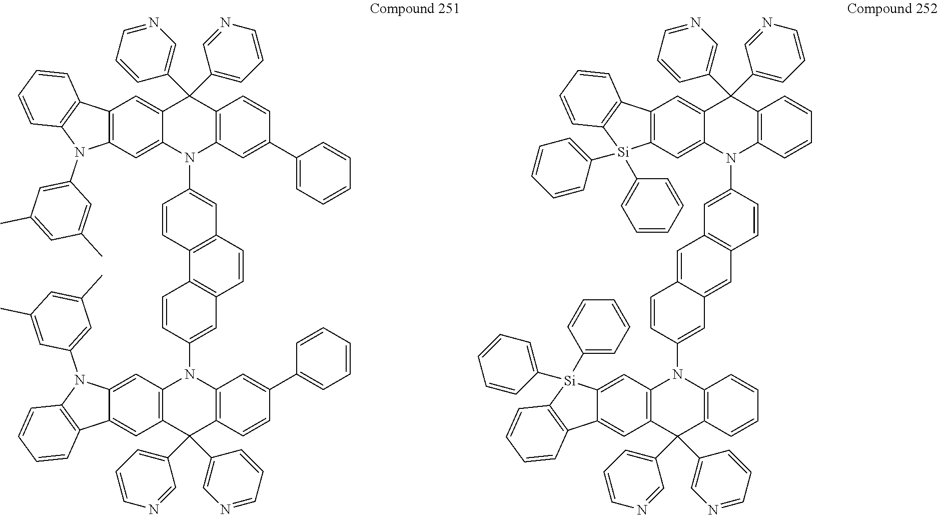 [SMILES: Cc1cc(C)cc(-n2c3ccccc3c3cc4c(cc32)N(c2ccc3c(ccc5cc(N6c7cc(-c8ccccc8)ccc7C(c7cccnc7)(c7cccnc7)c7cc8c9ccccc9n(-c9cc(C)cc(C)c9)c8cc76)ccc53)c2)c2cc(-c3ccccc3)ccc2C4(c2cccnc2)c2cccnc2)c1 ; c1ccc([Si]2(c3ccccc3)c3ccccc3-c3cc4c(cc32)N(c2ccc3cc5cc(N6c7ccccc7C(c7cccnc7)(c7cccnc7)c7cc8c(cc76)[Si](c6ccccc6)(c6ccccc6)c6ccccc6-8)ccc5cc3c2)c2ccccc2C4(c2cccnc2)c2cccnc2)cc1]

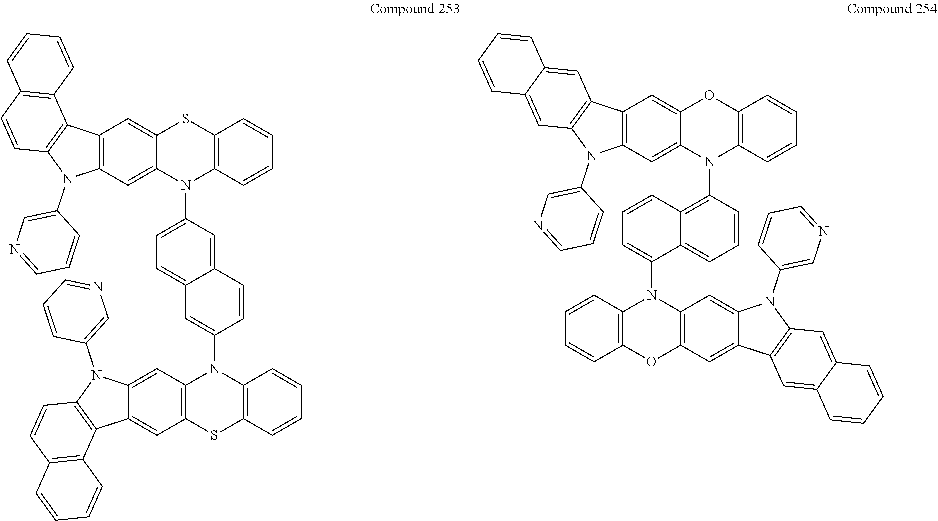 [SMILES: c1cncc(-n2c3cc4c(cc3c3c5ccccc5ccc32)Sc2ccccc2N4c2ccc3cc(N4c5ccccc5Sc5cc6c7c8ccccc8ccc7n(-c7cccnc7)c6cc54)ccc3c2)c1 ; c1cncc(-n2c3cc4c(cc3c3cc5ccccc5cc32)Oc2ccccc2N4c2cccc3c(N4c5ccccc5Oc5cc6c7cc8ccccc8cc7n(-c7cccnc7)c6cc54)cccc23)c1]

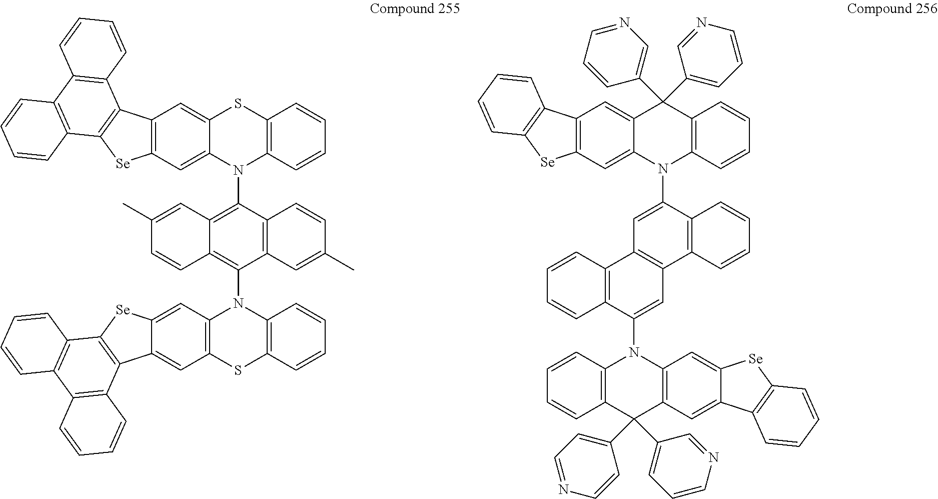 [SMILES: Cc1ccc2c(N3c4ccccc4Sc4cc5c(cc43)[se]c3c4ccccc4c4ccccc4c53)c3cc(C)ccc3c(N3c4ccccc4Sc4cc5c(cc43)[se]c3c4ccccc4c4ccccc4c53)c2c1 ; c1cncc(C2(c3ccncc3)c3ccccc3N(c3cc4c5ccccc5c(N5c6ccccc6C(c6cccnc6)(c6cccnc6)c6cc7c(cc65)[se]c5ccccc57)cc4c4ccccc34)c3cc4[se]c5ccccc5c4cc32)c1]

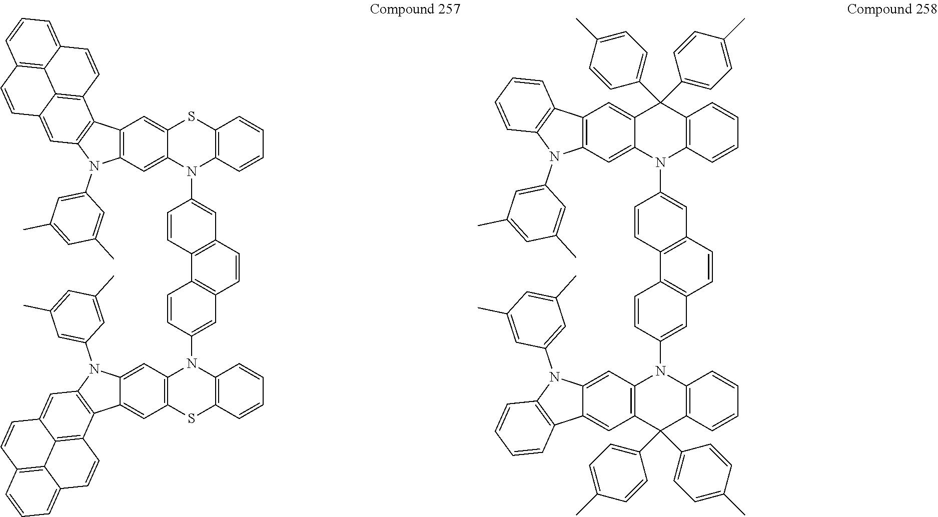

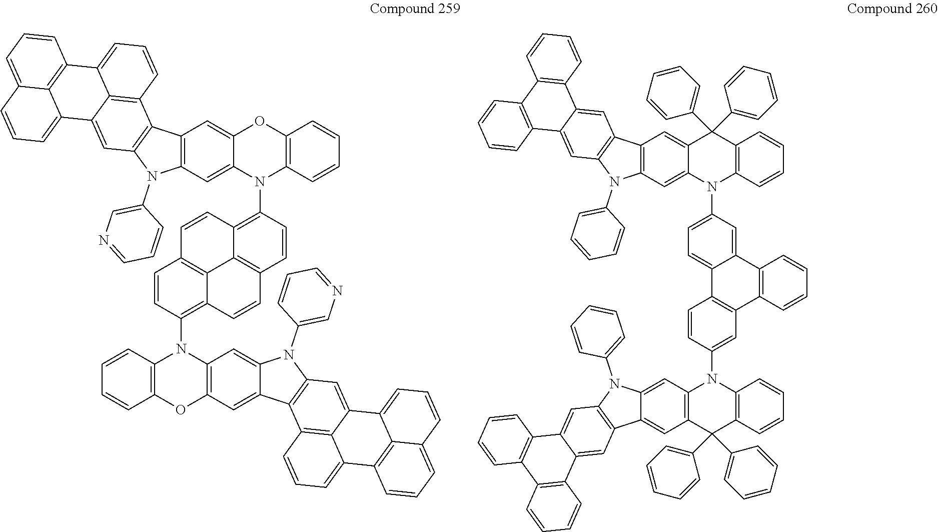 [SMILES: c1cncc(-n2c3cc4c(cc3c3c5cccc6c7cccc8cccc(c(cc32)c65)c87)oc2ccccc2n4-c2ccc3ccc4c(-n5c6ccccc6oc6cc7c8c9cccc%10c%11cccc%12cccc(c(cc8n(-c8cccnc8)c7cc65)c%109)c%12%11)ccc5ccc2c3c54)c1 ; c1ccc(-n2c3cc4c(cc3c3cc5c6ccccc6c6ccccc6c5cc32)C(c2ccccc2)(c2ccccc2)c2ccccc2N4c2ccc3c4ccc(N5c6ccccc6C(c6ccccc6)(c6ccccc6)c6cc7c8cc9c%10ccccc%10c%10ccccc%10c9cc8n(-c8ccccc8)c7cc65)cc4c4ccccc4c3c2)cc1]

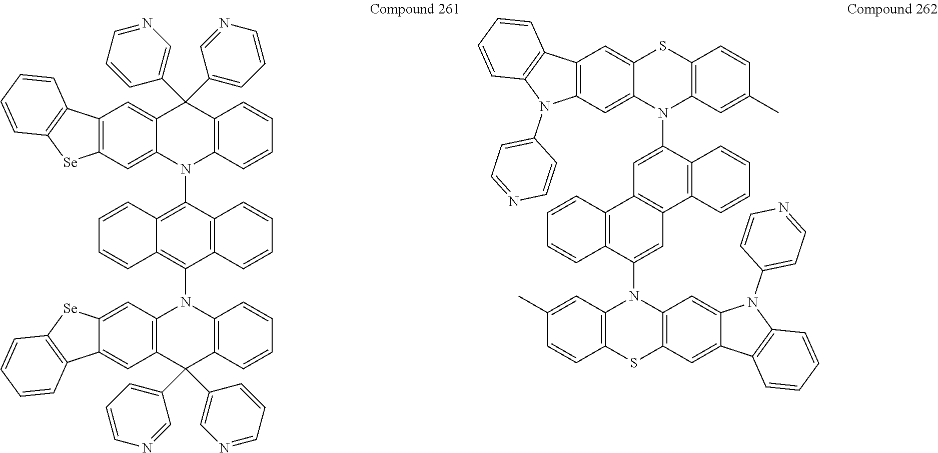 [SMILES: c1cncc(C2(c3cccnc3)c3ccccc3N(c3c4ccccc4c(N4c5ccccc5C(c5cccnc5)(c5cccnc5)c5cc6c(cc54)[se]c4ccccc46)c4ccccc34)c3cc4[se]c5ccccc5c4cc32)c1 ; Cc1ccc2c(c1)N(c1cc3c4ccccc4c(N4c5cc(C)ccc5Sc5cc6c7ccccc7n(-c7ccncc7)c6cc54)cc3c3ccccc13)c1cc3c(cc1S2)c1ccccc1n3-c1ccncc1]

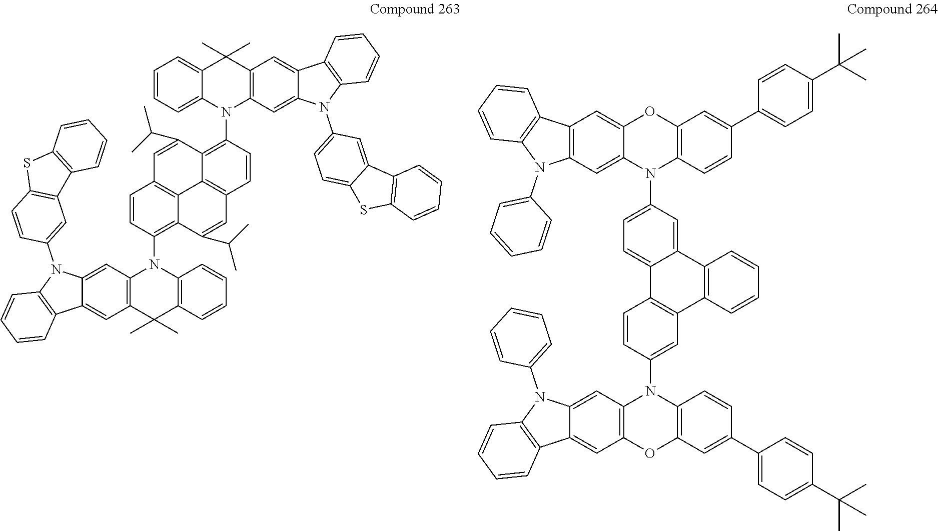 [SMILES: CC(C)c1cc2ccc(N3c4ccccc4C(C)(C)c4cc5c6ccccc6n(-c6ccc7sc8ccccc8c7c6)c5cc43)c3c(C(C)C)cc4ccc(N5c6ccccc6C(C)(C)c6cc7c8ccccc8n(-c8ccc9sc%10ccccc%10c9c8)c7cc65)c1c4c23 ; CC(C)(C)c1ccc(-c2ccc3c(c2)Oc2cc4c5ccccc5n(-c5ccccc5)c4cc2N3c2ccc3c4ccc(N5c6ccc(-c7ccc(C(C)(C)C)cc7)cc6Oc6cc7c8ccccc8n(-c8ccccc8)c7cc65)cc4c4ccccc4c3c2)cc1]

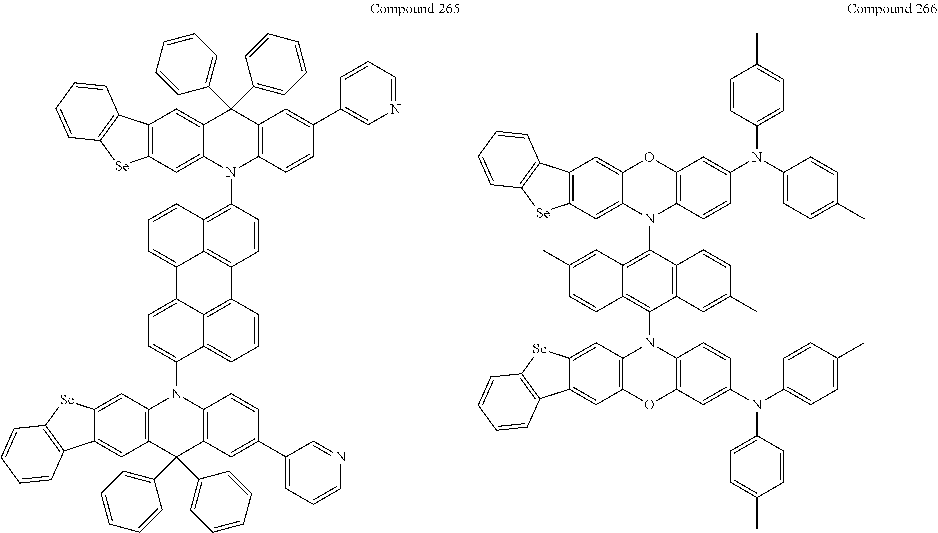 [SMILES: c1ccc(C2(c3ccccc3)c3cc(-c4cccnc4)ccc3N(c3ccc4c5cccc6c(N7c8ccc(-c9cccnc9)cc8C(c8ccccc8)(c8ccccc8)c8cc9c(cc87)[se]c7ccccc79)ccc(c7cccc3c74)c65)c3cc4[se]c5ccccc5c4cc32)cc1 ; Cc1ccc(N(c2ccc(C)cc2)c2ccc3c(c2)Oc2cc4c(cc2N3c2c3ccc(C)cc3c(N3c5ccc(N(c6ccc(C)cc6)c6ccc(C)cc6)cc5Oc5cc6c(cc53)[se]c3ccccc36)c3ccc(C)cc23)[se]c2ccccc24)cc1]

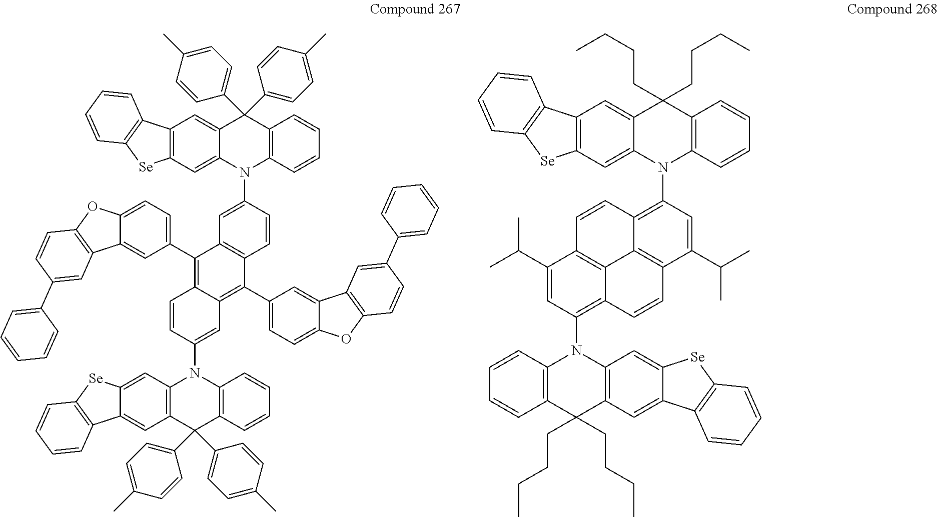 [SMILES: Cc1ccc(C2(c3ccc(C)cc3)c3ccccc3N(c3ccc4c(-c5ccc6oc7ccc(-c8ccccc8)cc7c6c5)c5cc(N6c7ccccc7C(c7ccc(C)cc7)(c7ccc(C)cc7)c7cc8c(cc76)[se]c6ccccc68)ccc5c(-c5ccc6oc7ccc(-c8ccccc8)cc7c6c5)c4c3)c3cc4[se]c5ccccc5c4cc32)cc1 ; CCCCC1(CCCC)c2ccccc2N(c2cc(C(C)C)c3ccc4c(N5c6ccccc6C(CCCC)(CCCC)c6cc7c(cc65)[se]c5ccccc57)cc(C(C)C)c5ccc2c3c54)c2cc3[se]c4ccccc4c3cc21]

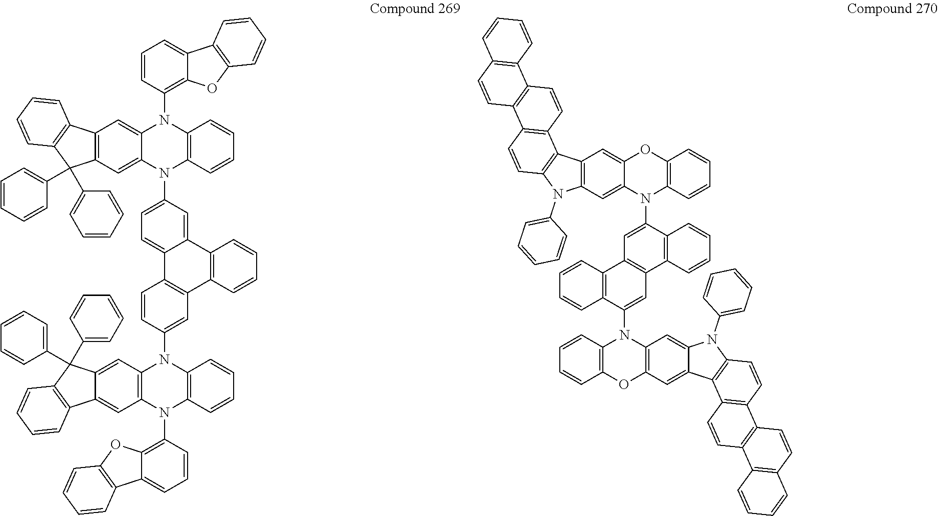 [SMILES: c1ccc(C2(c3ccccc3)c3ccccc3-c3cc4c(cc32)N(c2ccc3c5ccc(N6c7ccccc7N(c7cccc8c7oc7ccccc78)c7cc8c(cc76)C(c6ccccc6)(c6ccccc6)c6ccccc6-8)cc5c5ccccc5c3c2)c2ccccc2N4c2cccc3c2oc2ccccc23)cc1 ; c1ccc(-n2c3cc4c(cc3c3c5ccc6c7ccccc7ccc6c5ccc32)Oc2ccccc2N4c2cc3c4ccccc4c(N4c5ccccc5Oc5cc6c7c8ccc9c%10ccccc%10ccc9c8ccc7n(-c7ccccc7)c6cc54)cc3c3ccccc23)cc1]

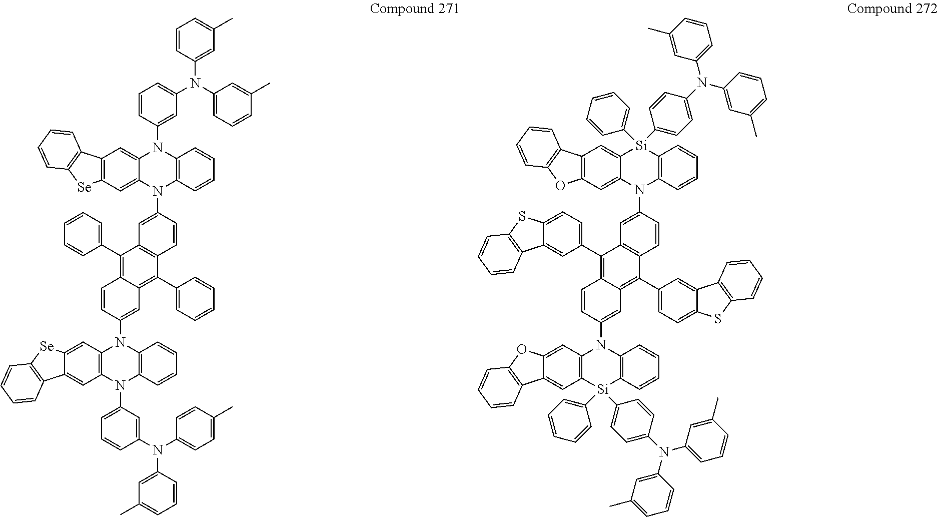 [SMILES: Cc1ccc(N(c2cccc(C)c2)c2cccc(N3c4ccccc4N(c4ccc5c(-c6ccccc6)c6cc(N7c8ccccc8N(c8cccc(N(c9cccc(C)c9)c9cccc(C)c9)c8)c8cc9c(cc87)[se]c7ccccc79)ccc6c(-c6ccccc6)c5c4)c4cc5[se]c6ccccc6c5cc43)c2)cc1 ; Cc1cccc(N(c2ccc([Si]3(c4ccccc4)c4ccccc4N(c4ccc5c(-c6ccc7sc8ccccc8c7c6)c6cc(N7c8ccccc8[Si](c8ccccc8)(c8ccc(N(c9cccc(C)c9)c9cccc(C)c9)cc8)c8cc9c(cc87)oc7ccccc79)ccc6c(-c6ccc7sc8ccccc8c7c6)c5c4)c4cc5oc6ccccc6c5cc43)cc2)c2cccc(C)c2)c1]

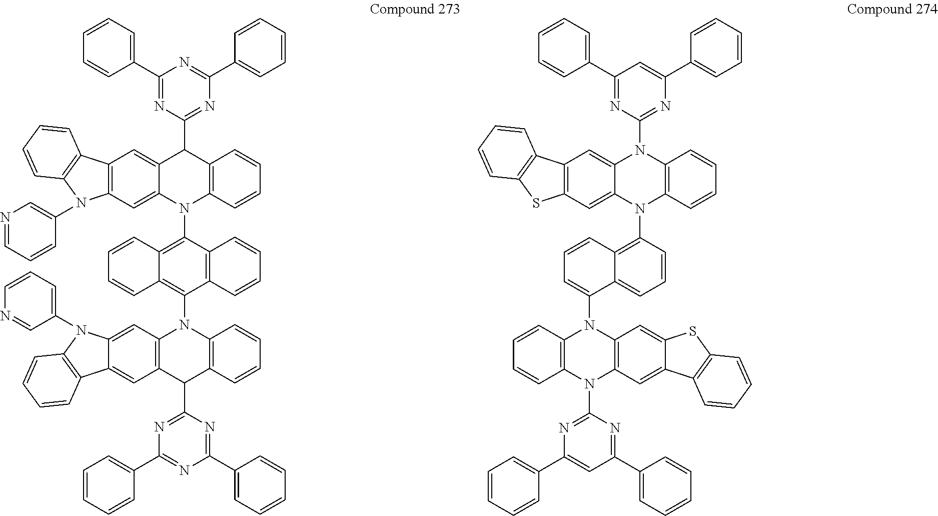 [SMILES: c1ccc(-c2nc(-c3ccccc3)nc(C3c4ccccc4N(c4c5ccccc5c(N5c6ccccc6C(c6nc(-c7ccccc7)nc(-c7ccccc7)n6)c6cc7c8ccccc8n(-c8cccnc8)c7cc65)c5ccccc45)c4cc5c(cc43)c3ccccc3n5-c3cccnc3)n2)cc1 ; c1ccc(-c2cc(-c3ccccc3)nc(N3c4ccccc4N(c4cccc5c(N6c7ccccc7N(c7nc(-c8ccccc8)cc(-c8ccccc8)n7)c7cc8c(cc76)sc6ccccc68)cccc45)c4cc5sc6ccccc6c5cc43)n2)cc1]

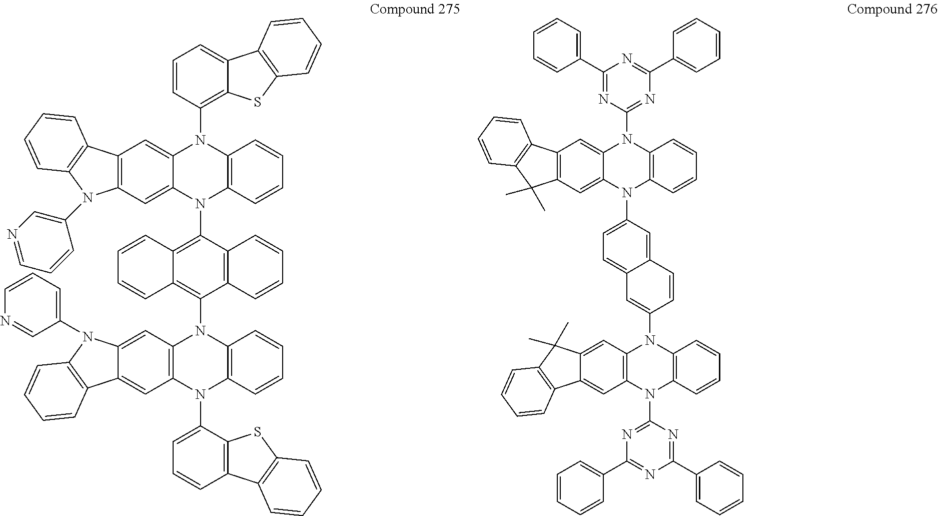 [SMILES: c1cncc(-n2c3ccccc3c3cc4c(cc32)N(c2c3ccccc3c(N3c5ccccc5N(c5cccc6c5sc5ccccc56)c5cc6c7ccccc7n(-c7cccnc7)c6cc53)c3ccccc23)c2ccccc2N4c2cccc3c2sc2ccccc23)c1 ; CC1(C)c2ccccc2-c2cc3c(cc21)N(c1ccc2cc(N4c5ccccc5N(c5nc(-c6ccccc6)nc(-c6ccccc6)n5)c5cc6c(cc54)C(C)(C)c4ccccc4-6)ccc2c1)c1ccccc1N3c1nc(-c2ccccc2)nc(-c2ccccc2)n1]

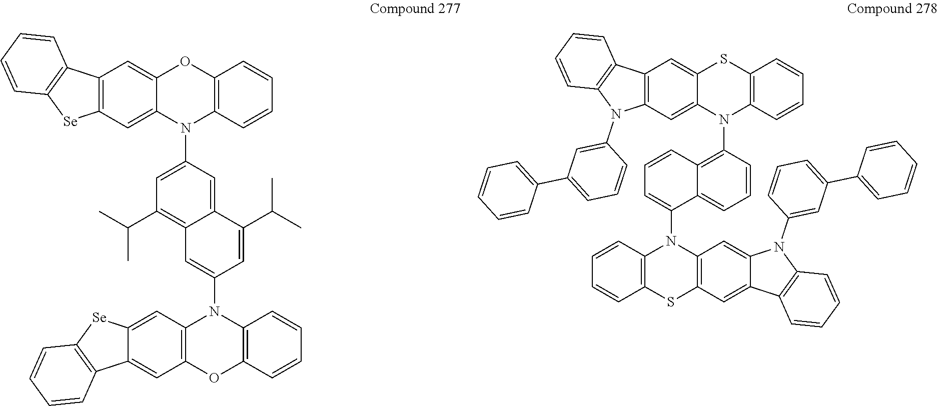 [SMILES: CC(C)c1cc(N2c3ccccc3Oc3cc4c(cc32)[se]c2ccccc24)cc2c(C(C)C)cc(N3c4ccccc4Oc4cc5c(cc43)[se]c3ccccc35)cc12 ; c1ccc(-c2cccc(-n3c4ccccc4c4cc5c(cc43)N(c3cccc4c(N6c7ccccc7Sc7cc8c9ccccc9n(-c9cccc(-c%10ccccc%10)c9)c8cc76)cccc34)c3ccccc3S5)c2)cc1]

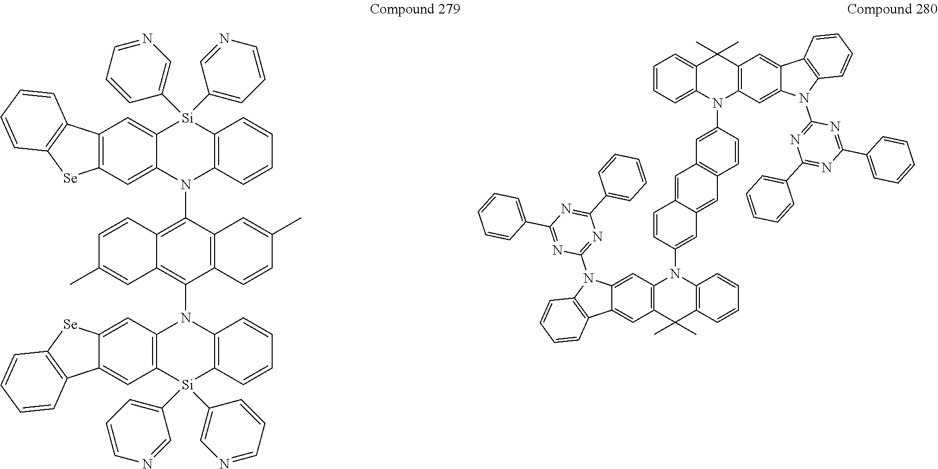 [SMILES: Cc1ccc2c(N3c4ccccc4[Si](c4cccnc4)(c4cccnc4)c4cc5c(cc43)[se]c3ccccc35)c3cc(C)ccc3c(N3c4ccccc4[Si](c4cccnc4)(c4cccnc4)c4cc5c(cc43)[se]c3ccccc35)c2c1 ; CC1(C)c2ccccc2N(c2ccc3cc4cc(N5c6ccccc6C(C)(C)c6cc7c8ccccc8n(-c8nc(-c9ccccc9)nc(-c9ccccc9)n8)c7cc65)ccc4cc3c2)c2cc3c(cc21)c1ccccc1n3-c1nc(-c2ccccc2)nc(-c2ccccc2)n1]

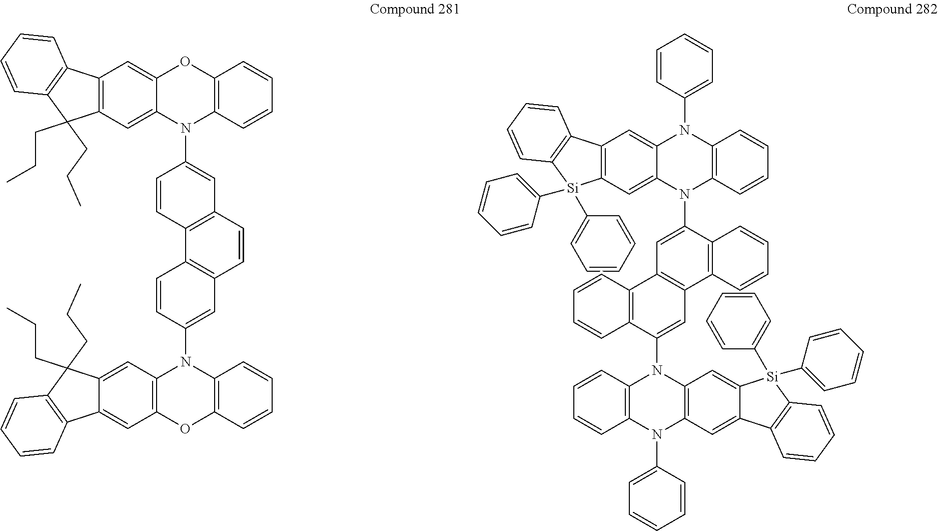 [SMILES: CCCC1(CCC)c2ccccc2-c2cc3c(cc21)N(c1ccc2c(ccc4cc(N5c6ccccc6Oc6cc7c(cc65)C(CCC)(CCC)c5ccccc5-7)ccc42)c1)c1ccccc1O3 ; c1ccc(N2c3ccccc3N(c3cc4c5ccccc5c(N5c6ccccc6N(c6ccccc6)c6cc7c(cc65)[Si](c5ccccc5)(c5ccccc5)c5ccccc5-7)cc4c4ccccc34)c3cc4c(cc32)-c2ccccc2[Si]4(c2ccccc2)c2ccccc2)cc1]

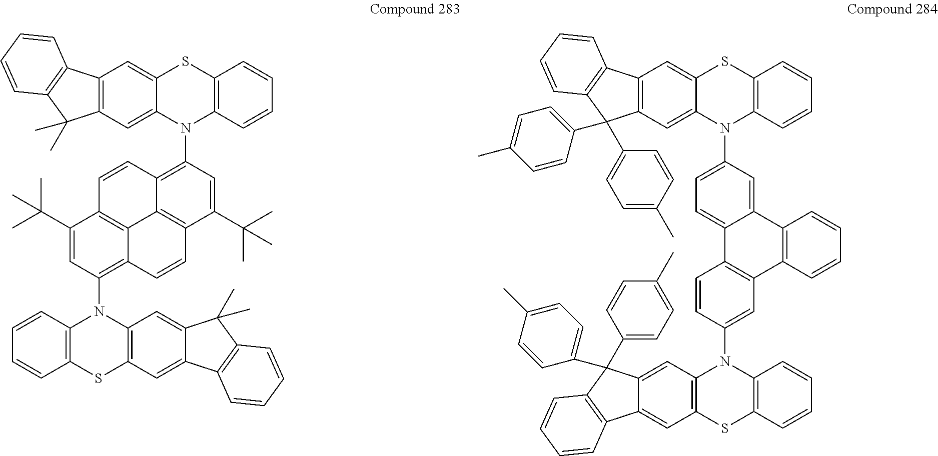 [SMILES: CC(C)(C)c1cc(N2c3ccccc3Sc3cc4c(cc32)C(C)(C)c2ccccc2-4)c2ccc3c(C(C)(C)C)cc(N4c5ccccc5Sc5cc6c(cc54)C(C)(C)c4ccccc4-6)c4ccc1c2c43 ; Cc1ccc(C2(c3ccc(C)cc3)c3ccccc3-c3cc4c(cc32)N(c2ccc3c5ccc(N6c7ccccc7Sc7cc8c(cc76)C(c6ccc(C)cc6)(c6ccc(C)cc6)c6ccccc6-8)cc5c5ccccc5c3c2)c2ccccc2S4)cc1]

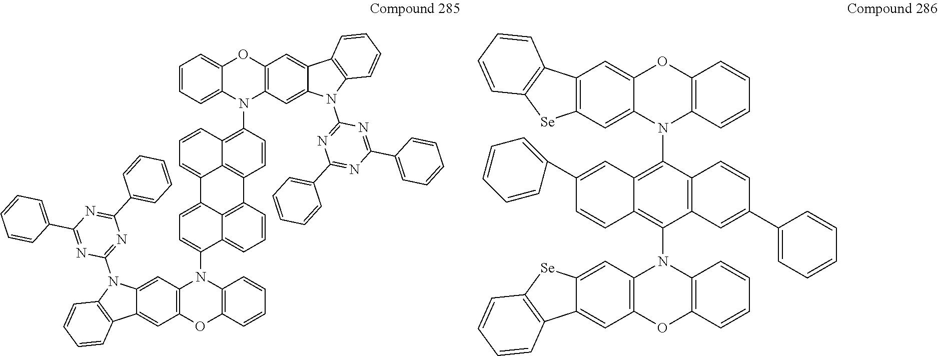 [SMILES: c1ccc(-c2nc(-c3ccccc3)nc(-n3c4ccccc4c4cc5c(cc43)N(c3ccc4c6cccc7c(N8c9ccccc9Oc9cc%10c%11ccccc%11n(-c%11nc(-c%12ccccc%12)nc(-c%12ccccc%12)n%11)c%10cc98)ccc(c8cccc3c84)c76)c3ccccc3O5)n2)cc1 ; c1ccc(-c2ccc3c(N4c5ccccc5Oc5cc6c(cc54)[se]c4ccccc46)c4cc(-c5ccccc5)ccc4c(N4c5ccccc5Oc5cc6c(cc54)[se]c4ccccc46)c3c2)cc1]

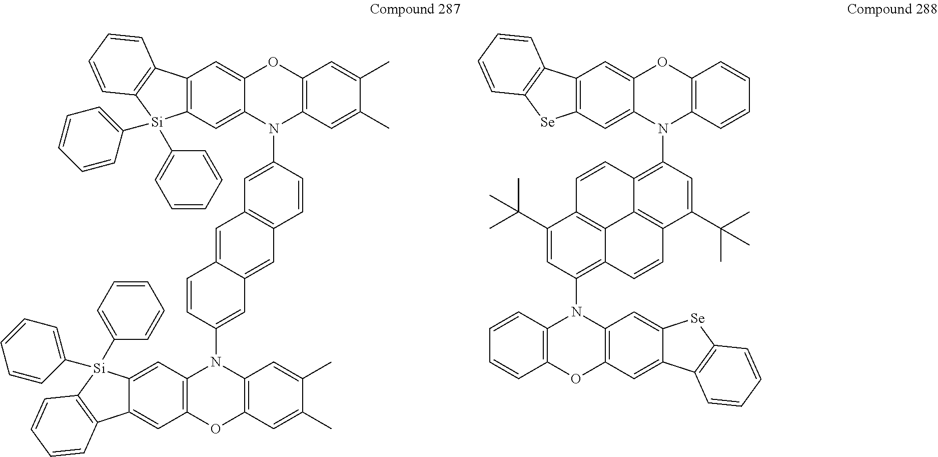 [SMILES: Cc1cc2c(cc1C)N(c1ccc3cc4cc(N5c6cc(C)c(C)cc6Oc6cc7c(cc65)[Si](c5ccccc5)(c5ccccc5)c5ccccc5-7)ccc4cc3c1)c1cc3c(cc1O2)-c1ccccc1[Si]3(c1ccccc1)c1ccccc1 ; CC(C)(C)c1cc(N2c3ccccc3Oc3cc4c(cc32)[se]c2ccccc24)c2ccc3c(C(C)(C)C)cc(N4c5ccccc5Oc5cc6c(cc54)[se]c4ccccc46)c4ccc1c2c43]

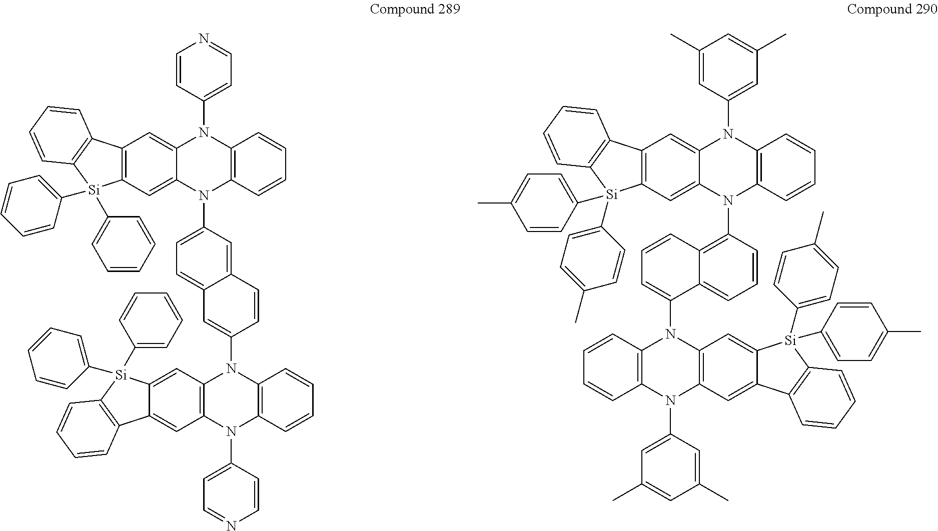

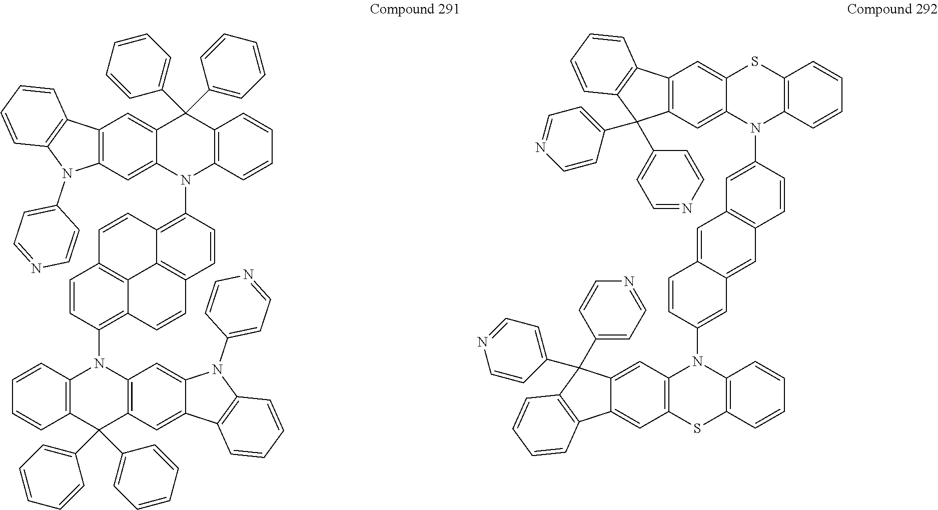 [SMILES: c1ccc(C2(c3ccccc3)c3ccccc3N(c3ccc4ccc5c(N6c7ccccc7C(c7ccccc7)(c7ccccc7)c7cc8c9ccccc9n(-c9ccncc9)c8cc76)ccc6ccc3c4c65)c3cc4c(cc32)c2ccccc2n4-c2ccncc2)cc1 ; c1ccc2c(c1)Sc1cc3c(cc1N2c1ccc2cc4cc(N5c6ccccc6Sc6cc7c(cc65)C(c5ccncc5)(c5ccncc5)c5ccccc5-7)ccc4cc2c1)C(c1ccncc1)(c1ccncc1)c1ccccc1-3]

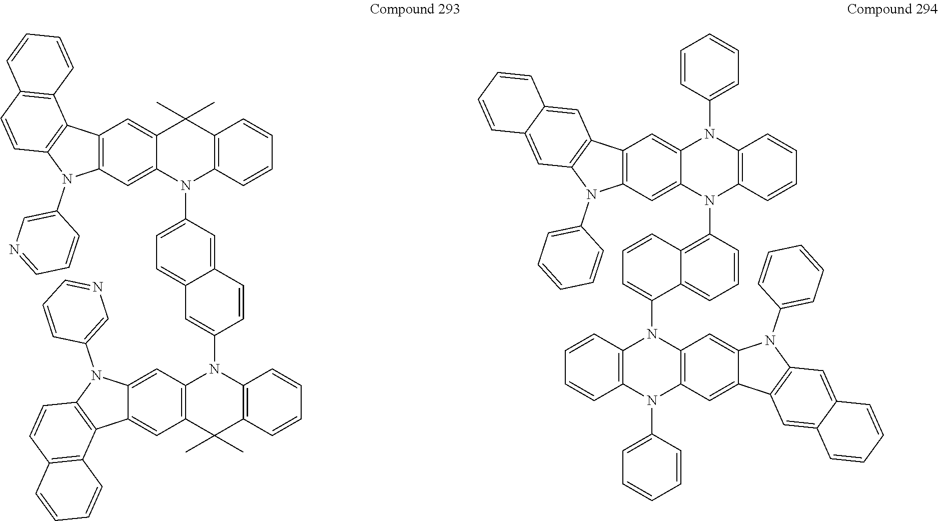 [SMILES: CC1(C)c2ccccc2N(c2ccc3cc(N4c5ccccc5C(C)(C)c5cc6c7c8ccccc8ccc7n(-c7cccnc7)c6cc54)ccc3c2)c2cc3c(cc21)c1c2ccccc2ccc1n3-c1cccnc1 ; c1ccc(N2c3ccccc3N(c3cccc4c(N5c6ccccc6N(c6ccccc6)c6cc7c8cc9ccccc9cc8n(-c8ccccc8)c7cc65)cccc34)c3cc4c(cc32)c2cc3ccccc3cc2n4-c2ccccc2)cc1]

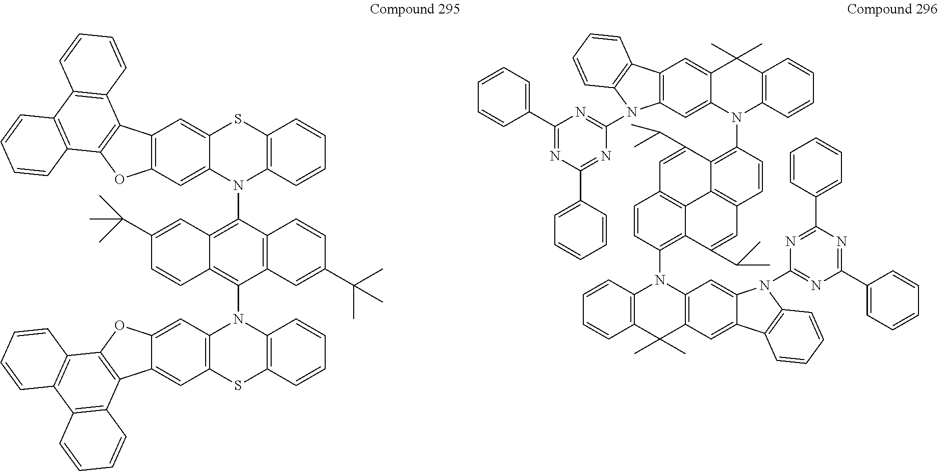 [SMILES: CC(C)(C)c1ccc2c(N3c4ccccc4Sc4cc5c(cc43)oc3c4ccccc4c4ccccc4c53)c3cc(C(C)(C)C)ccc3c(N3c4ccccc4Sc4cc5c(cc43)oc3c4ccccc4c4ccccc4c53)c2c1 ; CC(C)c1cc2ccc(N3c4ccccc4C(C)(C)c4cc5c6ccccc6n(-c6nc(-c7ccccc7)nc(-c7ccccc7)n6)c5cc43)c3c(C(C)C)cc4ccc(N5c6ccccc6C(C)(C)c6cc7c8ccccc8n(-c8nc(-c9ccccc9)nc(-c9ccccc9)n8)c7cc65)c1c4c23]

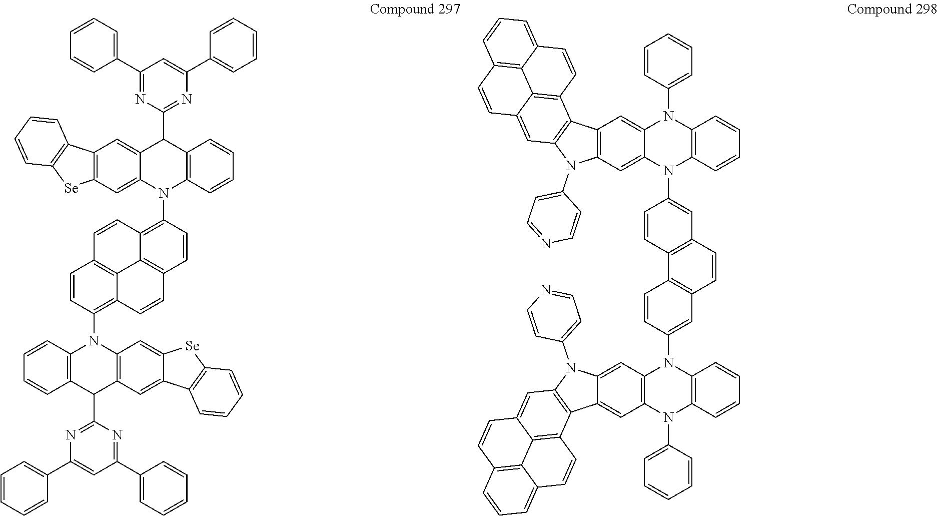 [SMILES: c1ccc(-c2cc(-c3ccccc3)nc(C3c4ccccc4N(c4ccc5ccc6c(N7c8ccccc8C(c8nc(-c9ccccc9)cc(-c9ccccc9)n8)c8cc9c(cc87)[se]c7ccccc79)ccc7ccc4c5c76)c4cc5[se]c6ccccc6c5cc43)n2)cc1 ; c1ccc(-n2c3ccccc3n(-c3ccc4c(ccc5cc(-n6c7ccccc7n(-c7ccccc7)c7cc8c9c%10ccc%11cccc%12ccc(cc9n(-c9ccncc9)c8cc76)c%10c%12%11)ccc54)c3)c3cc4c(cc32)c2c3ccc5cccc6ccc(cc2n4-c2ccncc2)c3c65)cc1]

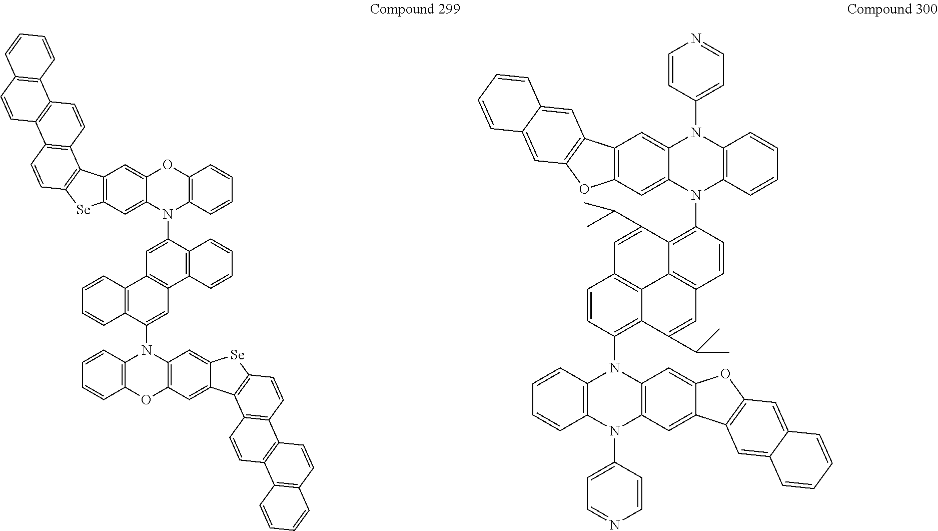 [SMILES: c1ccc2c(c1)Oc1cc3c(cc1N2c1cc2c4ccccc4c(N4c5ccccc5Oc5cc6c(cc54)[se]c4ccc5c7ccc8ccccc8c7ccc5c46)cc2c2ccccc12)[se]c1ccc2c4ccc5ccccc5c4ccc2c13 ; CC(C)c1cc2ccc(N3c4ccccc4N(c4ccncc4)c4cc5c(cc43)oc3cc4ccccc4cc35)c3c(C(C)C)cc4ccc(N5c6ccccc6N(c6ccncc6)c6cc7c(cc65)oc5cc6ccccc6cc57)c1c4c23]

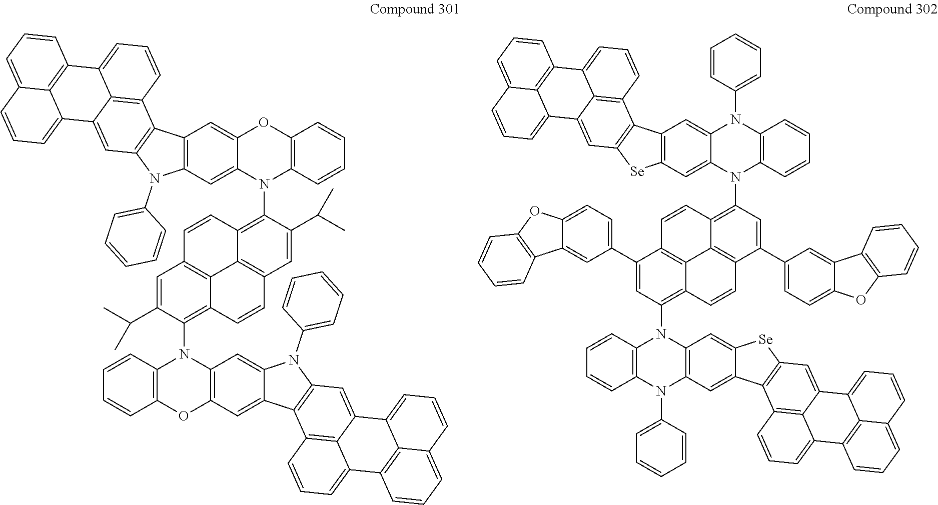 [SMILES: CC(C)c1cc2ccc3c(-n4c5ccccc5oc5cc6c7c8cccc9c%10cccc%11cccc(c(cc7n(-c7ccccc7)c6cc54)c98)c%11%10)c(C(C)C)cc4ccc(c1-n1c5ccccc5oc5cc6c7c8cccc9c%10cccc%11cccc(c(cc7n(-c7ccccc7)c6cc51)c98)c%11%10)c2c43 ; c1ccc(-n2c3ccccc3n(-c3cc(-c4ccc5oc6ccccc6c5c4)c4ccc5c(-n6c7ccccc7n(-c7ccccc7)c7cc8c(cc76)[se]c6cc7c9cccc%10cccc(c%11cccc(c68)c%117)c%109)cc(-c6ccc7oc8ccccc8c7c6)c6ccc3c4c65)c3cc4[se]c5cc6c7cccc8cccc(c9cccc(c5c4cc32)c96)c87)cc1]

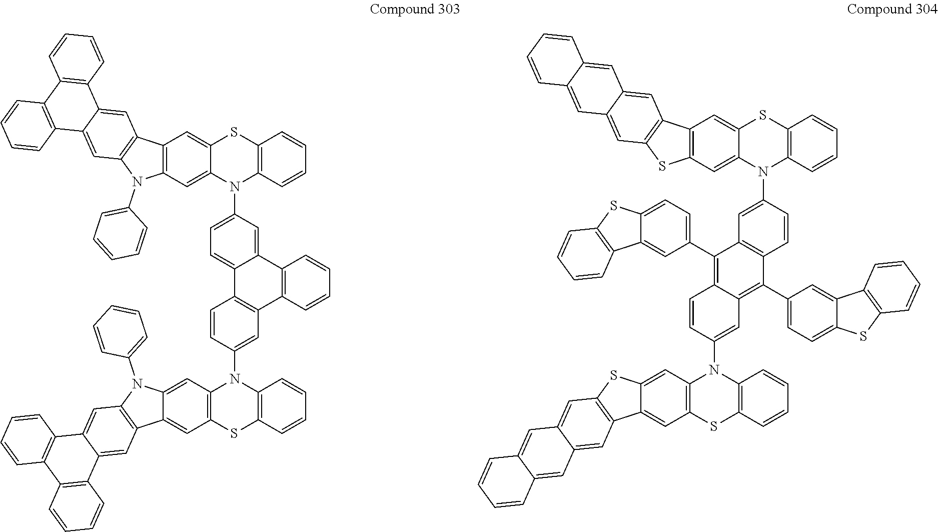 [SMILES: c1ccc(-n2c3cc4c(cc3c3cc5c6ccccc6c6ccccc6c5cc32)Sc2ccccc2N4c2ccc3c4ccc(N5c6ccccc6Sc6cc7c8cc9c%10ccccc%10c%10ccccc%10c9cc8n(-c8ccccc8)c7cc65)cc4c4ccccc4c3c2)cc1 ; c1ccc2c(c1)Sc1cc3c(cc1N2c1ccc2c(-c4ccc5sc6ccccc6c5c4)c4cc(N5c6ccccc6Sc6cc7c(cc65)sc5cc6cc8ccccc8cc6cc57)ccc4c(-c4ccc5sc6ccccc6c5c4)c2c1)sc1cc2cc4ccccc4cc2cc13]

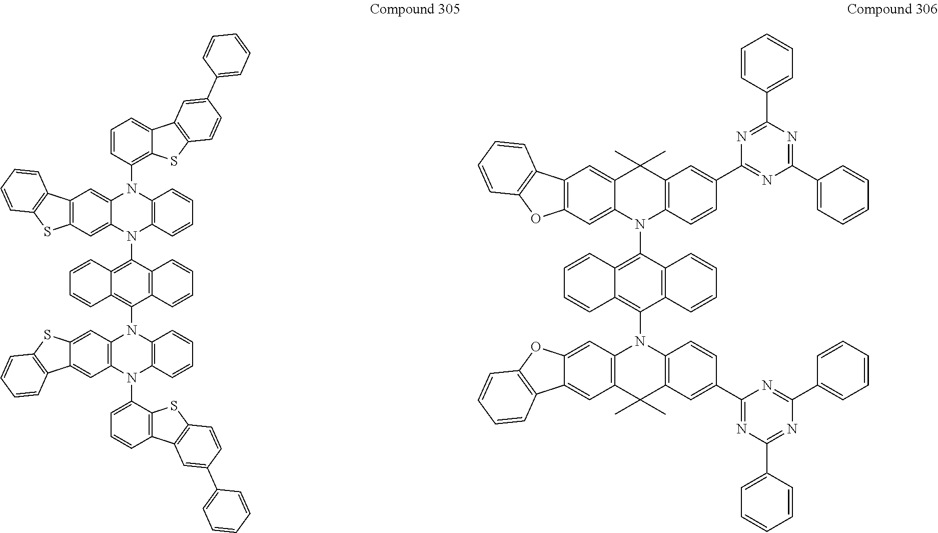 [SMILES: c1ccc(-c2ccc3sc4c(N5c6ccccc6N(c6c7ccccc7c(N7c8ccccc8N(c8cccc9c8sc8ccc(-c%10ccccc%10)cc89)c8cc9c(cc87)sc7ccccc79)c7ccccc67)c6cc7sc8ccccc8c7cc65)cccc4c3c2)cc1 ; CC1(C)c2cc(-c3nc(-c4ccccc4)nc(-c4ccccc4)n3)ccc2N(c2c3ccccc3c(N3c4ccc(-c5nc(-c6ccccc6)nc(-c6ccccc6)n5)cc4C(C)(C)c4cc5c(cc43)oc3ccccc35)c3ccccc23)c2cc3oc4ccccc4c3cc21]

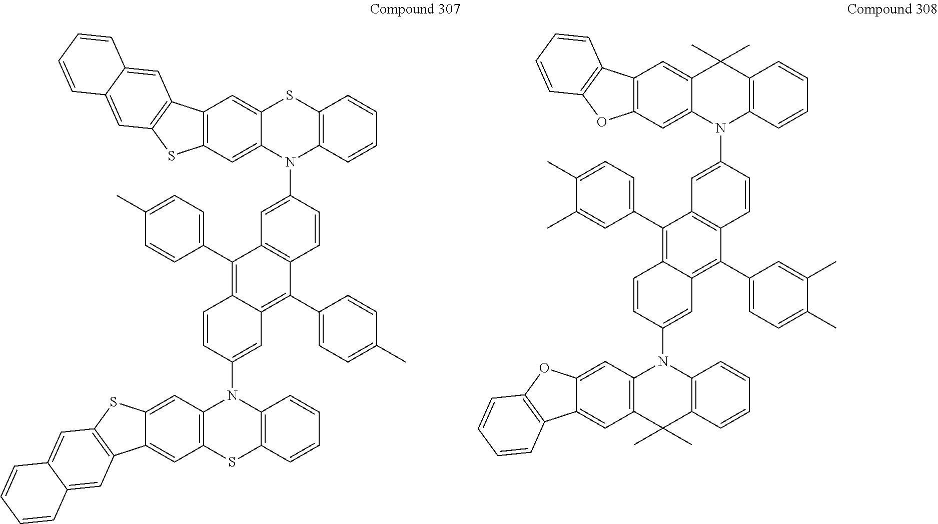 [SMILES: Cc1ccc(-c2c3ccc(N4c5ccccc5Sc5cc6c(cc54)sc4cc5ccccc5cc46)cc3c(-c3ccc(C)cc3)c3ccc(N4c5ccccc5Sc5cc6c(cc54)sc4cc5ccccc5cc46)cc23)cc1 ; Cc1ccc(-c2c3ccc(N4c5ccccc5C(C)(C)c5cc6c(cc54)oc4ccccc46)cc3c(-c3ccc(C)c(C)c3)c3ccc(N4c5ccccc5C(C)(C)c5cc6c(cc54)oc4ccccc46)cc23)cc1C]

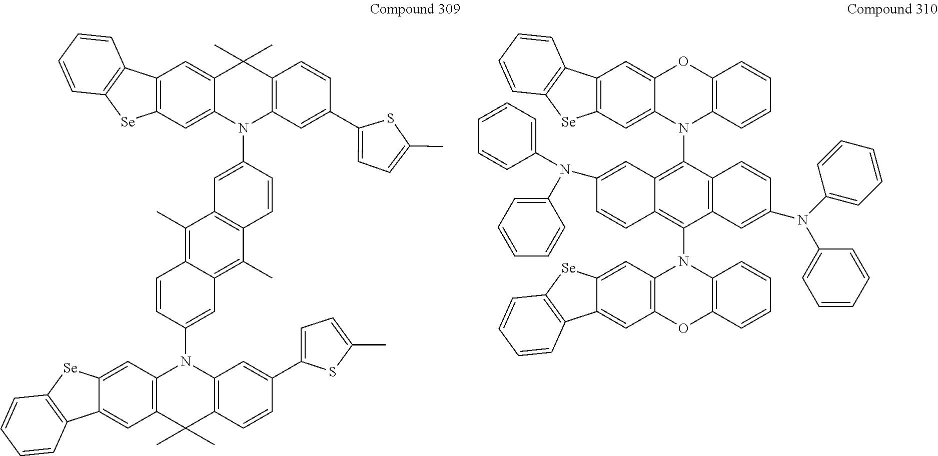

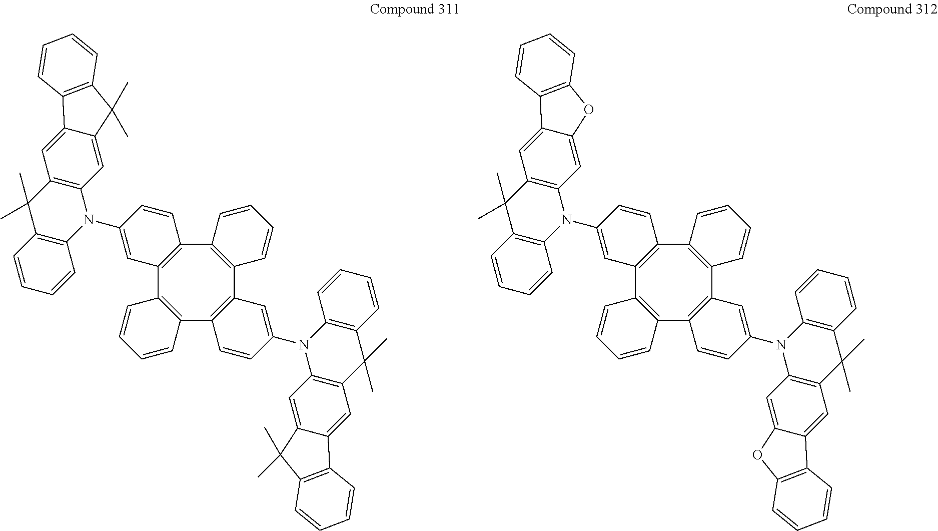

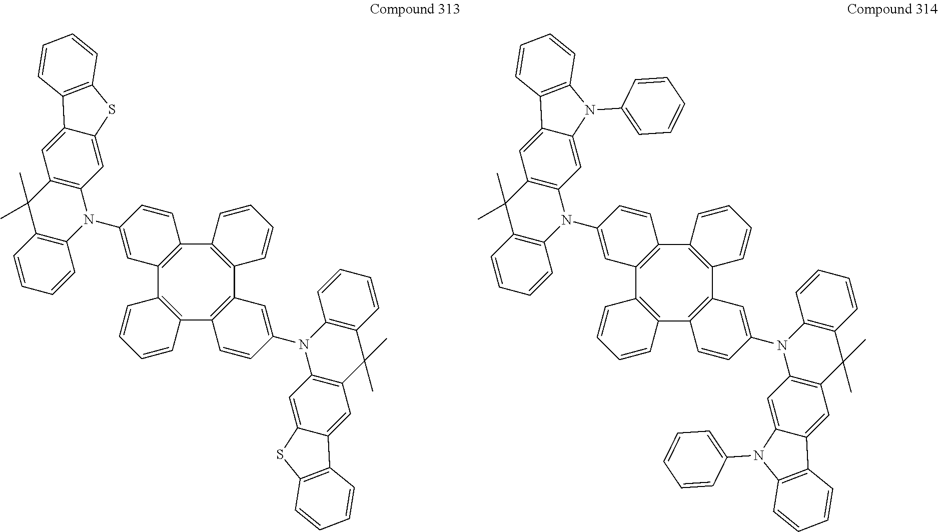

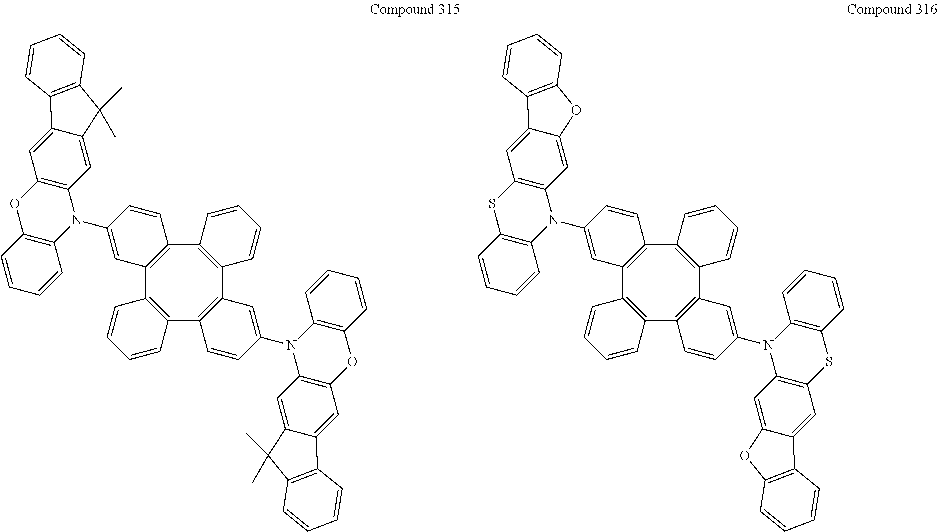 [SMILES: CC1(C)c2ccccc2-c2cc3c(cc21)N(c1ccc2c(c1)-c1ccccc1-c1ccc(N4c5ccccc5Oc5cc6c(cc54)C(C)(C)c4ccccc4-6)cc1-c1ccccc1-2)c1ccccc1O3 ; c1ccc2c(c1)Sc1cc3c(cc1N2c1ccc2c(c1)-c1ccccc1-c1ccc(N4c5ccccc5Sc5cc6c(cc54)oc4ccccc46)cc1-c1ccccc1-2)oc1ccccc13]

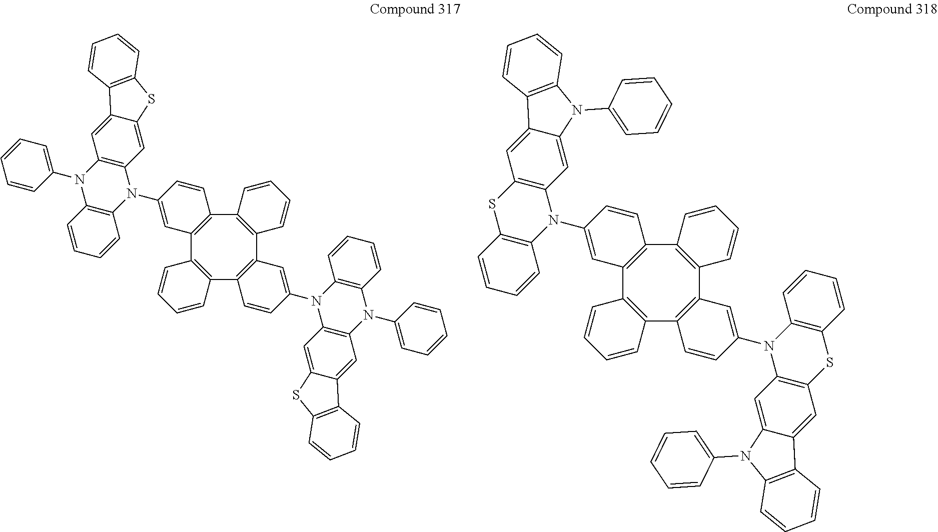 [SMILES: c1ccc(N2c3ccccc3N(c3ccc4c(c3)-c3ccccc3-c3ccc(N5c6ccccc6N(c6ccccc6)c6cc7c(cc65)sc5ccccc57)cc3-c3ccccc3-4)c3cc4sc5ccccc5c4cc32)cc1 ; c1ccc(-n2c3ccccc3c3cc4c(cc32)N(c2ccc3c(c2)-c2ccccc2-c2ccc(N5c6ccccc6Sc6cc7c8ccccc8n(-c8ccccc8)c7cc65)cc2-c2ccccc2-3)c2ccccc2S4)cc1]

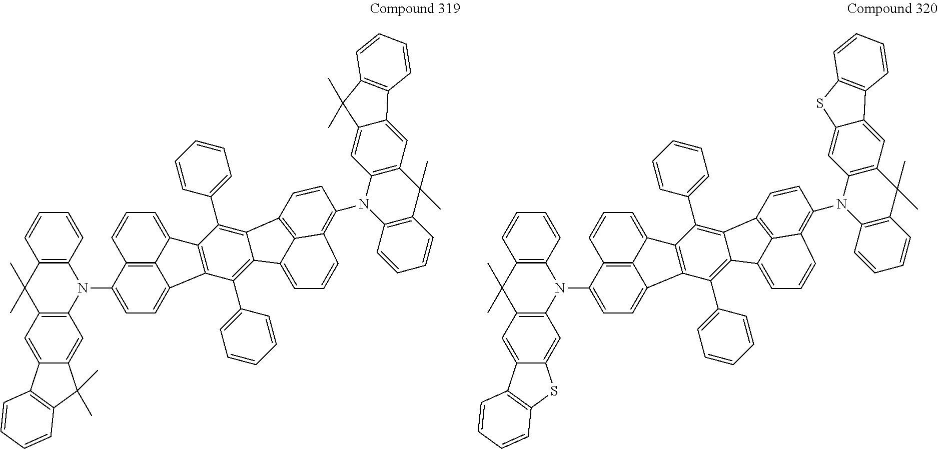 [SMILES: CC1(C)c2ccccc2-c2cc3c(cc21)N(c1ccc2c4c(-c5ccccc5)c5c6cccc7c(N8c9ccccc9C(C)(C)c9cc%10c(cc98)C(C)(C)c8ccccc8-%10)ccc(c5c(-c5ccccc5)c4c4cccc1c42)c76)c1ccccc1C3(C)C ; CC1(C)c2ccccc2N(c2ccc3c4c(-c5ccccc5)c5c6cccc7c(N8c9ccccc9C(C)(C)c9cc%10c(cc98)sc8ccccc8%10)ccc(c5c(-c5ccccc5)c4c4cccc2c43)c76)c2cc3sc4ccccc4c3cc21]

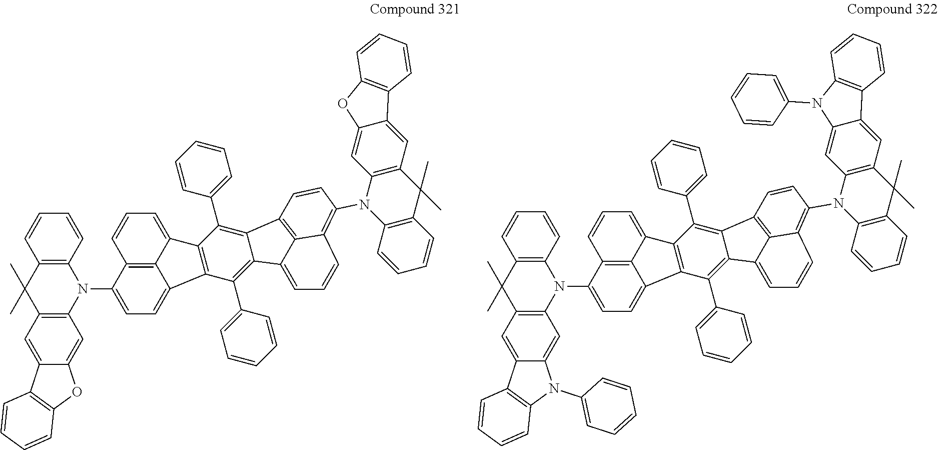 [SMILES: CC1(C)c2ccccc2N(c2ccc3c4c(-c5ccccc5)c5c6cccc7c(N8c9ccccc9C(C)(C)c9cc%10c(cc98)oc8ccccc8%10)ccc(c5c(-c5ccccc5)c4c4cccc2c43)c76)c2cc3oc4ccccc4c3cc21 ; CC1(C)c2ccccc2N(c2ccc3c4c(-c5ccccc5)c5c6cccc7c(N8c9ccccc9C(C)(C)c9cc%10c%11ccccc%11n(-c%11ccccc%11)c%10cc98)ccc(c5c(-c5ccccc5)c4c4cccc2c43)c76)c2cc3c(cc21)c1ccccc1n3-c1ccccc1]

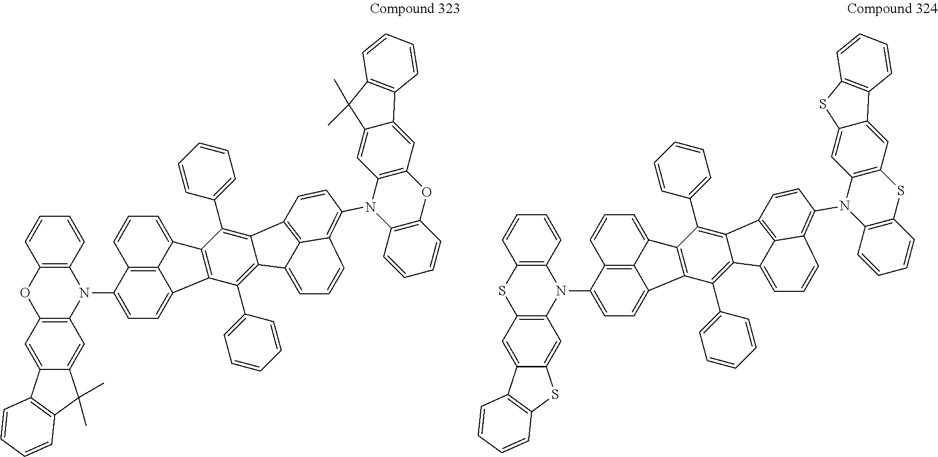 [SMILES: CC1(C)c2ccccc2-c2cc3c(cc21)N(c1ccc2c4c(-c5ccccc5)c5c6cccc7c(N8c9ccccc9Oc9cc%10c(cc98)C(C)(C)c8ccccc8-%10)ccc(c5c(-c5ccccc5)c4c4cccc1c42)c76)c1ccccc1O3 ; c1ccc(-c2c3c4cccc5c(N6c7ccccc7Sc7cc8c(cc76)sc6ccccc68)ccc(c3c(-c3ccccc3)c3c6cccc7c(N8c9ccccc9Sc9cc%10c(cc98)sc8ccccc8%10)ccc(c23)c76)c54)cc1]

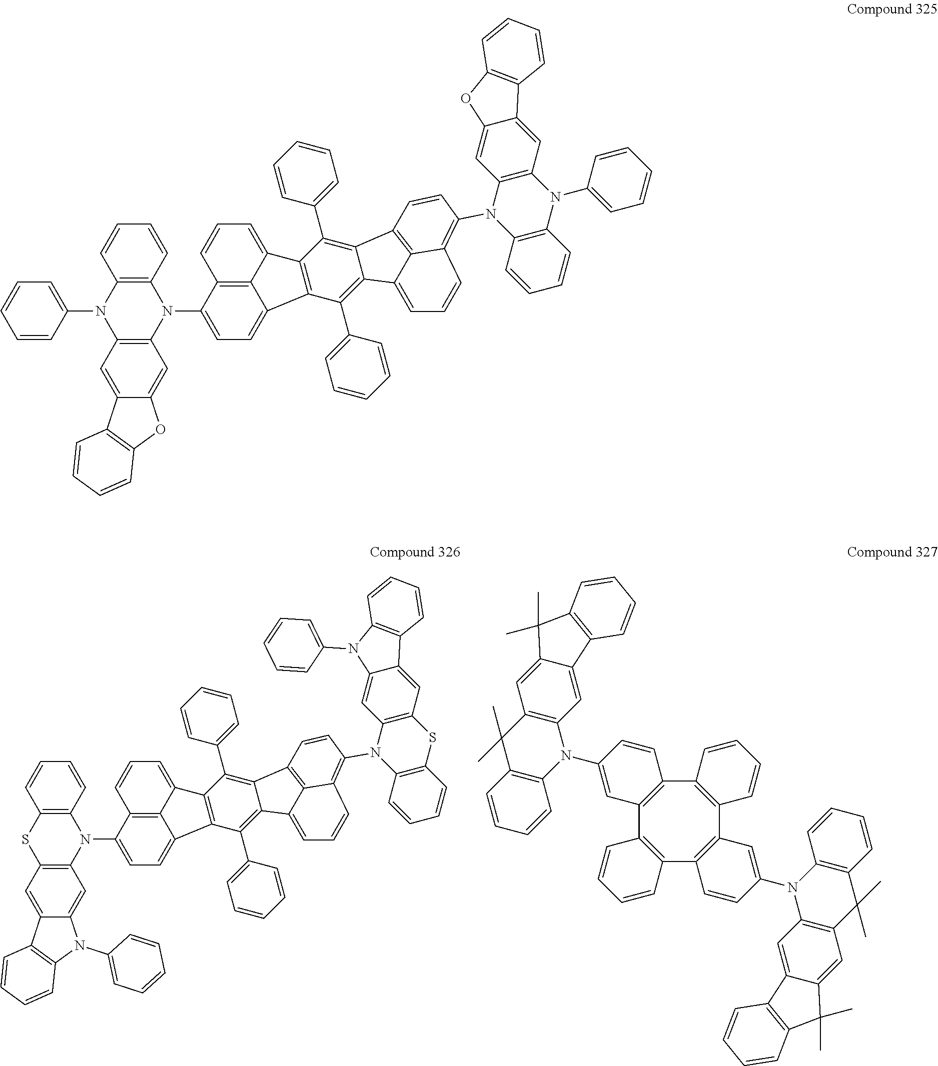

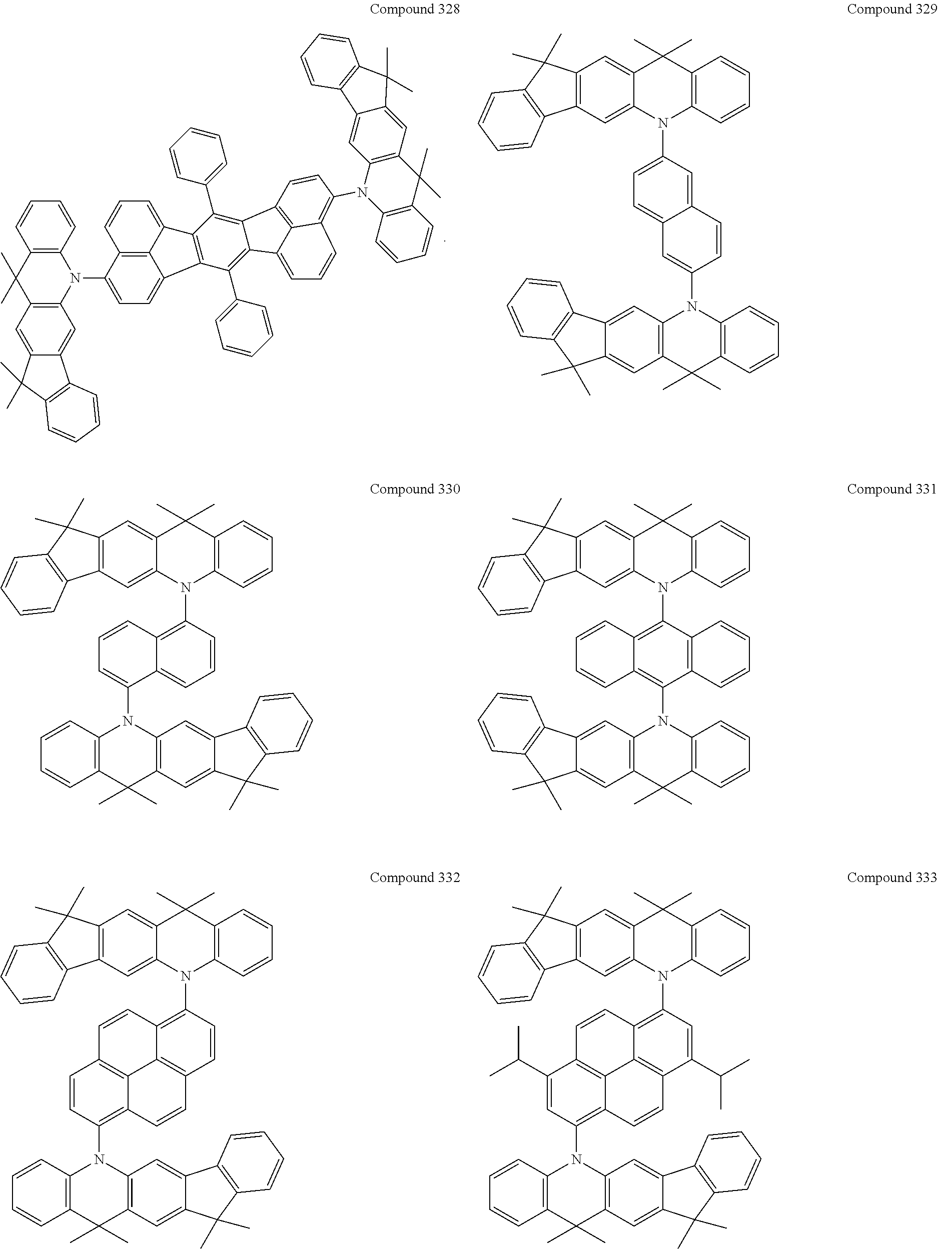

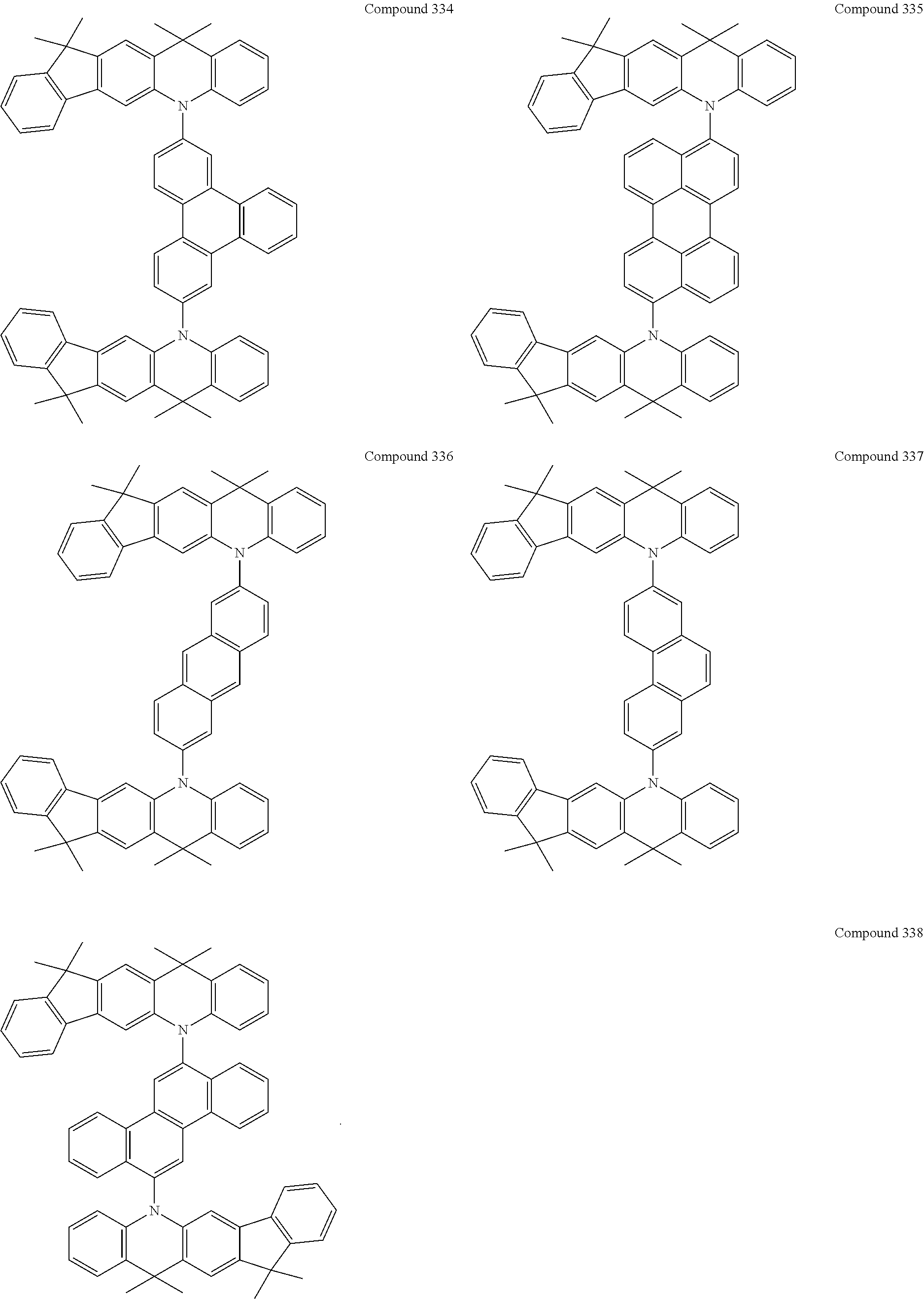

In another embodiment of the present invention, an organic electroluminescence device is disclosed. The organic electroluminescence device comprises a pair of electrodes composed of a cathode and an anode, and a light emitting layer between the pair of electrodes. The light emitting layer comprises the heteroaromatic compound of formula (1).

In some embodiments, the light emitting layer comprising the heteroaromatic compound of formula (1) is a fluorescent guest material. In particular, the light emitting layer emits blue, green, yellow, red, or orange fluorescence.

In a further embodiment of the present invention, the organic electroluminescence device is a lighting panel. In other embodiment of the present invention, the organic electroluminescence device is a backlight panel.

Detailed preparation of the heteroaromatic compounds of the present invention will be clarified by exemplary embodiments below, but the present invention is not limited thereto. EXAMPLES 1 to 15 show the preparation of the heteroaromatic compounds of the present invention, and EXAMPLE 16 shows the fabrication and test reports of the organic EL devices.

### EXAMPLE 1

### Synthesis of methyl 2-((9,9-dimethyl-9H-fluoren-2-yl)amino)-benzoate

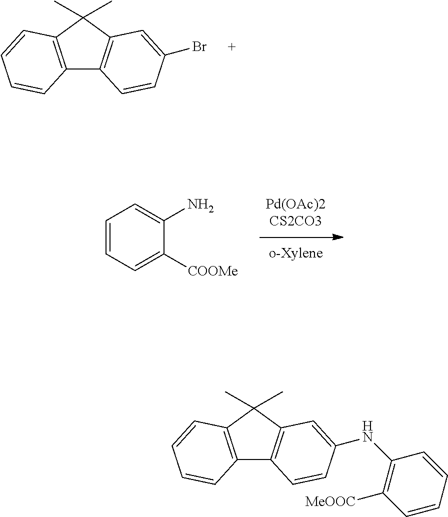 [SMILES: CC1(C)c2ccccc2-c2ccc(Br)cc21 ; COC(=O)c1ccccc1N ; COC(=O)c1ccccc1Nc1ccc2c(c1)C(C)(C)c1ccccc1-2]

A mixture of 10 g (36.6 mmol) of 2-bromo-9,9-dimethyl-9H-fluorene, 6.64 g (43.9 mmol) of methyl 2-aminobenzoate, 0.3 g (1.46 mmol) of Pd(OAc)₂, 17.9 g (54.9 mmol) of cesium carbonate, and 120 ml of o-xylene was degassed under nitrogen, and then heated to reflux for 12 hrs. After the reaction finished, the mixture was allowed to cool to room temperature. Subsequently, the solvent was removed under reduced pressure, and the crude product was purified by column chromatography, yielding 10 g of methyl 2-((9,9-dimethyl-9H-fluoren-2-yl)amino) benzoate as yellow oil (79.6%). ¹H NMR (CDCl₃, 400 MHz): chemical shift (ppm) 8.97(s, 1H),8.11(d, 1H), 7.87(d, 1H), 7.75-7.64(m, 3H), 7.41-7.29(m, 3H), 7.08(m, 1H), 6.92(d, 1H), 6.77(d, 1H), 3.79(s, 3H), 1.57(s, 3H), 1.54(s, 3H).

### Synthesis of 2-(2-((9,9-dimethyl-9H-fluoren-2-yl)amino)phenyl)-propan-2-ol

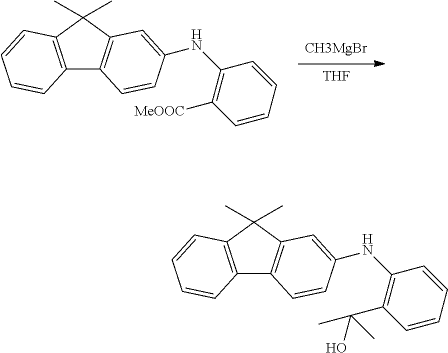 [SMILES: COC(=O)c1ccccc1Nc1ccc2c(c1)C(C)(C)c1ccccc1-2 ; "CH3MgBr" ; CC(C)(O)c1ccccc1Nc1ccc2c(c1)C(C)(C)c1ccccc1-2 ; "THF"]

The compound methyl 2-((9,9-dimethyl-9H-fluoren-2-yl)amino) benzoate (10 g, 29.1 mmol) was mixed with 100 ml of THF, to which 58 ml of 3 M methylmagnesium bromide was then added slowly at room temperature to obtain a mixture. The mixture was then stirred at room temperature to react for 3 hrs. After the reaction finished, the ammonium chloride solution was added to obtain a reaction mixture. Subsequently, the reaction mixture was extracted with ethyl acetate/water and the organic layer was separated. The solvent was removed from the organic layer under reduced pressure, and the crude product was purified by column chromatography, yielding 9 g of 2-(2-((9,9-dimethyl-9H-fluoren-2-yl)amino)phenyl)-propan-2-ol as yellow oil (90%). ¹H NMR (CDCl₃, 400 MHz): chemical shift (ppm) 8.91(s, 1H),7.81(d, 1H), 7.59(d, 1H), 7.54(d, 1H), 7.35-7.39(m, 3H), 7.29(m, 1H), 6.92(m, 1H), 6.76-6.74(d, 2H), 6.58(d, 1H), 3.88(s, 1H), 1.53(s, 3H), 1.51(s, 3H), 1.35(s, 6H).

### Synthesis of 7,7,13,13-tetramethyl-7,13-dihydro-5H-indeno-[1,2-b]-acridine

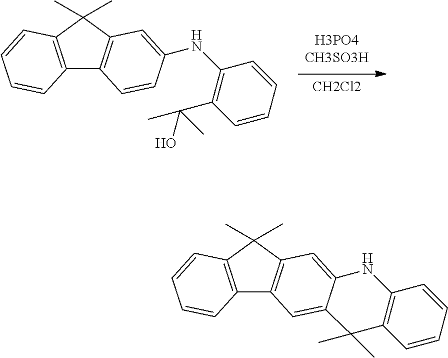

The compound 2-(2-((9,9-dimethyl-9H-fluoren-2-yl)amino)phenyl)-propan-2-ol (27 g, 78.6 mmol) was mixed with 400 ml of CH₂Cl₂, to which 51 ml of methane sulfonic acid and 37 ml of phosphoric acid was then added slowly at room temperature to obtain a mixture. The mixture was then stirred at room temperature to react for 12 hrs. After the reaction finished, ice-cold water was added to obtain a reaction mixture. Subsequently, 20% sodium hydroxide solution was added to the reaction mixture, which was then extracted with ethyl acetate/water and then the organic layer was separated. The solvent was removed from the organic layer under reduced pressure, and the crude product was purified by column chromatography, yielding 15 g of 7,7,13,13-tetramethyl-7,13-dihydro-5H-indeno-[1,2-b]acridine as yellow solid (58.8%). ¹H NMR (CDCl₃, 400 MHz): chemical shift (ppm) 8.95(s, 1H),7.79(s, 1H), 7.72(d, 1H), 7.43(d, 1H), 7.36(d, 1H), 7.25(m, 1H), 7.16(dd, 1H), 7.04(m, 1H), 6.87(s, 1H),6.81-6.78(m, 2H) 1.55(s, 6H), 1.38(s, 6H).

### Synthesis of 9,10-bis(7,7,13,13-tetramethyl-7,13-dihydro-5H-indeno-[1,2-b]acridin-5-yl)anthracene (Compound 3)

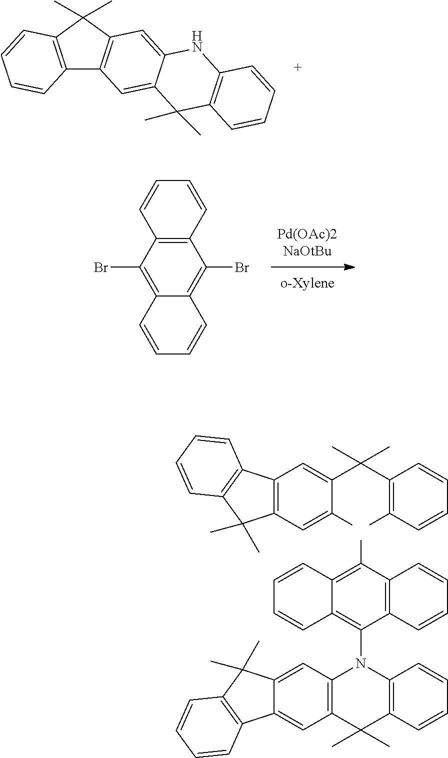

A mixture of 6.4 g (19.6 mmol) of 7,7,13,13-tetramethyl-7,13-dihydro-5H-indeno-[1,2-b]acridine, 3 g (8.9 mmol) of 9,10-dibromoanthracene, 0.08 g (0.36 mmol) of Pd(OAc)₂, 2.56 g (26.7 mmol) of sodium tert-butoxide, and 50 ml of o-xylene was degassed under nitrogen, and then heated at 140° C. for 12 hrs. After the reaction finished, the mixture was allowed to cool to room temperature. Subsequently, 150 ml of methanol was added to the mixture, and then the resulting precipitate was filtered and washed with methanol to get a yellow solid. Yield: 2.65 g, 36%. ¹H NMR (CDCl₃, 400 MHz): chemical shift (ppm) 7.88(d, 4H),7.78(s, 2H), 7.72(d, 2H), 7.44(d, 2H), 7.39-7.35(m, 6H), 7.23(m, 2H), 7.14(dd, 2H), 7.03(m, 2H), 6.87(s, 2H),6.81-6.78(m, 4H) 1.54(s, 12H), 1.36(s, 12H). MS(m/z, EI⁺): 825.4.

### EXAMPLE 2

### Synthesis of 1,6-bis(7,7,13,13-tetramethyl-7,13-dihydro-5H-indeno-[1,2-b]acridin-5-yl)pyrene (Compound 7)

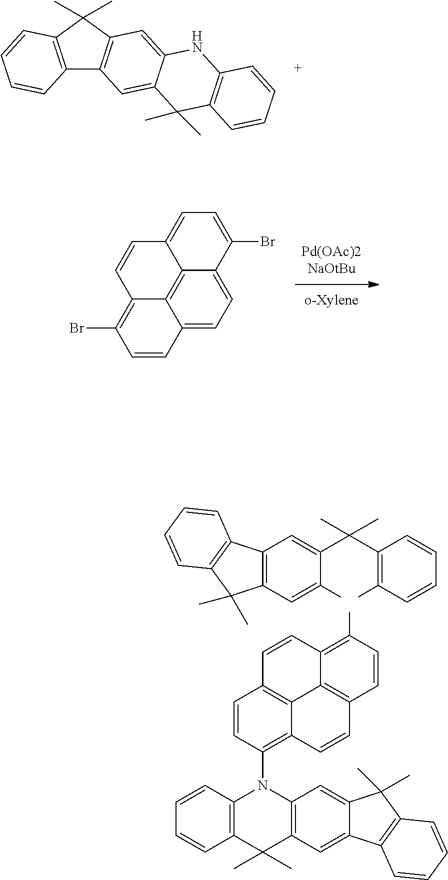

The same synthesis procedure as in EXAMPLE 1 was used, except that 1,6-dibromopyrene was used instead of 9,10-dibromoanthracene to obtain the desired compound of 1,6-bis(7,7,13,13-tetramethyl-7,13-dihydro-5H-indeno[1,2-b]acridin-5-yl)pyrene. MS(m/z,EI⁺): 849.3.

### EXAMPLE 3

### Synthesis of 2,7-bis(7,7,13,13-tetramethyl-7,13-dihydro-5H-indeno-[1,2-b]acridin-5-yl)triphenylene (Compound 8)

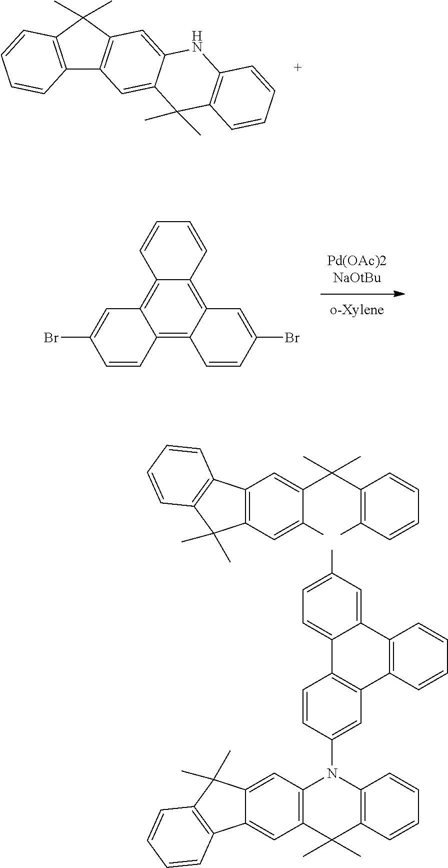

The same synthesis procedure as in EXAMPLE 1 was used, except that 2,7-dibromotriphenylene was used instead of 9,10-dibromoanthracene to obtain the desired compound of 2,7-bis(7,7,13,13-tetramethyl-7,13-dihydro-5H-indeno[1,2-b]acridin-5-yl)triphenylene. MS(m/z,EI⁺): 875.4.

### EXAMPLE 4

### Synthesis of 3,9-bis(7,7,13,13-tetramethyl-7,13-dihydro-5H-indeno-[1,2-b]acridin-5-yl)perylene (Compound 9)

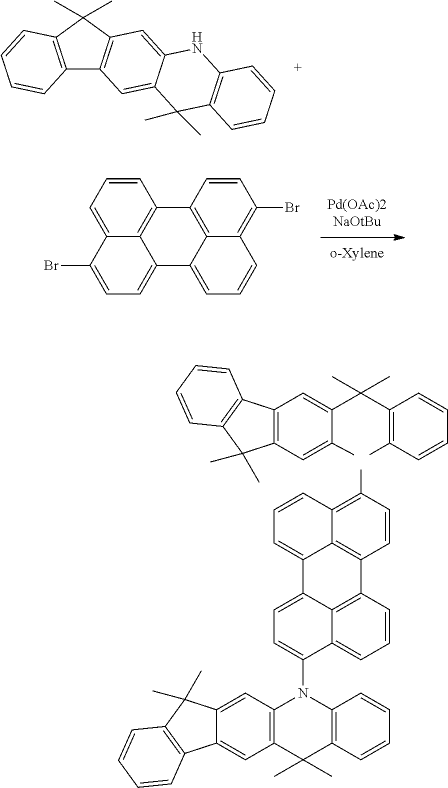

The same synthesis procedure as in EXAMPLE 1 was used, except that 3,9-dibromoperylene was used instead of 9,10-dibromoanthracene to obtain the desired compound of 3,9-bis(7,7,13,13-tetramethyl-7,13-dihydro-5H-indeno[1,2-b]acridin-5-yl)perylene. MS(m/z,EI⁺): 899.3.

### EXAMPLE 5

### Synthesis of 5,5′-(5,10-diisopropylpyrene-1,6-diyl)bis(7,7,13,13-tetramethyl-7,13-dihydro-5H-indeno[1,2-b]acridine) (Compound 35)

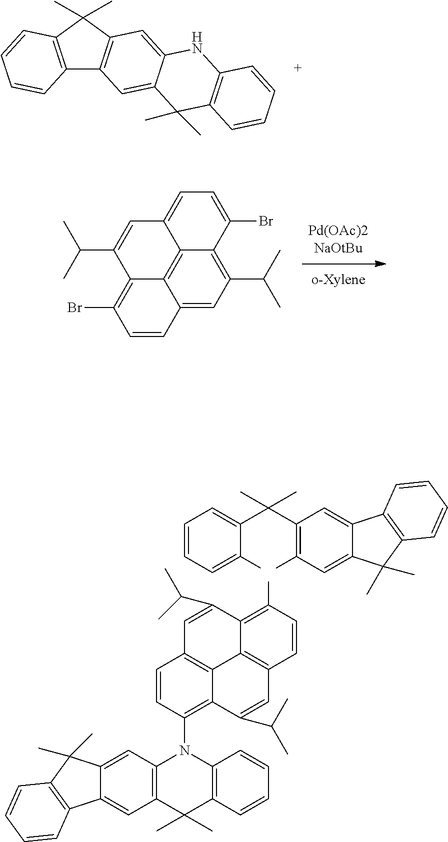

The same synthesis procedure as in EXAMPLE 1 was used, except that 1,6-dibromo-5,10-diisopropylpyrene was used instead of 9,10-dibromoanthracene to obtain the desired compound of 5, 5′-(5,10-diisopropylpyrene-1,6-diyl)bis(7,7,13,13-tetramethyl-7,13-dihydro-5H-indeno[1,2-b]acridine). MS(m/z,EI⁺): 933.6.

### EXAMPLE 6

### Synthesis of 2,6-bis(7,7,13,13-tetramethyl-7,13-dihydro-5H-indeno[1,2-b]acridin-5-yl)naphthalene (Compound 1)

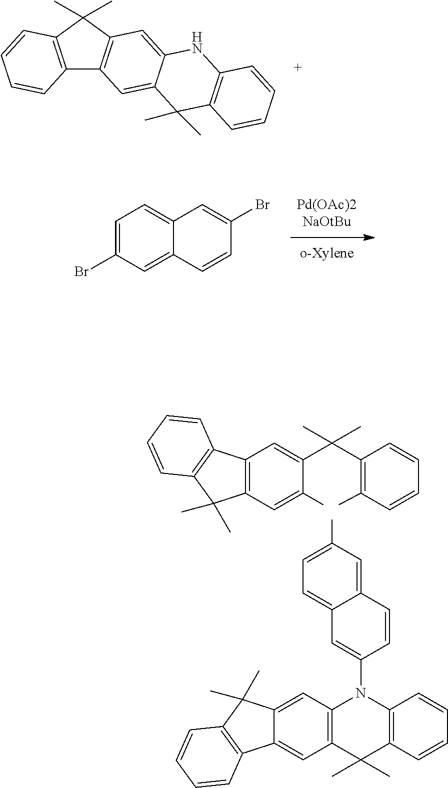

The same synthesis procedure as in EXAMPLE 1 was used, except that 2,6-dibromonaphthalene was used instead of 9,10-dibromoanthracene to obtain the desired compound of 2,6-bis(7,7,13,13-tetramethyl-7,13-dihydro-5H-indeno[1,2-b]acridin-5-yl)naphthalene. MS(m/z,EI⁺): 775.4.

### EXAMPLE 7

### Synthesis of 1,5-bis(7,7,13,13-tetramethyl-7,13-dihydro-5H-indeno-[1,2-b]acridin-5-yl)naphthalene (Compound 2)

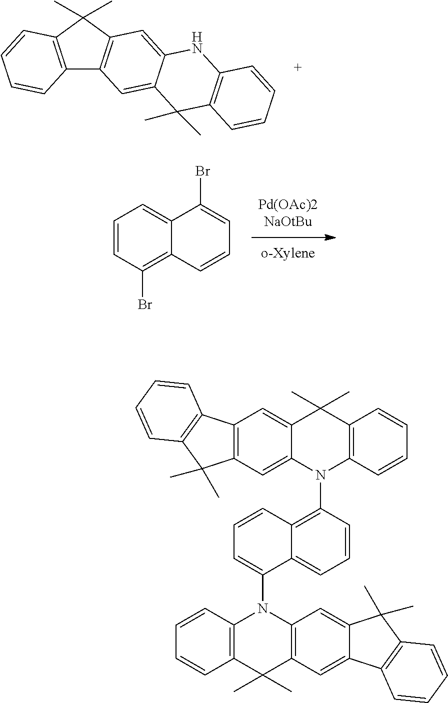

The same synthesis procedure as in EXAMPLE 1 was used, except that 1,5-dibromonaphthalene was used instead of 9,10-dibromoanthracene to obtain the desired compound of 1,5-bis(7,7,13,13-tetramethyl-7,13-dihydro-5H-indeno[1,2-b]acridin-5-yl)naphthalene. MS(m/z,EI⁺): 775.3.

### EXAMPLE 8

### Synthesis of 2,7-bis(7,7,13,13-tetramethyl-7,13-dihydro-5H-indeno-[1,2-b]acridin-5-yl)phenanthrene (Compound 5)

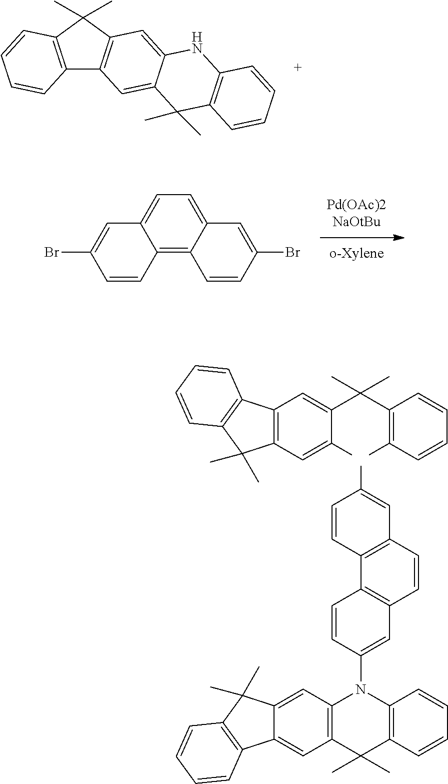

The same synthesis procedure as in EXAMPLE 1 was used, except that 2,7-dibromophenanthrene was used instead of 9,10-dibromoanthracene to obtain the desired compound of 2,7-bis(7,7,13,13-tetramethyl-7,13-dihydro-5H-indeno[1,2-b]acridin-5-yl)phenanthrene. MS(m/z,EI⁺): 825.4.

### EXAMPLE 9

### Synthesis of 6,12-bis(7,7,13,13-tetramethyl-7,13-dihydro-5H-indeno-[1,2-b]acridin-5-yl)chrysene (Compound 6)

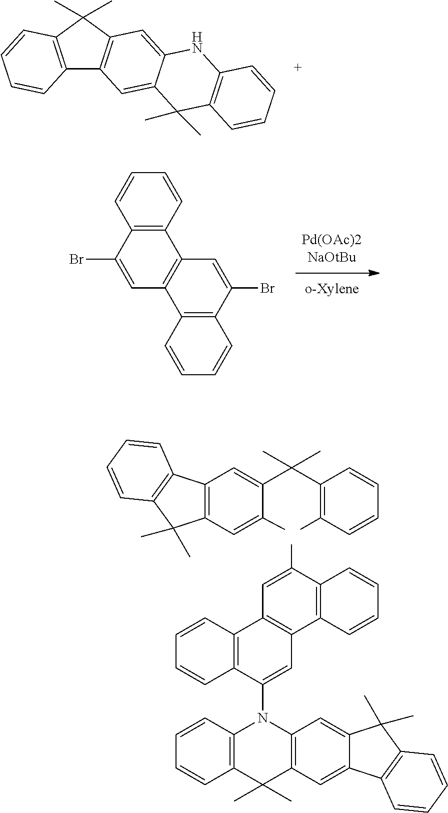

The same synthesis procedure as in EXAMPLE 1 was used, except that 6,12-dibromochrysene was used instead of 9,10-dibromoanthracene to obtain the desired compound of 6,12-bis(7,7,13,13-tetramethyl-7,13-dihydro-5H-indeno[1,2-b]acridin-5-yl)chrysene. MS(m/z,EI⁺): 875.3.

### EXAMPLE 10

### Synthesis of 5,5′-(7,14-diphenylacenaphtho[1,2-k]fluoranthene-3,10-diyl)bis(7,7,13,13-tetramethyl-7,13-dihydro-5H-indeno[1,2-b]acridine) (Compound 319)

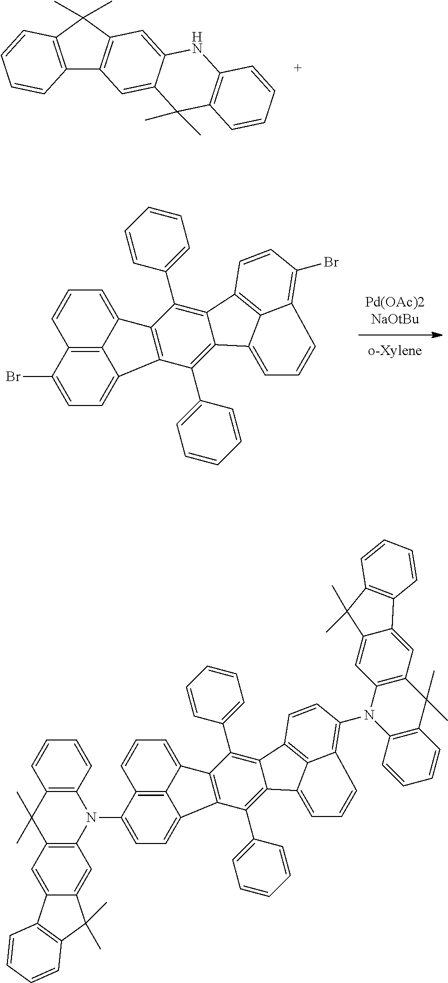 [SMILES: CC1(C)c2ccccc2Nc2cc3c(cc21)-c1ccccc1C3(C)C ; Brc1ccc2c3c(-c4ccccc4)c4c5cccc6c(Br)ccc(c4c(-c4ccccc4)c3c3cccc1c32)c65 ; CC1(C)c2ccccc2-c2cc3c(cc21)N(c1ccc2c4c(-c5ccccc5)c5c6cccc7c(N8c9ccccc9C(C)(C)c9cc%10c(cc98)C(C)(C)c8ccccc8-%10)ccc(c5c(-c5ccccc5)c4c4cccc1c42)c76)c1ccccc1C3(C)C]

The same synthesis procedure as in EXAMPLE 1 was used, except that 3,10-dibromo-7,14-diphenylacenaphtho[1,2-k]fluoranthene was used instead of 9,10-dibromoanthracene to obtain the desired compound of 5,5′-(7,14-diphenylacenaphtho[1,2-k]fluoranthene-3,10-diyl)bis(7,7,13,13-tetramethyl-7,13-dihydro-5H-indeno[1,2-b]acridine). MS(m/z,EI⁺): 1125.6.

### EXAMPLE 11

### Synthesis of methyl 2-(dibenzo[b,d]thiophen-3-ylamino)benzoate

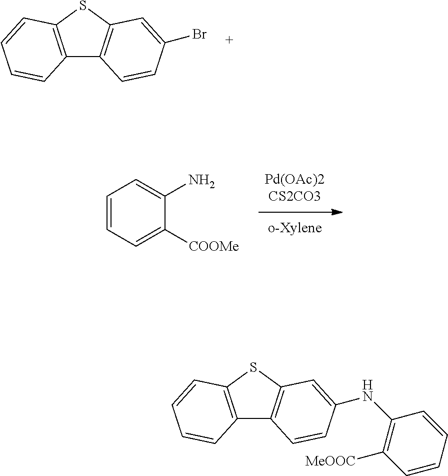 [SMILES: Brc1ccc2c(c1)sc1ccccc12 ; COC(=O)c1ccccc1N ; COC(=O)c1ccccc1Nc1ccc2c(c1)sc1ccccc12]

A mixture of 5 g (19 mmol) of 3-bromodibenzo[b,d]thiophene, 3.45 g (22.8 mmol) of methyl 2-aminobenzoate, 0.17 g (0.76 mmol) of Pd(OAc)₂, 9.28 g (28.5 mmol) of cesium carbonate, and 60 ml of o-xylene was degassed under nitrogen, and then heated to reflux for 12 hrs. After the reaction finished, the mixture was allowed to cool to room temperature. Subsequently, the solvent was removed under reduced pressure, and the crude product was purified by column chromatography, yielding 4.8 g of methyl 2-(dibenzo[b,d]thiophen-3-ylamino)benzoate as yellow oil (75.8%). ¹H NMR (CDCl₃, 400 MHz): chemical shift (ppm) 8.99(s, 1H),8.14(d, 1H), 7.89(d, 1H), 7.79-7.68(m, 3H), 7.45-7.33(m, 3H), 7.12(m, 1H), 6.97(d, 1H), 6.81(d, 1H), 3.79(s, 3H).

### Synthesis of 2-(2-(dibenzo[b,d]thiophen-3-ylamino)phenyl)propan-2-ol

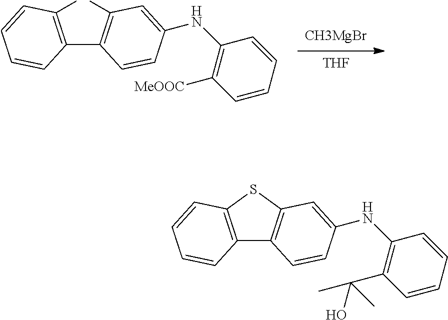

The compound methyl 2-(dibenzo[b,d]thiophen-3-ylamino)benzoate (4.8 g, 14.4 mmol) was mixed with 50 ml of THF, to which 28 ml of 3 M methylmagnesium bromide was then added slowly at room temperature to obtain a mixture. The mixture was then stirred at room temperature to react for 3 hrs. After the reaction finished, the ammonium chloride solution was added to obtain a reaction mixture. Subsequently, the reaction mixture was extracted with ethyl acetate/water and the organic layer was separated. The solvent was removed from the organic layer under reduced pressure, and the crude product was purified by column chromatography, yielding 4.5 g of 2-(2-(dibenzo[b,d]thiophen-3-ylamino)phenyl)propan-2-ol as yellow oil (93.7%). ¹H NMR (CDCl₃, 400 MHz): chemical shift (ppm) 8.94(s, 1H),7.83(d, 1H), 7.62(d, 1H), 7.55(d, 1H), 7.41-7.36(m, 3H), 7.29(m, 1H), 6.94(m, 1H), 6.78-6.75(d, 2H), 6.59(d, 1H), 3.91(s, 1H), 1.35(s, 6H).

### Synthesis of 12,12-dimethyl-7,12-dihydrobenzo[4,5]thieno[3,2-b]-acridine

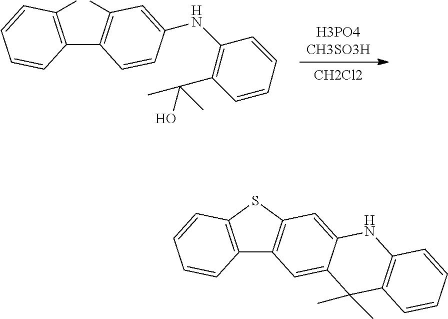

The compound 2-(2-(dibenzo[b,d]thiophen-3-ylamino)phenyl)-propan-2-ol (10 g, 30 mmol) was mixed with 150 ml of CH₂Cl₂, to which 19 ml of methane sulfonic acid and 14 ml of phosphoric acid was then added slowly at room temperature to obtain a mixture. The mixture was then stirred at room temperature to react for 12 hrs. After the reaction finished, ice-cold water was added to obtain a reaction mixture. Subsequently, 20% sodium hydroxide solution was added to the reaction mixture, which was then extracted with ethyl acetate/water and then the organic layer was separated. The solvent was removed from the organic layer under reduced pressure, and the crude product was purified by column chromatography, yielding 5.3 g of 12,12-dimethyl-7,12-dihydrobenzo[4,5]thieno[3,2-b]acridine as yellow solid (56%). ¹H NMR (CDCl₃, 400 MHz): chemical shift (ppm) 8.97(s, 1H),7.81(s, 1H), 7.73(d, 1H), 7.45(d, 1H), 7.38(d, 1H), 7.28(m, 1H), 7.18(dd, 1H), 7.07(m, 1H), 6.89(s, 1H),6.83-6.79(m, 2H), 1.38(s, 6H).

### Synthesis of 1,6-bis(12,12-dimethylbenzo[4,5]thieno [3,2-b]acridin-7(12H)-yl)pyrene (Compound 19)

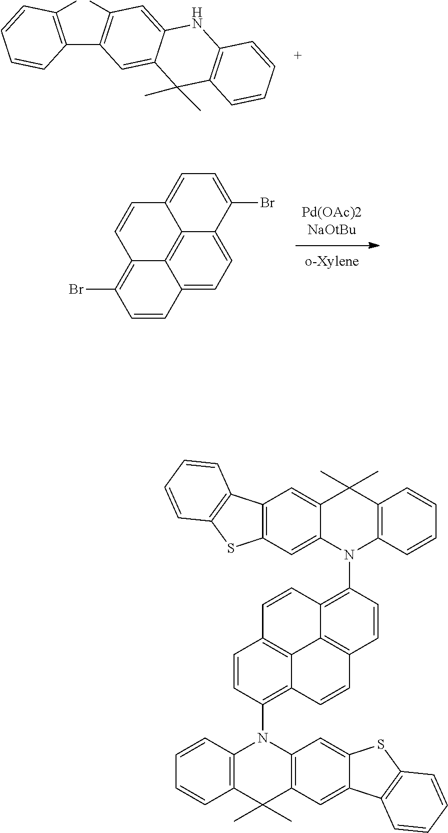 [SMILES: Cc1ccccc1-c1cc2c(cc1C)Nc1ccccc1C2(C)C ; Brc1ccc2ccc3c(Br)ccc4ccc1c2c43 ; CC1(C)c2ccccc2N(c2ccc3ccc4c(N5c6ccccc6C(C)(C)c6cc7c(cc65)sc5ccccc57)ccc5ccc2c3c54)c2cc3sc4ccccc4c3cc21]

A mixture of 3.85 g (12.2 mmol) of 12,12-dimethyl-7,12-dihydrobenzo[4,5]thieno[3,2-b]acridine, 2 g (5.55 mmol) of 1,6-dibromopyrene, 0.05 g (0.22 mmol) of Pd(OAc)₂, 1.6 g (16.7 mmol) of sodium tert-butoxide, and 40 ml of o-xylene was degassed under nitrogen, and then heated at 140° C. for 12 hrs. After the reaction finished, the mixture was allowed to cool to room temperature. Subsequently, 120 ml of methanol was added to the mixture, and then the resulting precipitate was filtered and washed with methanol to get a yellow solid. Yield: 1.75 g, 38%. ¹H NMR (CDCl₃, 400 MHz): chemical shift (ppm) 7.83(s, 2H), 7.79-7.71(m, 8H), 7.45(d, 2H), 7.39(d, 2H), 7.29(m, 2H), 7.21(dd, 2H), 7.08-7.03(m, 4H), 6.91(s, 2H),6.84-6.78(m, 4H), 1.39(s, 12H). MS(m/z, EI⁺): 829.3.

### EXAMPLE 12

### Synthesis of 9,10-bis(12,12-dimethylbenzo[4,5]thieno[3,2-b]acridin-7(12H)-yl)anthracene (Compound 23)

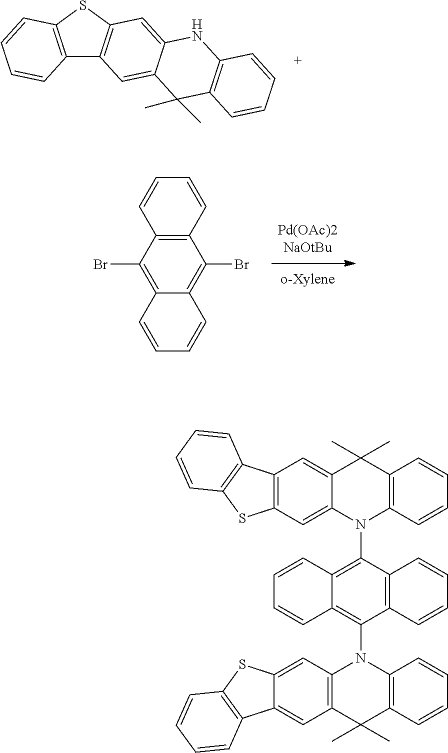 [SMILES: CC1(C)c2ccccc2Nc2cc3sc4ccccc4c3cc21 ; Brc1c2ccccc2c(Br)c2ccccc12 ; CC1(C)c2ccccc2N(c2c3ccccc3c(N3c4ccccc4C(C)(C)c4cc5c(cc43)sc3ccccc35)c3ccccc23)c2cc3sc4ccccc4c3cc21]

The same synthesis procedure as in EXAMPLE 10 was used, except that 9,10-dibromoanthracene was used instead of 1,6-dibromopyrene to obtain the desired compound of 9,10-bis(12,12-dimethylbenzo[4,5]-thieno[3,2-b]acridin-7(12H)-yl)anthracene. MS(m/z,EI⁺): 805.2.

### EXAMPLE 13

### Synthesis of 2,7-bis(12,12-dimethylbenzo[4,5]thieno[3,2-b]acridin-7(12H)-yl)triphenylene (Compound 55)

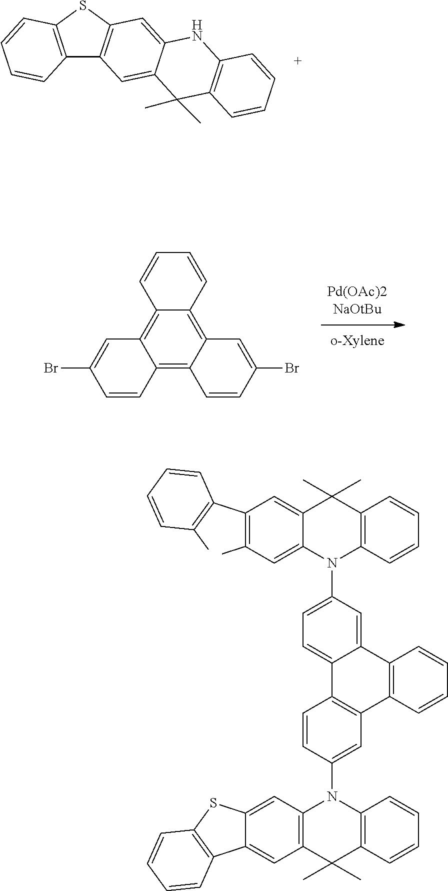 [SMILES: CC1(C)c2ccccc2Nc2cc3sc4ccccc4c3cc21 ; Brc1ccc2c3ccc(Br)cc3c3ccccc3c2c1 ; Cc1ccccc1-c1cc2c(cc1C)N(c1ccc3c4ccc(N5c6ccccc6C(C)(C)c6cc7c(cc65)sc5ccccc57)cc4c4ccccc4c3c1)c1ccccc1C2(C)C]

The same synthesis procedure as in EXAMPLE 10 was used, except that 2,7-dibromotriphenylene was used instead of 1,6-dibromopyrene to obtain the desired compound of 2,7-bis(12,12-dimethylbenzo[4,5]thieno[3,2-b]acridin-7(12H)-yl)triphenylene. MS(m/z,EI⁺): 855.5.

### EXAMPLE 14

### Synthesis of 7,7′-(9,10-diphenylanthracene-2,6-diyl)bis(12,12-dimethyl-7,12-dihydrobenzo[4,5]thieno[3,2-b]acridine) (Compound 59)

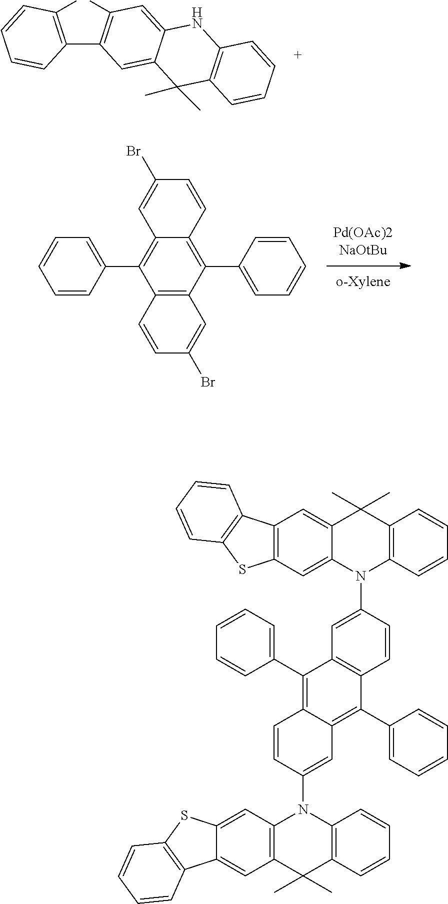 [SMILES: Cc1ccccc1-c1cc2c(cc1C)Nc1ccccc1C2(C)C ; Brc1ccc2c(-c3ccccc3)c3cc(Br)ccc3c(-c3ccccc3)c2c1 ; CC1(C)c2ccccc2N(c2ccc3c(-c4ccccc4)c4cc(N5c6ccccc6C(C)(C)c6cc7c(cc65)sc5ccccc57)ccc4c(-c4ccccc4)c3c2)c2cc3sc4ccccc4c3cc21]

The same synthesis procedure as in EXAMPLE 10 was used, except that 2,6-dibromo-9,10-diphenylanthracene was used instead of 1,6-dibromopyrene to obtain the desired compound of 7,7′-(9,10-diphenyl-anthracene-2,6-diyl)bis(12,12-dimethyl-7,12-dihydrobenzo[4,5]-thieno[3,2-b]acridine). MS(m/z,EI⁺): 957.6.

### EXAMPLE 15

### Synthesis of 7,7′-(5,10-diisopropylpyrene-1,6-diyl)bis(12,12-dimethyl-7,12-dihydrobenzo[4,5]thieno[3,2-b]acridine) (Compound 72)

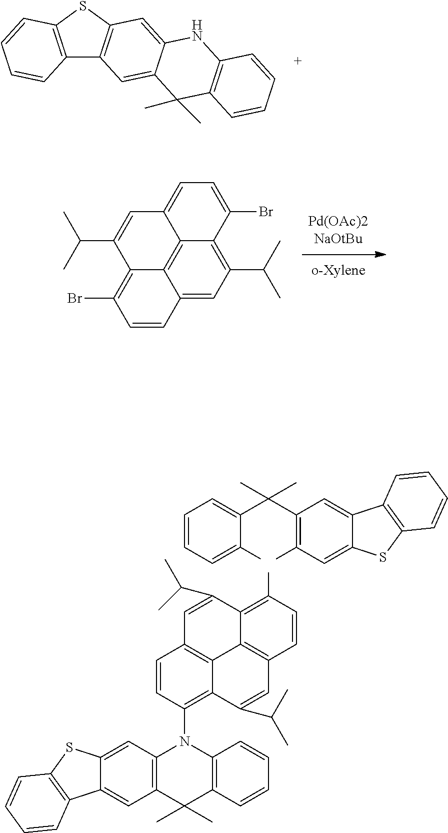

The same synthesis procedure as in EXAMPLE 10 was used, except that 1,6-dibromo-5,10-diisopropylpyrene was used instead of 1,6-dibromopyrene to obtain the desired compound of 7,7′-(5,10-diisopropylpyrene-1,6-diyl)bis(12,12-dimethyl-7,12-dihydrobenzo[4,5]thieno-[3,2-b]acridine). MS(m/z,EI⁺): 913.5.

### General Method of Producing Organic EL Device

ITO-coated glasses with 9-12 ohm/square in resistance and 120-160 nm in thickness are provided (hereinafter ITO substrate) and cleaned in a number of cleaning steps in an ultrasonic bath (e.g. detergent, deionized water). Before vapor deposition of the organic layers, cleaned ITO substrates are further treated by UV and ozone. All pre-treatment processes for ITO substrate are under clean room (class 100).

These organic layers are applied onto the ITO substrate in order by vapor deposition in a high-vacuum unit (10⁻⁷ Torr), such as: resistively heated quartz boats. The thickness of the respective layer and the vapor deposition rate (0.1-0.3 nm/sec) are precisely monitored or set with the aid of a quartz-crystal monitor. It is also possible, as described above, for individual layers to consist of more than one compound, i.e. in general a host material doped with a guest material and/or co-deposited with a co-host. This is successfully achieved by co-vaporization from two or more sources, which means the heteroaromatic compounds of the present invention are thermally stable.

Dipyrazino[2,3-f:2,3-]quinoxaline-2,3,6,7,10,11-hexacarbonitrile (HAT-CN) is used as hole injection layer in this organic EL device. N,N-bis(naphthalene-1-yl)-N,N-bis(phenyl)-benzidine (NPB) is most widely used as the hole transporting layer. 10,10-dimethyl-13-(3-(pyren-1-yl)phenyl) -10H-indeno[2,1-b]triphenylene (H1), 10,10-dimethyl-12-(4-(pyren-1-yl)phenyl)-10H-indeno[2,1-b]triphenylene (H2), 10,10-dimethyl-12-(10-(4-(naphthalene-1-yl)-phenyl)anthracen-9-yl)-10H-indeno[2,1-b]triphenylene (H3), and 10,10-dimethyl-13-(10-(3-(naphthalen-2-yl)phenyl)anthracen-9-yl)-10H-indeno-[2,1-b]triphenylene (H4) are used as emitting hosts in organic EL devices. D1 is used as blue guest, D2 is used as green guest, D3 is used as yellow guest, and D4 is used as red guest for comparison. HB3 (see the following chemical structure) is used as hole blocking material (HBM), and 2-(naphthalen-1-yl) -9-(4-(1-(4-(10-(naphthalene-2-yl)anthracen-9-yl)phenyl)-1H-benzo[d]imidazol-2-yl)phenyl)-1,10-phenanthroline (ET2) is used as electron transporting material to co-deposit with 8-hydroxyquinolato-lithium (LiQ) in organic EL device. The chemical structures of conventional OLED materials and the exemplary heteroaromatic compounds of the present invention for producing control and exemplary organic EL devices in this invention are shown as follows:

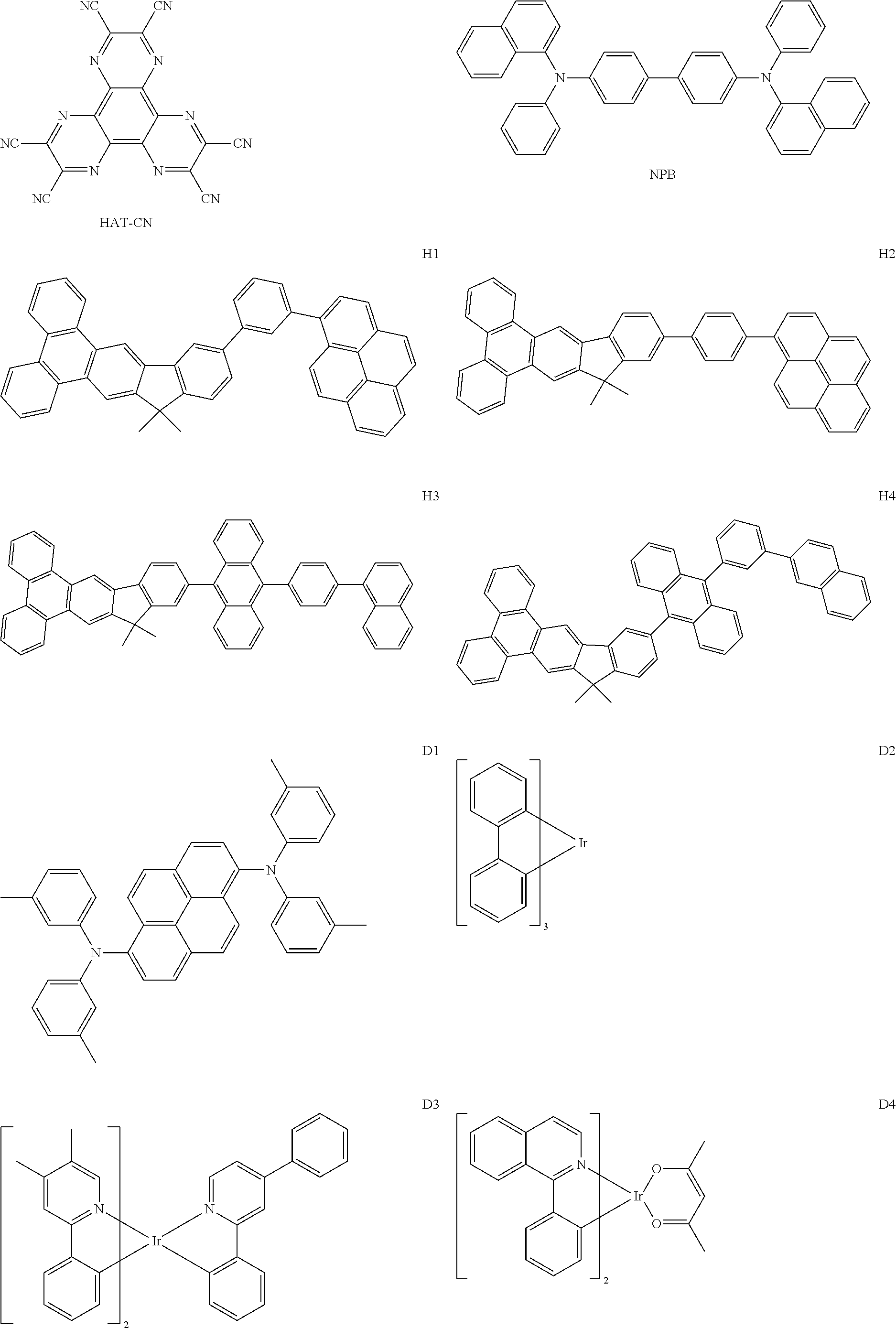

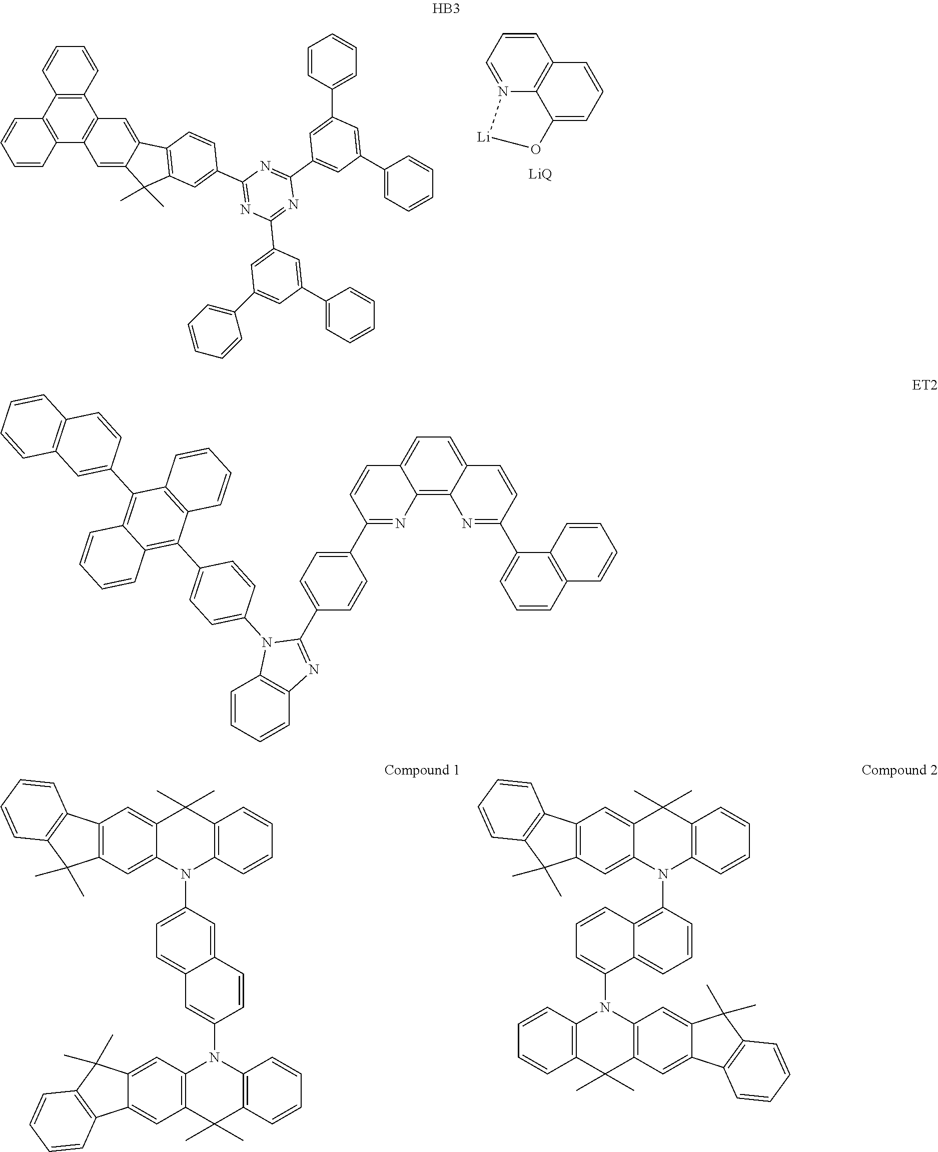

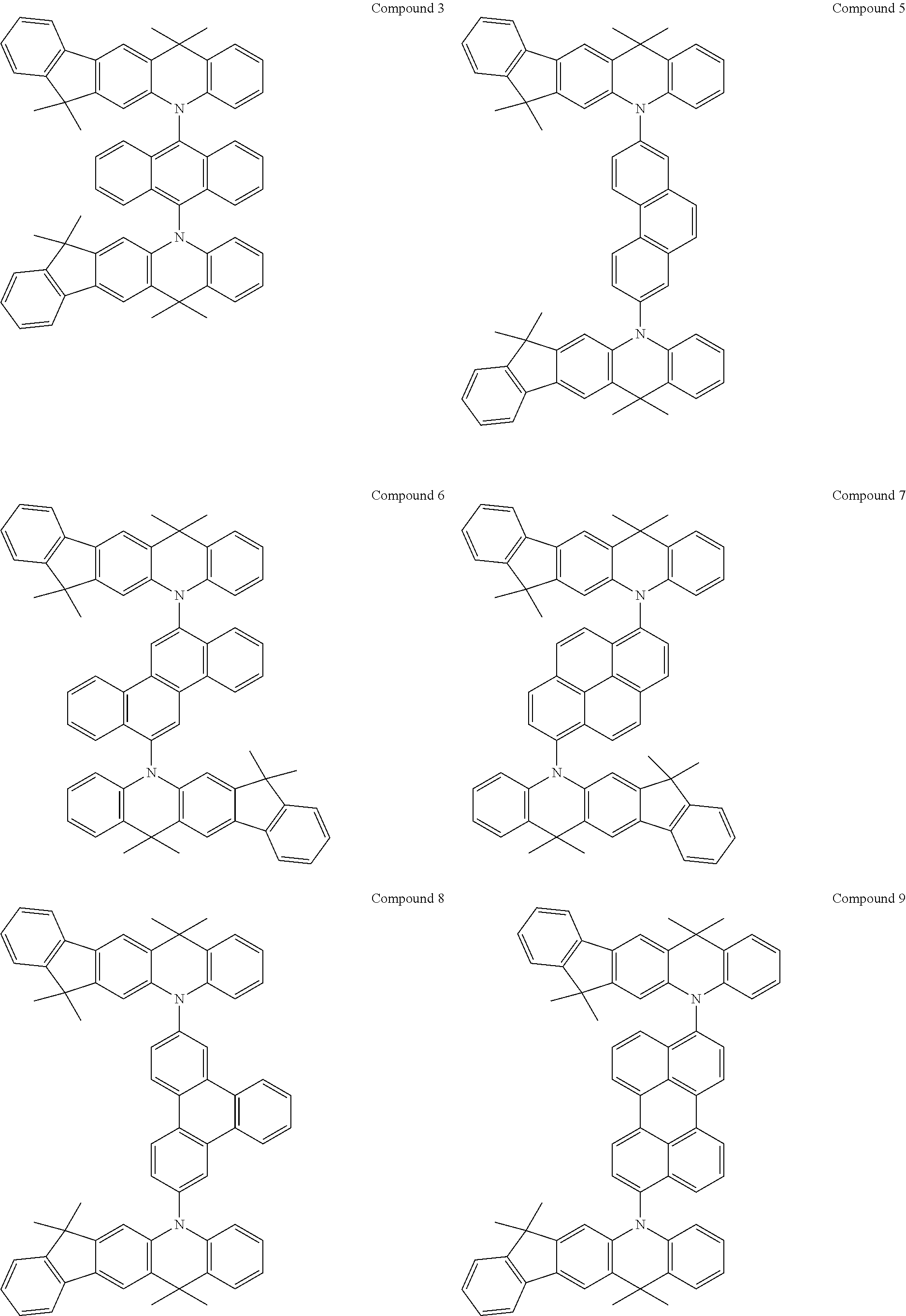

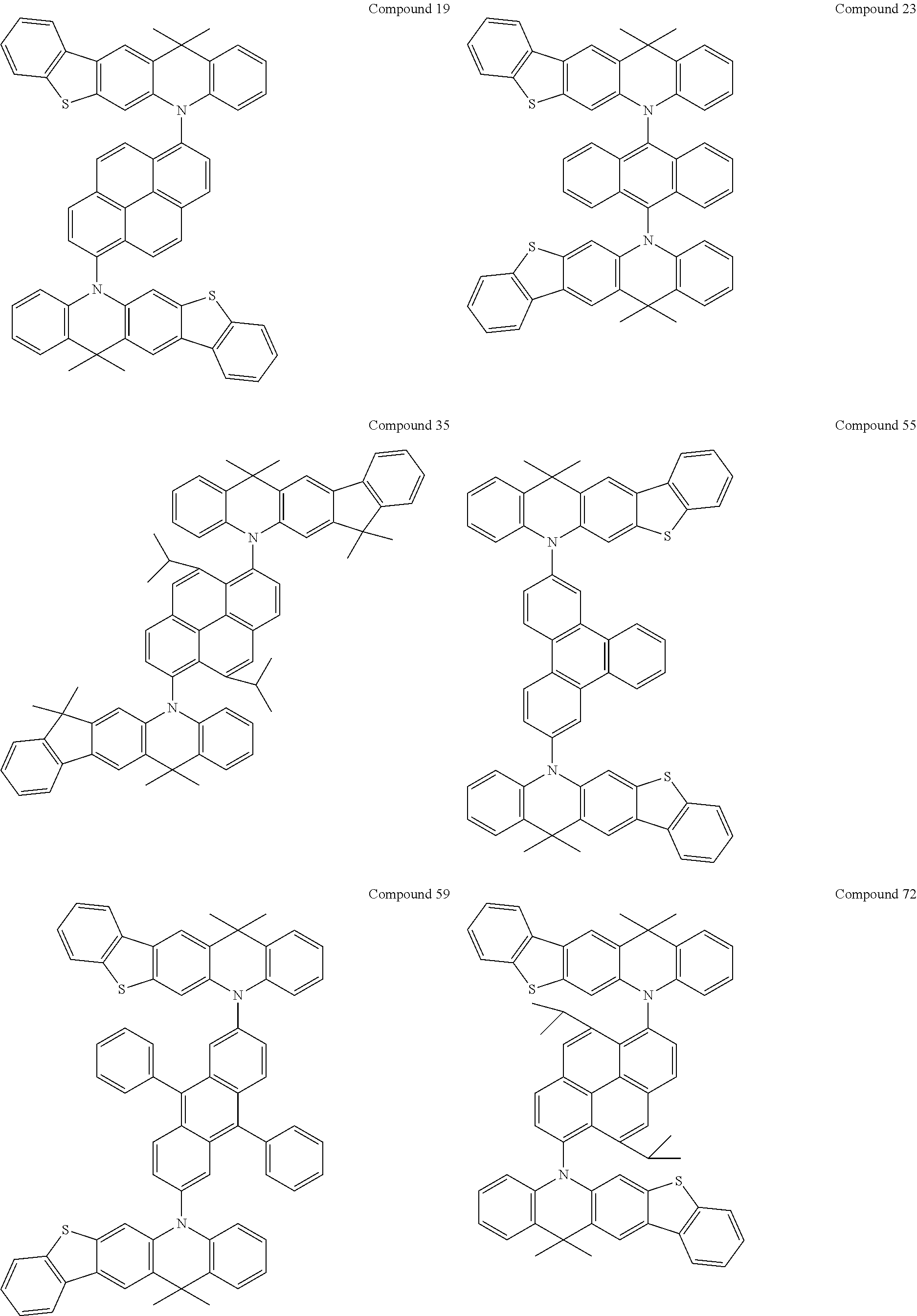

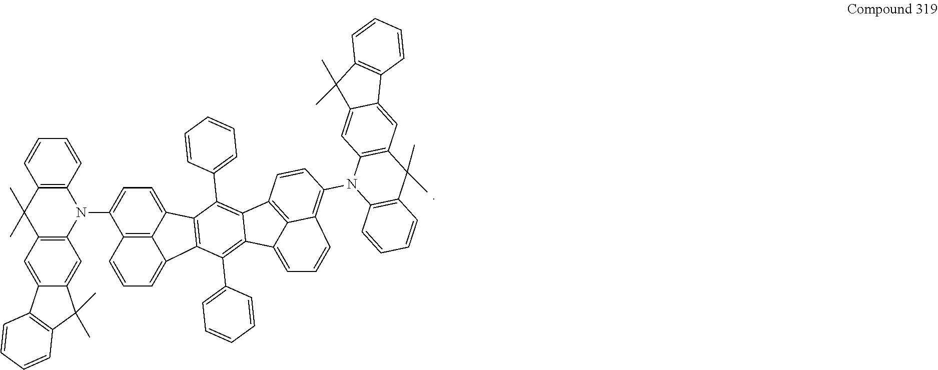 [SMILES: CC1(C)c2ccccc2-c2cc3c(cc21)N(c1ccc2c4c(-c5ccccc5)c5c6ccc(N7c8ccccc8C(C)(C)c8cc9c(cc87)C(C)(C)c7ccccc7-9)c7cccc(c5c(-c5ccccc5)c4c4cccc1c42)c76)c1ccccc1C3(C)C]

A typical organic EL device consists of low work function metals, such as Al, Mg, Ca, Li and K, as the cathode by thermal evaporation, and the low work function metals can help electrons injecting the electron transporting layer from cathode. In addition, for reducing the electron injection barrier and improving the organic EL device performance, a thin-film electron injecting layer is introduced between the cathode and the electron transporting layer. Conventional materials of electron injecting layer are metal halide or metal oxide with low work function, such as: LiF, LiQ, MgO, or Li₂O. On the other hand, after the organic EL device fabrication, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

### EXAMPLE 16

Using a procedure analogous to the above mentioned general method, organic EL devices emitting fluorescence and having the device structure as shown in the FIGURE and the following components were produced: ITO/HAT-CN(20 nm)/NPB (110 nm)/Emitting host doped with 5% Emitting guest(30 nm)/HB3/ET2 doped with 50% LiQ(35nm)/LiQ(1 nm)/Al(160 nm). In the device illustrated in the FIGURE, the hole injection layer 20 is deposited onto the transparent electrode 10, the hole transport layer 30 is deposited onto the hole injection layer 20, the fluorescence emitting layer 40 is deposited onto the hole transport layer 30, the hole blocking layer 50 is deposited onto the fluorescence emitting layer 40, the electron transport layer 60 is deposited onto the hole blocking layer 50, the electron injection layer 70 is deposited onto the electron transport layer 60, and the metal electrode 80 is deposited onto the electron injection layer 70. The I-V-B (at 1000 nits) test reports of these organic EL devices are summarized in Table 1 below. The half-life time is defined as the time the initial luminance of 1000 cd/m² has dropped to half.

TABLE 1

| Emitting | Emitting | Voltage | Efficiency | Device |
|---|---|---|---|---|
| Host | Guest | (V) | (cd/A) | color |
| H1 | D1 | 4.0 | 5.3 | blue |
| H1 | Compound 1 | 3.8 | 5.7 | blue |
| H1 | Compound 2 | 3.7 | 5.8 | blue |
| H2 | D2 | 5.7 | 46.2 | green |
| H2 | Compound 3 | 5.4 | 52.3 | green |
| H2 | Compound 5 | 5.6 | 47.5 | green |
| H2 | Compound 23 | 5.5 | 51.4 | green |
| H2 | Compound 59 | 5.6 | 49.8 | green |
| H3 | D3 | 4.5 | 30.5 | yellow |
| H3 | Compound 6 | 4.3 | 32.1 | yellow |
| H3 | Compound 7 | 4.1 | 33.9 | yellow |
| H3 | Compound 8 | 4.4 | 30.8 | yellow |
| H3 | Compound 19 | 4.2 | 32.7 | yellow |
| H3 | Compound 35 | 4.0 | 34.5 | yellow |
| H3 | Compound 55 | 4.3 | 31.6 | yellow |
| H3 | Compound 72 | 4.1 | 33.2 | yellow |
| H4 | D4 | 3.7 | 18.2 | red |
| H4 | Compound 9 | 3.4 | 24.7 | orange |
| H4 | Compound 319 | 3.5 | 21.3 | red |

From the above test report summary of the organic EL devices, it is obvious that the heteroaromatic compound of formula (1) used as the emitting guest material exhibits better performance than the prior art materials. In particular, the organic EL devices of the present invention employing the heteroaromatic compound of formula (1) as the emitting guest material to collocate with the emitting host material, such as H1, H2, H3 or H4, show lower power consumption, higher current efficiency, and longer half-life time.

To sum up, the present invention discloses a heteroaromatic compound, which can be used as the fluorescent guest material of the light emitting layer in organic EL devices. The mentioned heteroaromatic compound is represented by the following formula (1):

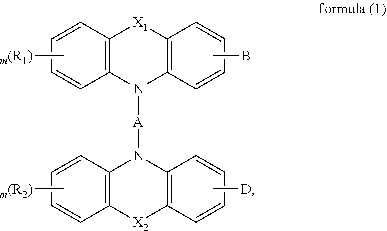

wherein X₁ and X₂ independently represent a divalent bridge selected from the group consisting of O, S, Se, CR₃R₄, NR₅, and SiR₆R₇; A represents a substituted or unsubstituted fused ring hydrocarbon unit with two to nine rings; m is an integer of 0 to 4; B and D independently represent formula (2) below:

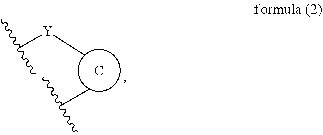

wherein ring C represents a phenyl ring or a fused ring hydrocarbon unit with two to five rings; Y represents a divalent bridge selected from the group consisting of O, S, Se, CR₈R₉, NR₁₀, and SiR₁₁R₁₂; and R₁ to R₁₂ are independently a hydrogen atom, a halide, a substituted or unsubstituted alkyl group having 1 to 60 carbon atoms, a substituted or unsubstituted aryl group having 6 to 60 carbon atoms, a substituted or unsubstituted aralkyl group having 6 to 60 carbon atoms, a substituted or unsubstituted heteroaryl group having 3 to 60 carbon atoms, or a substituted or unsubstituted arylamine group having 6 to 60 carbon atoms.

Obviously, many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A heteroaromatic compound of formula (1) below: 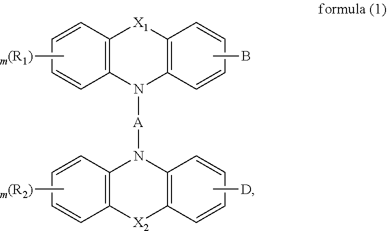
wherein X₁ and X₂ independently represent a divalent bridge selected from the group consisting of O, S, Se, CR₃R₄, NR₅, and SiR₆R₇; A represents a substituted or unsubstituted fused ring hydrocarbon unit with two to nine rings; m is an integer of 0 to 4; B and D independently represent formula (2) below: 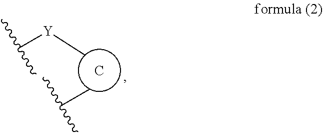
wherein ring C represents a phenyl ring or a fused ring hydrocarbon unit with two to five rings; Y represents a divalent bridge selected from the group consisting of O, S, Se, CR₈R₉, NR₁₀, and SiR₁₁R₁₂; and R₁ to R₁₂ are independently a hydrogen atom, a halide, a substituted or unsubstituted alkyl group having 1 to 60 carbon atoms, a substituted or unsubstituted aryl group having 6 to 60 carbon atoms, a substituted or unsubstituted aralkyl group having 6 to 60 carbon atoms, a substituted or unsubstituted heteroaryl group having 3 to 60 carbon atoms, or a substituted or unsubstituted arylamine group having 6 to 60 carbon atoms.

2. The heteroaromatic compound according to claim 1, wherein A represents a substituted or unsubstituted naphthyl group, a substituted or unsubstituted phenanthrenyl group, a substituted or unsubstituted anthracenyl group, a substituted or unsubstituted pyrenyl group, a substituted or unsubstituted chrysenyl group, a substituted or unsubstituted triphenylenyl group, a substituted or unsubstituted perylenyl group, a substituted or unsubstituted tetraphenylenyl group, or a substituted or unsubstituted 7,14-diphenylacenaphtho[1,2-k]fluoranthene group.

3. The heteroaromatic compound according to claim 1, wherein A represents one of the following formulas: 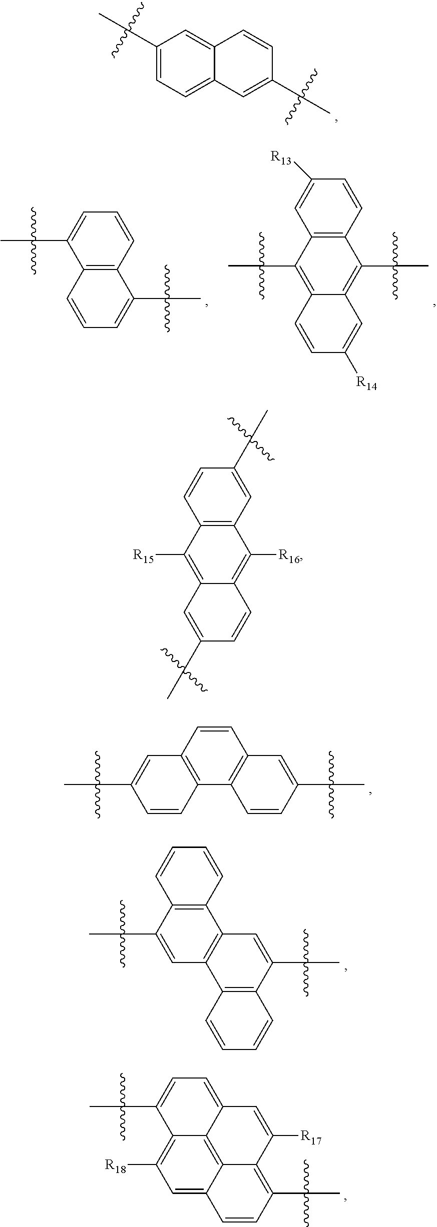 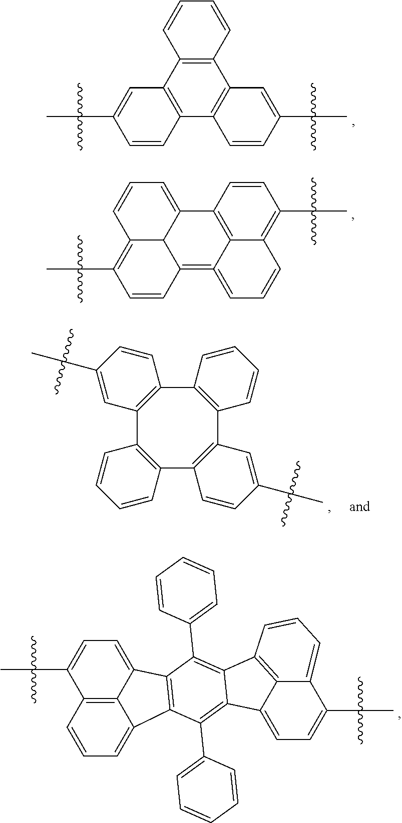
wherein R₁₃ to R₁₈ are independently a hydrogen atom, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, a substituted or unsubstituted heteroaryl group having 3 to 30 carbon atoms, or a substituted or unsubstituted arylamine group having 6 to 30 carbon atoms.

4. The heteroaromatic compound according to claim 1, wherein the heteroaromatic compound is represented by one of the following formula (3) to formula (13): 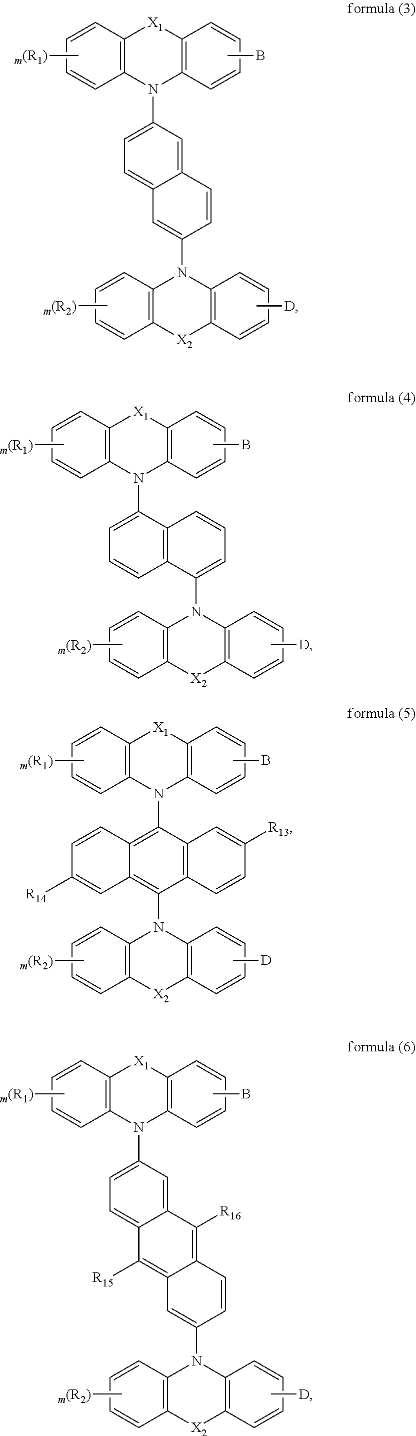 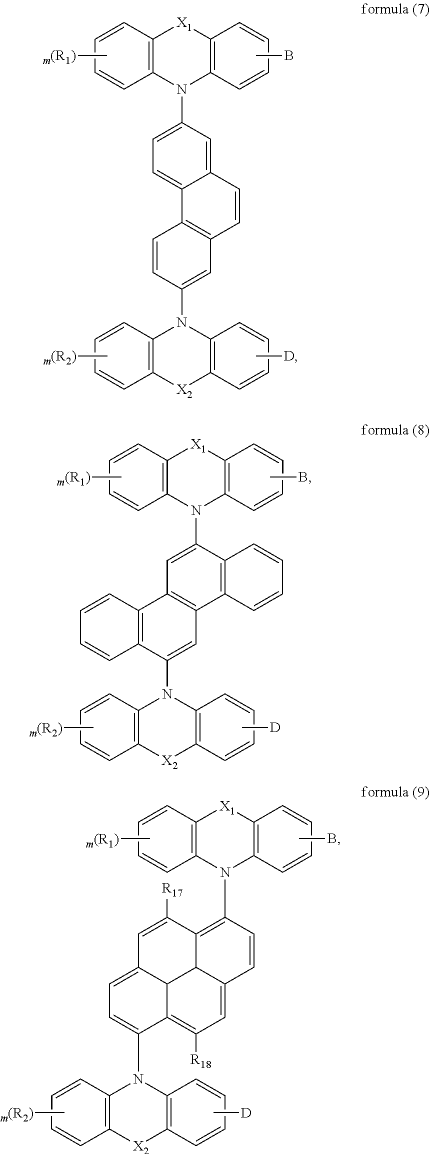 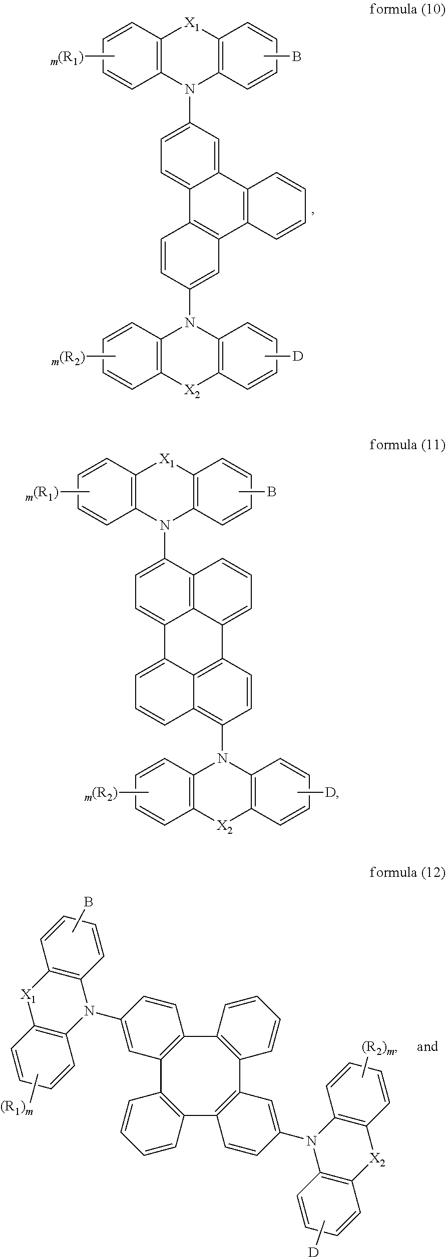 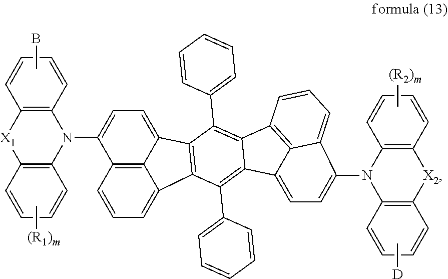
wherein X₁ and X₂ independently represent a divalent bridge selected from the group consisting of O, S, Se, CR₃R₄, NR₅, and SiR₆R₇; m is an integer of 0 to 4; B and D independently represent formula (2) below: 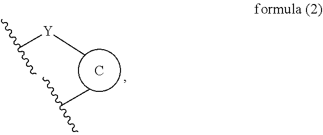
wherein ring C represents a phenyl ring or a fused ring hydrocarbon unit with two to five rings; Y represents a divalent bridge selected from the group consisting of O, S, Se, CR₈R₉, NR₁₀, and SiR₁₁R₁₂; R₁ to R₁₂ are independently a hydrogen atom, a halide, a substituted or unsubstituted alkyl group having 1 to 60 carbon atoms, a substituted or unsubstituted aryl group having 6 to 60 carbon atoms, a substituted or unsubstituted aralkyl group having 6 to 60 carbon atoms, a substituted or unsubstituted heteroaryl group having 3 to 60 carbon atoms, or a substituted or unsubstituted arylamine group having 6 to 60 carbon atoms; and R₁₃ to R₁₈ are independently a hydrogen atom, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, a substituted or unsubstituted heteroaryl group having 3 to 30 carbon atoms, or a substituted or unsubstituted arylamine group having 6 to 30 carbon atoms.

5. The heteroaromatic compound according to claim 1, wherein the heteroaromatic compound is one of the following compounds:

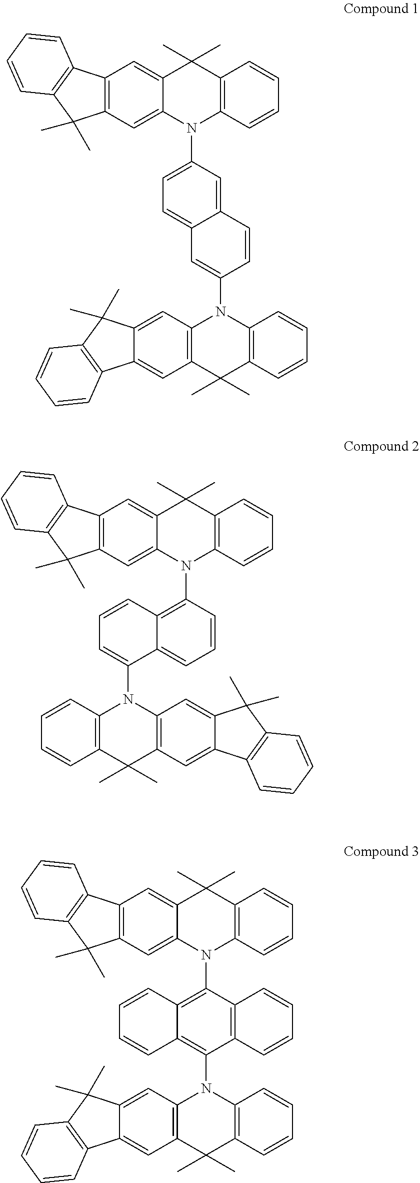 [SMILES: CC1(C)c2ccccc2-c2cc3c(cc21)N(c1ccc2cc(N4c5ccccc5C(C)(C)c5cc6c(cc54)C(C)(C)c4ccccc4-6)ccc2c1)c1ccccc1C3(C)C ; CC1(C)c2ccccc2-c2cc3c(cc21)N(c1cccc2c(N4c5ccccc5C(C)(C)c5cc6c(cc54)C(C)(C)c4ccccc4-6)cccc12)c1ccccc1C3(C)C ; CC1(C)c2ccccc2-c2cc3c(cc21)N(c1c2ccccc2c(N2c4ccccc4C(C)(C)c4cc5c(cc42)C(C)(C)c2ccccc2-5)c2ccccc12)c1ccccc1C3(C)C]

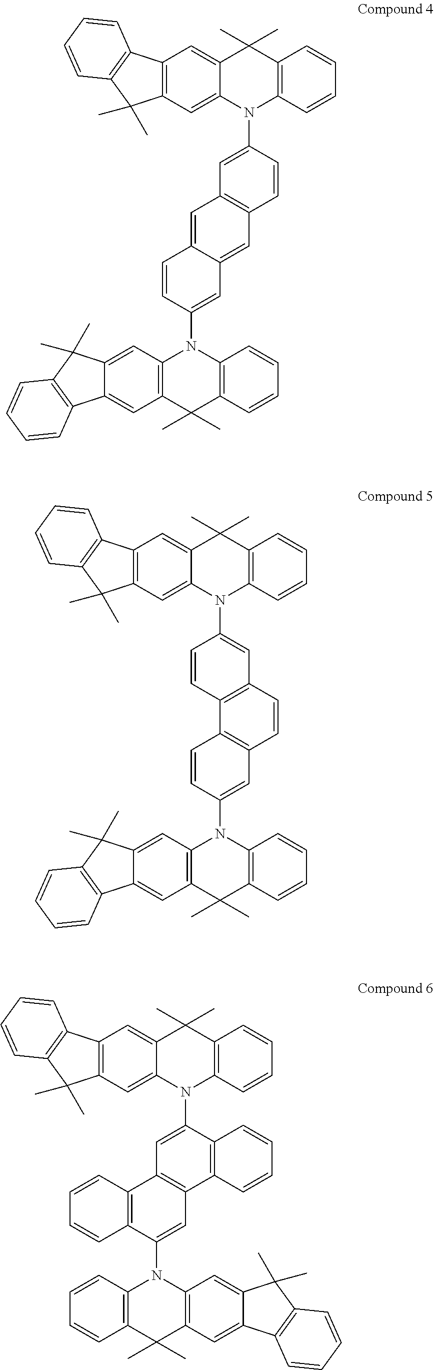 [SMILES: CC1(C)c2ccccc2-c2cc3c(cc21)N(c1ccc2cc4cc(N5c6ccccc6C(C)(C)c6cc7c(cc65)C(C)(C)c5ccccc5-7)ccc4cc2c1)c1ccccc1C3(C)C ; CC1(C)c2ccccc2-c2cc3c(cc21)N(c1ccc2c(ccc4cc(N5c6ccccc6C(C)(C)c6cc7c(cc65)C(C)(C)c5ccccc5-7)ccc42)c1)c1ccccc1C3(C)C ; CC1(C)c2ccccc2-c2cc3c(cc21)N(c1cc2c4ccccc4c(N4c5ccccc5C(C)(C)c5cc6c(cc54)C(C)(C)c4ccccc4-6)cc2c2ccccc12)c1ccccc1C3(C)C]

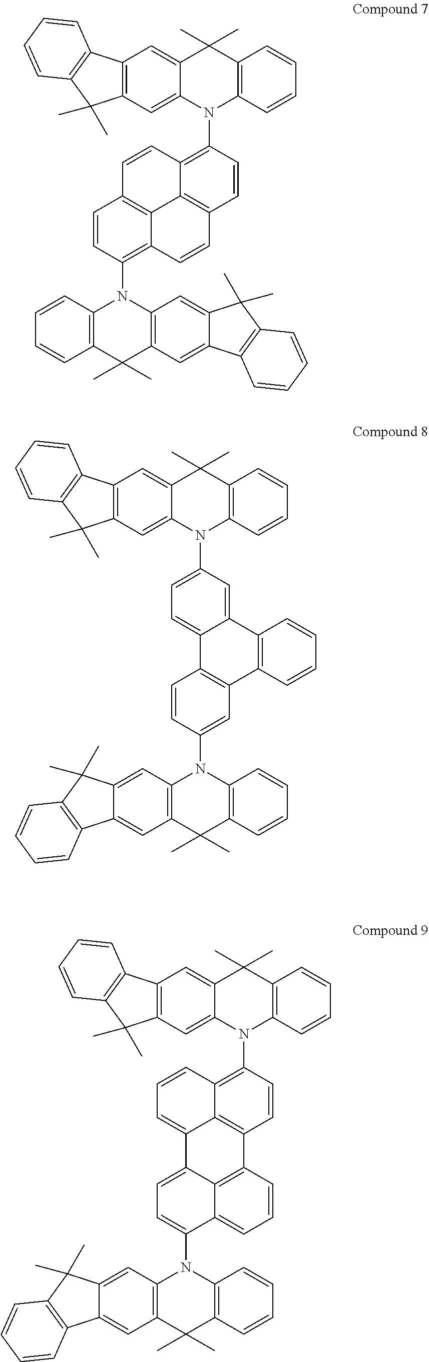 [SMILES: CC1(C)c2ccccc2-c2cc3c(cc21)N(c1ccc2ccc4c(N5c6ccccc6C(C)(C)c6cc7c(cc65)C(C)(C)c5ccccc5-7)ccc5ccc1c2c54)c1ccccc1C3(C)C ; CC1(C)c2ccccc2-c2cc3c(cc21)N(c1ccc2c4ccc(N5c6ccccc6C(C)(C)c6cc7c(cc65)C(C)(C)c5ccccc5-7)cc4c4ccccc4c2c1)c1ccccc1C3(C)C ; CC1(C)c2ccccc2-c2cc3c(cc21)N(c1ccc2c4cccc5c(N6c7ccccc7C(C)(C)c7cc8c(cc76)C(C)(C)c6ccccc6-8)ccc(c6cccc1c62)c54)c1ccccc1C3(C)C]

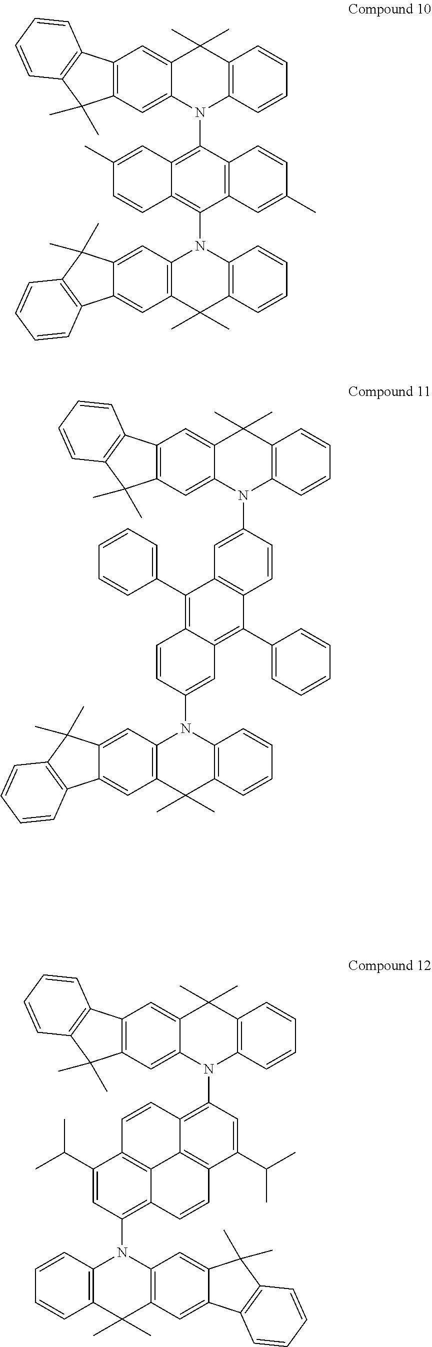 [SMILES: Cc1ccc2c(N3c4ccccc4C(C)(C)c4cc5c(cc43)C(C)(C)c3ccccc3-5)c3cc(C)ccc3c(N3c4ccccc4C(C)(C)c4cc5c(cc43)C(C)(C)c3ccccc3-5)c2c1 ; CC1(C)c2ccccc2-c2cc3c(cc21)N(c1ccc2c(-c4ccccc4)c4cc(N5c6ccccc6C(C)(C)c6cc7c(cc65)C(C)(C)c5ccccc5-7)ccc4c(-c4ccccc4)c2c1)c1ccccc1C3(C)C ; CC(C)c1cc(N2c3ccccc3C(C)(C)c3cc4c(cc32)C(C)(C)c2ccccc2-4)c2ccc3c(C(C)C)cc(N4c5ccccc5C(C)(C)c5cc6c(cc54)C(C)(C)c4ccccc4-6)c4ccc1c2c34]

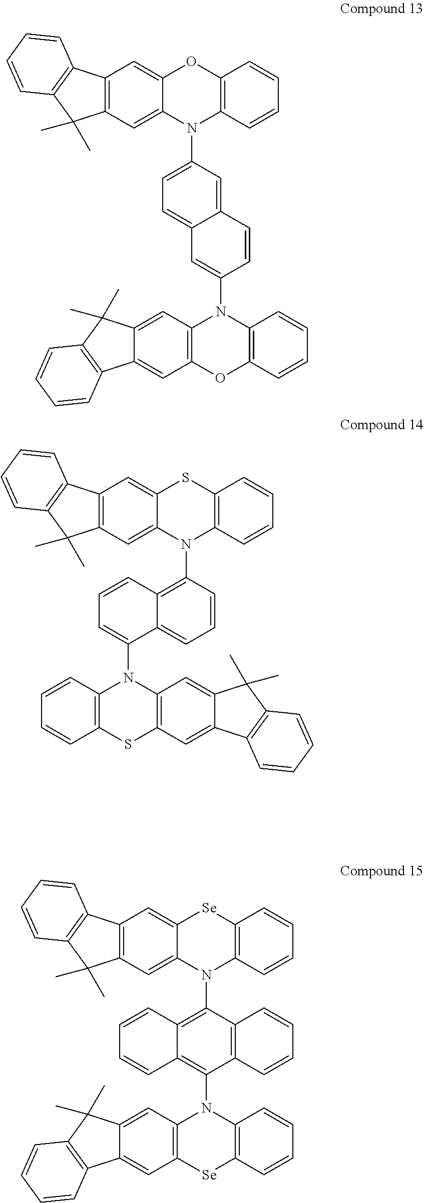 [SMILES: CC1(C)c2ccccc2-c2cc3c(cc21)N(c1ccc2cc(N4c5ccccc5Oc5cc6c(cc54)C(C)(C)c4ccccc4-6)ccc2c1)c1ccccc1O3 ; CC1(C)c2ccccc2-c2cc3c(cc21)N(c1cccc2c(N4c5ccccc5Sc5cc6c(cc54)C(C)(C)c4ccccc4-6)cccc12)c1ccccc1S3 ; CC1(C)c2ccccc2-c2cc3c(cc21)N(c1c2ccccc2c(N2c4ccccc4[Se]c4cc5c(cc42)C(C)(C)c2ccccc2-5)c2ccccc12)c1ccccc1[Se]3]

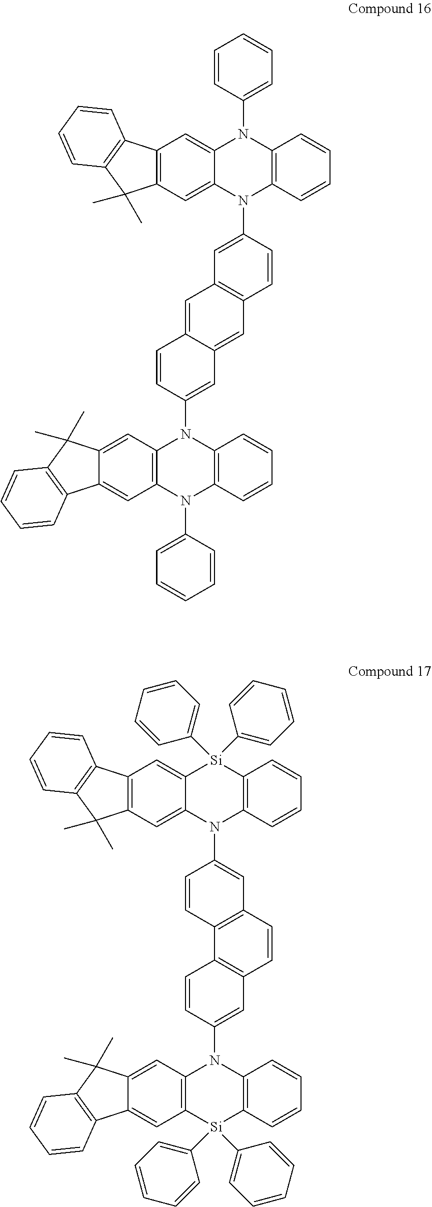 [SMILES: CC1(C)c2ccccc2-c2cc3c(cc21)N(c1ccc2cc4cc(N5c6ccccc6N(c6ccccc6)c6cc7c(cc65)C(C)(C)c5ccccc5-7)ccc4cc2c1)c1ccccc1N3c1ccccc1 ; CC1(C)c2ccccc2-c2cc3c(cc21)N(c1ccc2c(ccc4cc(N5c6ccccc6[Si](c6ccccc6)(c6ccccc6)c6cc7c(cc65)C(C)(C)c5ccccc5-7)ccc42)c1)c1ccccc1[Si]3(c1ccccc1)c1ccccc1]

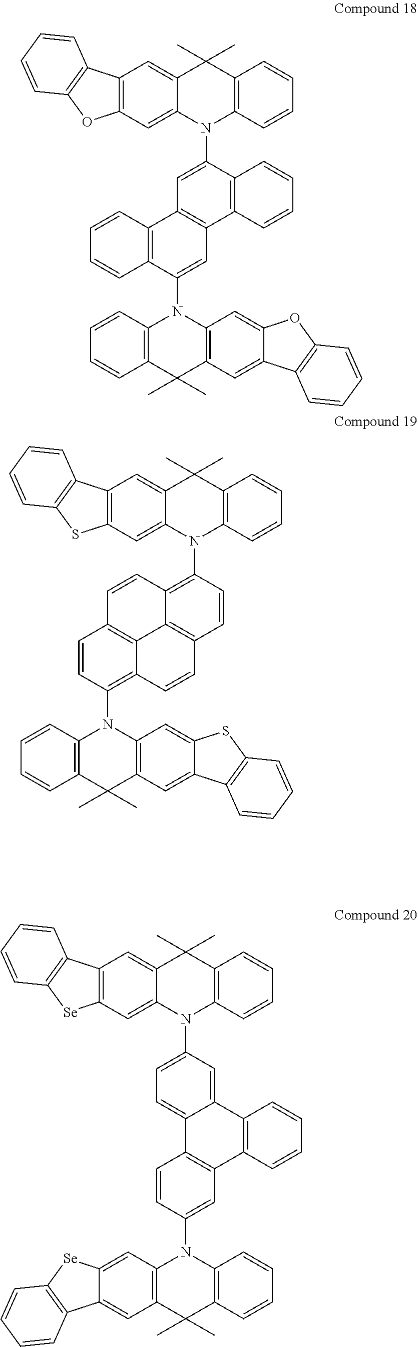 [SMILES: CC1(C)c2ccccc2N(c2cc3c4ccccc4c(N4c5ccccc5C(C)(C)c5cc6c(cc54)oc4ccccc46)cc3c3ccccc23)c2cc3oc4ccccc4c3cc21 ; CC1(C)c2ccccc2N(c2ccc3ccc4c(N5c6ccccc6C(C)(C)c6cc7c(cc65)sc5ccccc57)ccc5ccc2c3c54)c2cc3sc4ccccc4c3cc21 ; CC1(C)c2ccccc2N(c2ccc3c4ccc(N5c6ccccc6C(C)(C)c6cc7c(cc65)[se]c5ccccc57)cc4c4ccccc4c3c2)c2cc3[se]c4ccccc4c3cc21]

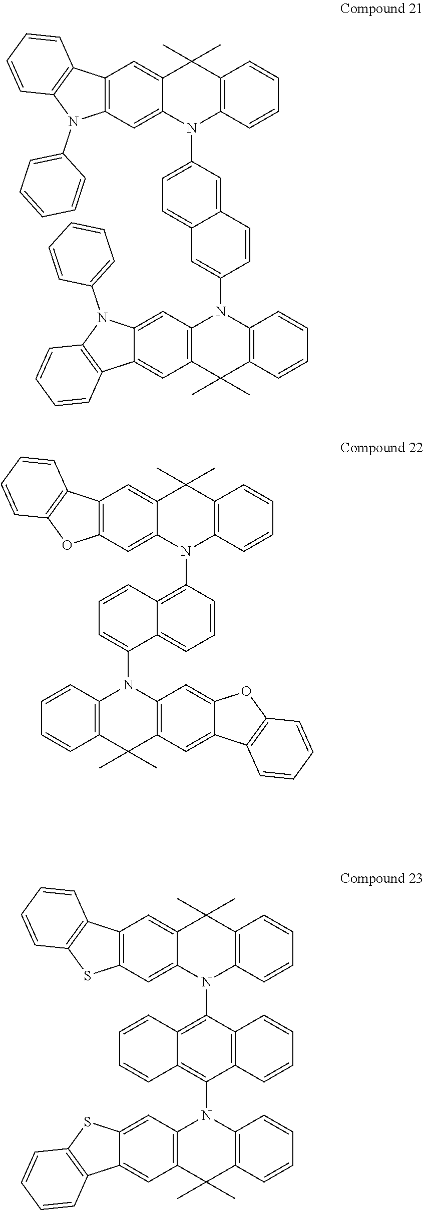 [SMILES: CC1(C)c2ccccc2N(c2ccc3cc(N4c5ccccc5C(C)(C)c5cc6c7ccccc7n(-c7ccccc7)c6cc54)ccc3c2)c2cc3c(cc21)c1ccccc1n3-c1ccccc1 ; CC1(C)c2ccccc2N(c2cccc3c(N4c5ccccc5C(C)(C)c5cc6c(cc54)oc4ccccc46)cccc23)c2cc3oc4ccccc4c3cc21 ; CC1(C)c2ccccc2N(c2c3ccccc3c(N3c4ccccc4C(C)(C)c4cc5c(cc43)sc3ccccc35)c3ccccc23)c2cc3sc4ccccc4c3cc21]

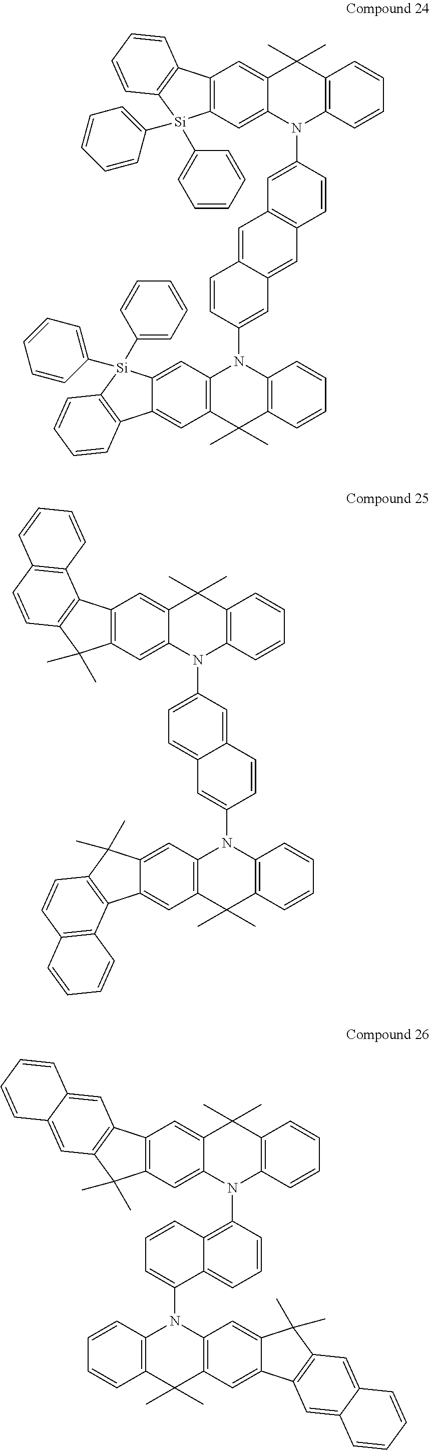 [SMILES: CC1(C)c2ccccc2N(c2ccc3cc4cc(N5c6ccccc6C(C)(C)c6cc7c(cc65)[Si](c5ccccc5)(c5ccccc5)c5ccccc5-7)ccc4cc3c2)c2cc3c(cc21)-c1ccccc1[Si]3(c1ccccc1)c1ccccc1 ; CC1(C)c2ccccc2N(c2ccc3cc(N4c5ccccc5C(C)(C)c5cc6c(cc54)C(C)(C)c4ccc5ccccc5c4-6)ccc3c2)c2cc3c(cc21)-c1c(ccc2ccccc12)C3(C)C ; CC1(C)c2cc3c(cc2-c2cc4ccccc4cc21)C(C)(C)c1ccccc1N3c1cccc2c(N3c4ccccc4C(C)(C)c4cc5c(cc43)C(C)(C)c3cc4ccccc4cc3-5)cccc12]

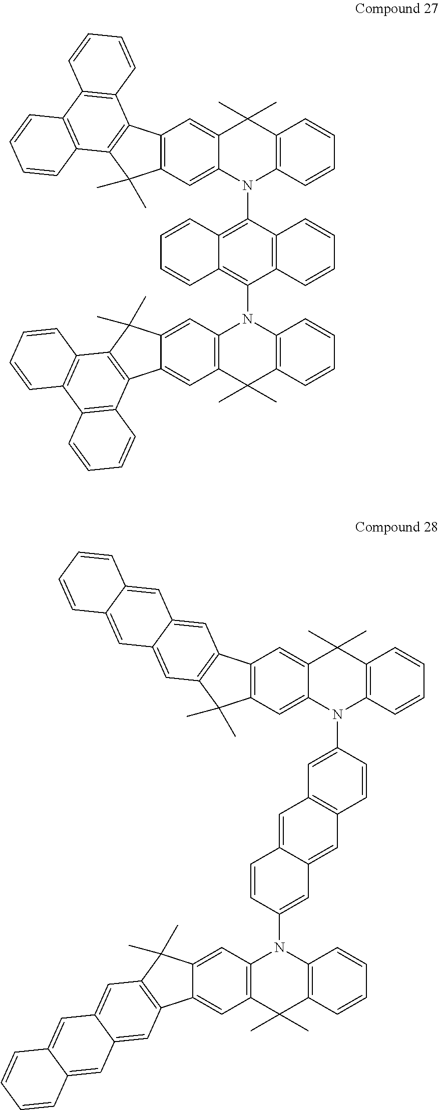 [SMILES: CC1(C)c2ccccc2N(c2c3ccccc3c(N3c4ccccc4C(C)(C)c4cc5c(cc43)C(C)(C)c3c-5c4ccccc4c4ccccc34)c3ccccc23)c2cc3c(cc21)-c1c(c2ccccc2c2ccccc12)C3(C)C ; CC1(C)c2cc3c(cc2-c2cc4cc5ccccc5cc4cc21)C(C)(C)c1ccccc1N3c1ccc2cc3cc(N4c5ccccc5C(C)(C)c5cc6c(cc54)C(C)(C)c4cc5cc7ccccc7cc5cc4-6)ccc3cc2c1]

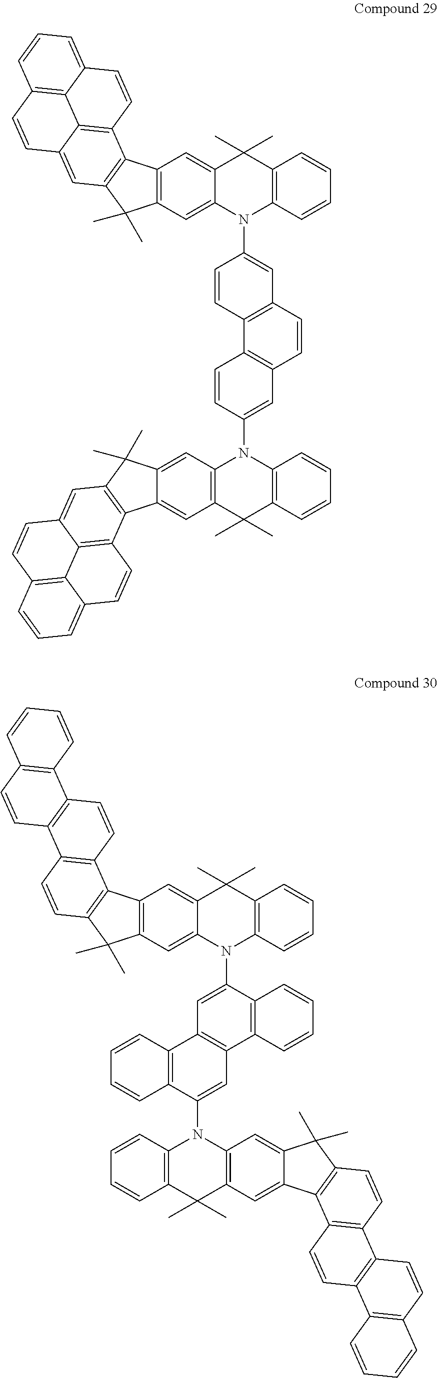 [SMILES: CC1(C)c2ccccc2N(c2ccc3c(ccc4cc(N5c6ccccc6C(C)(C)c6cc7c(cc65)C(C)(C)c5cc6ccc8cccc9ccc(c5-7)c6c89)ccc43)c2)c2cc3c(cc21)-c1c(cc2ccc4cccc5ccc1c2c45)C3(C)C ; CC1(C)c2ccccc2N(c2cc3c4ccccc4c(N4c5ccccc5C(C)(C)c5cc6c(cc54)C(C)(C)c4ccc5c(ccc7c8ccccc8ccc57)c4-6)cc3c3ccccc23)c2cc3c(cc21)-c1c(ccc2c1ccc1c4ccccc4ccc21)C3(C)C]

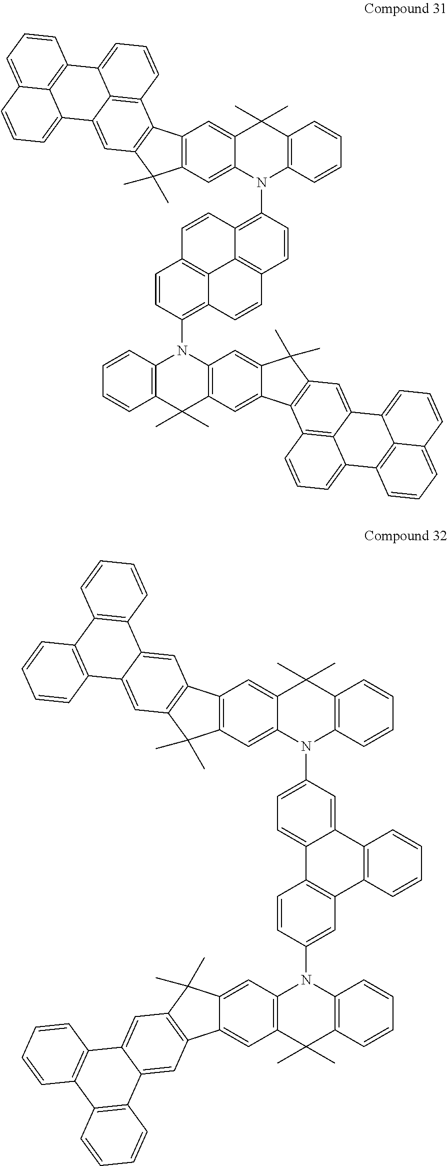 [SMILES: CC1(C)c2ccccc2N(c2ccc3ccc4c(N5c6ccccc6C(C)(C)c6cc7c(cc65)C(C)(C)c5cc6c8cccc9cccc(c%10cccc(c5-7)c%106)c98)ccc5ccc2c3c54)c2cc3c(cc21)-c1c(cc2c4cccc5cccc(c6cccc1c62)c54)C3(C)C ; CC1(C)c2cc3c(cc2-c2cc4c5ccccc5c5ccccc5c4cc21)C(C)(C)c1ccccc1N3c1ccc2c3ccc(N4c5ccccc5C(C)(C)c5cc6c(cc54)C(C)(C)c4cc5c7ccccc7c7ccccc7c5cc4-6)cc3c3ccccc3c2c1]

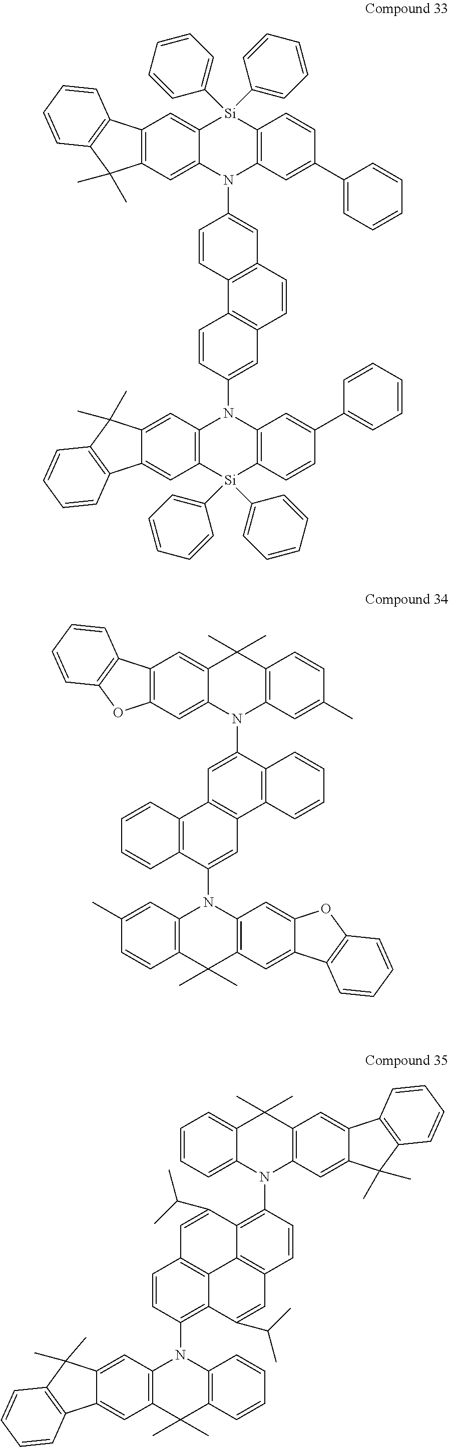 [SMILES: CC1(C)c2ccccc2-c2cc3c(cc21)N(c1ccc2c(ccc4cc(N5c6cc(-c7ccccc7)ccc6[Si](c6ccccc6)(c6ccccc6)c6cc7c(cc65)C(C)(C)c5ccccc5-7)ccc42)c1)c1cc(-c2ccccc2)ccc1[Si]3(c1ccccc1)c1ccccc1 ; Cc1ccc2c(c1)N(c1cc3c4ccccc4c(N4c5cc(C)ccc5C(C)(C)c5cc6c(cc54)oc4ccccc46)cc3c3ccccc13)c1cc3oc4ccccc4c3cc1C2(C)C ; CC(C)c1cc2ccc(N3c4ccccc4C(C)(C)c4cc5c(cc43)C(C)(C)c3ccccc3-5)c3c(C(C)C)cc4ccc(N5c6ccccc6C(C)(C)c6cc7c(cc65)C(C)(C)c5ccccc5-7)c1c4c23]

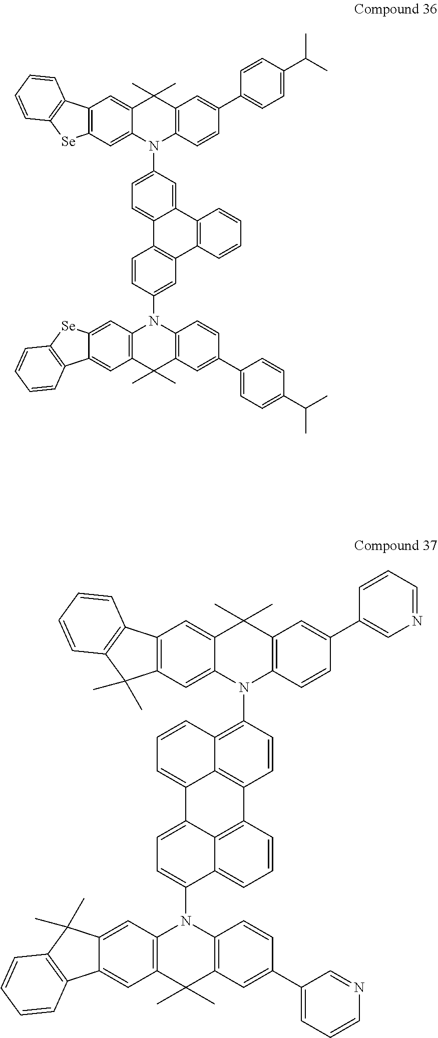 [SMILES: CC(C)c1ccc(-c2ccc3c(c2)C(C)(C)c2cc4c(cc2N3c2ccc3c5ccc(N6c7ccc(-c8ccc(C(C)C)cc8)cc7C(C)(C)c7cc8c(cc76)[se]c6ccccc68)cc5c5ccccc5c3c2)[se]c2ccccc24)cc1 ; CC1(C)c2ccccc2-c2cc3c(cc21)N(c1ccc2c4cccc5c(N6c7ccc(-c8cccnc8)cc7C(C)(C)c7cc8c(cc76)C(C)(C)c6ccccc6-8)ccc(c6cccc1c62)c54)c1ccc(-c2cccnc2)cc1C3(C)C]

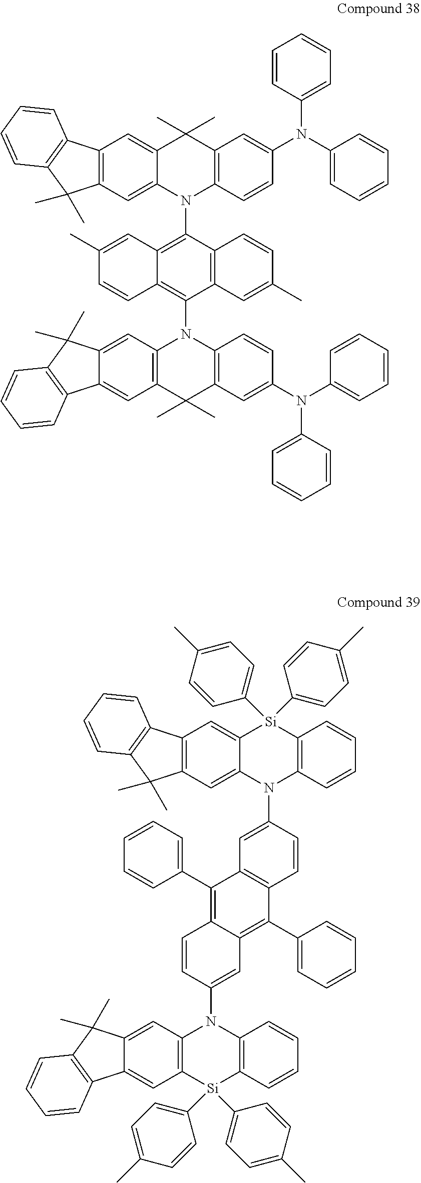 [SMILES: Cc1ccc2c(N3c4ccc(N(c5ccccc5)c5ccccc5)cc4C(C)(C)c4cc5c(cc43)C(C)(C)c3ccccc3-5)c3cc(C)ccc3c(N3c4ccc(N(c5ccccc5)c5ccccc5)cc4C(C)(C)c4cc5c(cc43)C(C)(C)c3ccccc3-5)c2c1 ; Cc1ccc([Si]2(c3ccc(C)cc3)c3ccccc3N(c3ccc4c(-c5ccccc5)c5cc(N6c7ccccc7[Si](c7ccc(C)cc7)(c7ccc(C)cc7)c7cc8c(cc76)C(C)(C)c6ccccc6-8)ccc5c(-c5ccccc5)c4c3)c3cc4c(cc32)-c2ccccc2C4(C)C)cc1]

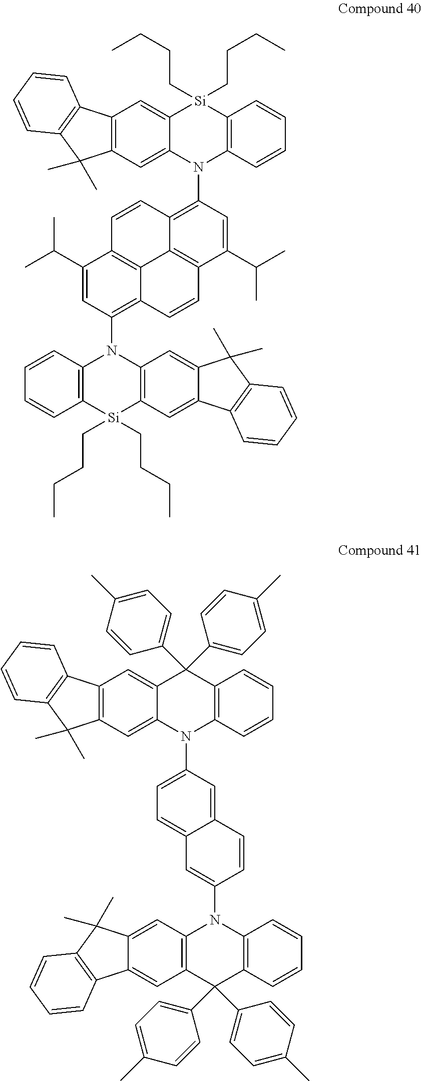 [SMILES: CCCC[Si]1(CCCC)c2ccccc2N(c2cc(C(C)C)c3ccc4c(N5c6ccccc6[Si](CCCC)(CCCC)c6cc7c(cc65)C(C)(C)c5ccccc5-7)cc(C(C)C)c5ccc2c3c54)c2cc3c(cc21)-c1ccccc1C3(C)C ; Cc1ccc(C2(c3ccc(C)cc3)c3ccccc3N(c3ccc4cc(N5c6ccccc6C(c6ccc(C)cc6)(c6ccc(C)cc6)c6cc7c(cc65)C(C)(C)c5ccccc5-7)ccc4c3)c3cc4c(cc32)-c2ccccc2C4(C)C)cc1]

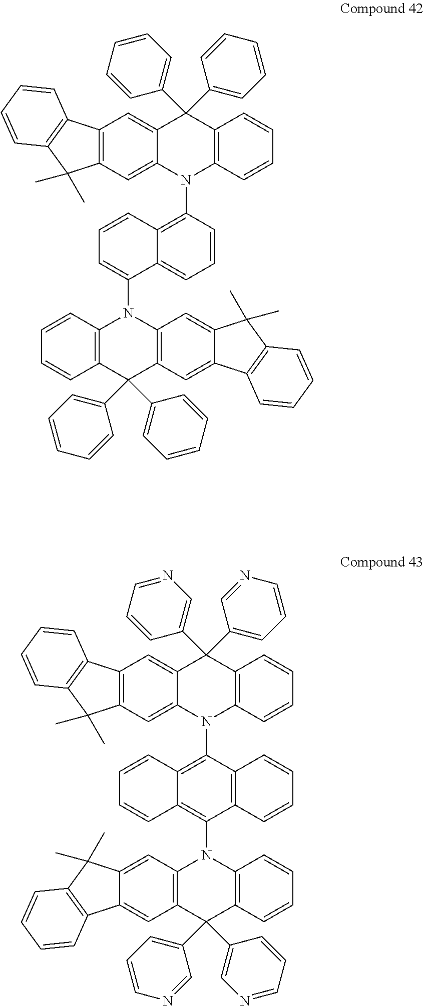 [SMILES: CC1(C)c2ccccc2-c2cc3c(cc21)N(c1cccc2c(N4c5ccccc5C(c5ccccc5)(c5ccccc5)c5cc6c(cc54)C(C)(C)c4ccccc4-6)cccc12)c1ccccc1C3(c1ccccc1)c1ccccc1 ; CC1(C)c2ccccc2-c2cc3c(cc21)N(c1c2ccccc2c(N2c4ccccc4C(c4cccnc4)(c4cccnc4)c4cc5c(cc42)C(C)(C)c2ccccc2-5)c2ccccc12)c1ccccc1C3(c1cccnc1)c1cccnc1]

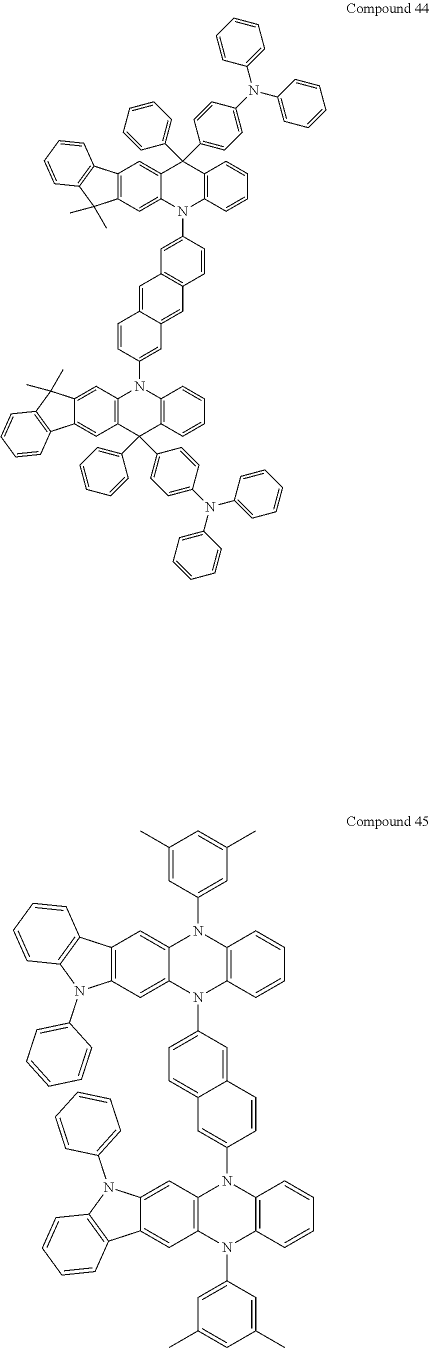 [SMILES: CC1(C)c2ccccc2-c2cc3c(cc21)N(c1ccc2cc4cc(N5c6ccccc6C(c6ccccc6)(c6ccc(N(c7ccccc7)c7ccccc7)cc6)c6cc7c(cc65)C(C)(C)c5ccccc5-7)ccc4cc2c1)c1ccccc1C3(c1ccccc1)c1ccc(N(c2ccccc2)c2ccccc2)cc1 ; Cc1cc(C)cc(N2c3ccccc3N(c3ccc4cc(N5c6ccccc6N(c6cc(C)cc(C)c6)c6cc7c8ccccc8n(-c8ccccc8)c7cc65)ccc4c3)c3cc4c(cc32)c2ccccc2n4-c2ccccc2)c1]

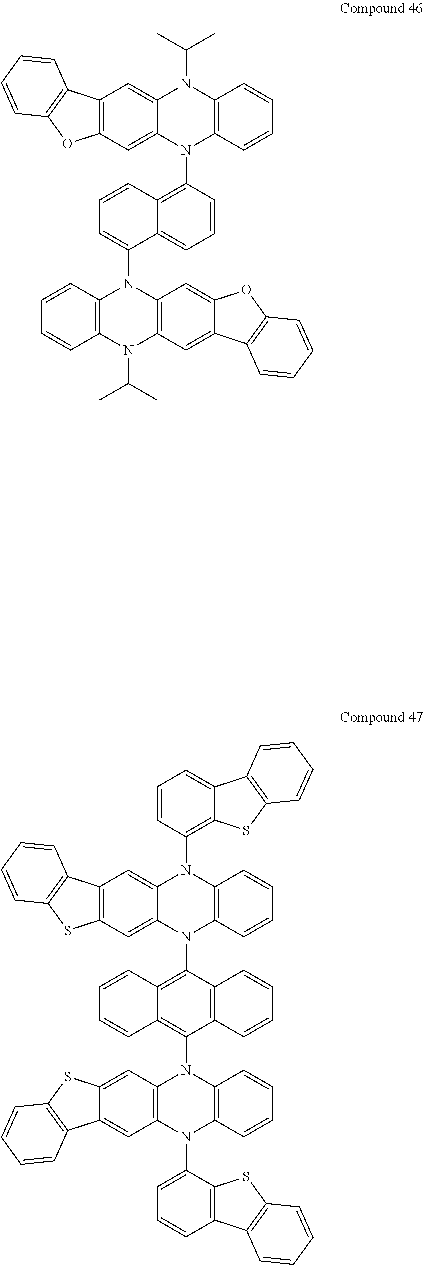 [SMILES: CC(C)N1c2ccccc2N(c2cccc3c(N4c5ccccc5N(C(C)C)c5cc6c(cc54)oc4ccccc46)cccc23)c2cc3oc4ccccc4c3cc21 ; c1ccc2c(c1)N(c1c3ccccc3c(N3c4ccccc4N(c4cccc5c4sc4ccccc45)c4cc5c(cc43)sc3ccccc35)c3ccccc13)c1cc3sc4ccccc4c3cc1N2c1cccc2c1sc1ccccc12]

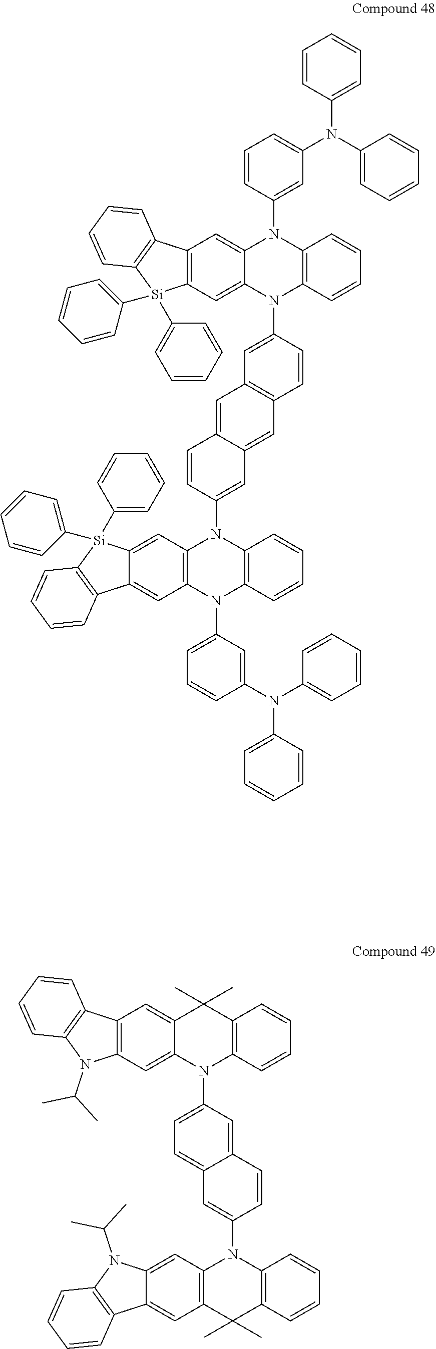 [SMILES: c1ccc(N(c2ccccc2)c2cccc(N3c4ccccc4N(c4ccc5cc6cc(N7c8ccccc8N(c8cccc(N(c9ccccc9)c9ccccc9)c8)c8cc9c(cc87)[Si](c7ccccc7)(c7ccccc7)c7ccccc7-9)ccc6cc5c4)c4cc5c(cc43)-c3ccccc3[Si]5(c3ccccc3)c3ccccc3)c2)cc1 ; CC(C)n1c2ccccc2c2cc3c(cc21)N(c1ccc2cc(N4c5ccccc5C(C)(C)c5cc6c7ccccc7n(C(C)C)c6cc54)ccc2c1)c1ccccc1C3(C)C]

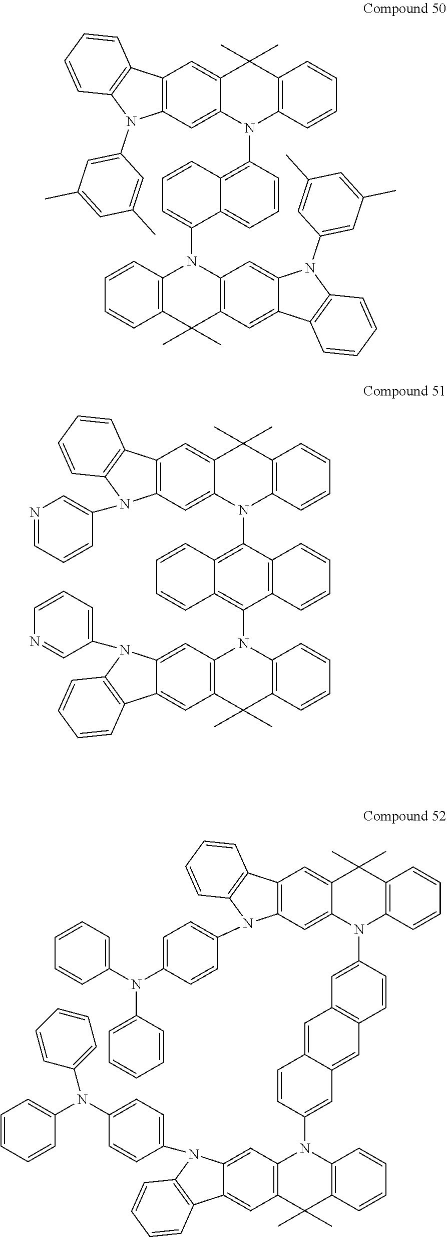

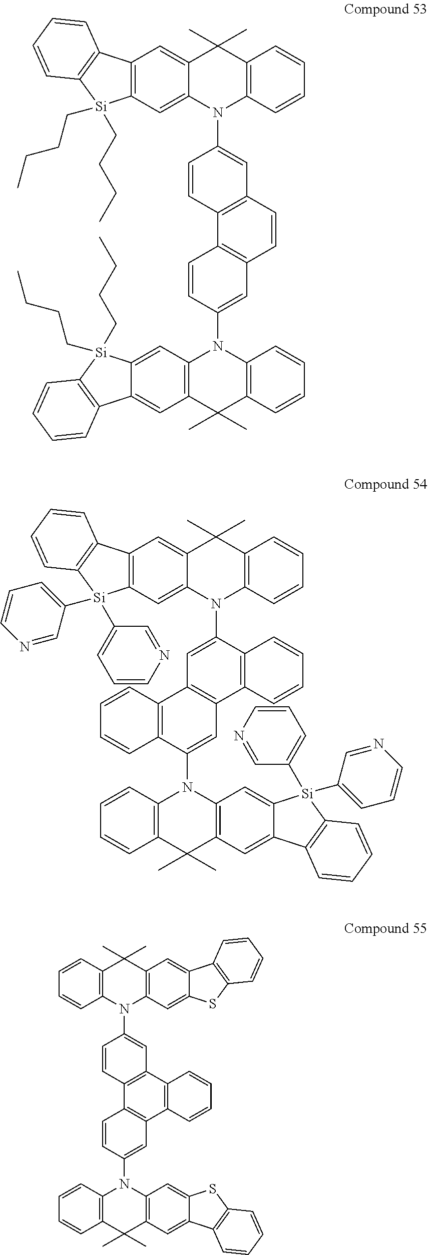 [SMILES: CCCC[Si]1(CCCC)c2ccccc2-c2cc3c(cc21)N(c1ccc2c(ccc4cc(N5c6ccccc6C(C)(C)c6cc7c(cc65)[Si](CCCC)(CCCC)c5ccccc5-7)ccc42)c1)c1ccccc1C3(C)C ; CC1(C)c2ccccc2N(c2cc3c4ccccc4c(N4c5ccccc5C(C)(C)c5cc6c(cc54)[Si](c4cccnc4)(c4cccnc4)c4ccccc4-6)cc3c3ccccc23)c2cc3c(cc21)-c1ccccc1[Si]3(c1cccnc1)c1cccnc1 ; CC1(C)c2ccccc2N(c2ccc3c4ccc(N5c6ccccc6C(C)(C)c6cc7c(cc65)sc5ccccc57)cc4c4ccccc4c3c2)c2cc3sc4ccccc4c3cc21]

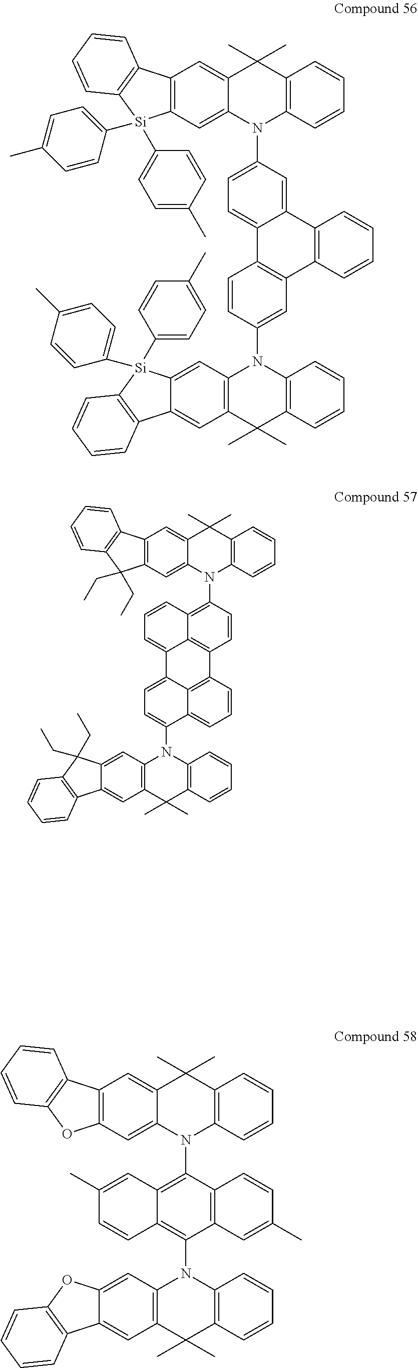 [SMILES: Cc1ccc([Si]2(c3ccc(C)cc3)c3ccccc3-c3cc4c(cc32)N(c2ccc3c5ccc(N6c7ccccc7C(C)(C)c7cc8c(cc76)[Si](c6ccc(C)cc6)(c6ccc(C)cc6)c6ccccc6-8)cc5c5ccccc5c3c2)c2ccccc2C4(C)C)cc1 ; CCC1(CC)c2ccccc2-c2cc3c(cc21)N(c1ccc2c4cccc5c(N6c7ccccc7C(C)(C)c7cc8c(cc76)C(CC)(CC)c6ccccc6-8)ccc(c6cccc1c62)c54)c1ccccc1C3(C)C ; Cc1ccc2c(N3c4ccccc4C(C)(C)c4cc5c(cc43)oc3ccccc35)c3cc(C)ccc3c(N3c4ccccc4C(C)(C)c4cc5c(cc43)oc3ccccc35)c2c1]

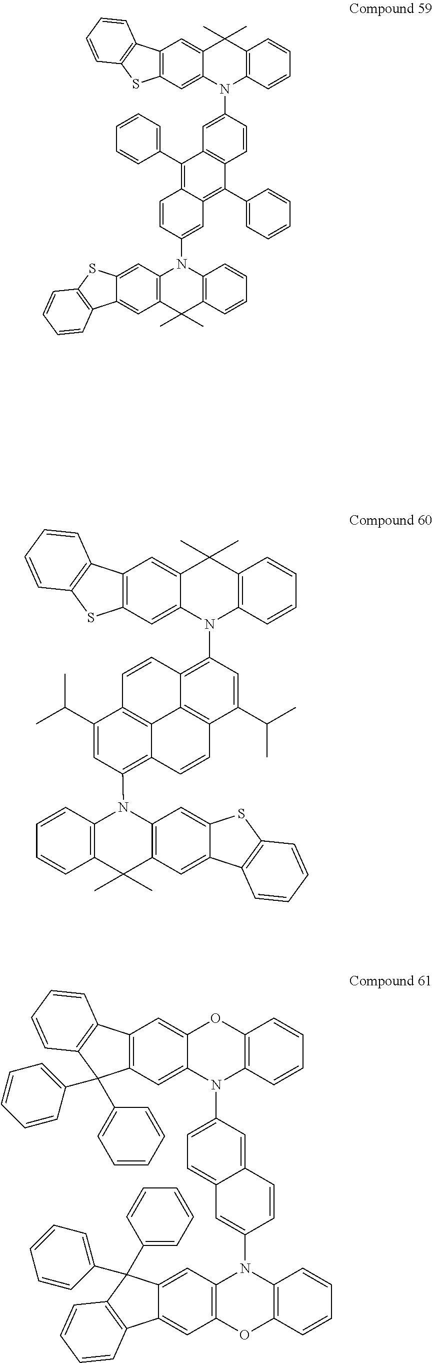 [SMILES: CC1(C)c2ccccc2N(c2ccc3c(-c4ccccc4)c4cc(N5c6ccccc6C(C)(C)c6cc7c(cc65)sc5ccccc57)ccc4c(-c4ccccc4)c3c2)c2cc3sc4ccccc4c3cc21 ; CC(C)c1cc(N2c3ccccc3C(C)(C)c3cc4c(cc32)sc2ccccc24)c2ccc3c(C(C)C)cc(N4c5ccccc5C(C)(C)c5cc6c(cc54)sc4ccccc46)c4ccc1c2c34 ; c1ccc(C2(c3ccccc3)c3ccccc3-c3cc4c(cc32)N(c2ccc3cc(N5c6ccccc6Oc6cc7c(cc65)C(c5ccccc5)(c5ccccc5)c5ccccc5-7)ccc3c2)c2ccccc2O4)cc1]

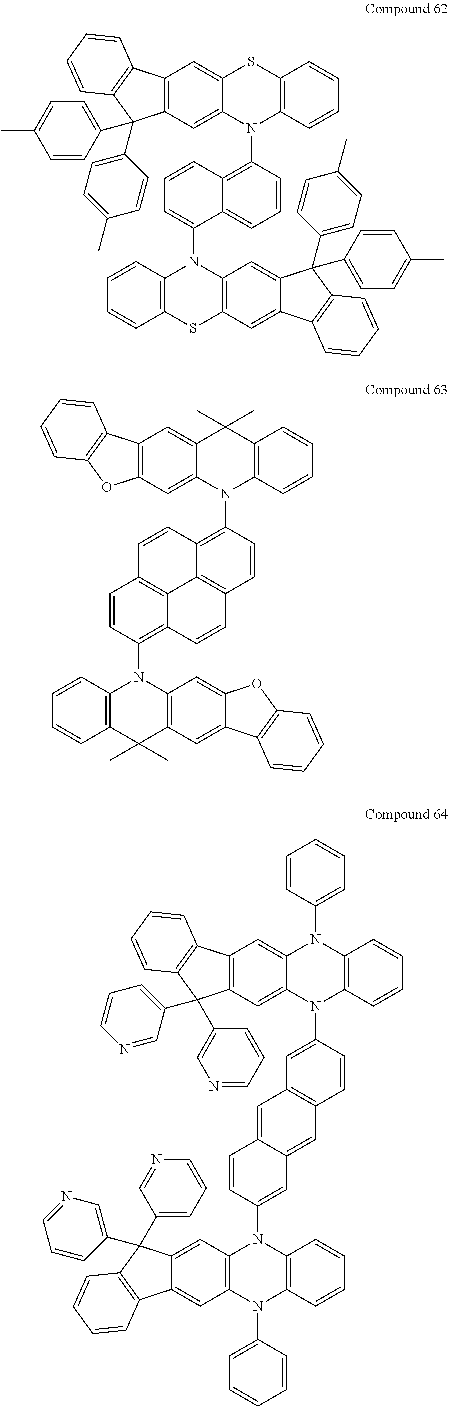

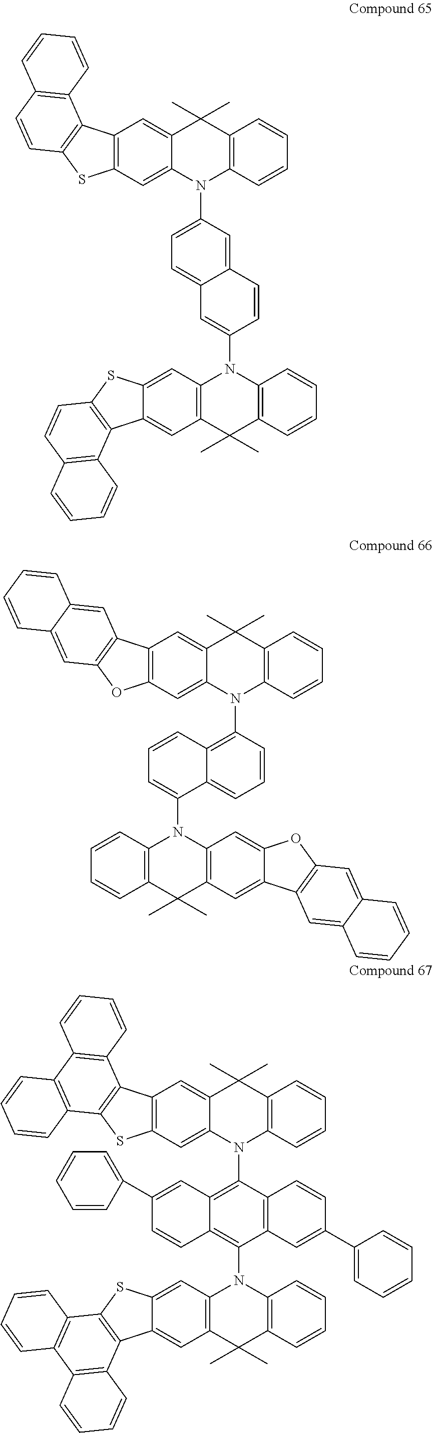 [SMILES: CC1(C)c2ccccc2N(c2ccc3cc(N4c5ccccc5C(C)(C)c5cc6c(cc54)sc4ccc5ccccc5c46)ccc3c2)c2cc3sc4ccc5ccccc5c4c3cc21 ; CC1(C)c2ccccc2N(c2cccc3c(N4c5ccccc5C(C)(C)c5cc6c(cc54)oc4cc5ccccc5cc46)cccc23)c2cc3oc4cc5ccccc5cc4c3cc21 ; CC1(C)c2ccccc2N(c2c3ccc(-c4ccccc4)cc3c(N3c4ccccc4C(C)(C)c4cc5c(cc43)sc3c4ccccc4c4ccccc4c53)c3ccc(-c4ccccc4)cc23)c2cc3sc4c5ccccc5c5ccccc5c4c3cc21]

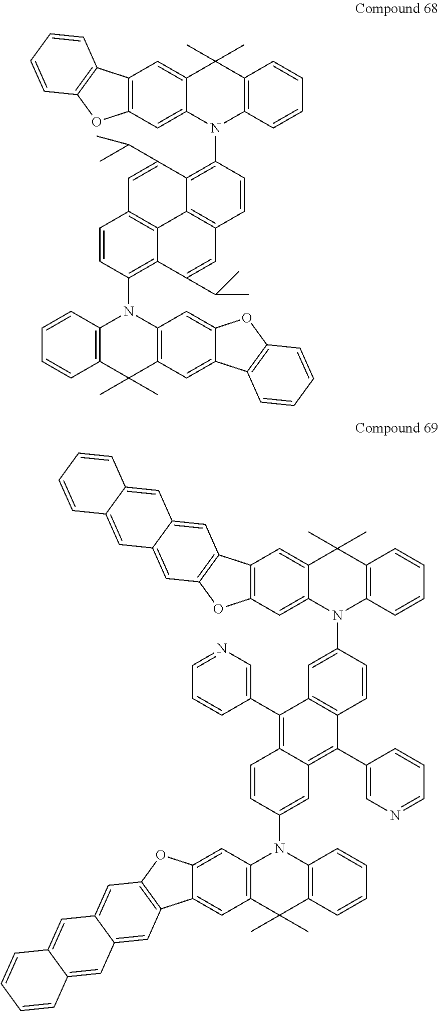 [SMILES: CC(C)c1cc2ccc(N3c4ccccc4C(C)(C)c4cc5c(cc43)oc3ccccc35)c3c(C(C)C)cc4ccc(N5c6ccccc6C(C)(C)c6cc7c(cc65)oc5ccccc57)c1c4c23 ; CC1(C)c2ccccc2N(c2ccc3c(-c4cccnc4)c4cc(N5c6ccccc6C(C)(C)c6cc7c(cc65)oc5cc6cc8ccccc8cc6cc57)ccc4c(-c4cccnc4)c3c2)c2cc3oc4cc5cc6ccccc6cc5cc4c3cc21]

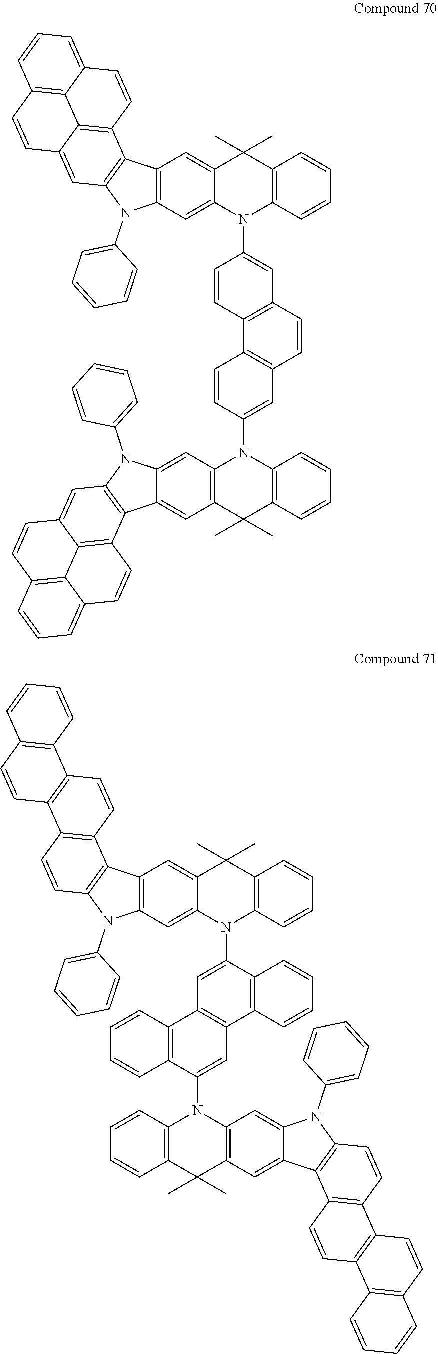 [SMILES: CC1(C)c2ccccc2N(c2ccc3c(ccc4cc(N5c6ccccc6C(C)(C)c6cc7c8c9ccc%10cccc%11ccc(cc8n(-c8ccccc8)c7cc65)c9c%11%10)ccc43)c2)c2cc3c(cc21)c1c2ccc4cccc5ccc(cc1n3-c1ccccc1)c2c54 ; CC1(C)c2ccccc2N(c2cc3c4ccccc4c(N4c5ccccc5C(C)(C)c5cc6c7c8ccc9c%10ccccc%10ccc9c8ccc7n(-c7ccccc7)c6cc54)cc3c3ccccc23)c2cc3c(cc21)c1c2ccc4c5ccccc5ccc4c2ccc1n3-c1ccccc1]

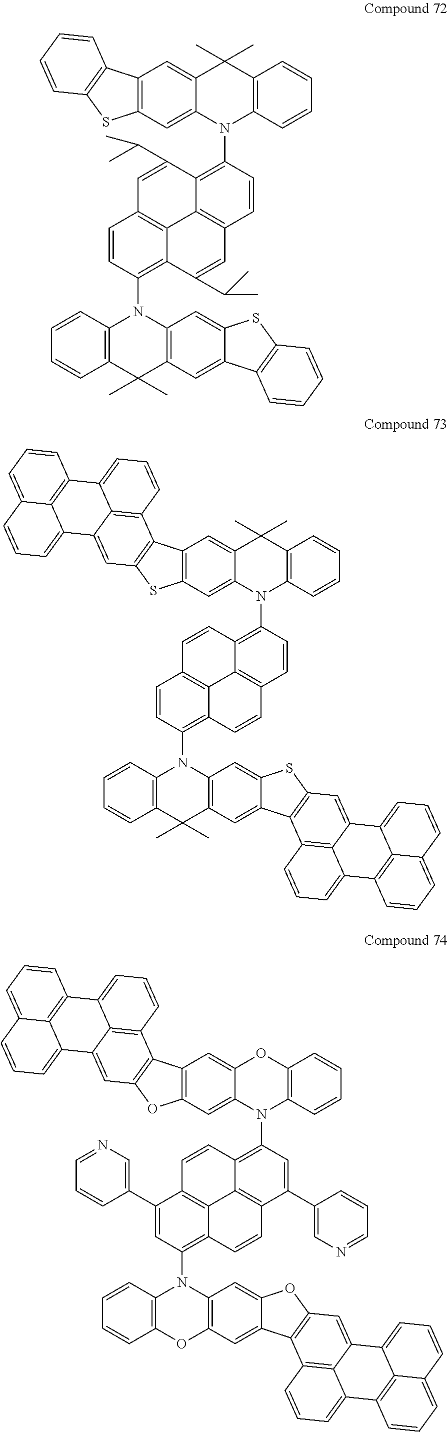 [SMILES: CC(C)c1cc2ccc(N3c4ccccc4C(C)(C)c4cc5c(cc43)sc3ccccc35)c3c(C(C)C)cc4ccc(N5c6ccccc6C(C)(C)c6cc7c(cc65)sc5ccccc57)c1c4c23 ; CC1(C)c2ccccc2N(c2ccc3ccc4c(N5c6ccccc6C(C)(C)c6cc7c(cc65)sc5cc6c8cccc9cccc(c%10cccc(c57)c%106)c98)ccc5ccc2c3c54)c2cc3sc4cc5c6cccc7cccc(c8cccc(c4c3cc21)c85)c76 ; c1cncc(-c2cc(-n3c4ccccc4oc4cc5c(cc43)oc3cc4c6cccc7cccc(c8cccc(c35)c84)c76)c3ccc4c(-c5cccnc5)cc(-n5c6ccccc6oc6cc7c(cc65)oc5cc6c8cccc9cccc(c%10cccc(c57)c%106)c98)c5ccc2c3c45)c1]

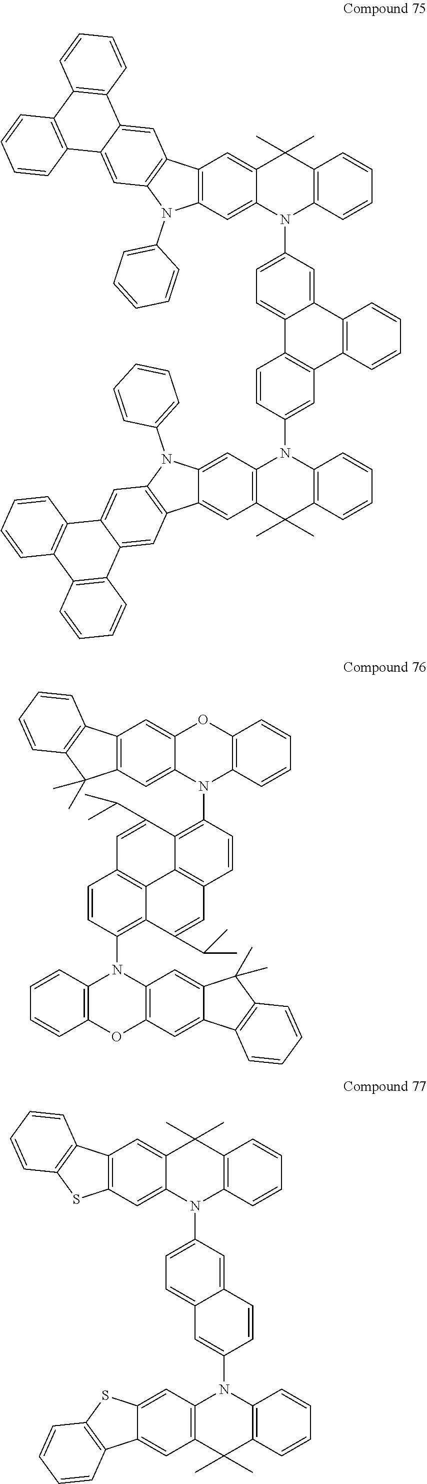 [SMILES: CC1(C)c2ccccc2N(c2ccc3c4ccc(N5c6ccccc6C(C)(C)c6cc7c8cc9c%10ccccc%10c%10ccccc%10c9cc8n(-c8ccccc8)c7cc65)cc4c4ccccc4c3c2)c2cc3c(cc21)c1cc2c4ccccc4c4ccccc4c2cc1n3-c1ccccc1 ; CC(C)c1cc2ccc(N3c4ccccc4Oc4cc5c(cc43)C(C)(C)c3ccccc3-5)c3c(C(C)C)cc4ccc(N5c6ccccc6Oc6cc7c(cc65)C(C)(C)c5ccccc5-7)c1c4c23 ; CC1(C)c2ccccc2N(c2ccc3cc(N4c5ccccc5C(C)(C)c5cc6c(cc54)sc4ccccc46)ccc3c2)c2cc3sc4ccccc4c3cc21]

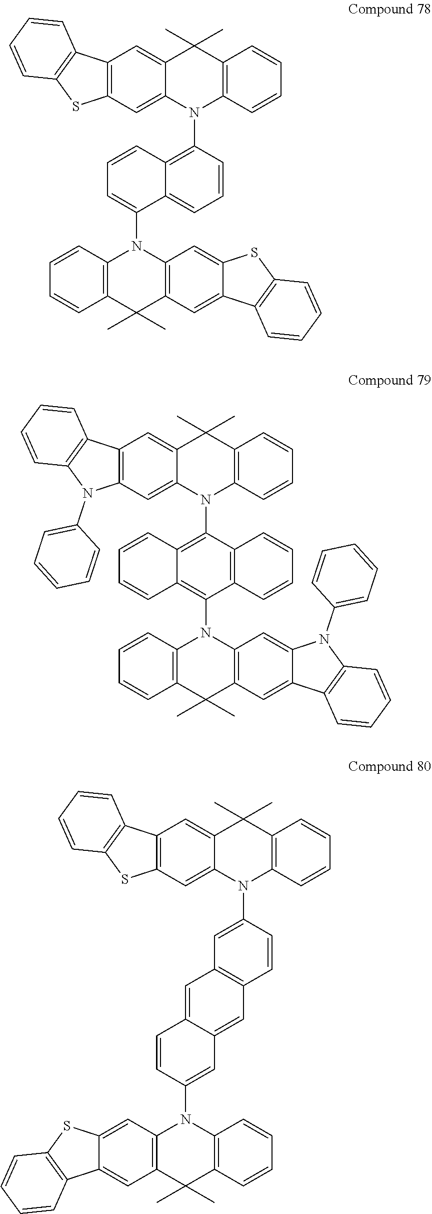 [SMILES: CC1(C)c2ccccc2N(c2cccc3c(N4c5ccccc5C(C)(C)c5cc6c(cc54)sc4ccccc46)cccc23)c2cc3sc4ccccc4c3cc21 ; CC1(C)c2ccccc2N(c2c3ccccc3c(N3c4ccccc4C(C)(C)c4cc5c6ccccc6n(-c6ccccc6)c5cc43)c3ccccc23)c2cc3c(cc21)c1ccccc1n3-c1ccccc1 ; CC1(C)c2ccccc2N(c2ccc3cc4cc(N5c6ccccc6C(C)(C)c6cc7c(cc65)sc5ccccc57)ccc4cc3c2)c2cc3sc4ccccc4c3cc21]

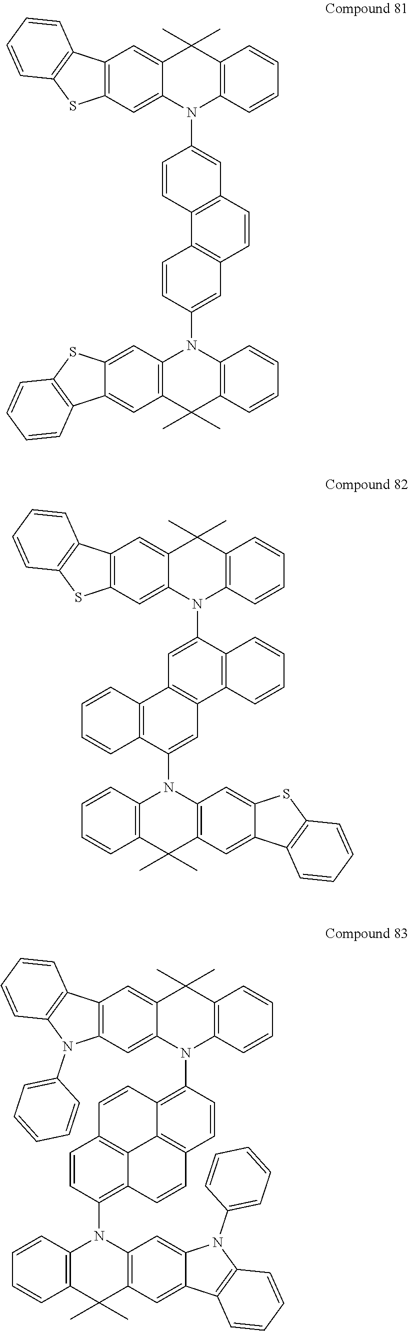 [SMILES: CC1(C)c2ccccc2N(c2ccc3c(ccc4cc(N5c6ccccc6C(C)(C)c6cc7c(cc65)sc5ccccc57)ccc43)c2)c2cc3sc4ccccc4c3cc21 ; CC1(C)c2ccccc2N(c2cc3c4ccccc4c(N4c5ccccc5C(C)(C)c5cc6c(cc54)sc4ccccc46)cc3c3ccccc23)c2cc3sc4ccccc4c3cc21 ; CC1(C)c2ccccc2N(c2ccc3ccc4c(N5c6ccccc6C(C)(C)c6cc7c8ccccc8n(-c8ccccc8)c7cc65)ccc5ccc2c3c54)c2cc3c(cc21)c1ccccc1n3-c1ccccc1]

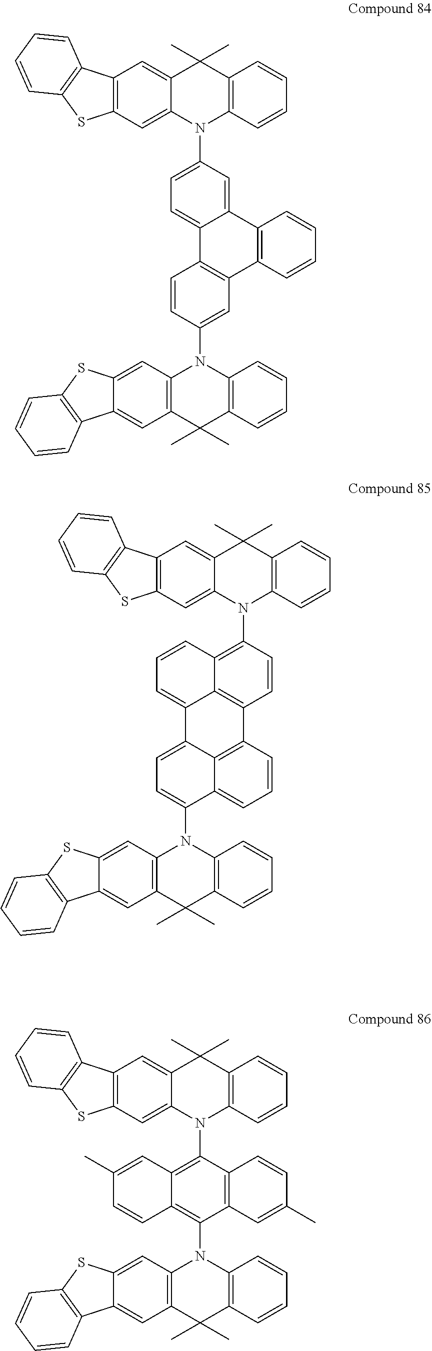 [SMILES: CC1(C)c2ccccc2N(c2ccc3c4ccc(N5c6ccccc6C(C)(C)c6cc7c(cc65)sc5ccccc57)cc4c4ccccc4c3c2)c2cc3sc4ccccc4c3cc21 ; CC1(C)c2ccccc2N(c2ccc3c4cccc5c(N6c7ccccc7C(C)(C)c7cc8c(cc76)sc6ccccc68)ccc(c6cccc2c63)c54)c2cc3sc4ccccc4c3cc21 ; Cc1ccc2c(N3c4ccccc4C(C)(C)c4cc5c(cc43)sc3ccccc35)c3cc(C)ccc3c(N3c4ccccc4C(C)(C)c4cc5c(cc43)sc3ccccc35)c2c1]

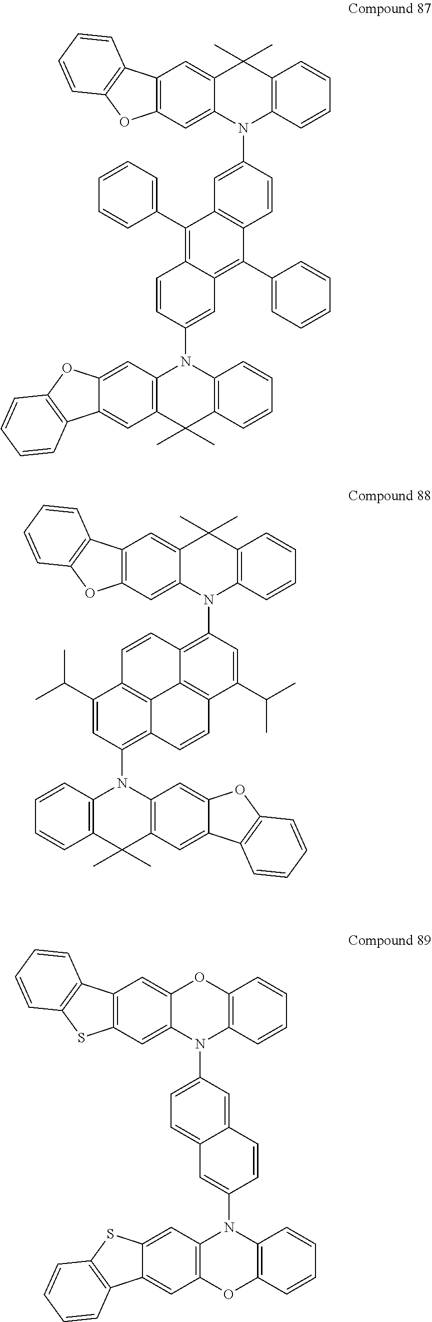 [SMILES: CC1(C)c2ccccc2N(c2ccc3c(-c4ccccc4)c4cc(N5c6ccccc6C(C)(C)c6cc7c(cc65)oc5ccccc57)ccc4c(-c4ccccc4)c3c2)c2cc3oc4ccccc4c3cc21 ; CC(C)c1cc(N2c3ccccc3C(C)(C)c3cc4c(cc32)oc2ccccc24)c2ccc3c(C(C)C)cc(N4c5ccccc5C(C)(C)c5cc6c(cc54)oc4ccccc46)c4ccc1c2c34 ; c1ccc2c(c1)Oc1cc3c(cc1N2c1ccc2cc(N4c5ccccc5Oc5cc6c(cc54)sc4ccccc46)ccc2c1)sc1ccccc13]

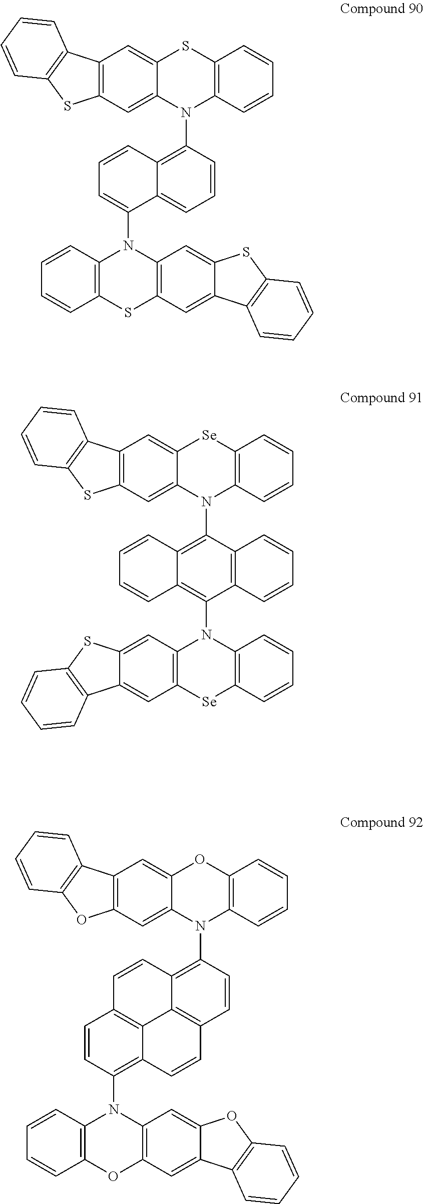 [SMILES: c1ccc2c(c1)Sc1cc3c(cc1N2c1cccc2c(N4c5ccccc5Sc5cc6c(cc54)sc4ccccc46)cccc12)sc1ccccc13 ; c1ccc2c(c1)[Se]c1cc3c(cc1N2c1c2ccccc2c(N2c4ccccc4[Se]c4cc5c(cc42)sc2ccccc25)c2ccccc12)sc1ccccc13 ; c1ccc2c(c1)Oc1cc3c(cc1N2c1ccc2ccc4c(N5c6ccccc6Oc6cc7c(cc65)oc5ccccc57)ccc5ccc1c2c54)oc1ccccc13]

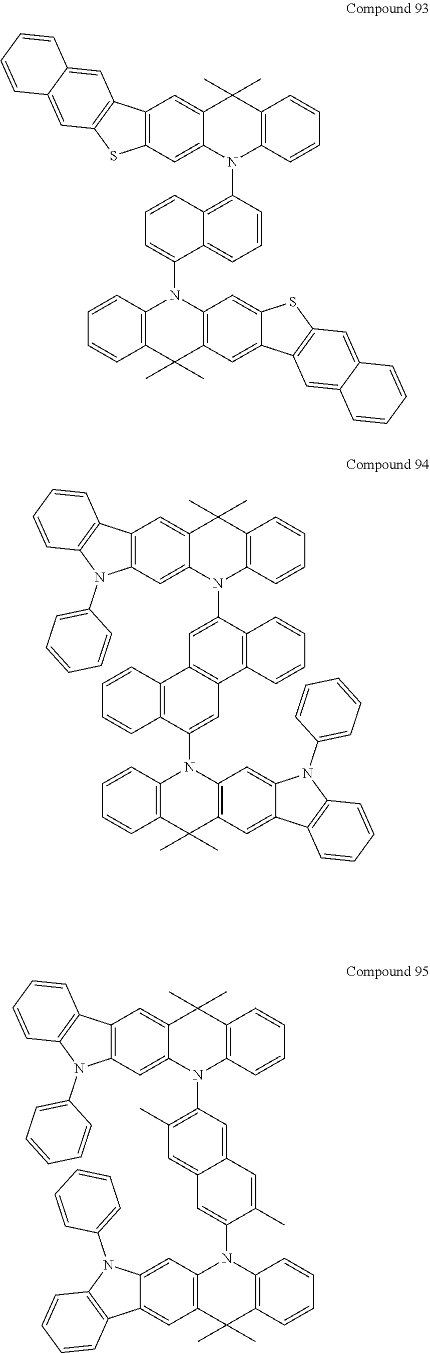 [SMILES: CC1(C)c2ccccc2N(c2cccc3c(N4c5ccccc5C(C)(C)c5cc6c(cc54)sc4cc5ccccc5cc46)cccc23)c2cc3sc4cc5ccccc5cc4c3cc21 ; CC1(C)c2ccccc2N(c2cc3c4ccccc4c(N4c5ccccc5C(C)(C)c5cc6c7ccccc7n(-c7ccccc7)c6cc54)cc3c3ccccc23)c2cc3c(cc21)c1ccccc1n3-c1ccccc1 ; Cc1cc2cc(N3c4ccccc4C(C)(C)c4cc5c6ccccc6n(-c6ccccc6)c5cc43)c(C)cc2cc1N1c2ccccc2C(C)(C)c2cc3c4ccccc4n(-c4ccccc4)c3cc21]

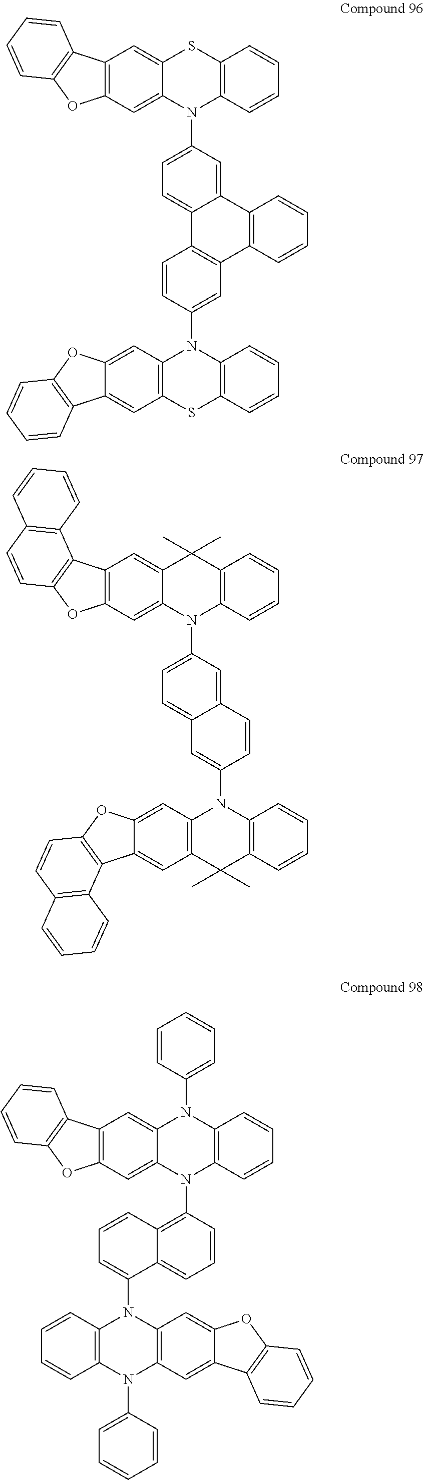

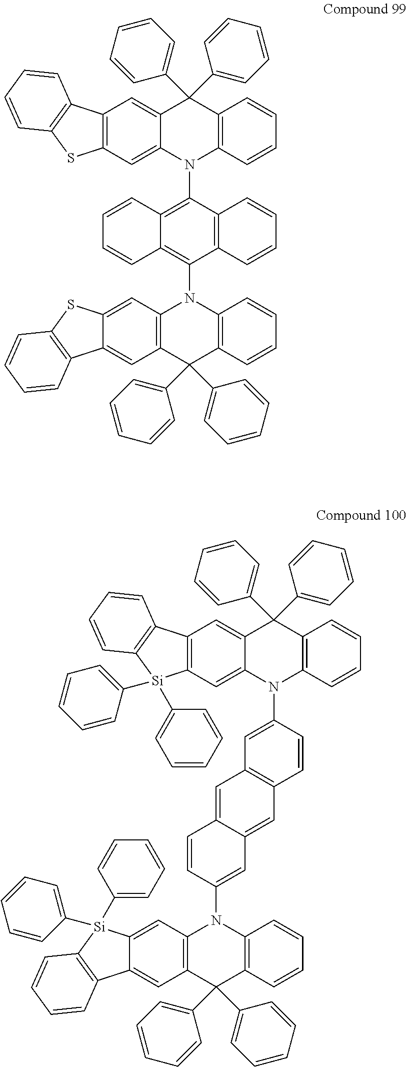 [SMILES: c1ccc(C2(c3ccccc3)c3ccccc3N(c3c4ccccc4c(N4c5ccccc5C(c5ccccc5)(c5ccccc5)c5cc6c(cc54)sc4ccccc46)c4ccccc34)c3cc4sc5ccccc5c4cc32)cc1 ; c1ccc(C2(c3ccccc3)c3ccccc3N(c3ccc4cc5cc(N6c7ccccc7C(c7ccccc7)(c7ccccc7)c7cc8c(cc76)[Si](c6ccccc6)(c6ccccc6)c6ccccc6-8)ccc5cc4c3)c3cc4c(cc32)-c2ccccc2[Si]4(c2ccccc2)c2ccccc2)cc1]

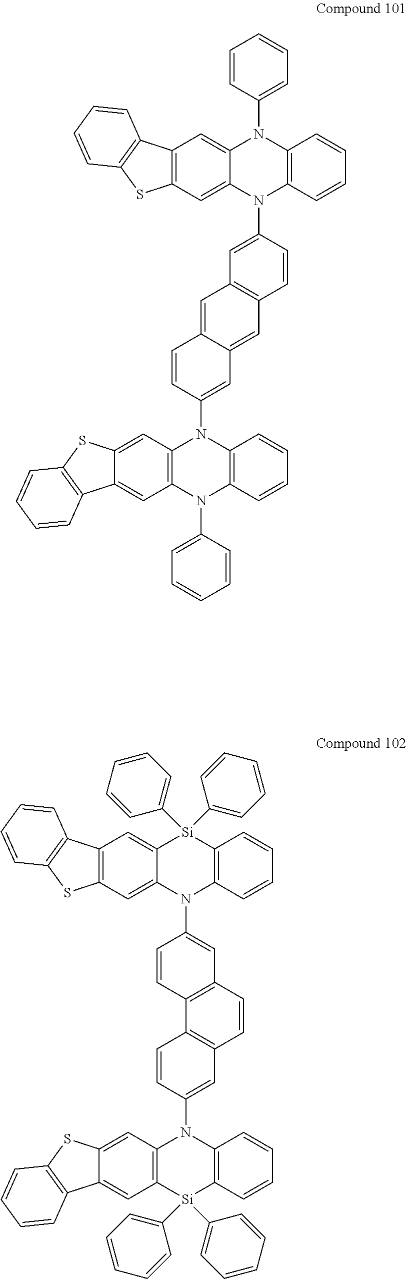 [SMILES: c1ccc(N2c3ccccc3N(c3ccc4cc5cc(N6c7ccccc7N(c7ccccc7)c7cc8c(cc76)sc6ccccc68)ccc5cc4c3)c3cc4sc5ccccc5c4cc32)cc1 ; c1ccc([Si]2(c3ccccc3)c3ccccc3N(c3ccc4c(ccc5cc(N6c7ccccc7[Si](c7ccccc7)(c7ccccc7)c7cc8c(cc76)sc6ccccc68)ccc54)c3)c3cc4sc5ccccc5c4cc32)cc1]

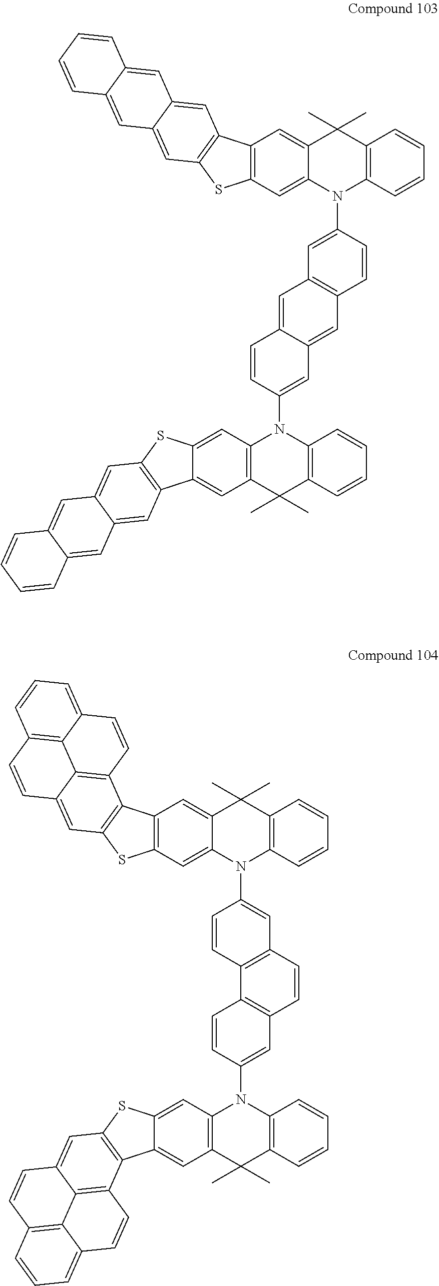 [SMILES: CC1(C)c2ccccc2N(c2ccc3cc4cc(N5c6ccccc6C(C)(C)c6cc7c(cc65)sc5cc6cc8ccccc8cc6cc57)ccc4cc3c2)c2cc3sc4cc5cc6ccccc6cc5cc4c3cc21 ; CC1(C)c2ccccc2N(c2ccc3c(ccc4cc(N5c6ccccc6C(C)(C)c6cc7c(cc65)sc5cc6ccc8cccc9ccc(c57)c6c89)ccc43)c2)c2cc3sc4cc5ccc6cccc7ccc(c4c3cc21)c5c67]

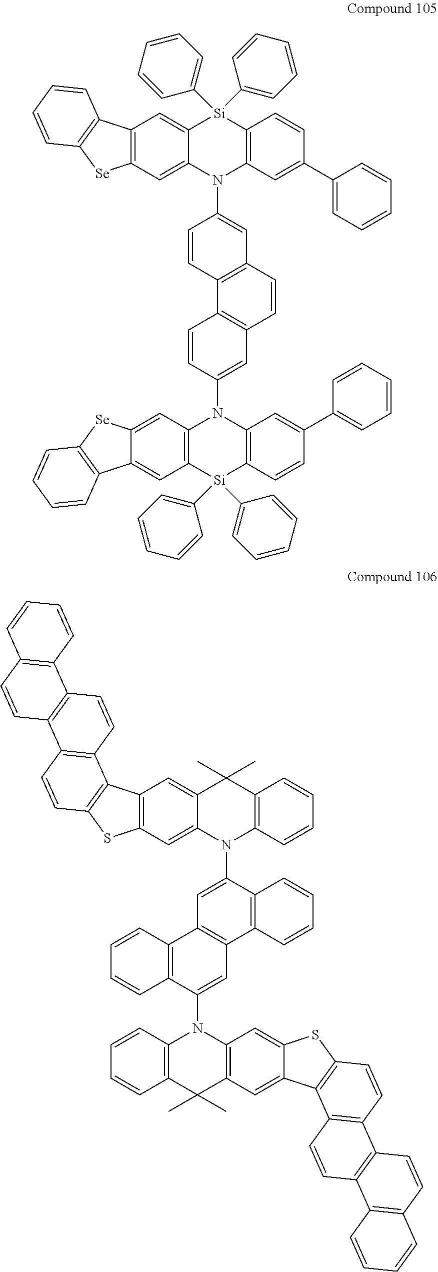 [SMILES: c1ccc(-c2ccc3c(c2)N(c2ccc4c(ccc5cc(N6c7cc(-c8ccccc8)ccc7[Si](c7ccccc7)(c7ccccc7)c7cc8c(cc76)[se]c6ccccc68)ccc54)c2)c2cc4[se]c5ccccc5c4cc2[Si]3(c2ccccc2)c2ccccc2)cc1 ; CC1(C)c2ccccc2N(c2cc3c4ccccc4c(N4c5ccccc5C(C)(C)c5cc6c(cc54)sc4ccc5c7ccc8ccccc8c7ccc5c46)cc3c3ccccc23)c2cc3sc4ccc5c6ccc7ccccc7c6ccc5c4c3cc21]

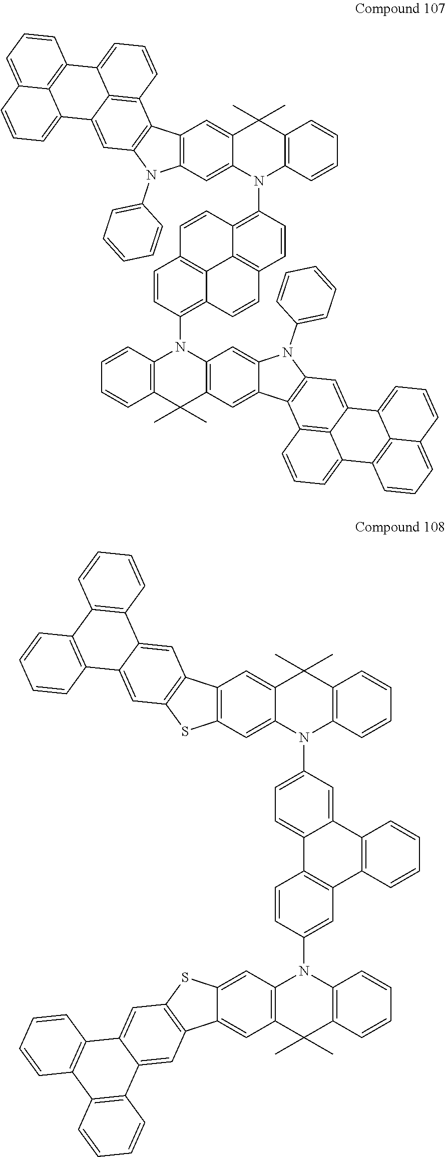 [SMILES: CC1(C)c2ccccc2N(c2ccc3ccc4c(N5c6ccccc6C(C)(C)c6cc7c8c9cccc%10c%11cccc%12cccc(c(cc8n(-c8ccccc8)c7cc65)c%109)c%12%11)ccc5ccc2c3c54)c2cc3c(cc21)c1c2cccc4c5cccc6cccc(c(cc1n3-c1ccccc1)c42)c65 ; CC1(C)c2ccccc2N(c2ccc3c4ccc(N5c6ccccc6C(C)(C)c6cc7c(cc65)sc5cc6c8ccccc8c8ccccc8c6cc57)cc4c4ccccc4c3c2)c2cc3sc4cc5c6ccccc6c6ccccc6c5cc4c3cc21]

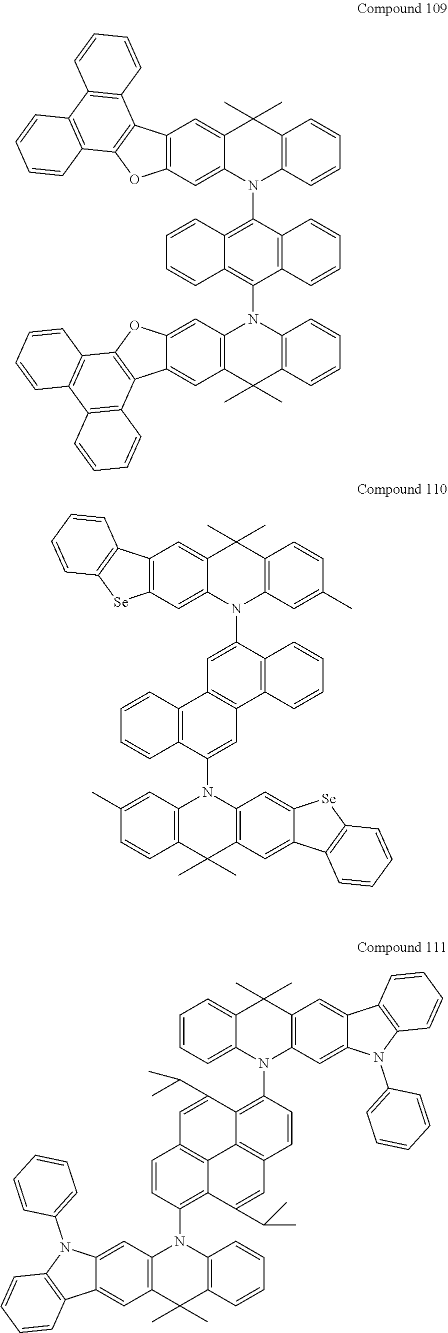 [SMILES: CC1(C)c2ccccc2N(c2c3ccccc3c(N3c4ccccc4C(C)(C)c4cc5c(cc43)oc3c4ccccc4c4ccccc4c53)c3ccccc23)c2cc3oc4c5ccccc5c5ccccc5c4c3cc21 ; Cc1ccc2c(c1)N(c1cc3c4ccccc4c(N4c5cc(C)ccc5C(C)(C)c5cc6c(cc54)[se]c4ccccc46)cc3c3ccccc13)c1cc3[se]c4ccccc4c3cc1C2(C)C ; CC(C)c1cc2ccc(N3c4ccccc4C(C)(C)c4cc5c6ccccc6n(-c6ccccc6)c5cc43)c3c(C(C)C)cc4ccc(N5c6ccccc6C(C)(C)c6cc7c8ccccc8n(-c8ccccc8)c7cc65)c1c4c23]

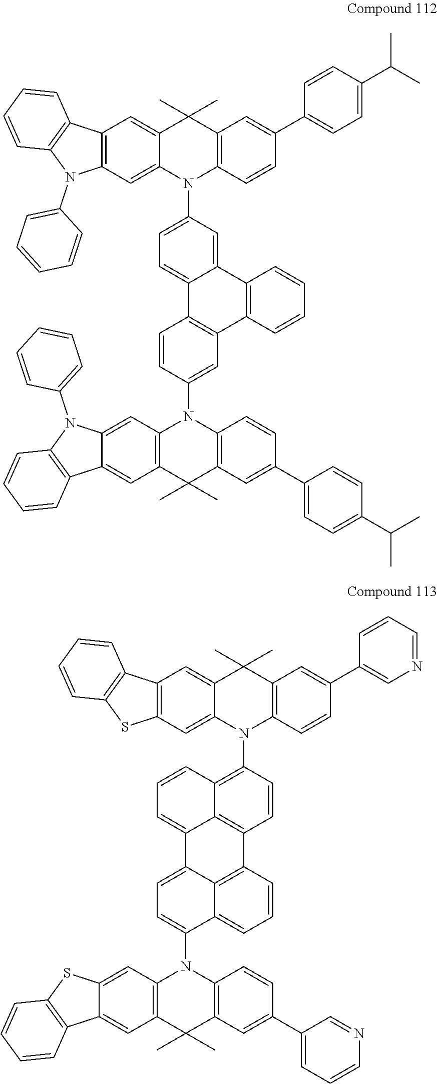 [SMILES: CC(C)c1ccc(-c2ccc3c(c2)C(C)(C)c2cc4c5ccccc5n(-c5ccccc5)c4cc2N3c2ccc3c4ccc(N5c6ccc(-c7ccc(C(C)C)cc7)cc6C(C)(C)c6cc7c8ccccc8n(-c8ccccc8)c7cc65)cc4c4ccccc4c3c2)cc1 ; CC1(C)c2cc(-c3cccnc3)ccc2N(c2ccc3c4cccc5c(N6c7ccc(-c8cccnc8)cc7C(C)(C)c7cc8c(cc76)sc6ccccc68)ccc(c6cccc2c63)c54)c2cc3sc4ccccc4c3cc21]

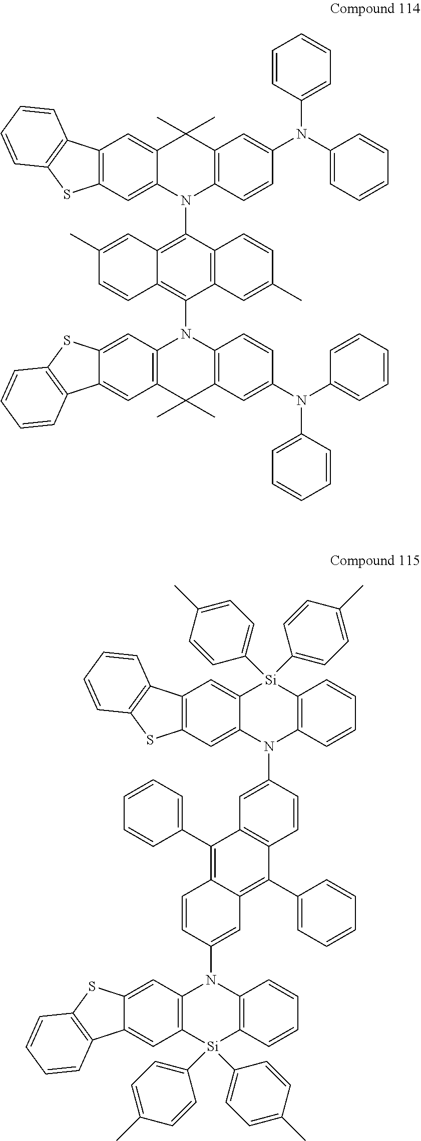 [SMILES: Cc1ccc2c(N3c4ccc(N(c5ccccc5)c5ccccc5)cc4C(C)(C)c4cc5c(cc43)sc3ccccc35)c3cc(C)ccc3c(N3c4ccc(N(c5ccccc5)c5ccccc5)cc4C(C)(C)c4cc5c(cc43)sc3ccccc35)c2c1 ; Cc1ccc([Si]2(c3ccc(C)cc3)c3ccccc3N(c3ccc4c(-c5ccccc5)c5cc(N6c7ccccc7[Si](c7ccc(C)cc7)(c7ccc(C)cc7)c7cc8c(cc76)sc6ccccc68)ccc5c(-c5ccccc5)c4c3)c3cc4sc5ccccc5c4cc32)cc1]

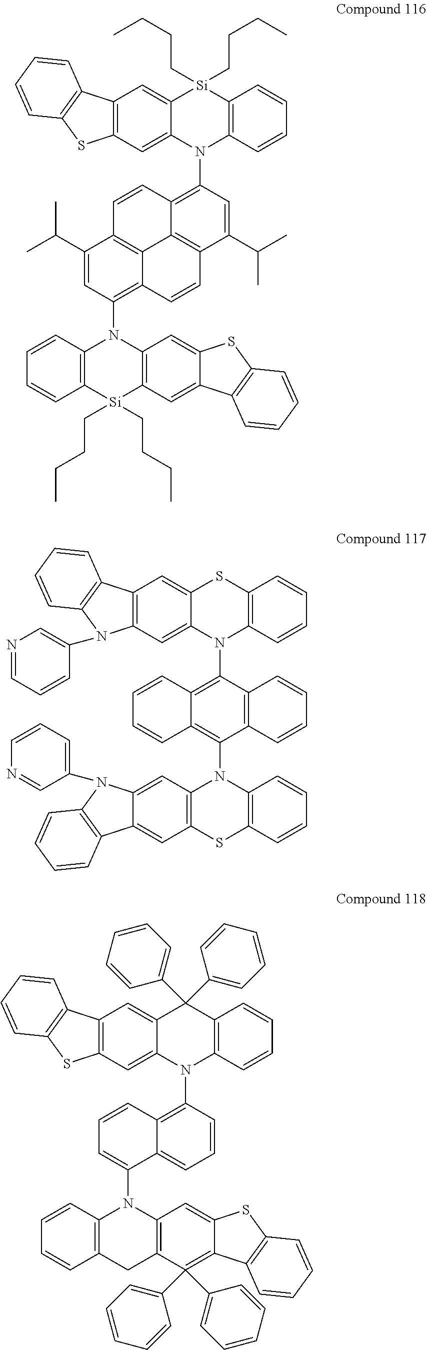

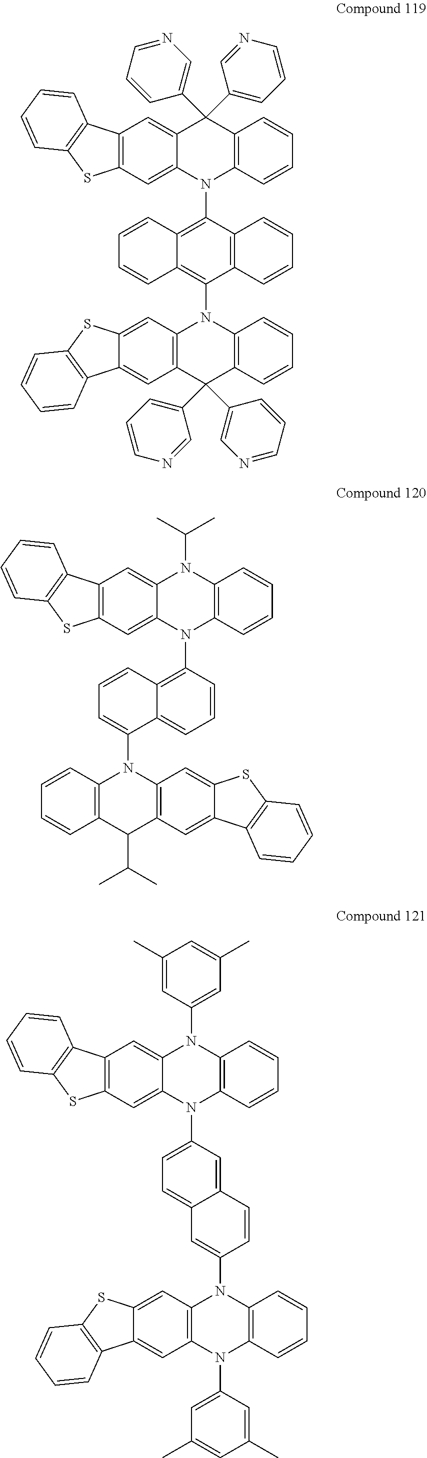 [SMILES: c1cncc(C2(c3cccnc3)c3ccccc3N(c3c4ccccc4c(N4c5ccccc5C(c5cccnc5)(c5cccnc5)c5cc6c(cc54)sc4ccccc46)c4ccccc34)c3cc4sc5ccccc5c4cc32)c1 ; CC(C)C1c2ccccc2N(c2cccc3c(N4c5ccccc5N(C(C)C)c5cc6c(cc54)sc4ccccc46)cccc23)c2cc3sc4ccccc4c3cc21 ; Cc1cc(C)cc(N2c3ccccc3N(c3ccc4cc(N5c6ccccc6N(c6cc(C)cc(C)c6)c6cc7c(cc65)sc5ccccc57)ccc4c3)c3cc4sc5ccccc5c4cc32)c1]

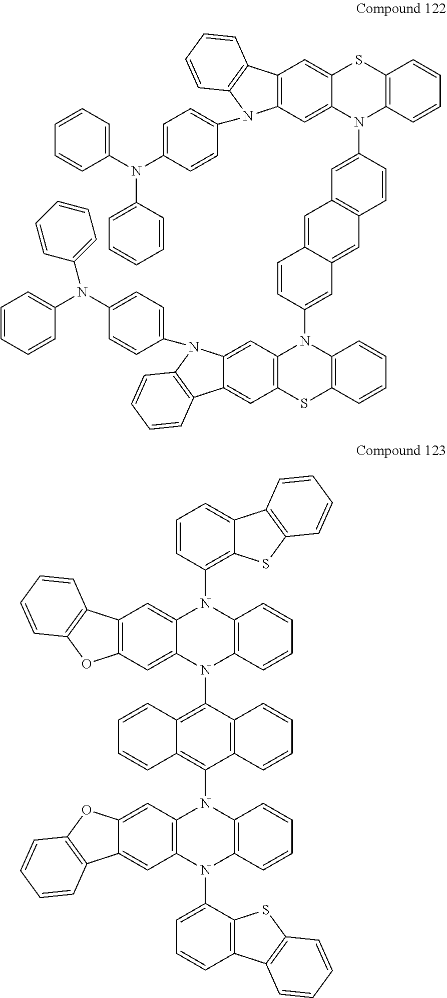 [SMILES: c1ccc(N(c2ccccc2)c2ccc(-n3c4ccccc4c4cc5c(cc43)N(c3ccc4cc6cc(N7c8ccccc8Sc8cc9c%10ccccc%10n(-c%10ccc(N(c%11ccccc%11)c%11ccccc%11)cc%10)c9cc87)ccc6cc4c3)c3ccccc3S5)cc2)cc1 ; c1ccc2c(c1)N(c1c3ccccc3c(N3c4ccccc4N(c4cccc5c4sc4ccccc45)c4cc5c(cc43)oc3ccccc35)c3ccccc13)c1cc3oc4ccccc4c3cc1N2c1cccc2c1sc1ccccc12]

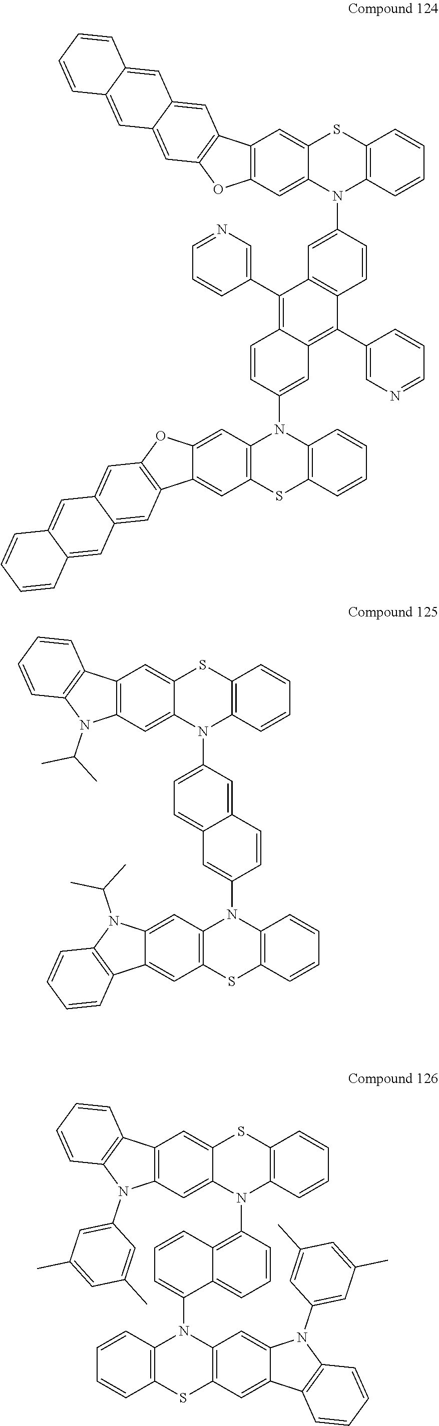

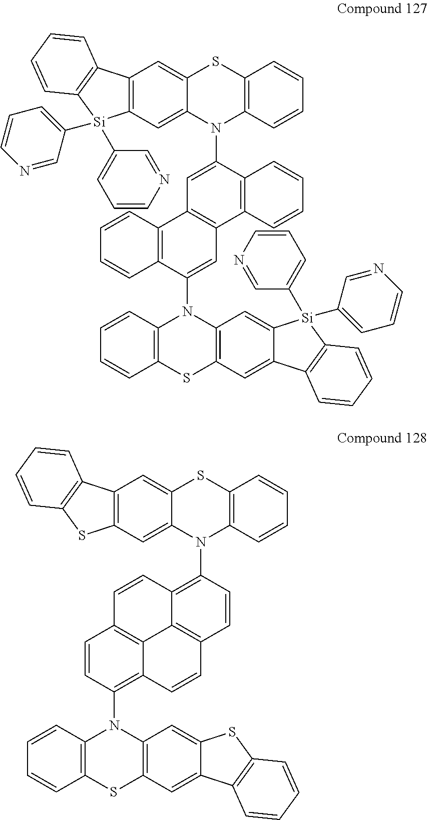 [SMILES: c1cncc([Si]2(c3cccnc3)c3ccccc3-c3cc4c(cc32)N(c2cc3c5ccccc5c(N5c6ccccc6Sc6cc7c(cc65)[Si](c5cccnc5)(c5cccnc5)c5ccccc5-7)cc3c3ccccc23)c2ccccc2S4)c1 ; c1ccc2c(c1)Sc1cc3c(cc1N2c1ccc2ccc4c(N5c6ccccc6Sc6cc7c(cc65)sc5ccccc57)ccc5ccc1c2c54)sc1ccccc13]

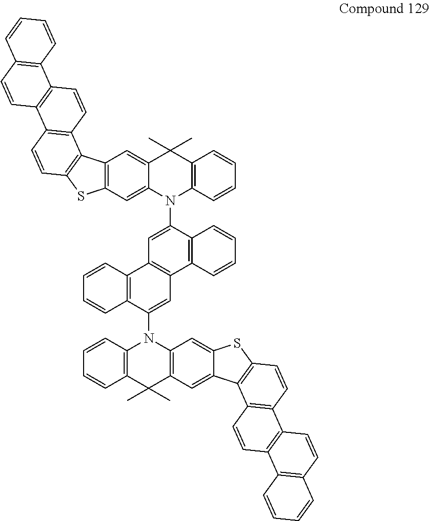 [SMILES: CC1(C)c2ccccc2N(c2cc3c4ccccc4c(N4c5ccccc5C(C)(C)c5cc6c(cc54)sc4ccc5c7ccc8ccccc8c7ccc5c46)cc3c3ccccc23)c2cc3sc4ccc5c6ccc7ccccc7c6ccc5c4c3cc21]

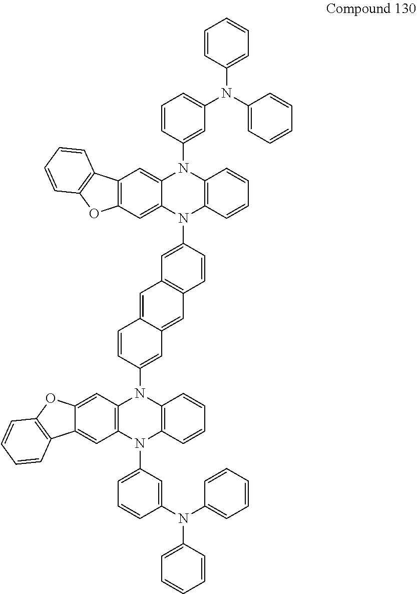 [SMILES: c1ccc(N(c2ccccc2)c2cccc(N3c4ccccc4N(c4ccc5cc6cc(N7c8ccccc8N(c8cccc(N(c9ccccc9)c9ccccc9)c8)c8cc9c(cc87)oc7ccccc79)ccc6cc5c4)c4cc5oc6ccccc6c5cc43)c2)cc1]

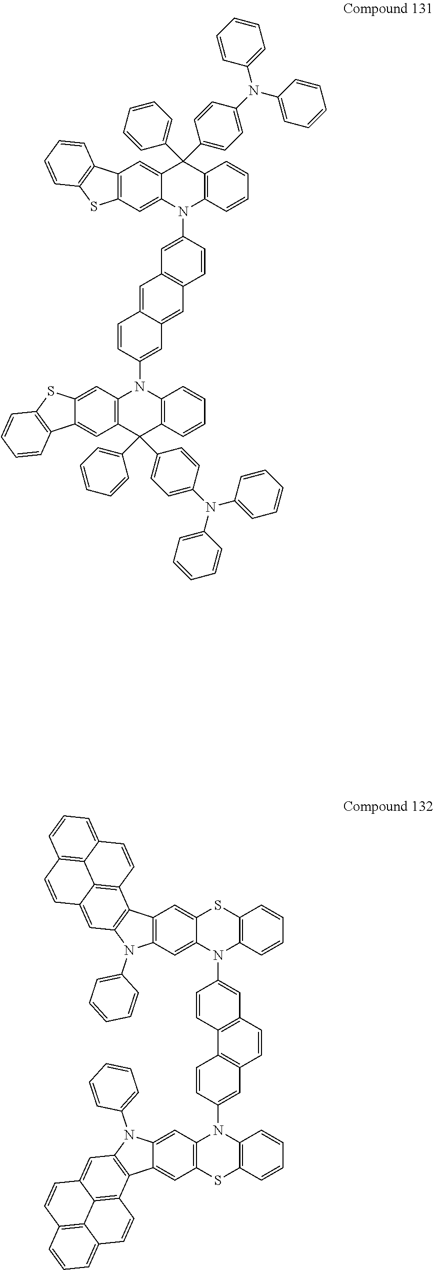 [SMILES: c1ccc(N(c2ccccc2)c2ccc(C3(c4ccccc4)c4ccccc4N(c4ccc5cc6cc(N7c8ccccc8C(c8ccccc8)(c8ccc(N(c9ccccc9)c9ccccc9)cc8)c8cc9c(cc87)sc7ccccc79)ccc6cc5c4)c4cc5sc6ccccc6c5cc43)cc2)cc1 ; c1ccc(-n2c3cc4c(cc3c3c5ccc6cccc7ccc(cc32)c5c76)sc2ccccc2n4-c2ccc3c(ccc4cc(-n5c6ccccc6sc6cc7c8c9ccc%10cccc%11ccc(cc8n(-c8ccccc8)c7cc65)c9c%11%10)ccc43)c2)cc1]

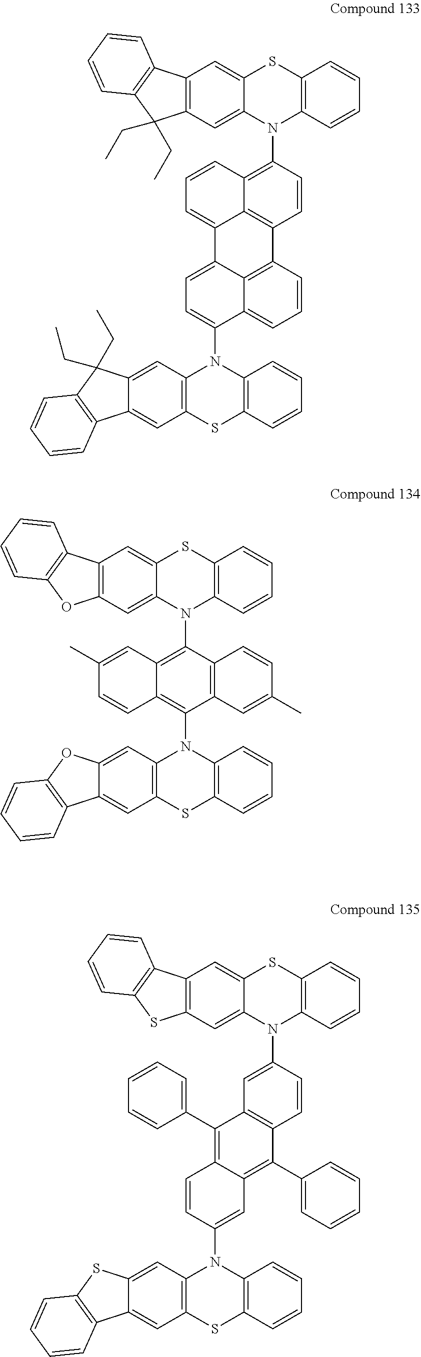 [SMILES: CCC1(CC)c2ccccc2-c2cc3c(cc21)N(c1ccc2c4cccc5c(N6c7ccccc7Sc7cc8c(cc76)C(CC)(CC)c6ccccc6-8)ccc(c6cccc1c62)c54)c1ccccc1S3 ; Cc1ccc2c(N3c4ccccc4Sc4cc5c(cc43)oc3ccccc35)c3cc(C)ccc3c(N3c4ccccc4Sc4cc5c(cc43)oc3ccccc35)c2c1 ; c1ccc(-c2c3ccc(N4c5ccccc5Sc5cc6c(cc54)sc4ccccc46)cc3c(-c3ccccc3)c3ccc(N4c5ccccc5Sc5cc6c(cc54)sc4ccccc46)cc23)cc1]

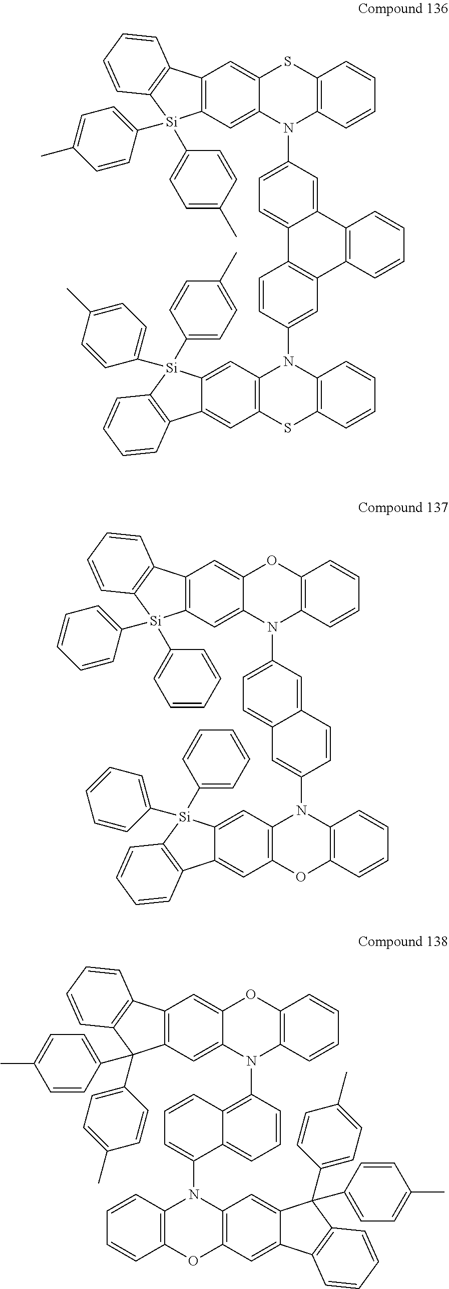

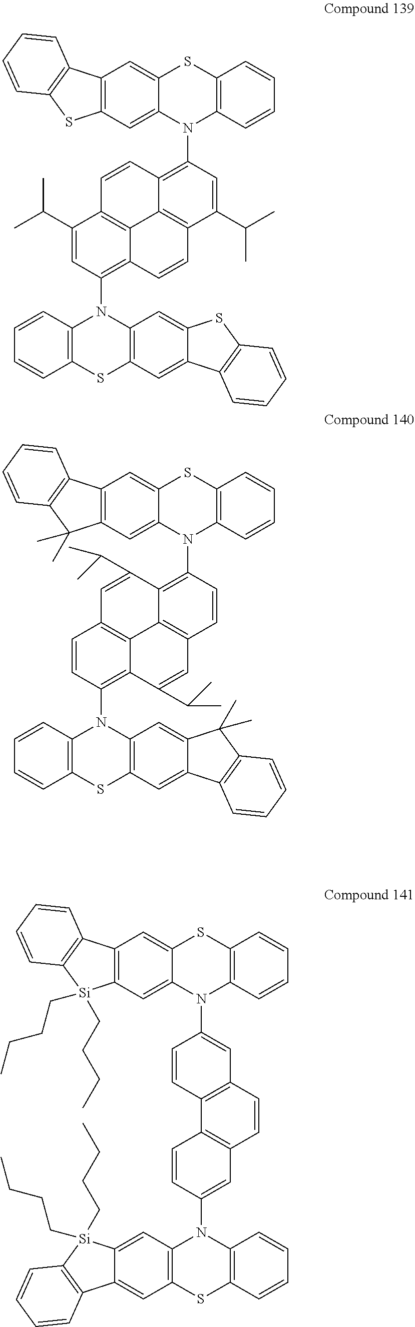 [SMILES: CC(C)c1cc(N2c3ccccc3Sc3cc4c(cc32)sc2ccccc24)c2ccc3c(C(C)C)cc(N4c5ccccc5Sc5cc6c(cc54)sc4ccccc46)c4ccc1c2c34 ; CC(C)c1cc2ccc(N3c4ccccc4Sc4cc5c(cc43)C(C)(C)c3ccccc3-5)c3c(C(C)C)cc4ccc(N5c6ccccc6Sc6cc7c(cc65)C(C)(C)c5ccccc5-7)c1c4c23 ; CCCC[Si]1(CCCC)c2ccccc2-c2cc3c(cc21)N(c1ccc2c(ccc4cc(N5c6ccccc6Sc6cc7c(cc65)[Si](CCCC)(CCCC)c5ccccc5-7)ccc42)c1)c1ccccc1S3]

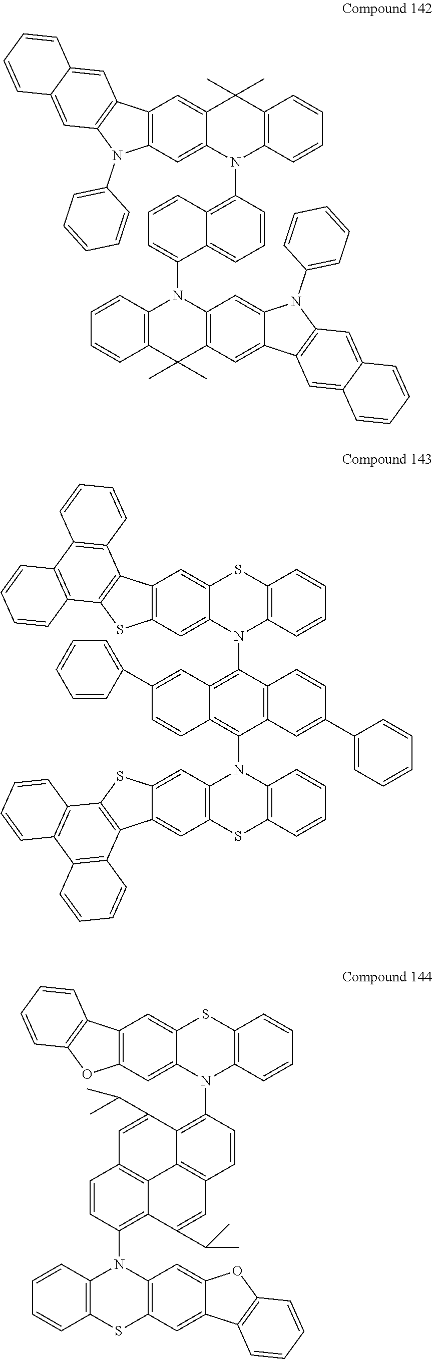 [SMILES: CC1(C)c2ccccc2N(c2cccc3c(N4c5ccccc5C(C)(C)c5cc6c7cc8ccccc8cc7n(-c7ccccc7)c6cc54)cccc23)c2cc3c(cc21)c1cc2ccccc2cc1n3-c1ccccc1 ; c1ccc(-c2ccc3c(N4c5ccccc5Sc5cc6c(cc54)sc4c5ccccc5c5ccccc5c64)c4cc(-c5ccccc5)ccc4c(N4c5ccccc5Sc5cc6c(cc54)sc4c5ccccc5c5ccccc5c64)c3c2)cc1 ; CC(C)c1cc2ccc(N3c4ccccc4Sc4cc5c(cc43)oc3ccccc35)c3c(C(C)C)cc4ccc(N5c6ccccc6Sc6cc7c(cc65)oc5ccccc57)c1c4c23]

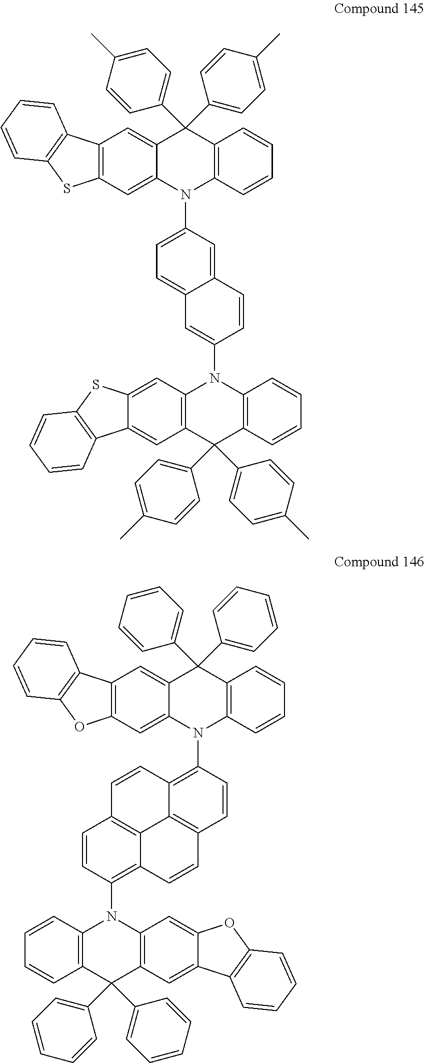 [SMILES: Cc1ccc(C2(c3ccc(C)cc3)c3ccccc3N(c3ccc4cc(N5c6ccccc6C(c6ccc(C)cc6)(c6ccc(C)cc6)c6cc7c(cc65)sc5ccccc57)ccc4c3)c3cc4sc5ccccc5c4cc32)cc1 ; c1ccc(C2(c3ccccc3)c3ccccc3N(c3ccc4ccc5c(N6c7ccccc7C(c7ccccc7)(c7ccccc7)c7cc8c(cc76)oc6ccccc68)ccc6ccc3c4c65)c3cc4oc5ccccc5c4cc32)cc1]

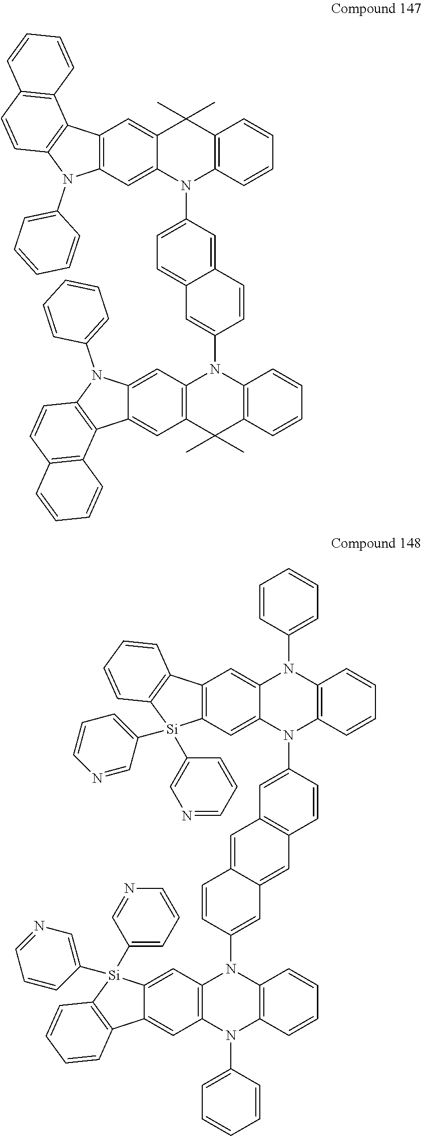 [SMILES: CC1(C)c2ccccc2N(c2ccc3cc(N4c5ccccc5C(C)(C)c5cc6c7c8ccccc8ccc7n(-c7ccccc7)c6cc54)ccc3c2)c2cc3c(cc21)c1c2ccccc2ccc1n3-c1ccccc1 ; c1ccc(N2c3ccccc3N(c3ccc4cc5cc(N6c7ccccc7N(c7ccccc7)c7cc8c(cc76)[Si](c6cccnc6)(c6cccnc6)c6ccccc6-8)ccc5cc4c3)c3cc4c(cc32)-c2ccccc2[Si]4(c2cccnc2)c2cccnc2)cc1]

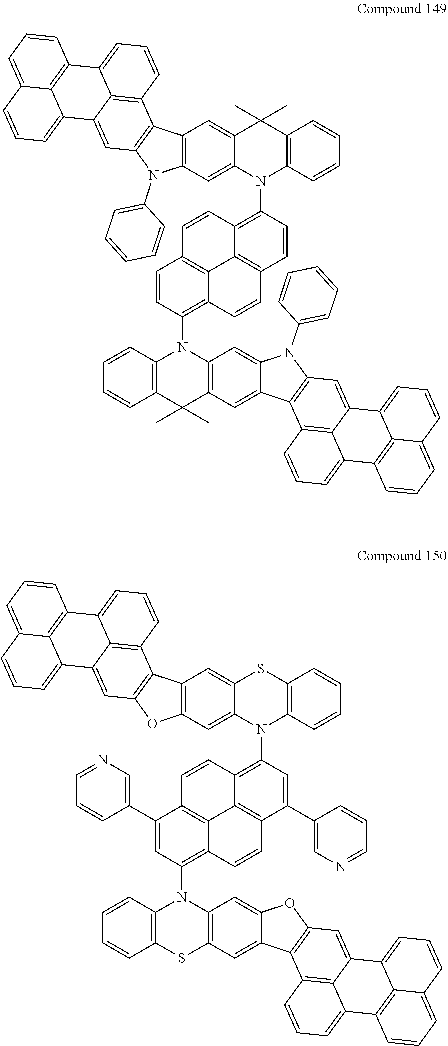 [SMILES: CC1(C)c2ccccc2N(c2ccc3ccc4c(N5c6ccccc6C(C)(C)c6cc7c8c9cccc%10c%11cccc%12cccc(c(cc8n(-c8ccccc8)c7cc65)c%109)c%12%11)ccc5ccc2c3c54)c2cc3c(cc21)c1c2cccc4c5cccc6cccc(c(cc1n3-c1ccccc1)c42)c65 ; c1cncc(-c2cc(-n3c4ccccc4sc4cc5c(cc43)oc3cc4c6cccc7cccc(c8cccc(c35)c84)c76)c3ccc4c(-c5cccnc5)cc(-n5c6ccccc6sc6cc7c(cc65)oc5cc6c8cccc9cccc(c%10cccc(c57)c%106)c98)c5ccc2c3c45)c1]

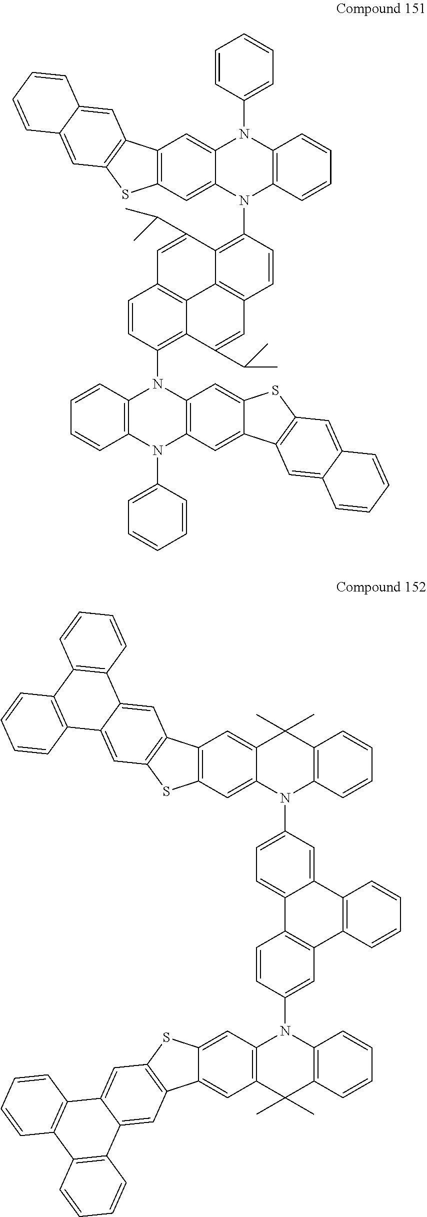 [SMILES: CC(C)c1cc2ccc(N3c4ccccc4N(c4ccccc4)c4cc5c(cc43)sc3cc4ccccc4cc35)c3c(C(C)C)cc4ccc(N5c6ccccc6N(c6ccccc6)c6cc7c(cc65)sc5cc6ccccc6cc57)c1c4c23 ; CC1(C)c2ccccc2N(c2ccc3c4ccc(N5c6ccccc6C(C)(C)c6cc7c(cc65)sc5cc6c8ccccc8c8ccccc8c6cc57)cc4c4ccccc4c3c2)c2cc3sc4cc5c6ccccc6c6ccccc6c5cc4c3cc21]

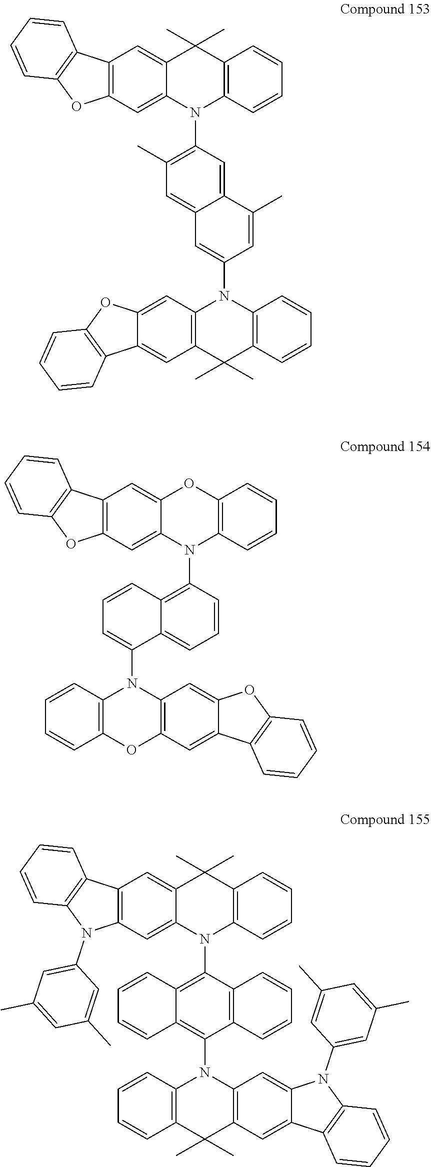

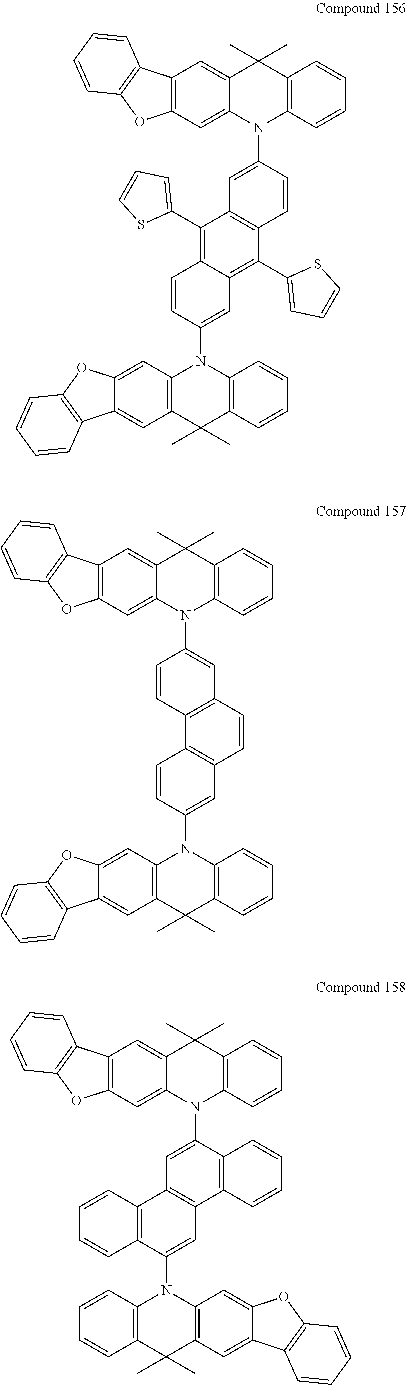 [SMILES: CC1(C)c2ccccc2N(c2ccc3c(-c4cccs4)c4cc(N5c6ccccc6C(C)(C)c6cc7c(cc65)oc5ccccc57)ccc4c(-c4cccs4)c3c2)c2cc3oc4ccccc4c3cc21 ; CC1(C)c2ccccc2N(c2ccc3c(ccc4cc(N5c6ccccc6C(C)(C)c6cc7c(cc65)oc5ccccc57)ccc43)c2)c2cc3oc4ccccc4c3cc21 ; CC1(C)c2ccccc2N(c2cc3c4ccccc4c(N4c5ccccc5C(C)(C)c5cc6c(cc54)oc4ccccc46)cc3c3ccccc23)c2cc3oc4ccccc4c3cc21]

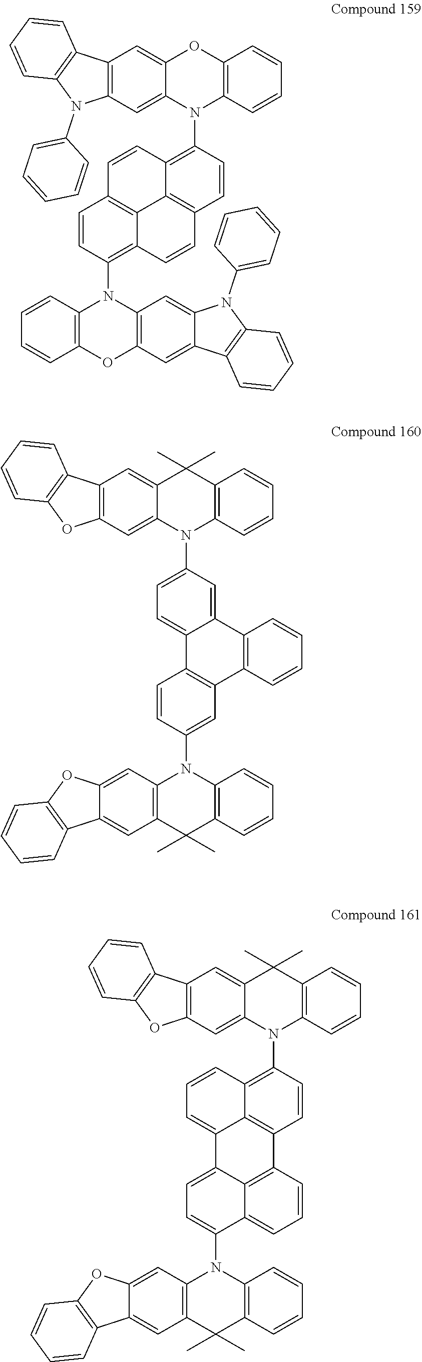 [SMILES: c1ccc(-n2c3ccccc3c3cc4c(cc32)N(c2ccc3ccc5c(N6c7ccccc7Oc7cc8c9ccccc9n(-c9ccccc9)c8cc76)ccc6ccc2c3c65)c2ccccc2O4)cc1 ; CC1(C)c2ccccc2N(c2ccc3c4ccc(N5c6ccccc6C(C)(C)c6cc7c(cc65)oc5ccccc57)cc4c4ccccc4c3c2)c2cc3oc4ccccc4c3cc21 ; CC1(C)c2ccccc2N(c2ccc3c4cccc5c(N6c7ccccc7C(C)(C)c7cc8c(cc76)oc6ccccc68)ccc(c6cccc2c63)c54)c2cc3oc4ccccc4c3cc21]

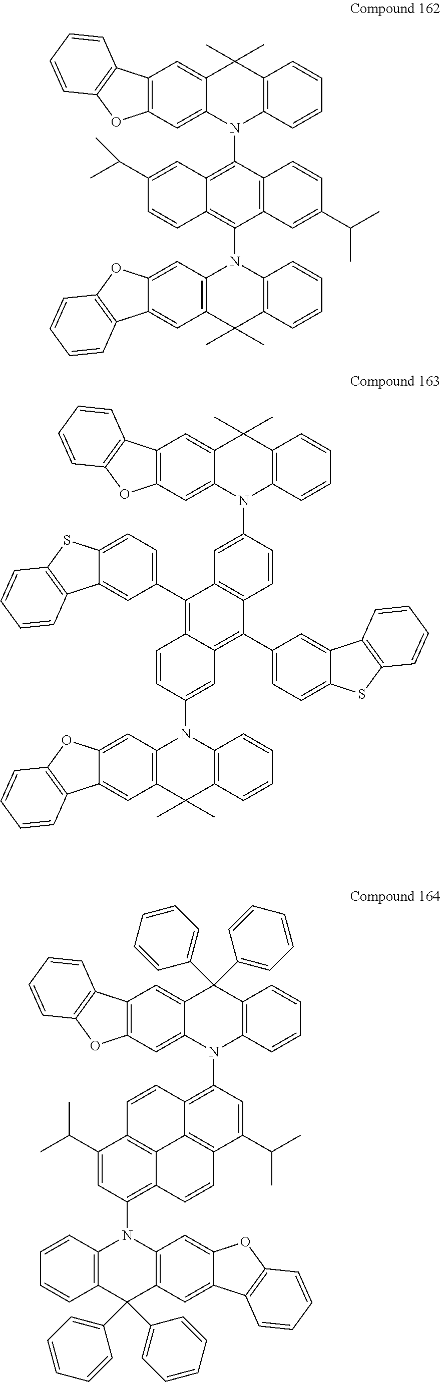 [SMILES: CC(C)c1ccc2c(N3c4ccccc4C(C)(C)c4cc5c(cc43)oc3ccccc35)c3cc(C(C)C)ccc3c(N3c4ccccc4C(C)(C)c4cc5c(cc43)oc3ccccc35)c2c1 ; CC1(C)c2ccccc2N(c2ccc3c(-c4ccc5sc6ccccc6c5c4)c4cc(N5c6ccccc6C(C)(C)c6cc7c(cc65)oc5ccccc57)ccc4c(-c4ccc5sc6ccccc6c5c4)c3c2)c2cc3oc4ccccc4c3cc21 ; CC(C)c1cc(N2c3ccccc3C(c3ccccc3)(c3ccccc3)c3cc4c(cc32)oc2ccccc24)c2ccc3c(C(C)C)cc(N4c5ccccc5C(c5ccccc5)(c5ccccc5)c5cc6c(cc54)oc4ccccc46)c4ccc1c2c34]

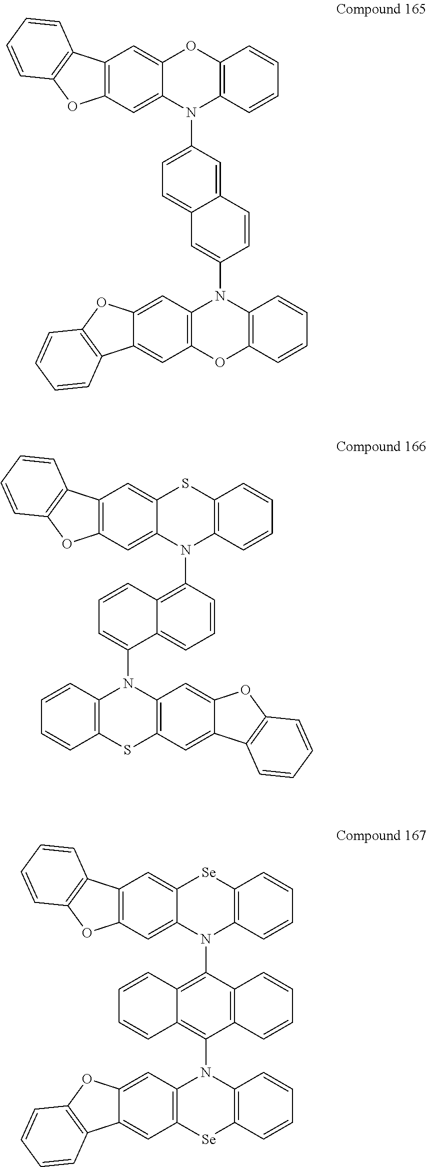 [SMILES: c1ccc2c(c1)Oc1cc3c(cc1N2c1ccc2cc(N4c5ccccc5Oc5cc6c(cc54)oc4ccccc46)ccc2c1)oc1ccccc13 ; c1ccc2c(c1)Sc1cc3c(cc1N2c1cccc2c(N4c5ccccc5Sc5cc6c(cc54)oc4ccccc46)cccc12)oc1ccccc13 ; c1ccc2c(c1)[Se]c1cc3c(cc1N2c1c2ccccc2c(N2c4ccccc4[Se]c4cc5c(cc42)oc2ccccc25)c2ccccc12)oc1ccccc13]

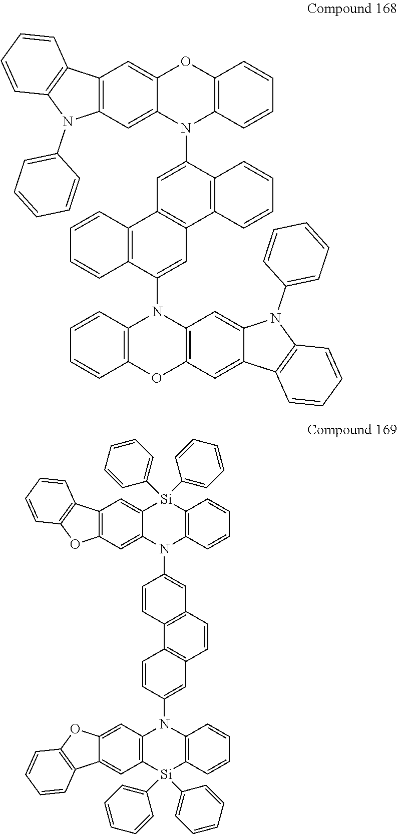 [SMILES: c1ccc(-n2c3ccccc3c3cc4c(cc32)N(c2cc3c5ccccc5c(N5c6ccccc6Oc6cc7c8ccccc8n(-c8ccccc8)c7cc65)cc3c3ccccc23)c2ccccc2O4)cc1 ; c1ccc([Si]2(c3ccccc3)c3ccccc3N(c3ccc4c(ccc5cc(N6c7ccccc7[Si](c7ccccc7)(c7ccccc7)c7cc8c(cc76)oc6ccccc68)ccc54)c3)c3cc4oc5ccccc5c4cc32)cc1]

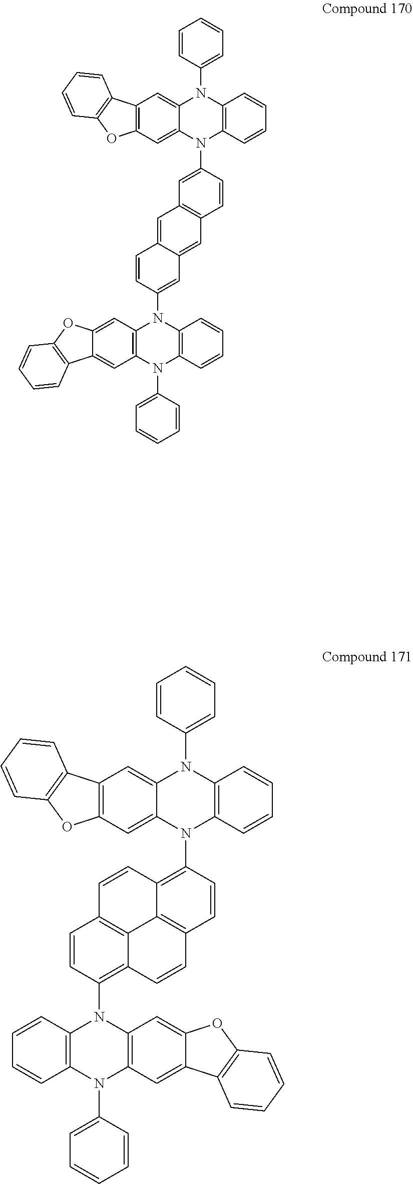 [SMILES: c1ccc(N2c3ccccc3N(c3ccc4cc5cc(N6c7ccccc7N(c7ccccc7)c7cc8c(cc76)oc6ccccc68)ccc5cc4c3)c3cc4oc5ccccc5c4cc32)cc1 ; c1ccc(N2c3ccccc3N(c3ccc4ccc5c(N6c7ccccc7N(c7ccccc7)c7cc8c(cc76)oc6ccccc68)ccc6ccc3c4c65)c3cc4oc5ccccc5c4cc32)cc1]

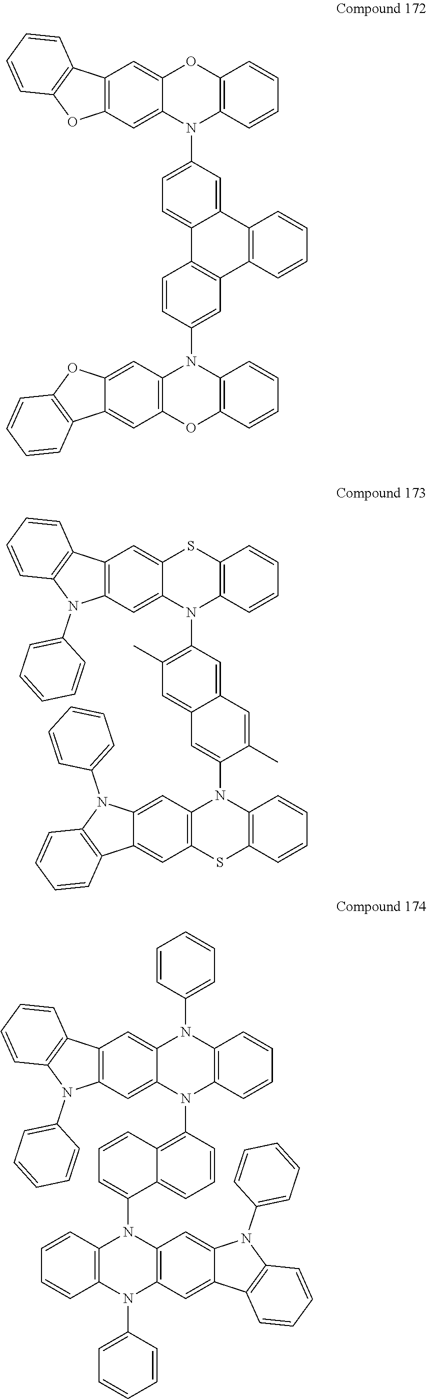 [SMILES: c1ccc2c(c1)Oc1cc3c(cc1N2c1ccc2c4ccc(N5c6ccccc6Oc6cc7c(cc65)oc5ccccc57)cc4c4ccccc4c2c1)oc1ccccc13 ; Cc1cc2cc(N3c4ccccc4Sc4cc5c6ccccc6n(-c6ccccc6)c5cc43)c(C)cc2cc1N1c2ccccc2Sc2cc3c4ccccc4n(-c4ccccc4)c3cc21 ; c1ccc(N2c3ccccc3N(c3cccc4c(N5c6ccccc6N(c6ccccc6)c6cc7c8ccccc8n(-c8ccccc8)c7cc65)cccc34)c3cc4c(cc32)c2ccccc2n4-c2ccccc2)cc1]

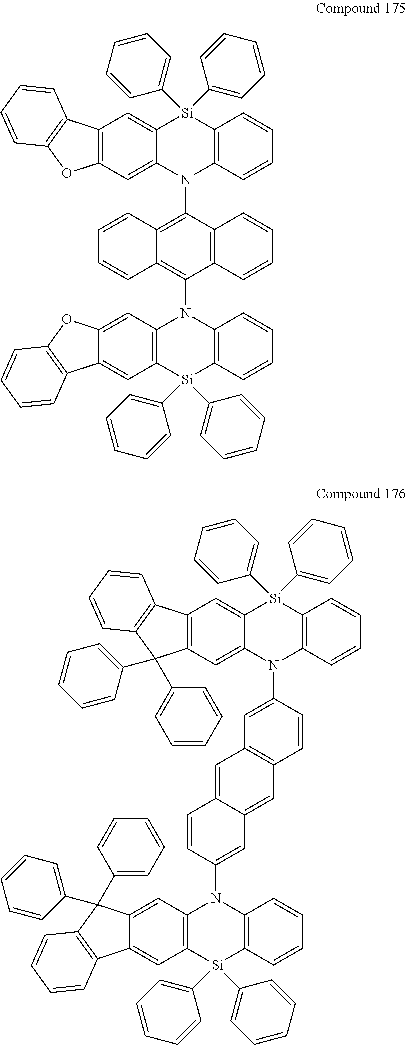 [SMILES: c1ccc([Si]2(c3ccccc3)c3ccccc3N(c3c4ccccc4c(N4c5ccccc5[Si](c5ccccc5)(c5ccccc5)c5cc6c(cc54)oc4ccccc46)c4ccccc34)c3cc4oc5ccccc5c4cc32)cc1 ; c1ccc(C2(c3ccccc3)c3ccccc3-c3cc4c(cc32)N(c2ccc3cc5cc(N6c7ccccc7[Si](c7ccccc7)(c7ccccc7)c7cc8c(cc76)C(c6ccccc6)(c6ccccc6)c6ccccc6-8)ccc5cc3c2)c2ccccc2[Si]4(c2ccccc2)c2ccccc2)cc1]

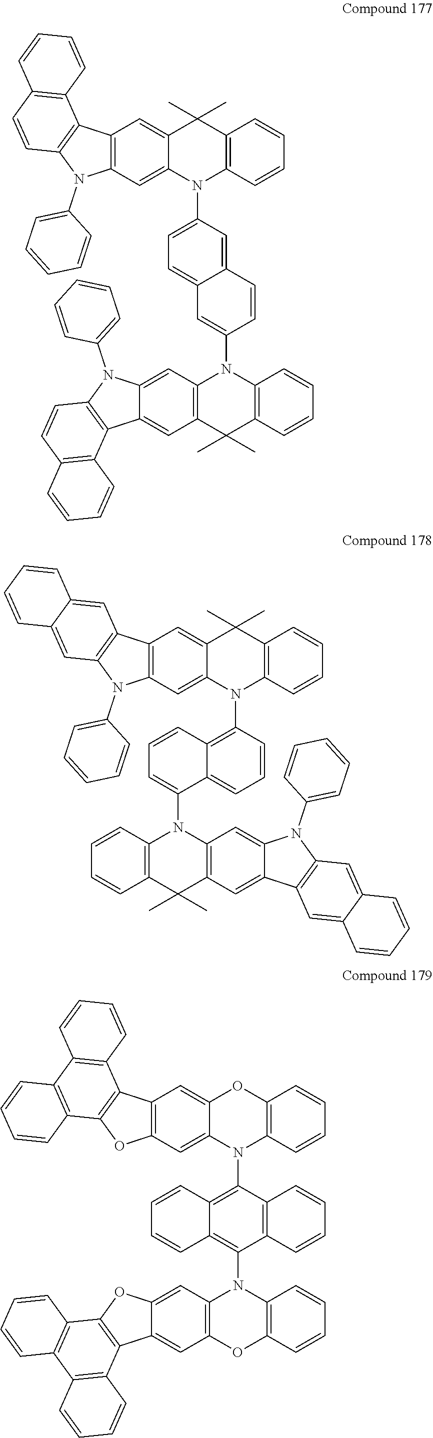 [SMILES: CC1(C)c2ccccc2N(c2ccc3cc(N4c5ccccc5C(C)(C)c5cc6c7c8ccccc8ccc7n(-c7ccccc7)c6cc54)ccc3c2)c2cc3c(cc21)c1c2ccccc2ccc1n3-c1ccccc1 ; CC1(C)c2ccccc2N(c2cccc3c(N4c5ccccc5C(C)(C)c5cc6c7cc8ccccc8cc7n(-c7ccccc7)c6cc54)cccc23)c2cc3c(cc21)c1cc2ccccc2cc1n3-c1ccccc1 ; c1ccc2c(c1)Oc1cc3c(cc1N2c1c2ccccc2c(N2c4ccccc4Oc4cc5c(cc42)oc2c4ccccc4c4ccccc4c52)c2ccccc12)oc1c2ccccc2c2ccccc2c31]

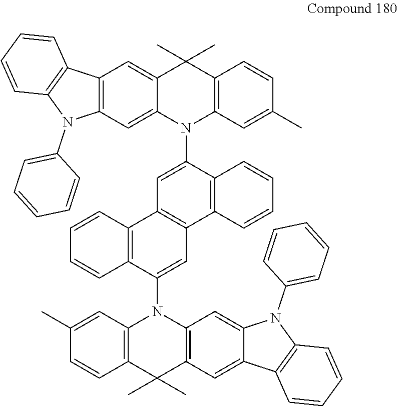 [SMILES: Cc1ccc2c(c1)N(c1cc3c4ccccc4c(N4c5cc(C)ccc5C(C)(C)c5cc6c7ccccc7n(-c7ccccc7)c6cc54)cc3c3ccccc13)c1cc3c(cc1C2(C)C)c1ccccc1n3-c1ccccc1]

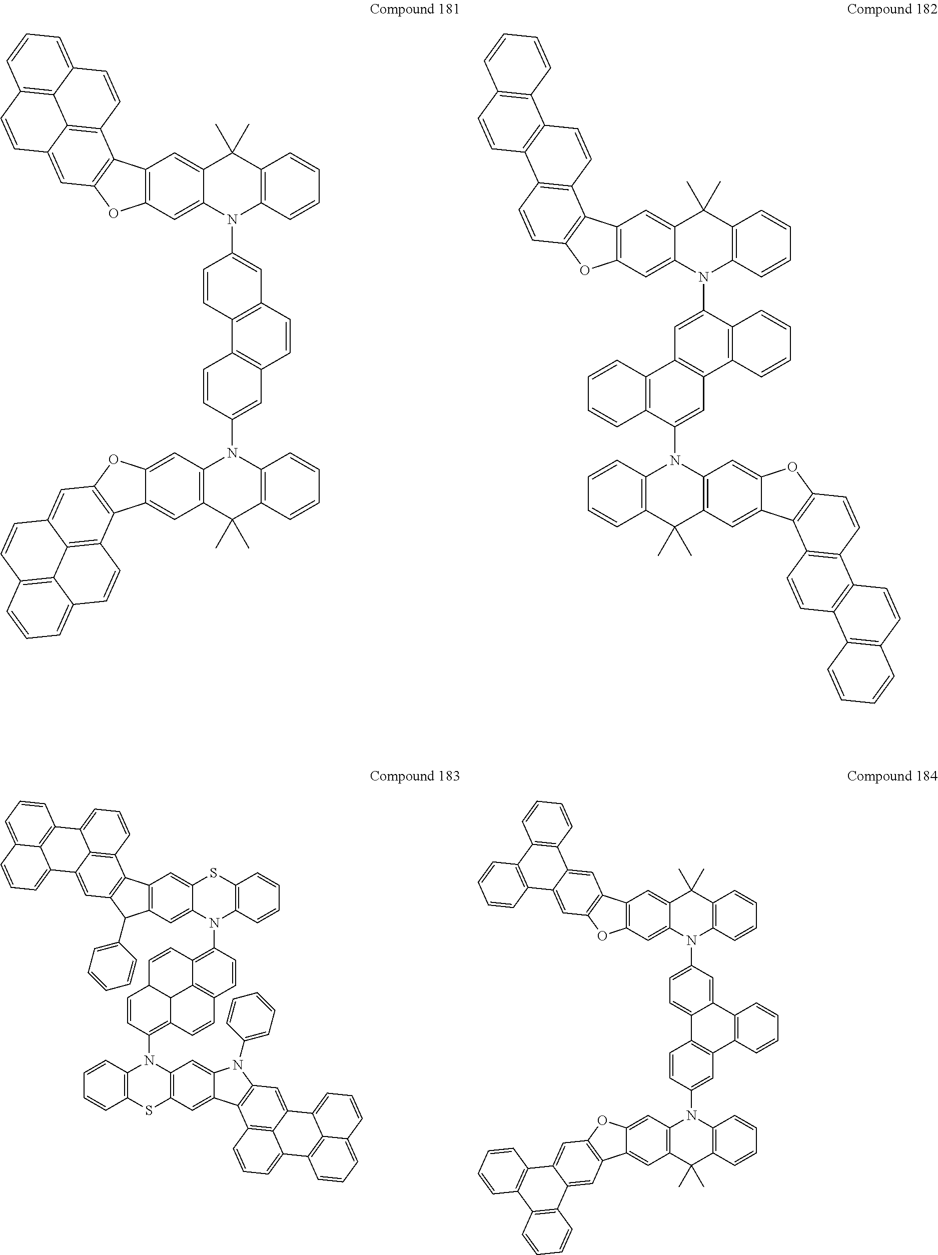

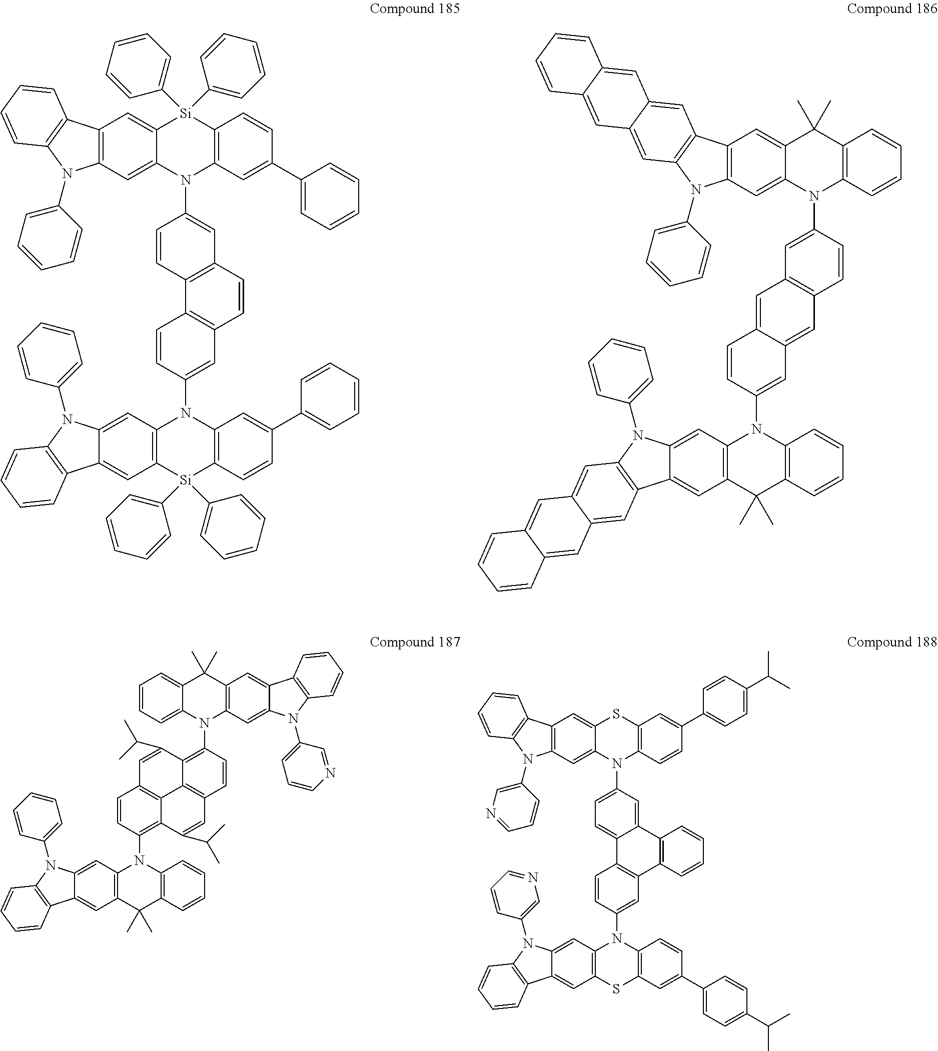

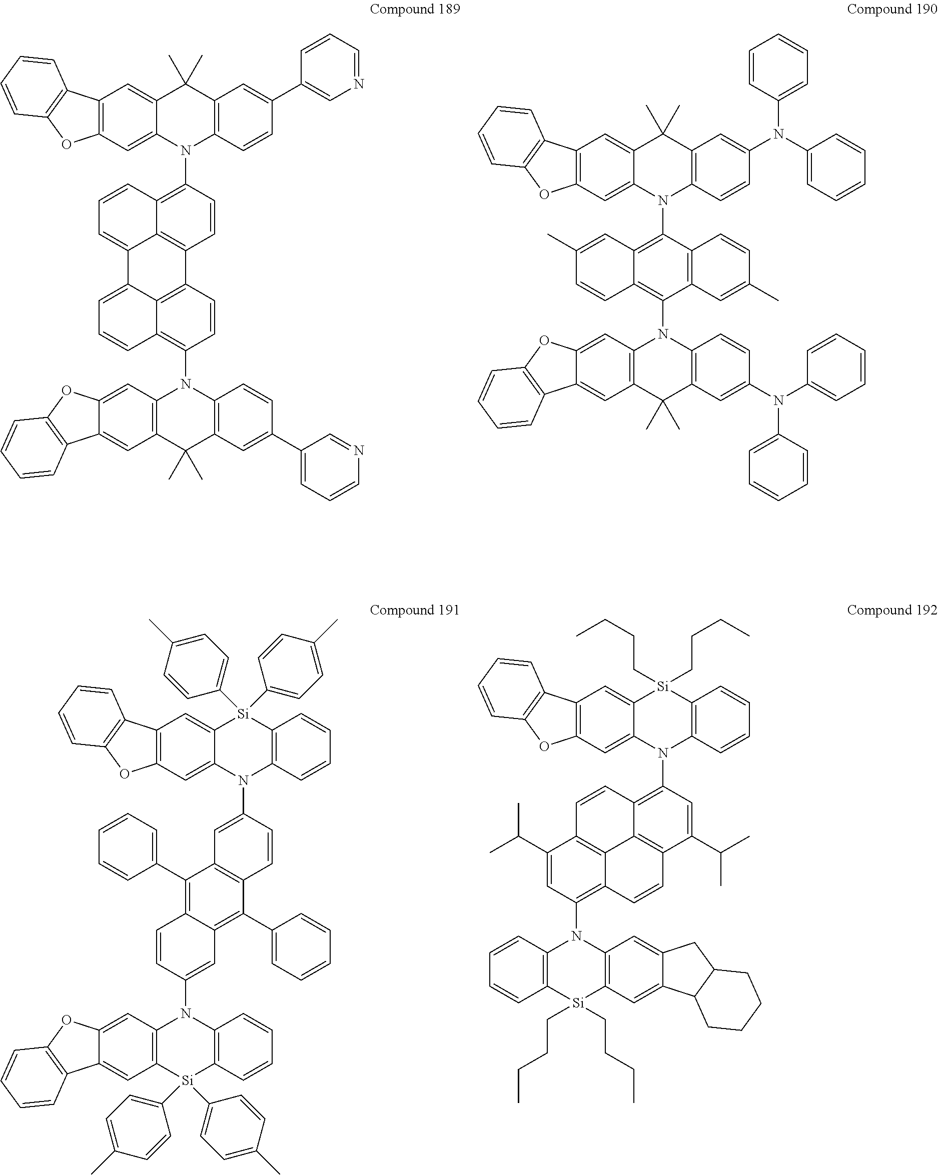

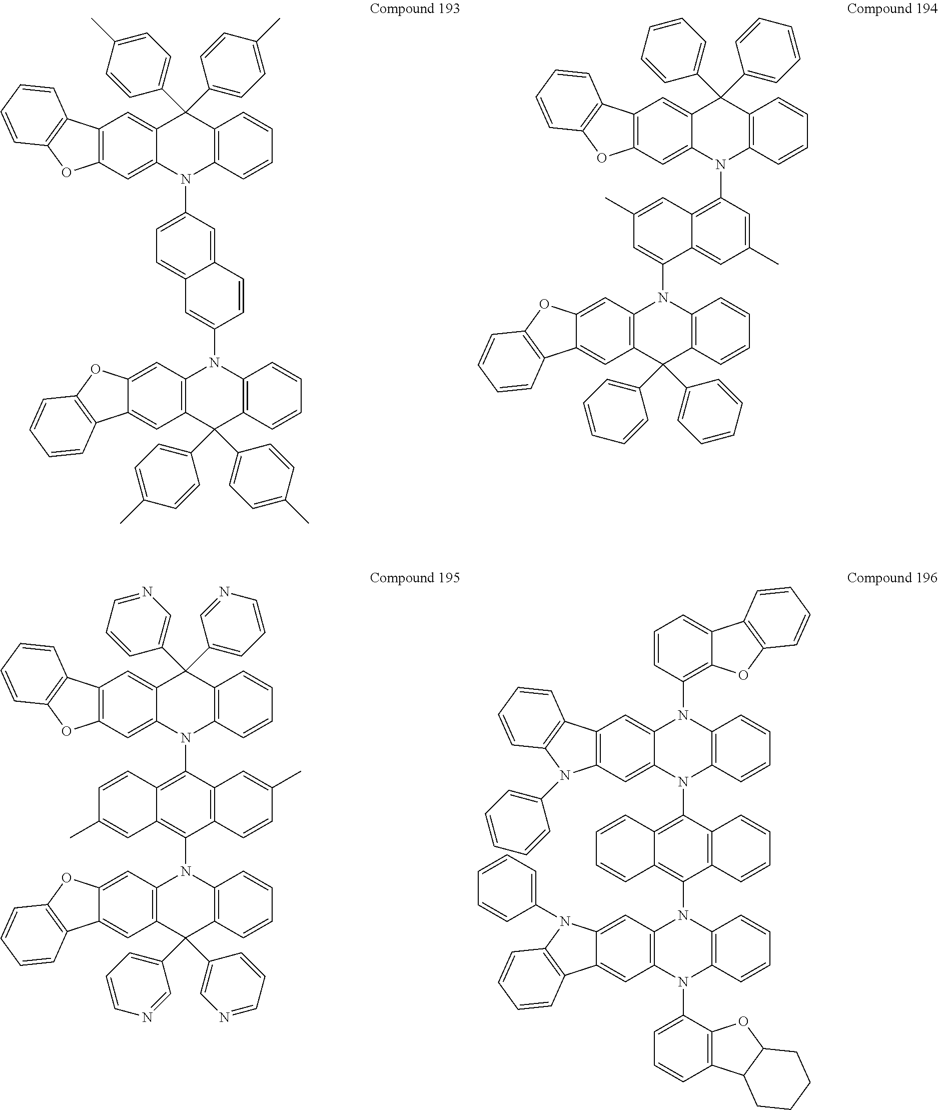

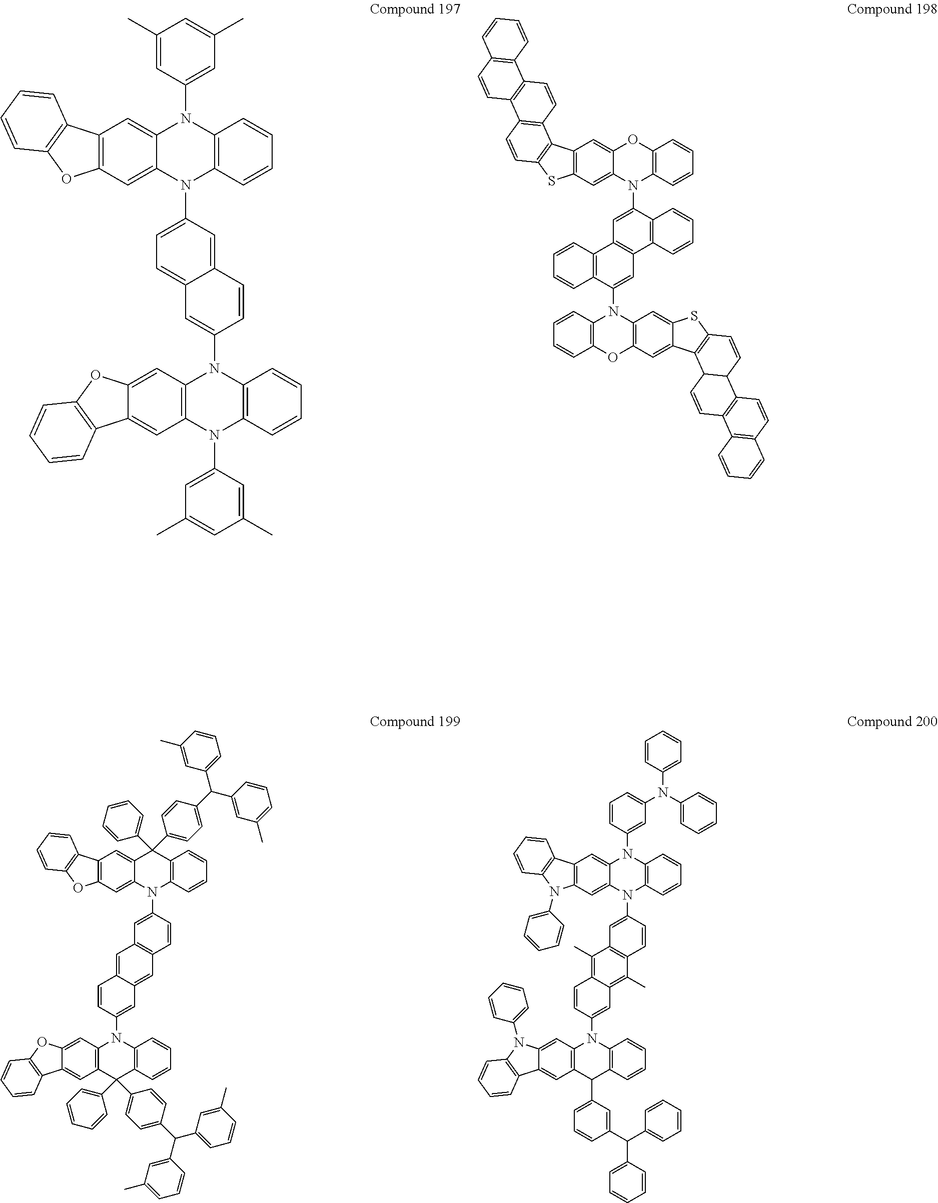

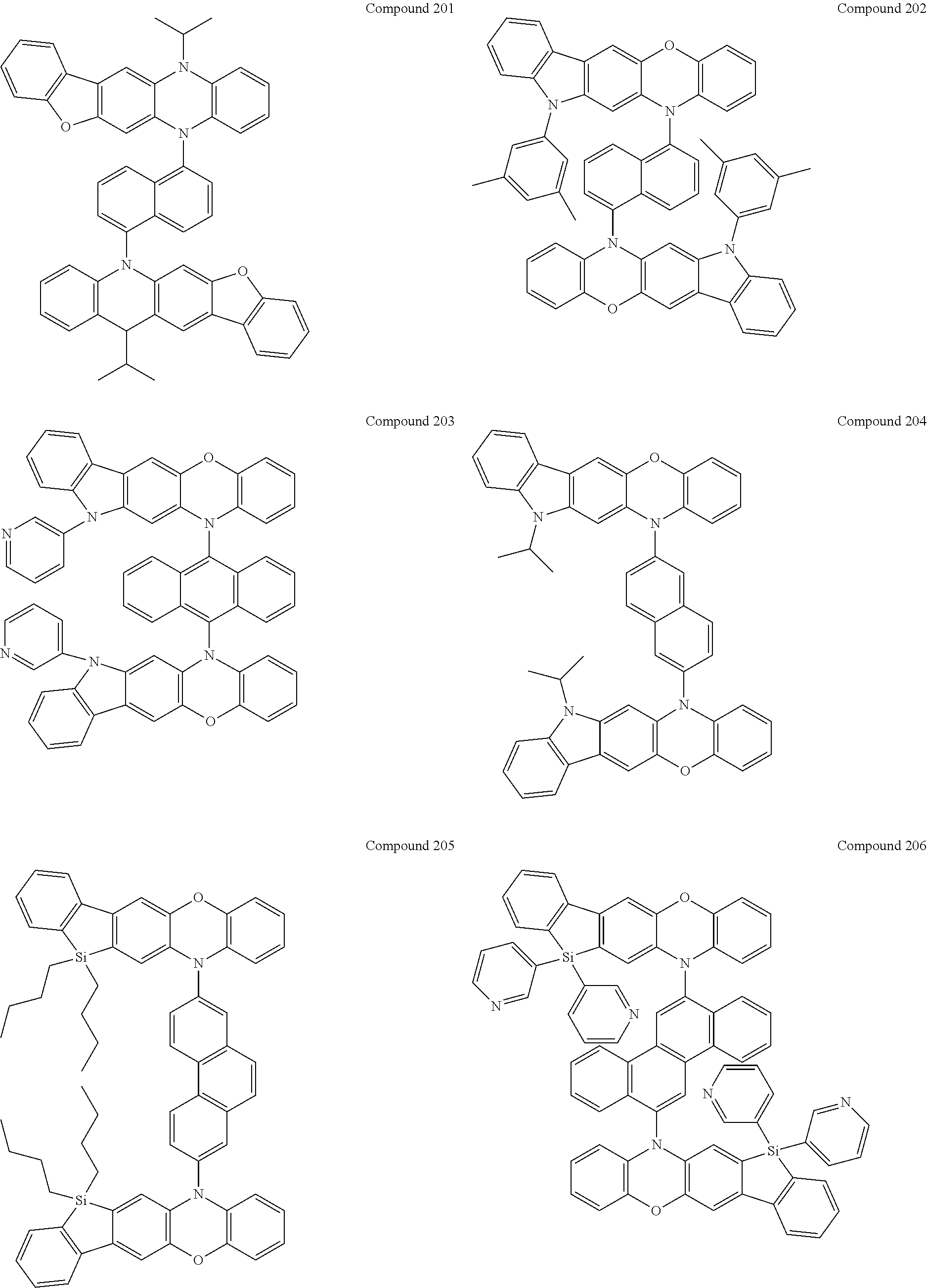

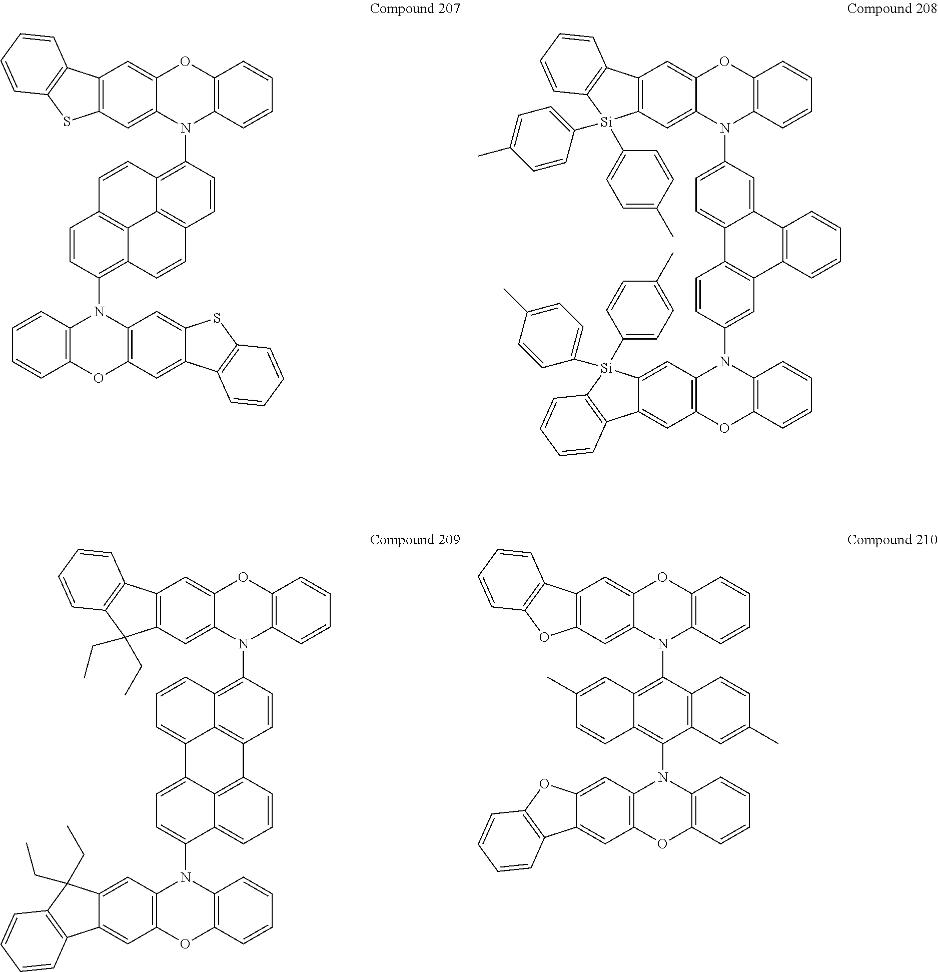 [SMILES: c1ccc2c(c1)Oc1cc3c(cc1N2c1ccc2ccc4c(N5c6ccccc6Oc6cc7c(cc65)sc5ccccc57)ccc5ccc1c2c54)sc1ccccc13 ; Cc1ccc([Si]2(c3ccc(C)cc3)c3ccccc3-c3cc4c(cc32)N(c2ccc3c5ccc(N6c7ccccc7Oc7cc8c(cc76)[Si](c6ccc(C)cc6)(c6ccc(C)cc6)c6ccccc6-8)cc5c5ccccc5c3c2)c2ccccc2O4)cc1 ; CCC1(CC)c2ccccc2-c2cc3c(cc21)N(c1ccc2c4cccc5c(N6c7ccccc7Oc7cc8c(cc76)C(CC)(CC)c6ccccc6-8)ccc(c6cccc1c62)c54)c1ccccc1O3 ; Cc1ccc2c(N3c4ccccc4Oc4cc5c(cc43)oc3ccccc35)c3cc(C)ccc3c(N3c4ccccc4Oc4cc5c(cc43)oc3ccccc35)c2c1]

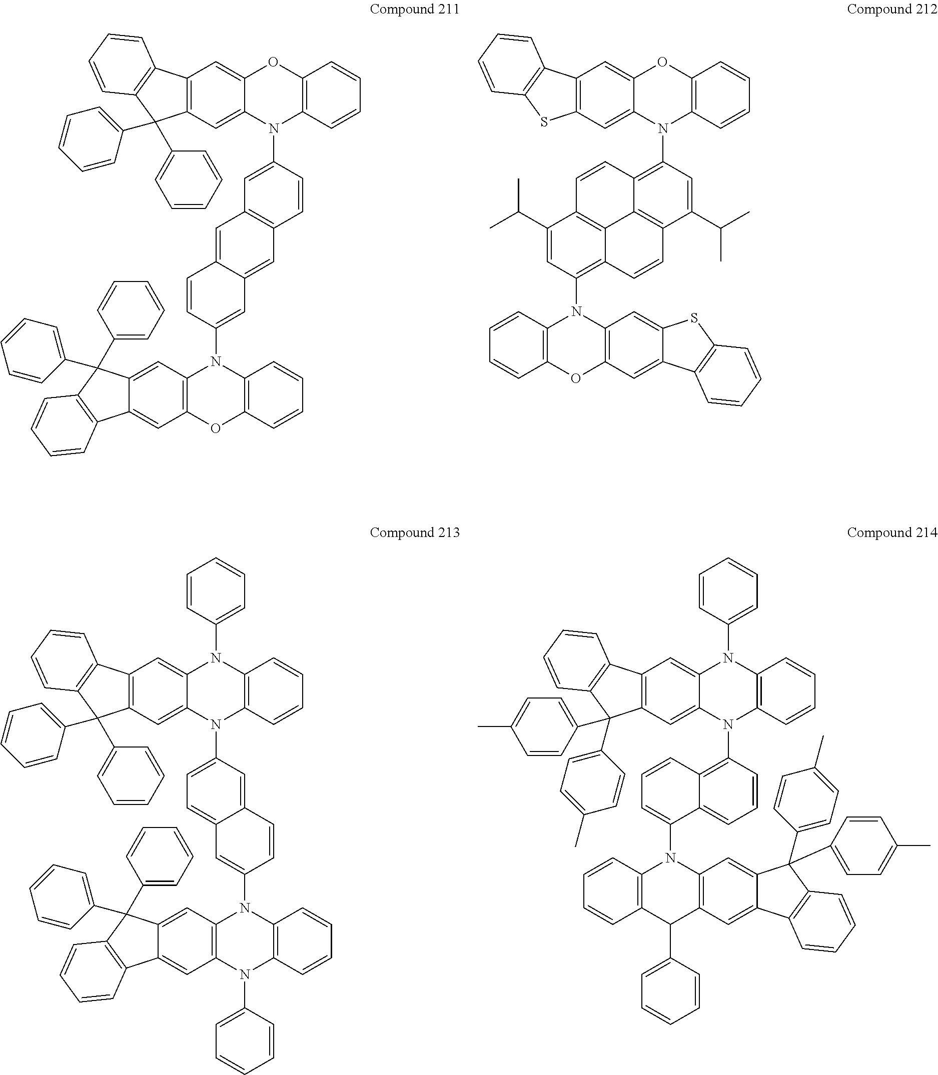

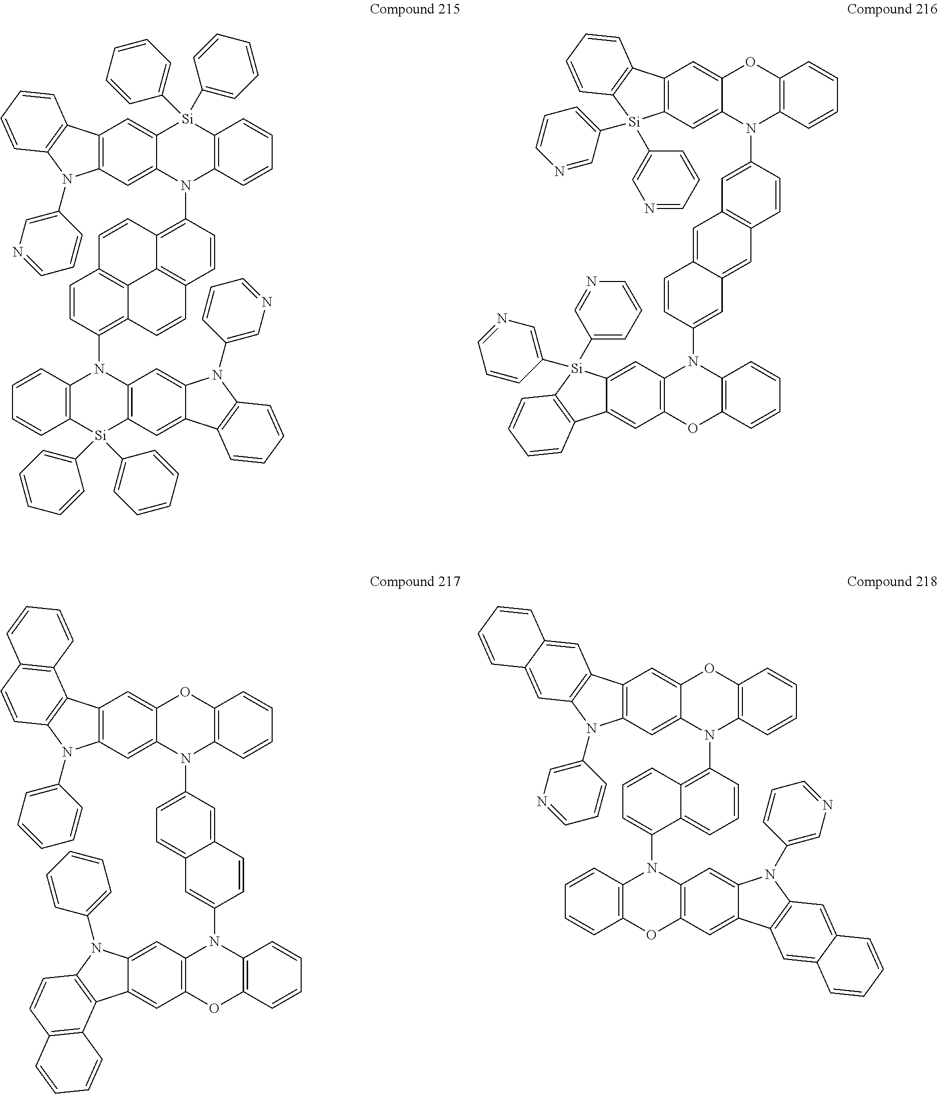

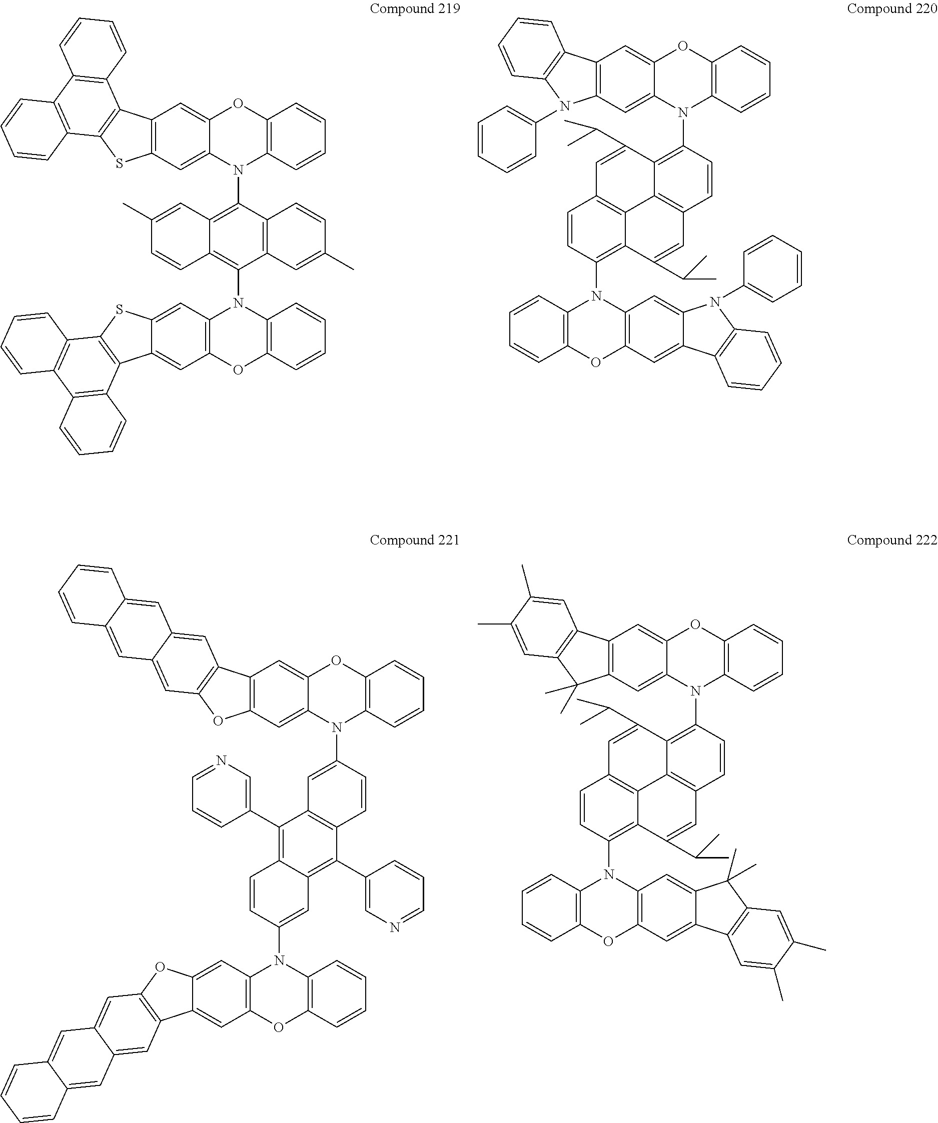

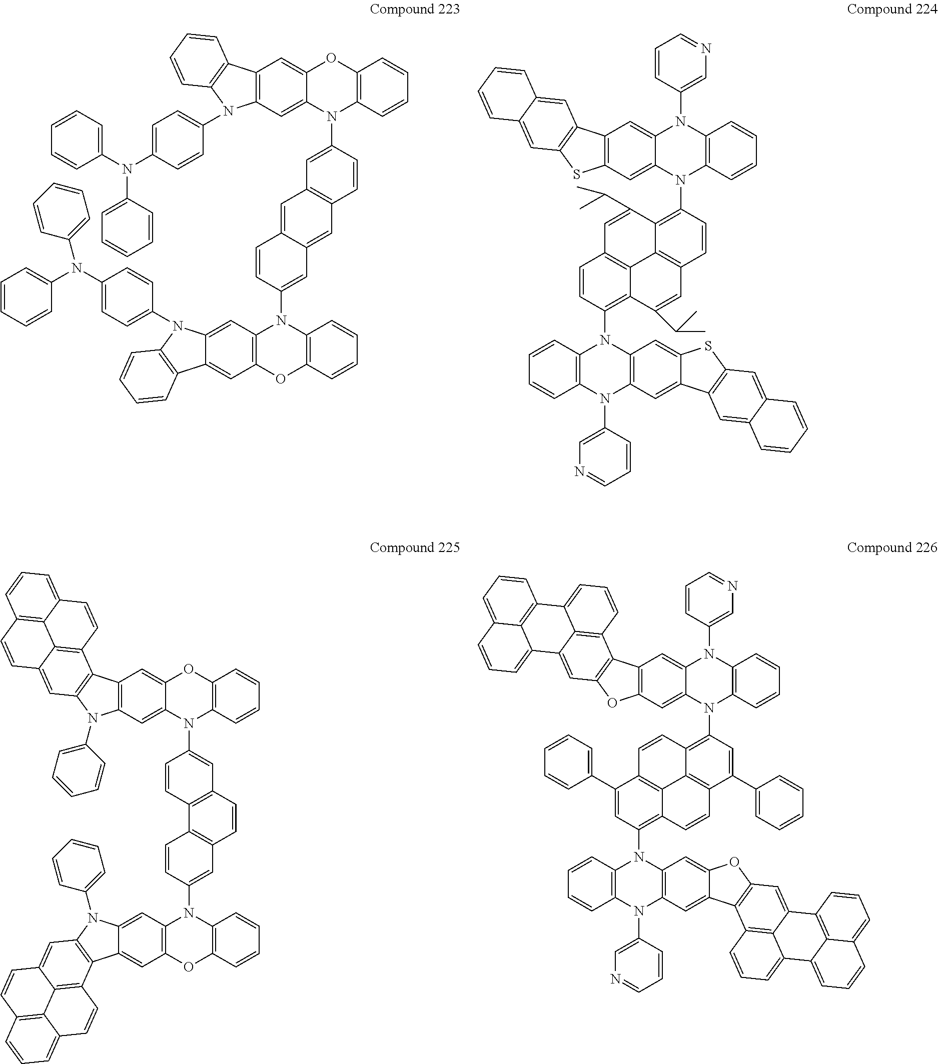

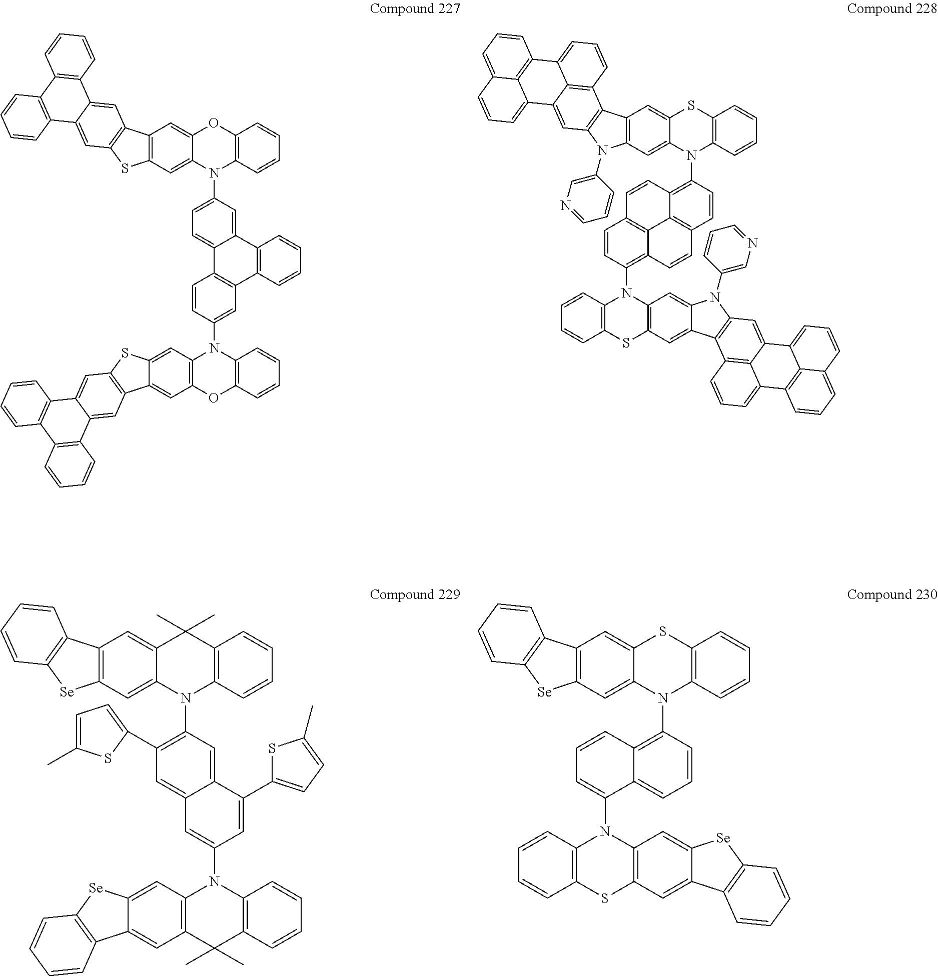

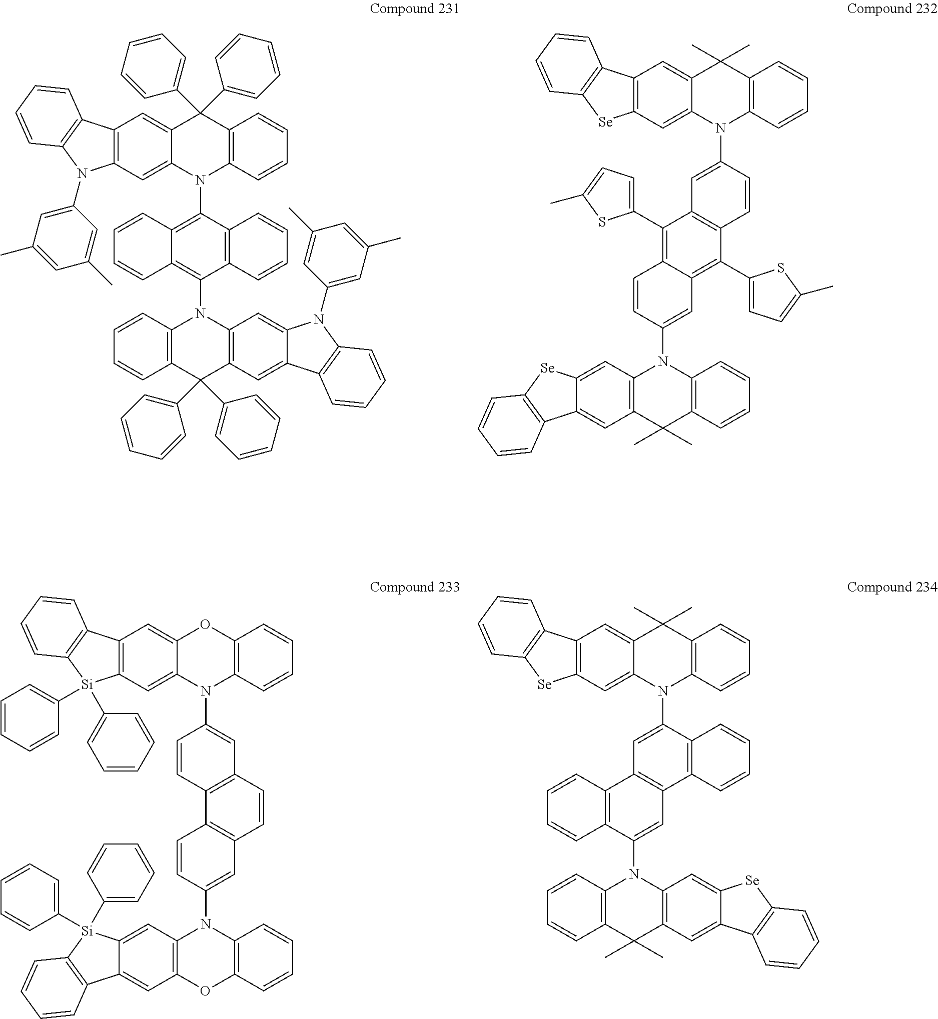

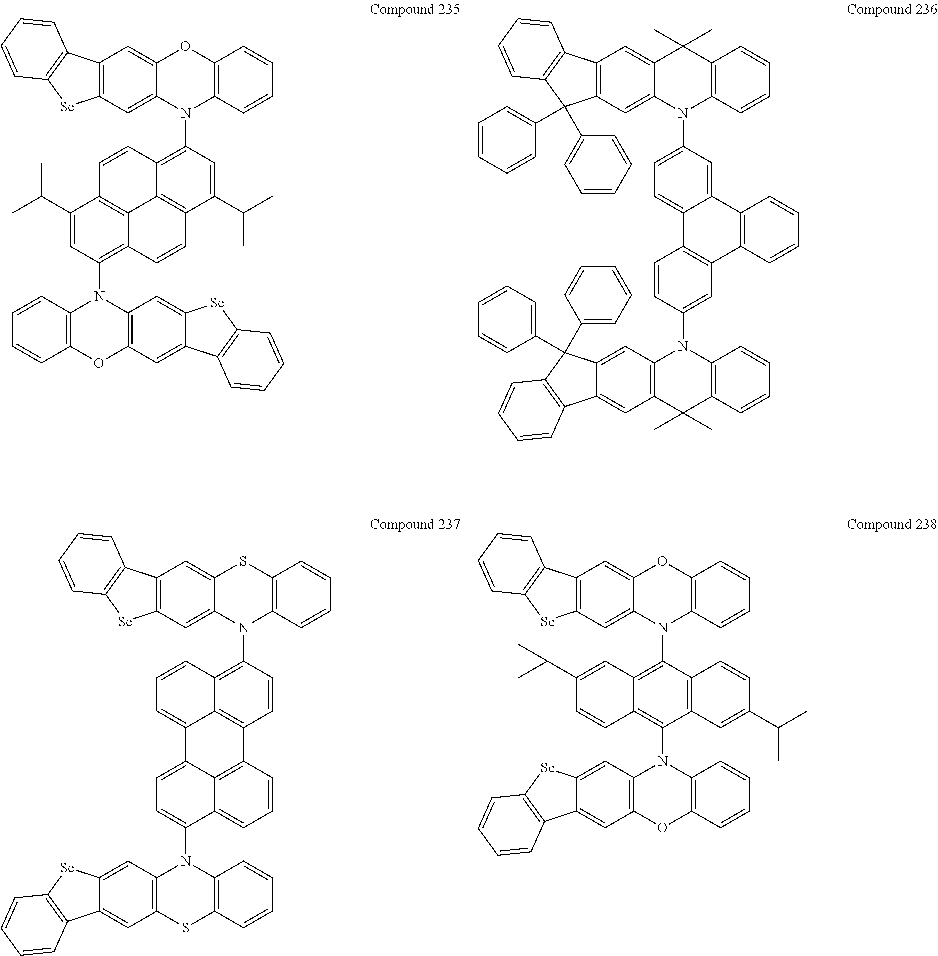 [SMILES: CC(C)c1cc(N2c3ccccc3Oc3cc4c(cc32)[se]c2ccccc24)c2ccc3c(C(C)C)cc(N4c5ccccc5Oc5cc6c(cc54)[se]c4ccccc46)c4ccc1c2c34 ; CC1(C)c2ccccc2N(c2ccc3c4ccc(N5c6ccccc6C(C)(C)c6cc7c(cc65)C(c5ccccc5)(c5ccccc5)c5ccccc5-7)cc4c4ccccc4c3c2)c2cc3c(cc21)-c1ccccc1C3(c1ccccc1)c1ccccc1 ; c1ccc2c(c1)Sc1cc3c(cc1N2c1ccc2c4cccc5c(N6c7ccccc7Sc7cc8c(cc76)[se]c6ccccc68)ccc(c6cccc1c62)c54)[se]c1ccccc13 ; CC(C)c1ccc2c(N3c4ccccc4Oc4cc5c(cc43)[se]c3ccccc35)c3cc(C(C)C)ccc3c(N3c4ccccc4Oc4cc5c(cc43)[se]c3ccccc35)c2c1]

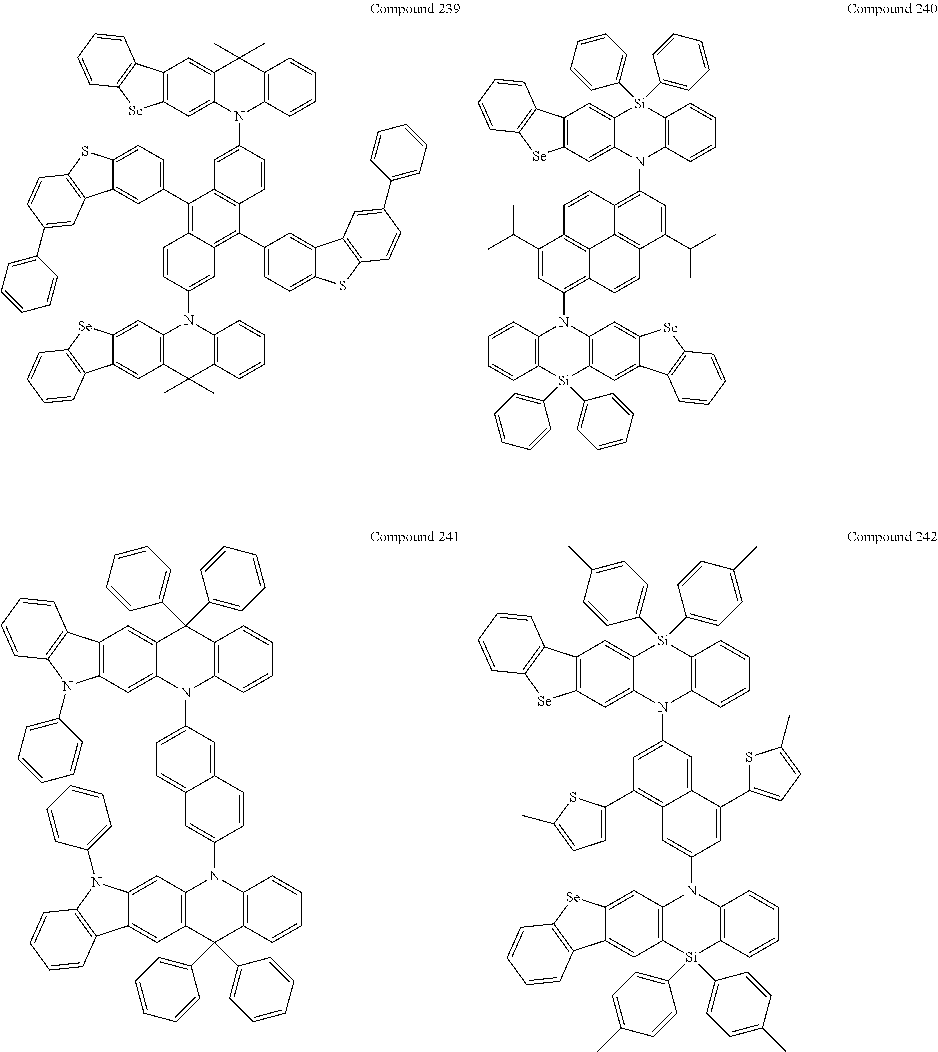

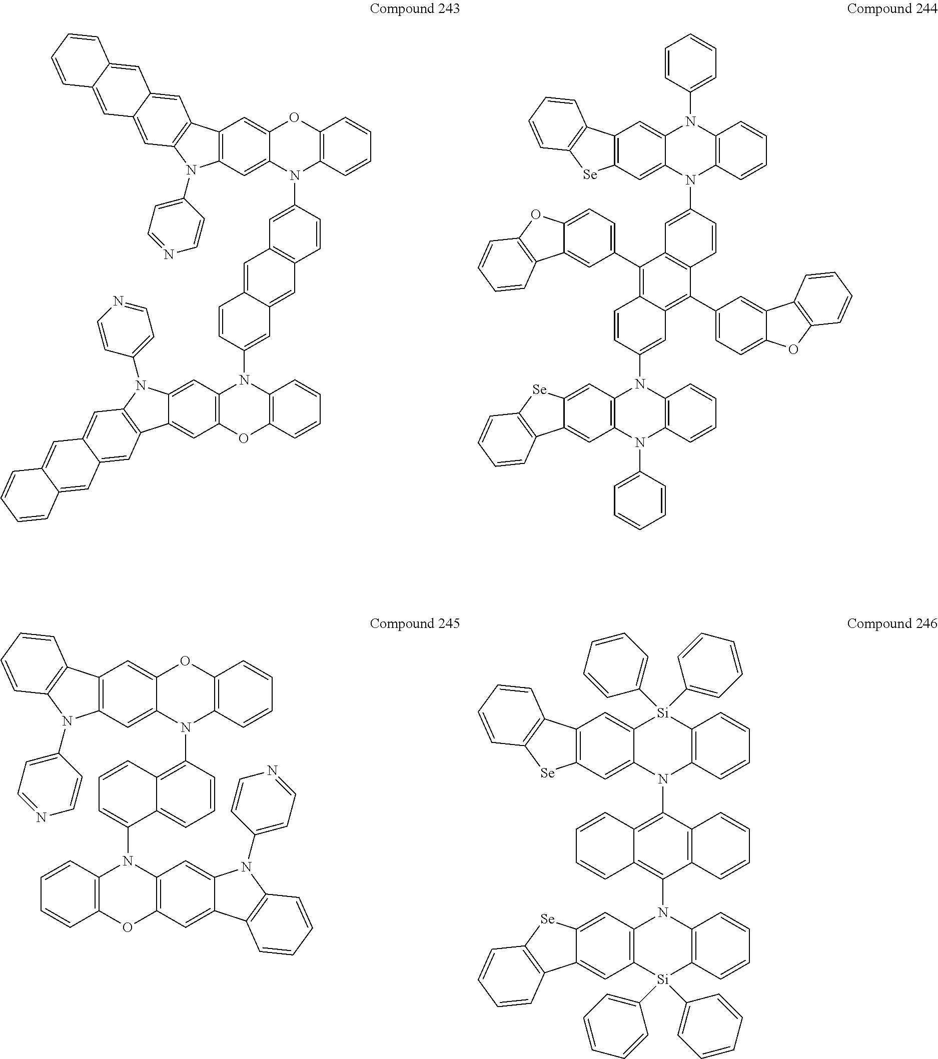

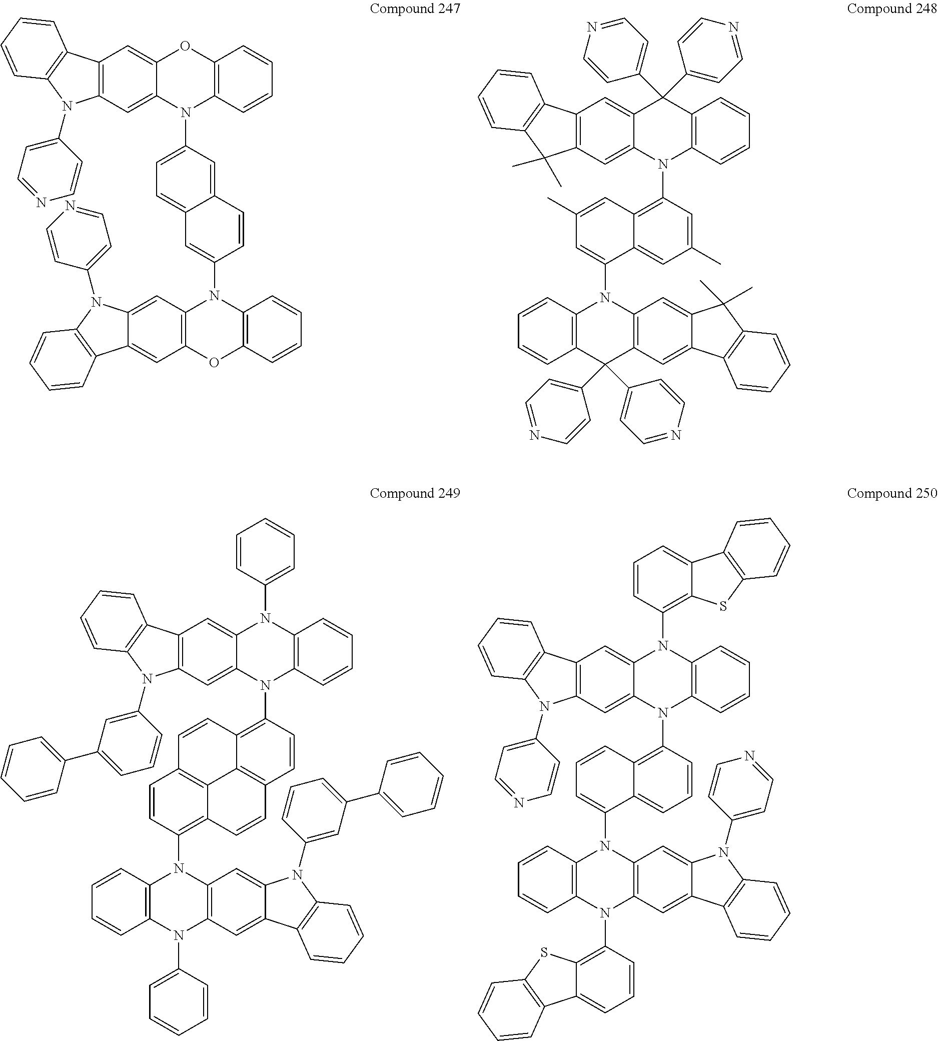

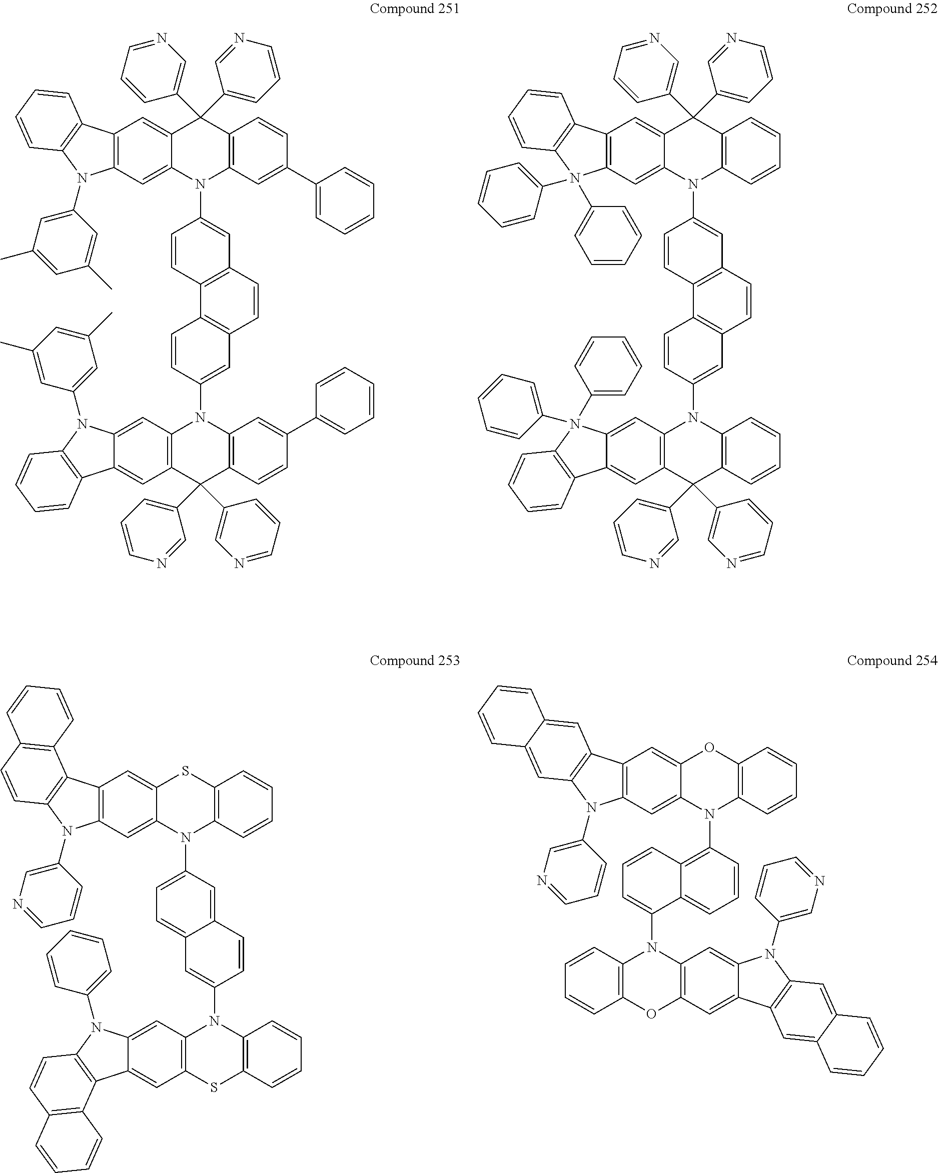

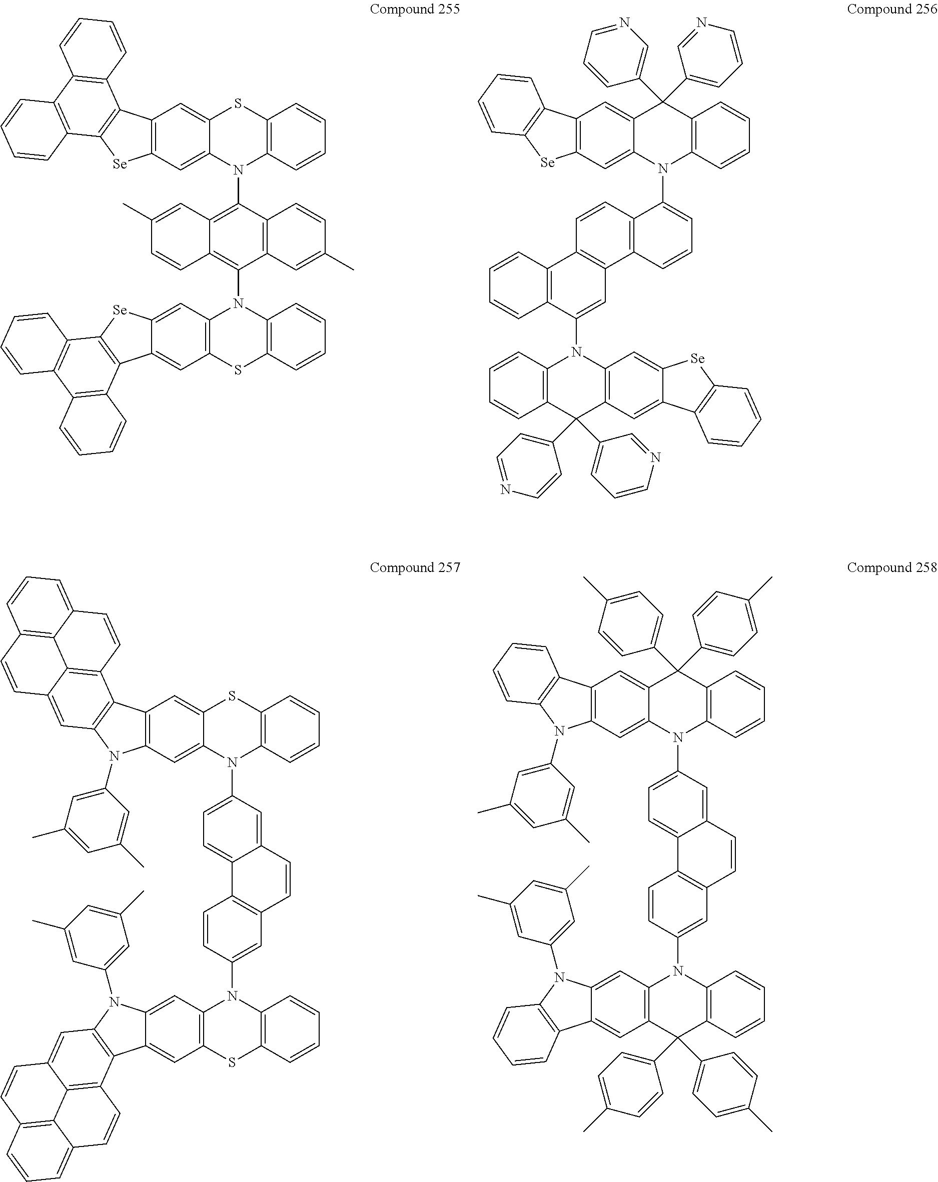

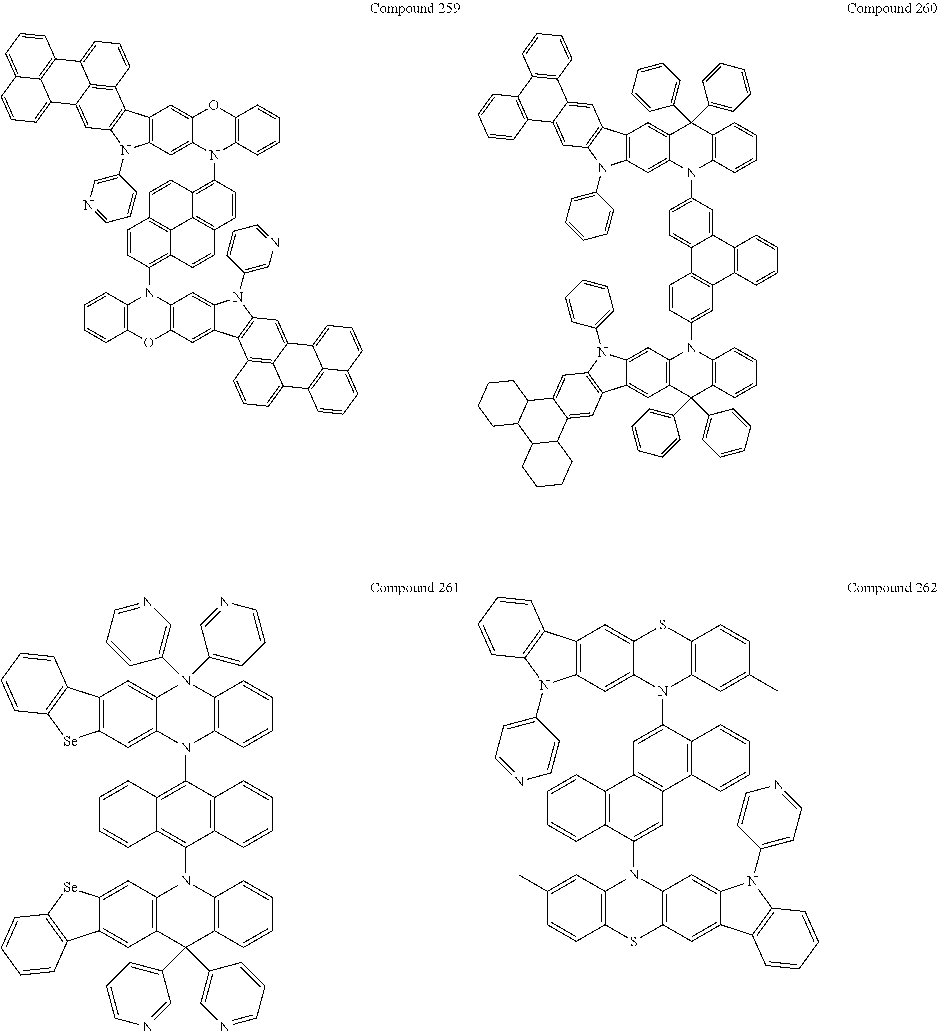

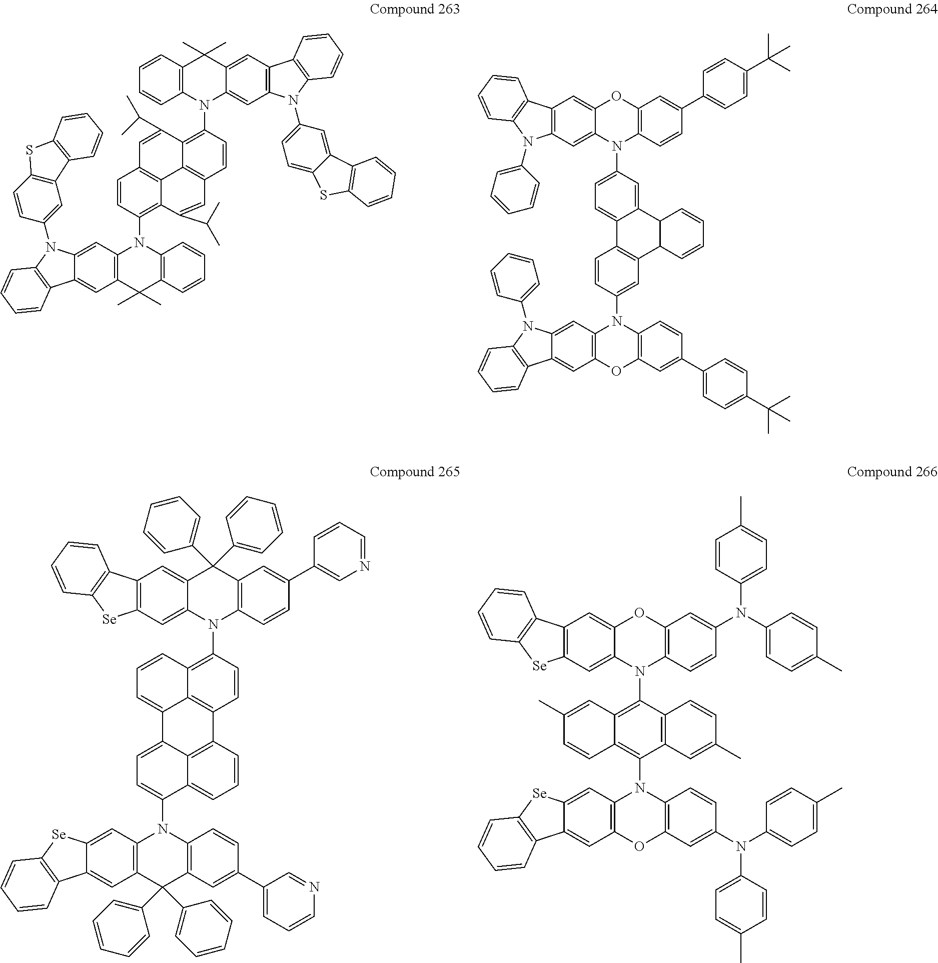

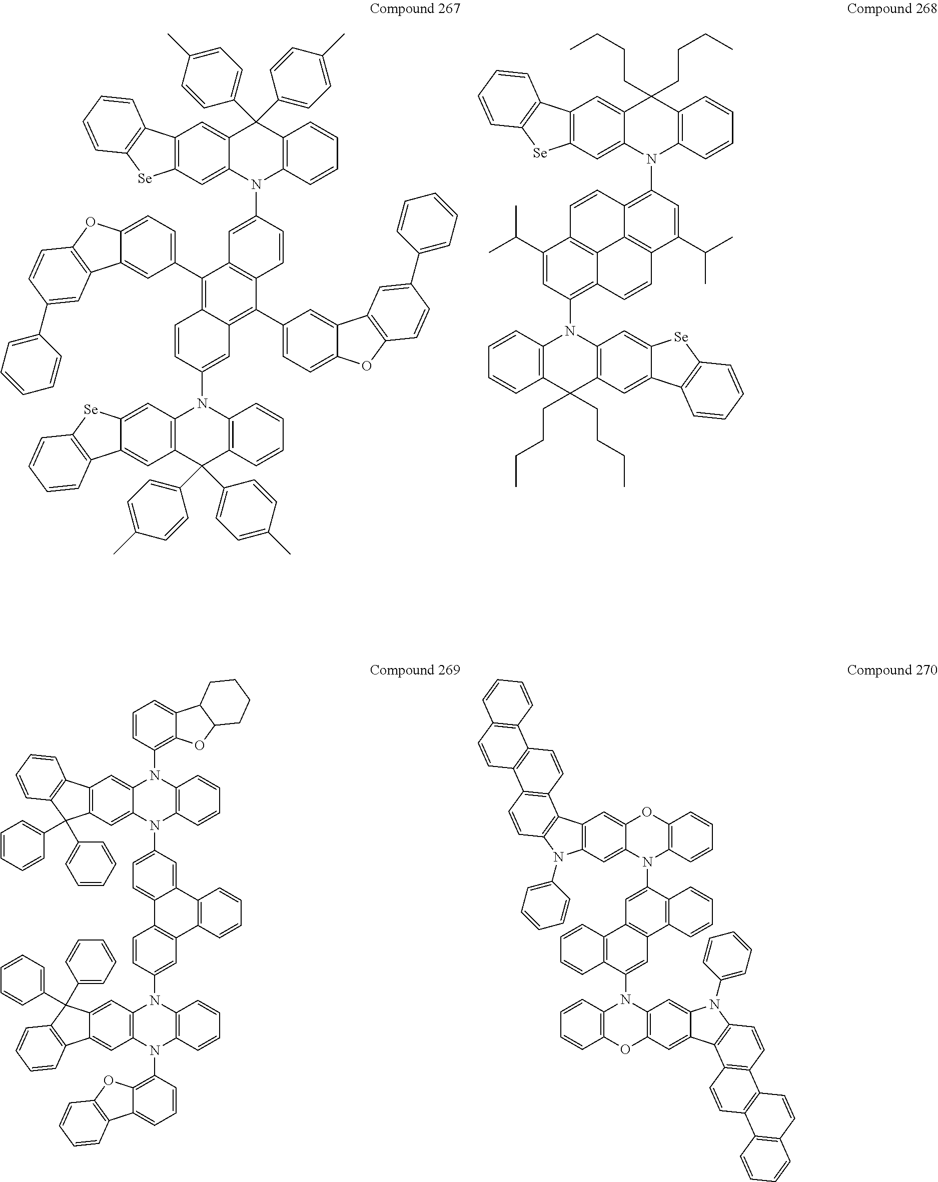

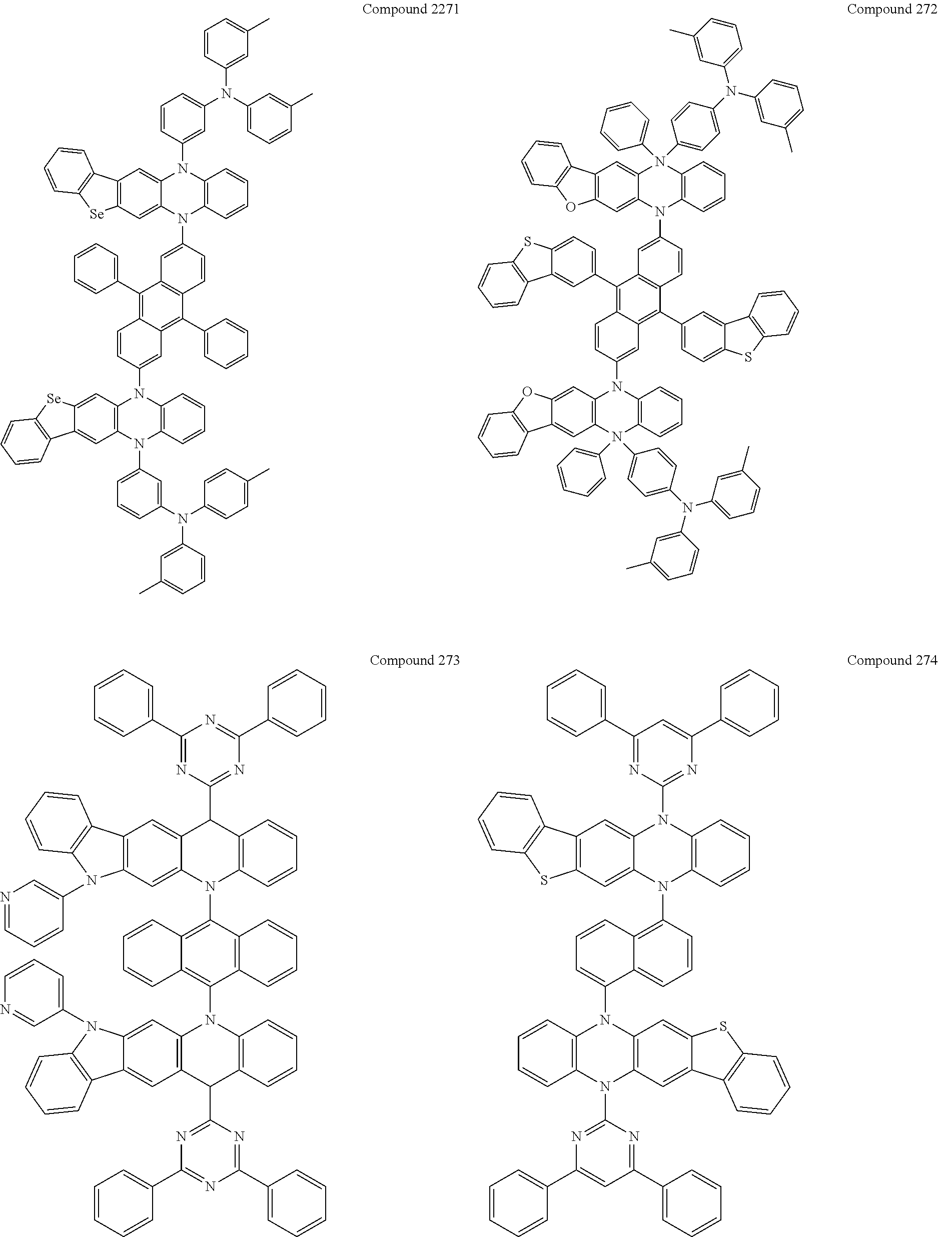

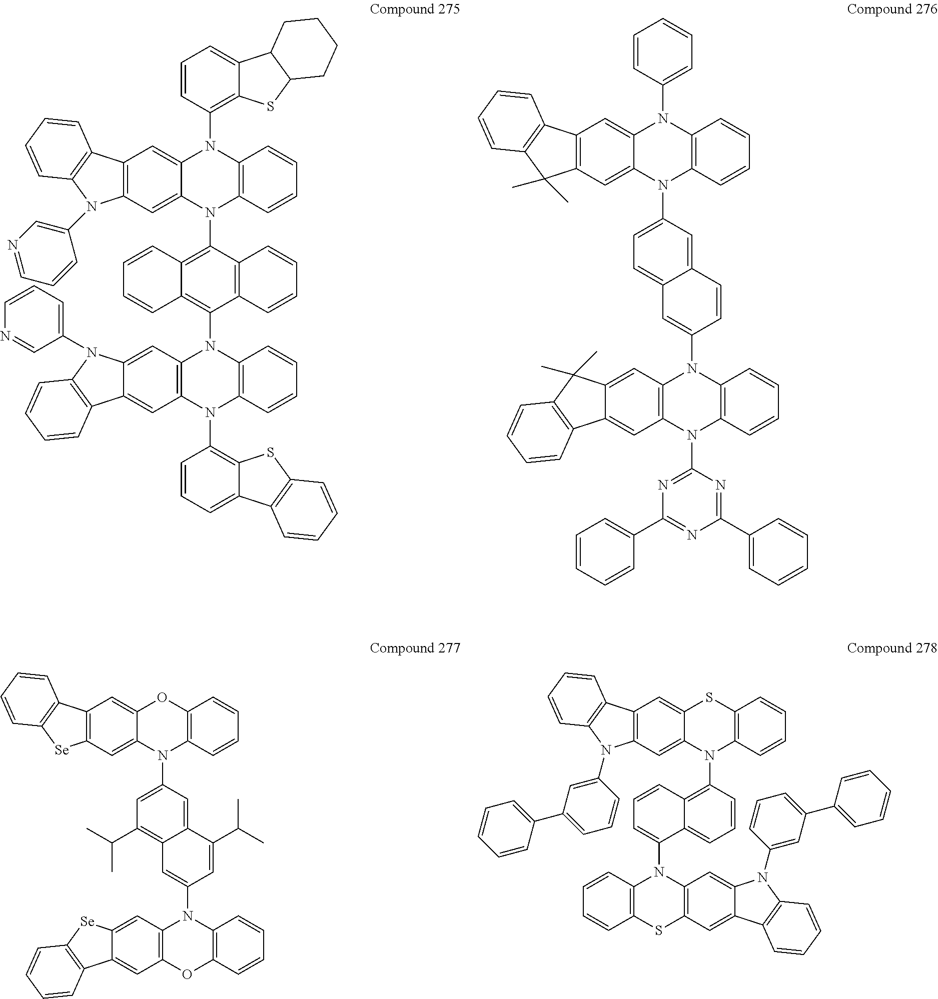

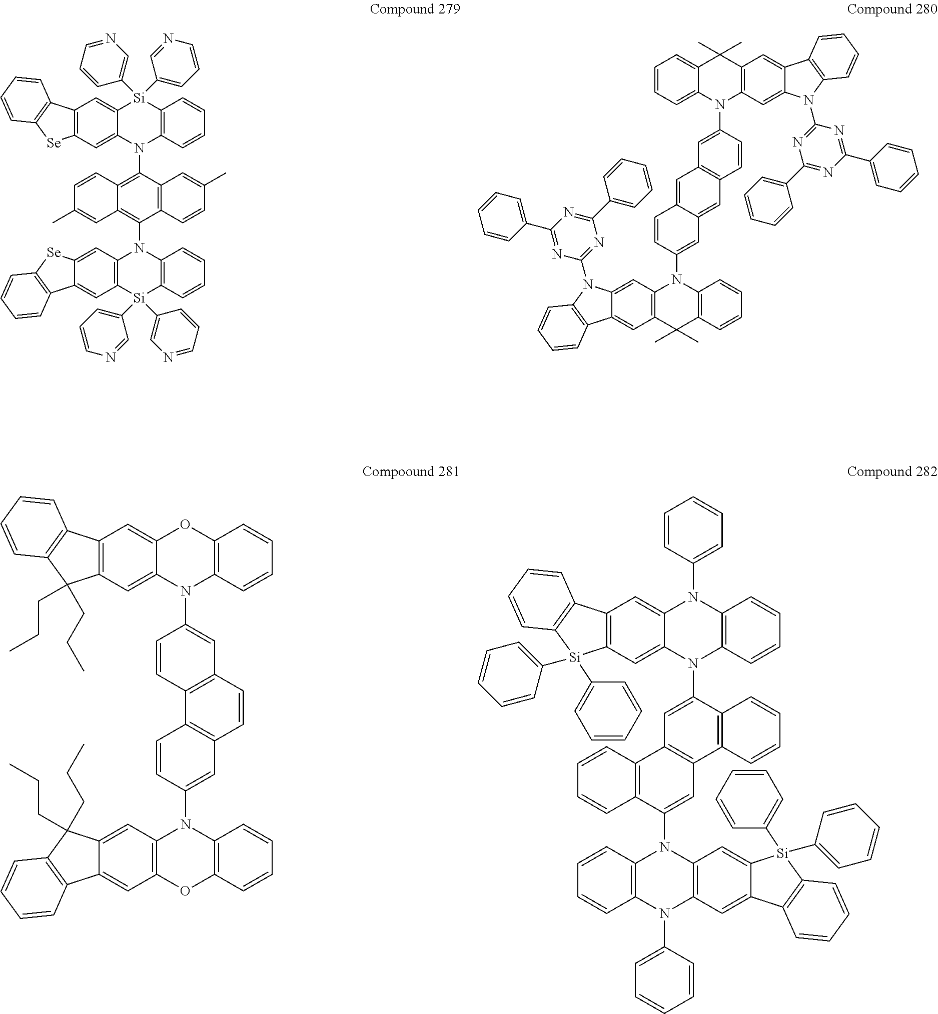

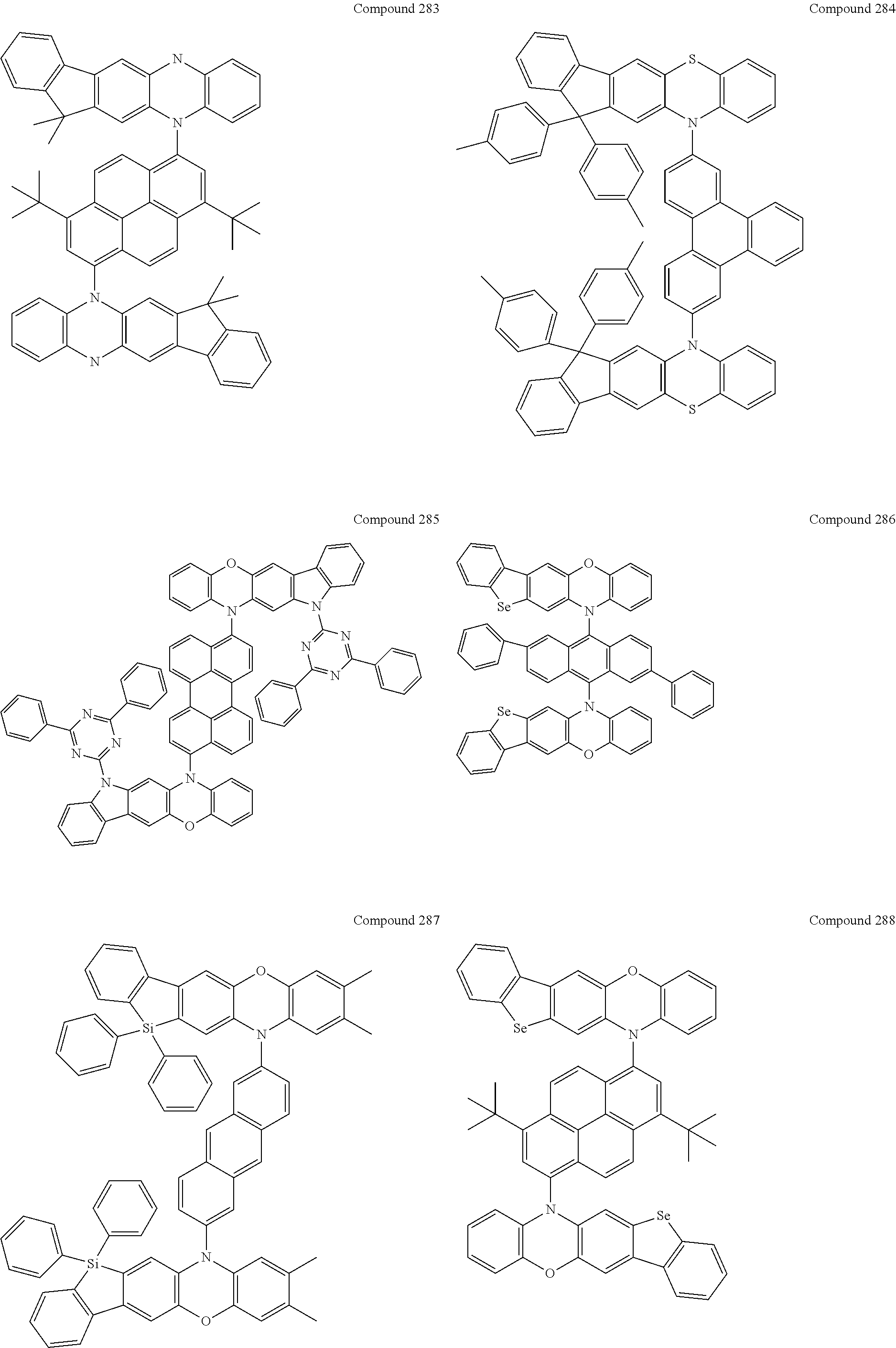

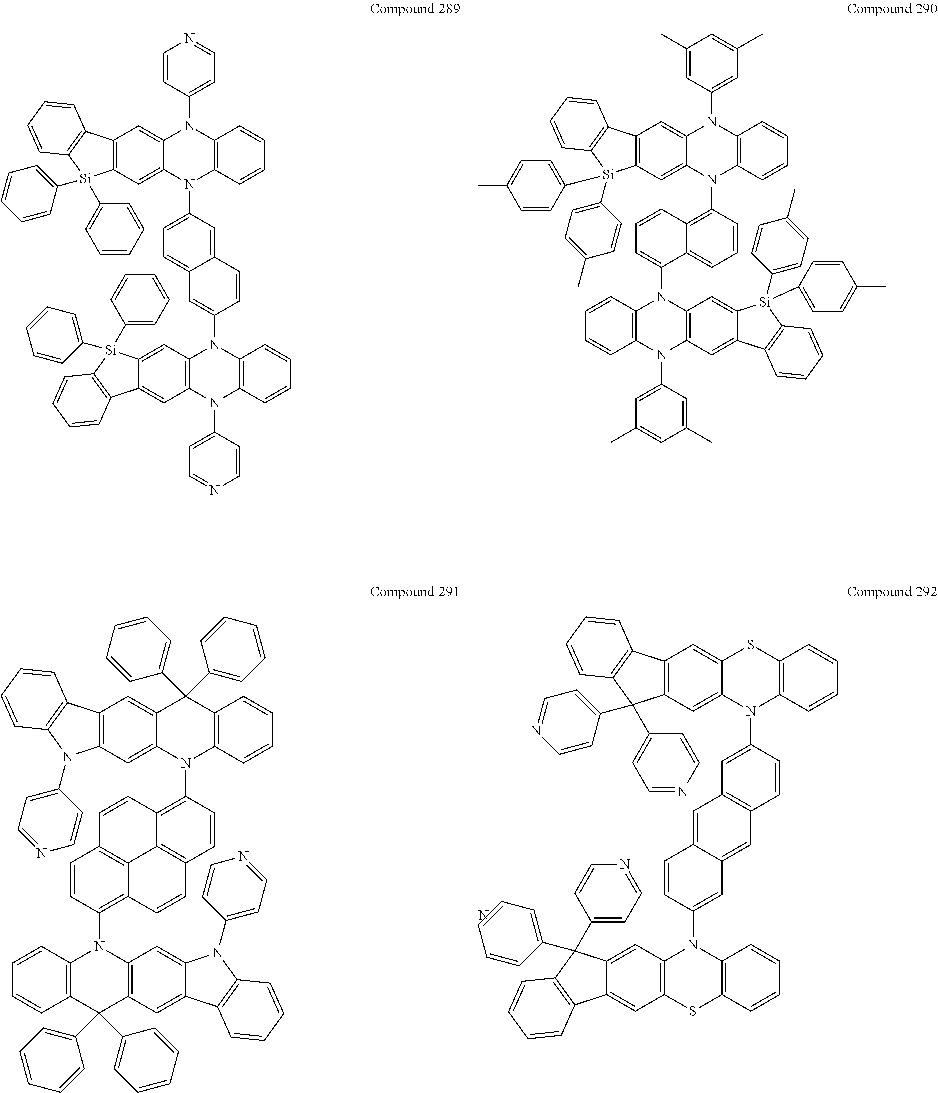

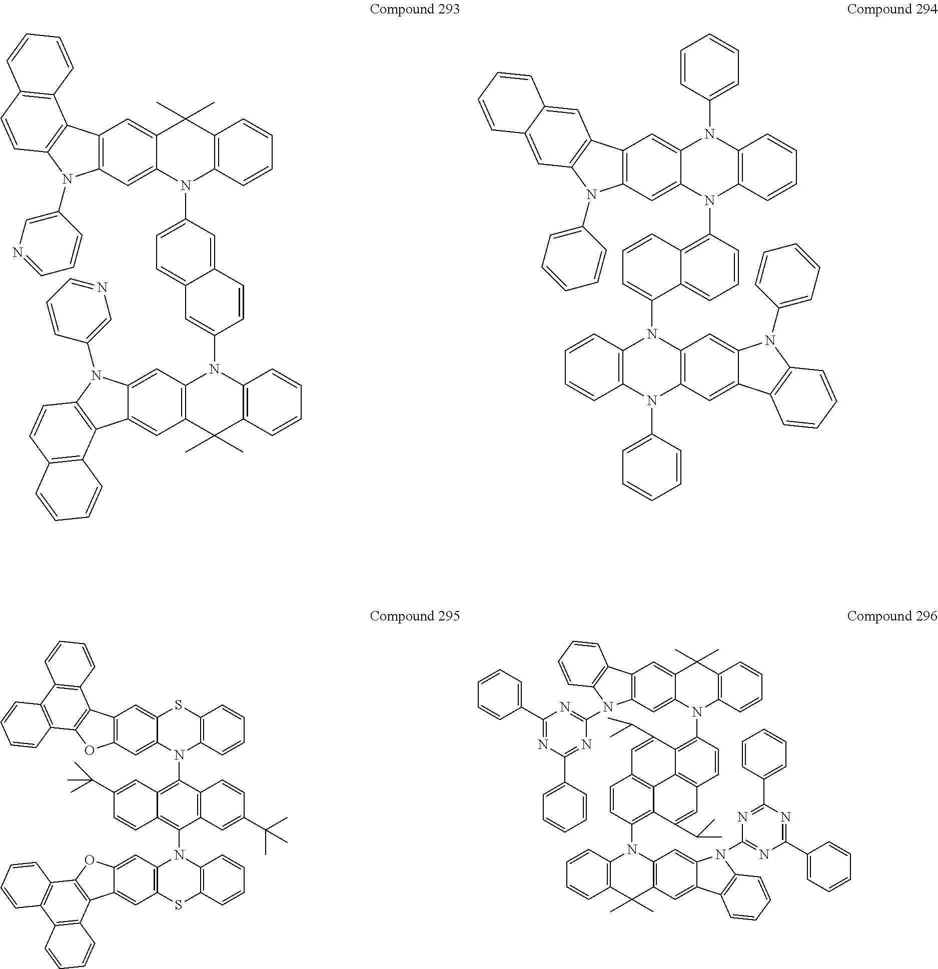

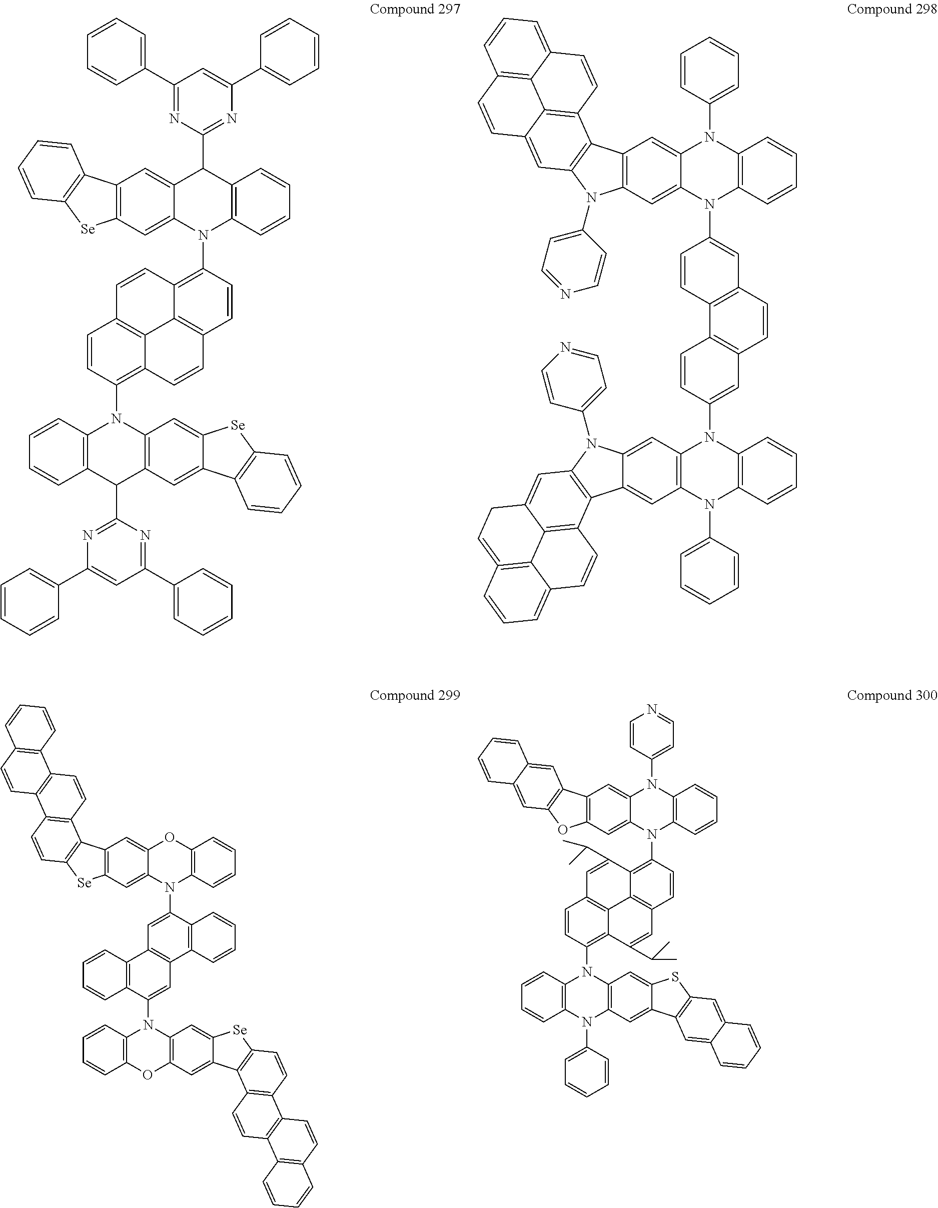

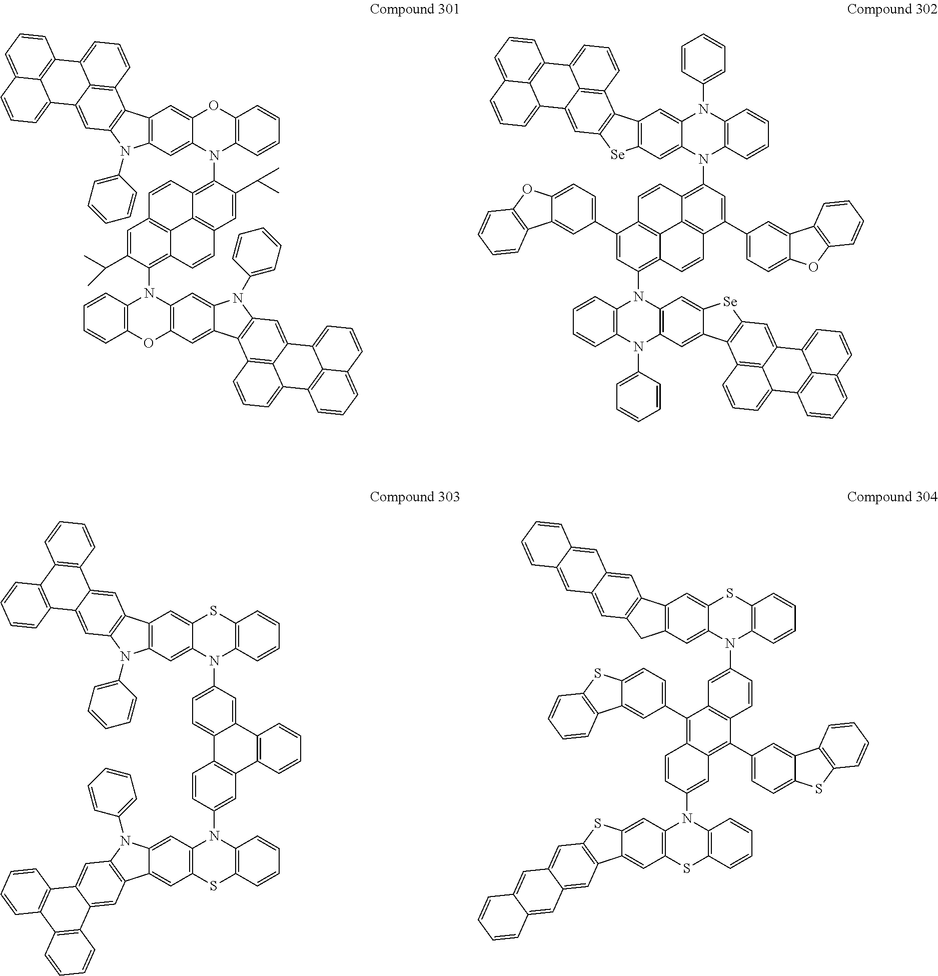

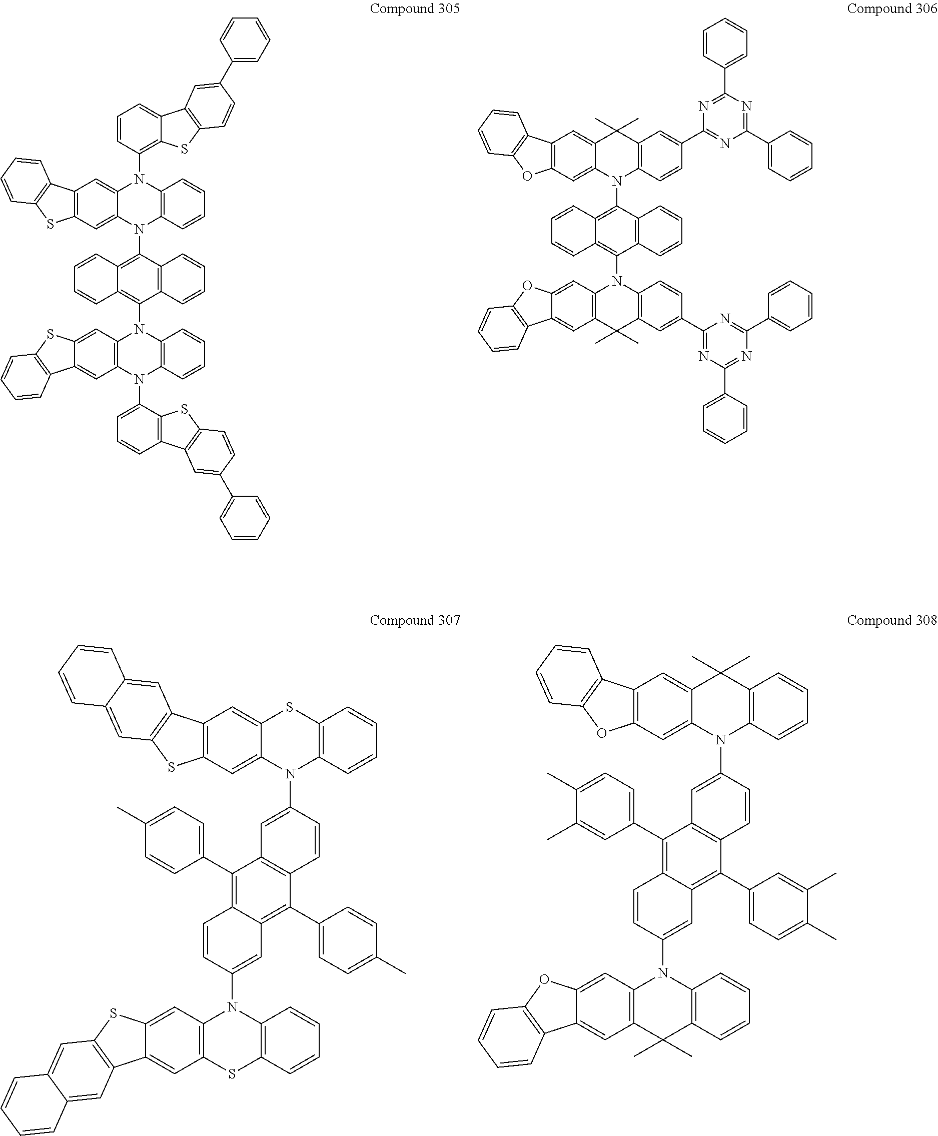

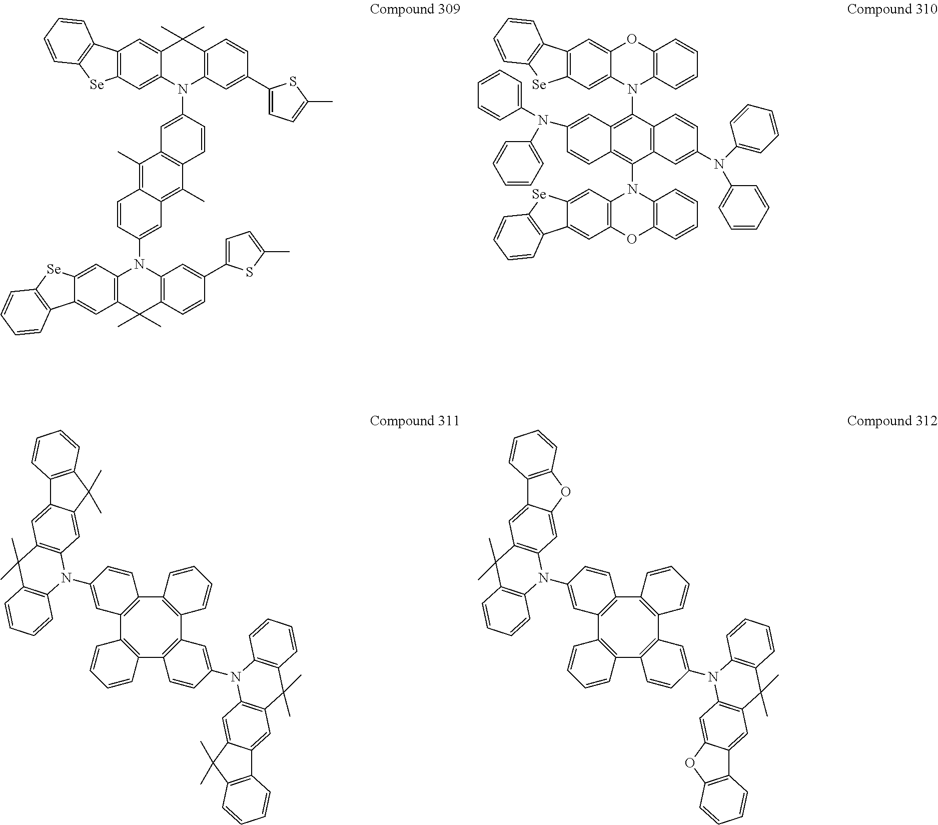

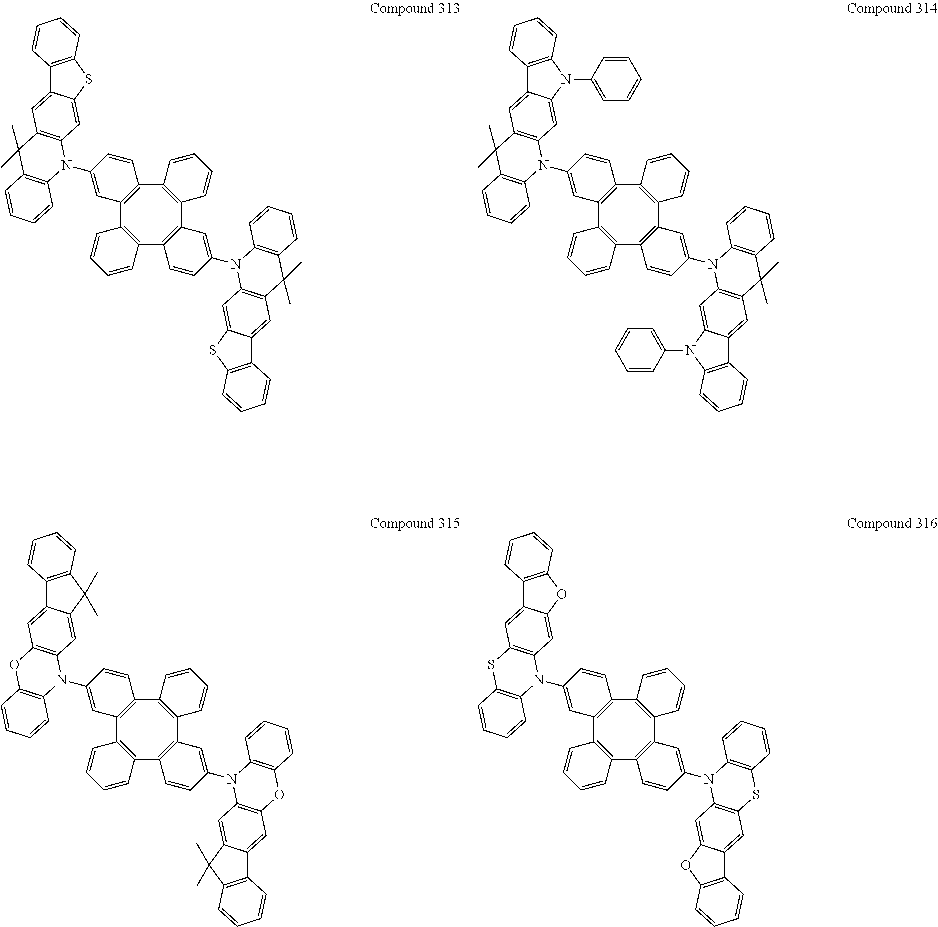

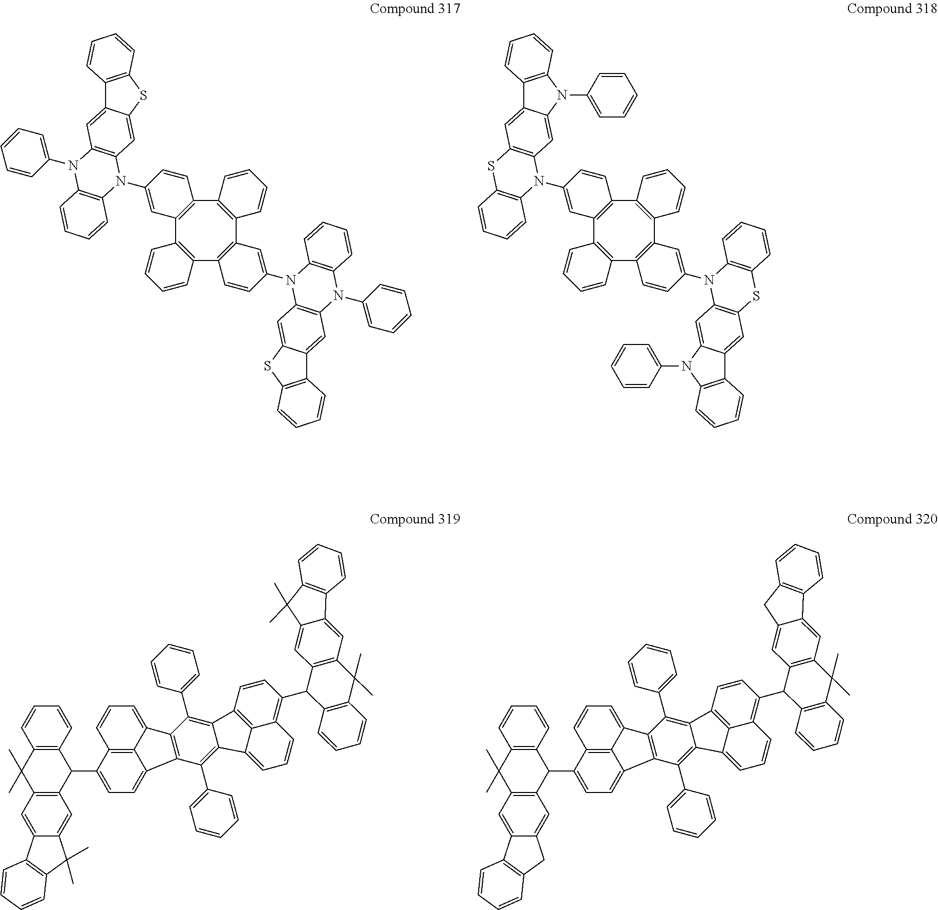

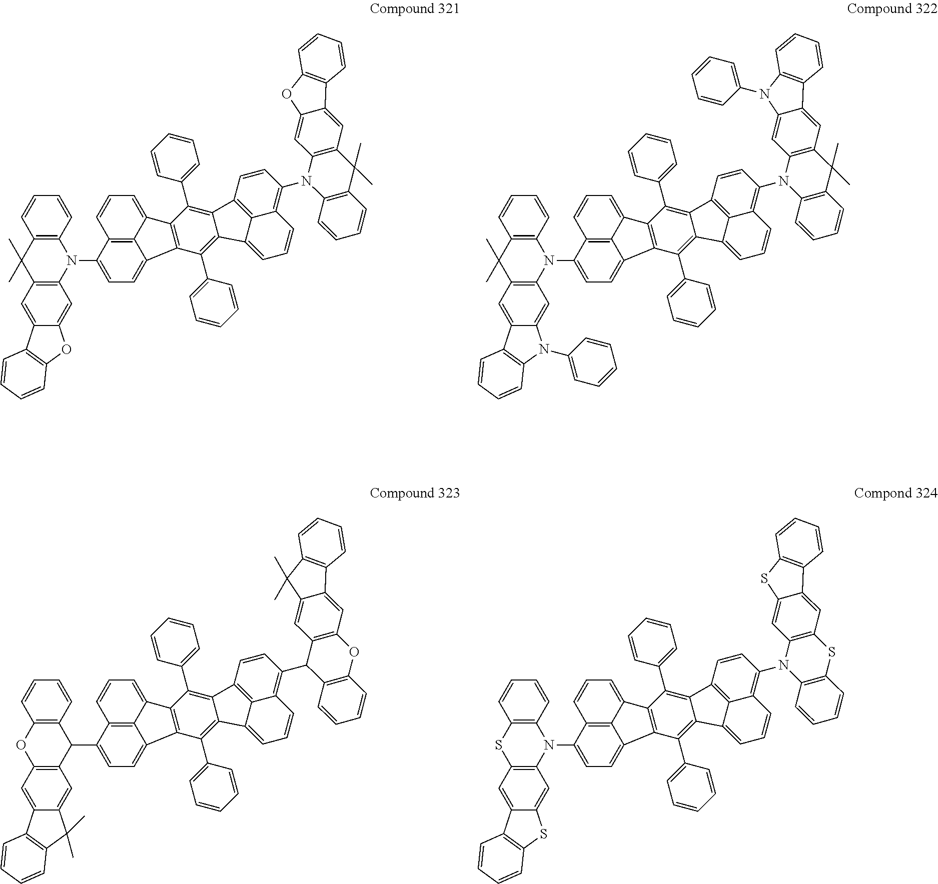

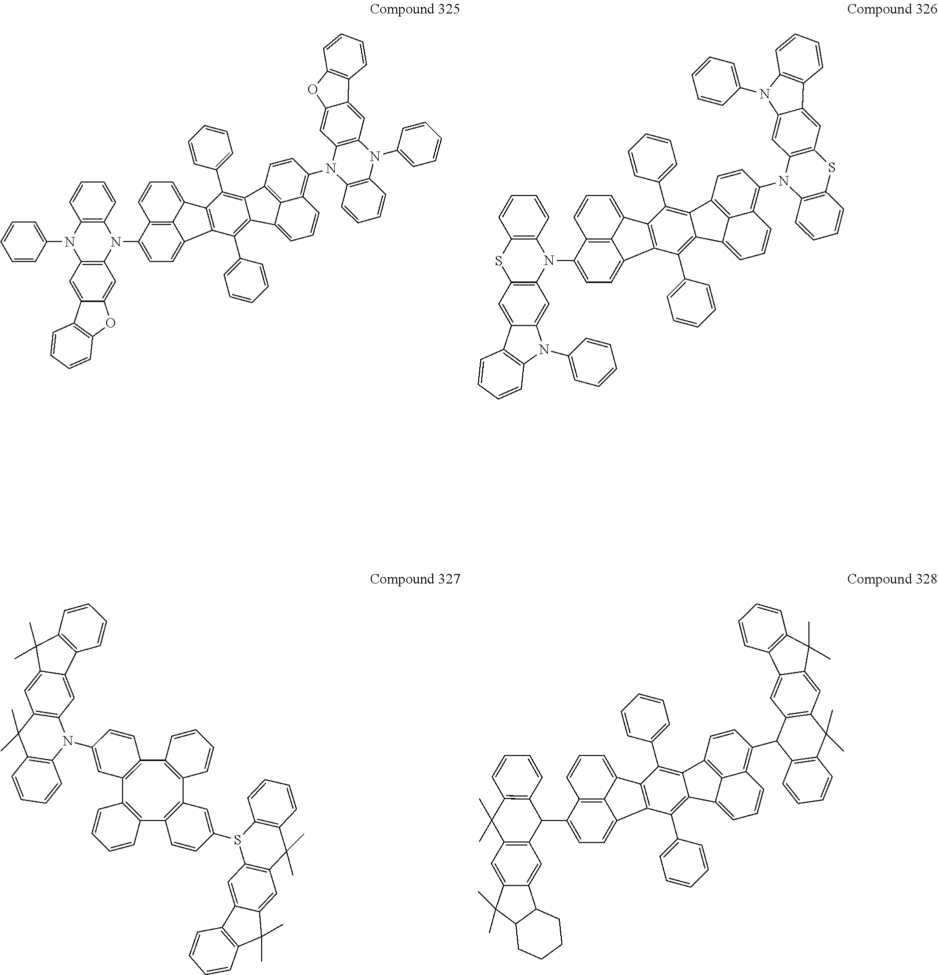

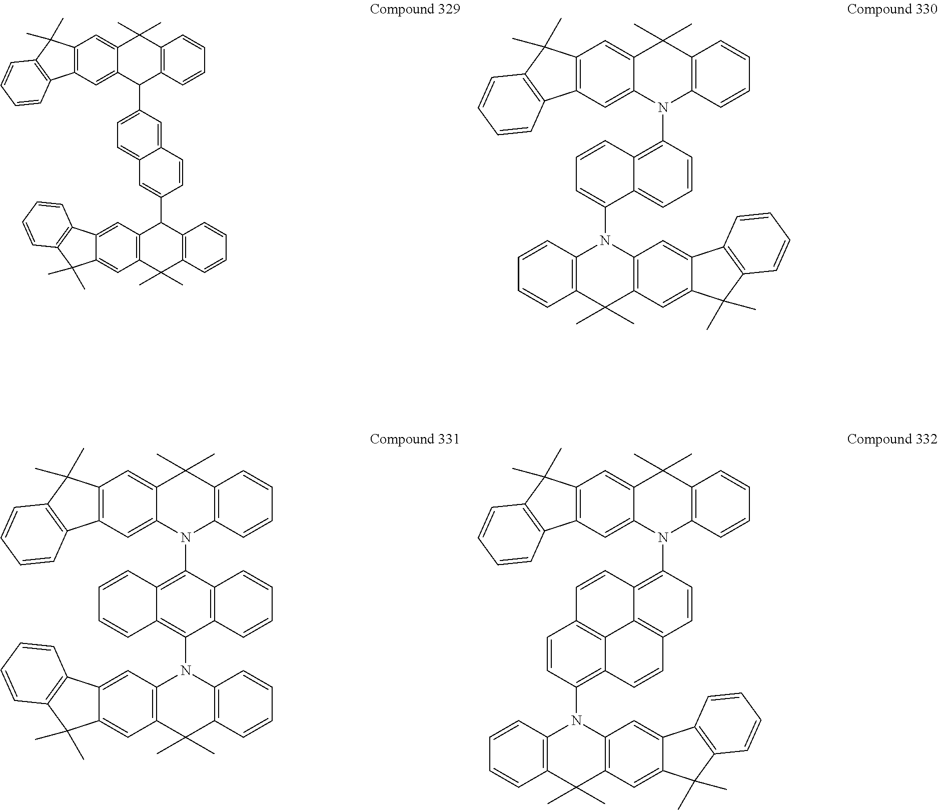

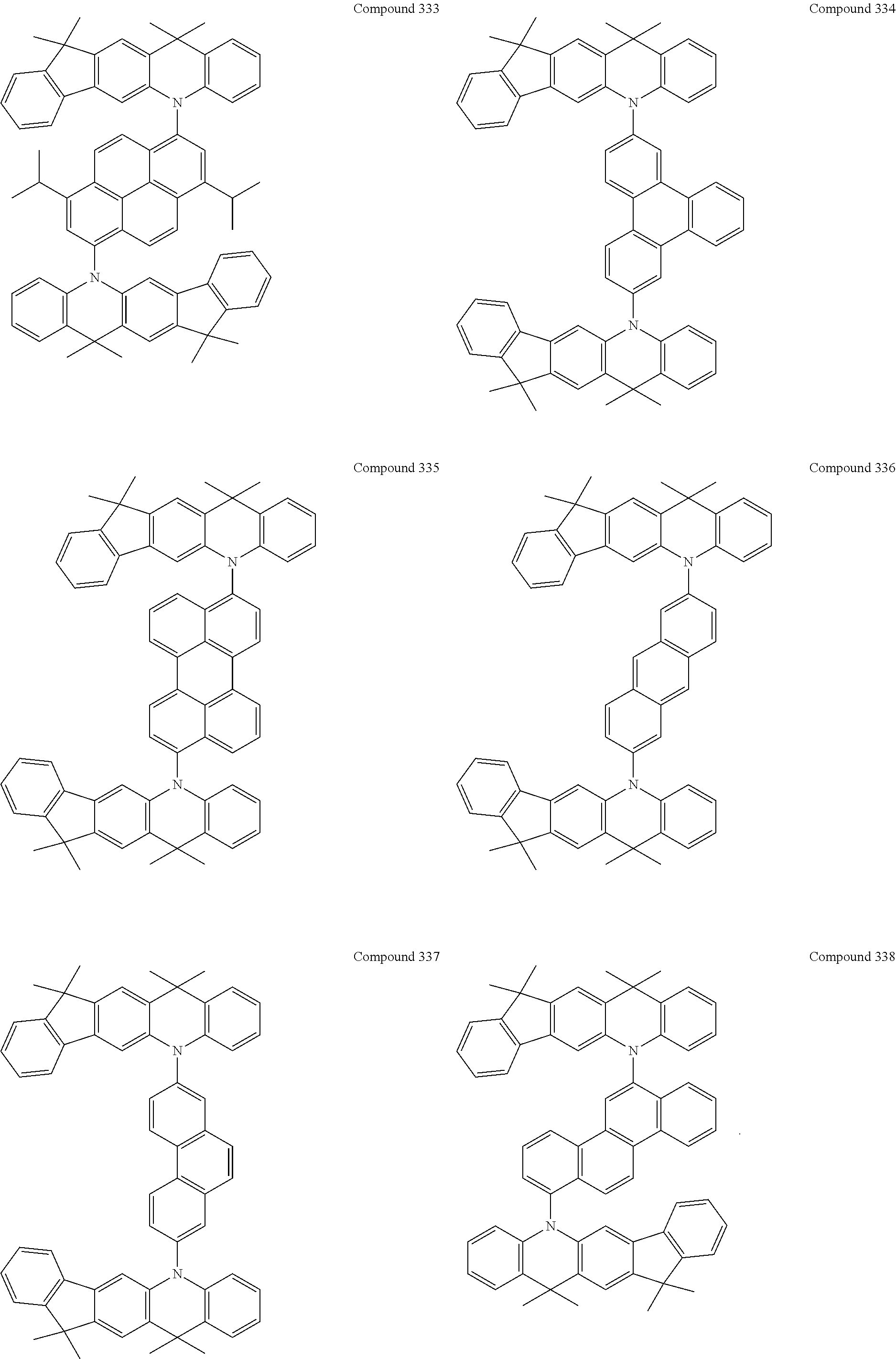

6. An organic electroluminescence device, comprising a pair of electrodes composed of a cathode and an anode, and a light emitting layer between the pair of electrodes, wherein the light emitting layer comprises the heteroaromatic compound according to claim 1.

7. The organic electroluminescence device according to claim 6, wherein the light emitting layer comprising the heteroaromatic compound of formula (1) is a fluorescent guest material.

8. The organic electroluminescence device according to claim 6, wherein the light emitting layer emits blue, green, yellow, red, or orange fluorescence.

9. The organic electroluminescence device according to claim 6, wherein the organic electroluminescence device is a lighting panel.

10. The organic electroluminescence device according to claim 6, wherein the organic electroluminescence device is a backlight panel.
